(19) United States
(12) Patent Application Publication
(10) Pub. No.: US 2021/0253724 A1
(43) Pub. Date: Aug. 19, 2021

(54) NOVEL BISPECIFIC AGONISTIC 4-1BB ANTIGEN BINDING MOLECULES

(71) Applicant: Hoffmann-La Roche Inc., Little Falls, NJ (US)

(72) Inventors: Christina CLAUS, Schlieren (CH); Claudia FERRARA KOLLER, Schlieren (CH); Christian KLEIN, Schlieren (CH); Ekkehard MOESSNER, Schlieren (CH); Pablo UMAÑA, Schlieren (CH)

(73) Assignee: Hoffmann-La Roche Inc., Little Falls, NJ (US)

(21) Appl. No.: 17/125,533

(22) Filed: Dec. 17, 2020

Related U.S. Application Data
(63) Continuation of application No. PCT/EP2019/067659, filed on Jul. 2, 2019.

(30) Foreign Application Priority Data
Jul. 4, 2018 (EP) 18181652.1

(51) Int. Cl. C07K 16/28; C07K 16/40; C07K 16/30; A61K 39/395; A61K 45/06

(52) U.S. Cl. CPC C07K 16/2878; C07K 2317/75; C07K 16/3007; C07K 16/2803; C07K 16/2827; A61K 39/39541; A61K 45/06; C07K 2317/31; C07K 2317/55; C07K 2317/52; C07K 2317/35; C07K 2317/71; C07K 2317/565; C07K 2317/51; C07K 2317/515; C07K 2317/24; C07K 2317/92; C07K 16/40

(57) ABSTRACT

The invention relates to new bispecific antigen binding molecules, comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein the Fab fragments (a) and (b) are fused to each other, and to methods of producing these molecules and to methods of using the same.

### CROSS-REFERENCE TO RELATED APPLICATIONS

This application is a Continuation of International Application No. PCT/EP2019/067659, filed Jul. 2, 2019, which claims benefit of and priority to European Application No. 18181652.1 filed Jul. 4, 2018, each of which is incorporated herein by reference in its entirety.

### SEQUENCE LISTING

This application contains a Sequence Listing which has been submitted electronically in ASCII format and is hereby incorporated by reference in its entirety. Said ASCII copy, created on Dec. 16, 2020, is named P34875USSEQLIST and is 323,380 bytes in size.

### FIELD OF THE INVENTION

The invention relates to new bispecific antigen binding molecules, comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein the Fab fragments (a) and (b) are fused to each other. The invention further relates to methods of producing these molecules and to methods of using the same.

### BACKGROUND

4-1BB (CD137), a member of the TNF receptor superfamily, has been first identified as a molecule whose expression is induced by T-cell activation (Kwon Y. H. and Weissman S. M. (1989), Proc. Natl. Acad. Sci. USA 86, 1963-1967). Subsequent studies demonstrated expression of 4-1BB in T- and B-lymphocytes (Snell L. M. et al. (2011) Immunol. Rev. 244, 197-217 or Zhang X. et al. (2010), J. Immunol. 184, 787-795), NK-cells (Lin W. et al. (2008), Blood 112, 699-707, NKT-cells (Kim D. H. et al. (2008), J. Immunol. 180, 2062-2068), monocytes (Kienzle G. and von Kempis J. (2000), Int. Immunol. 12, 73-82, or Schwarz H. et al. (1995), Blood 85, 1043-1052), neutrophils (Heinisch I. V. et al. (2000), Eur. J. Immunol. 30, 3441-3446), mast (Nishimoto H. et al. (2005), Blood 106, 4241-4248), and dendritic cells as well as cells of non-hematopoietic origin such as endothelial and smooth muscle cells (Broll K. et al. (2001), Am. J. Clin. Pathol. 115, 543-549 or Olofsson P. S. et al. (2008), Circulation 117, 1292-1301). Expression of 4-1BB in different cell types is mostly inducible and driven by various stimulatory signals, such as T-cell receptor (TCR) or B-cell receptor triggering, as well as signaling induced through co-stimulatory molecules or receptors of pro-inflammatory cytokines (Diehl L. et al. (2002), J. Immunol. 168, 3755-3762; von Kempis J. et al. (1997), Osteoarthritis Cartilage 5, 394-406; Zhang X. et al. (2010), J. Immunol. 184, 787-795).

CD137 signaling is known to stimulate IFNγ secretion and proliferation of NK cells (Buechele C. et al. (2012), Eur. J. Immunol. 42, 737-748; Lin W. et al. (2008), Blood 112, 699-707; Melero I. et al. (1998), Cell Immunol. 190, 167-172) as well as to promote DC activation as indicated by their increased survival and capacity to secret cytokines and upregulate co-stimulatory molecules (Choi B. K. et al. (2009), J. Immunol. 182, 4107-4115; Futagawa T. et al. (2002), Int. Immunol. 14, 275-286; Wilcox R. A. et al. (2002), J. Immunol. 168, 4262-4267). However, CD137 is best characterized as a co-stimulatory molecule which modulates TCR-induced activation in both the CD4⁺ and CD8⁺ subsets of T-cells. In combination with TCR triggering, agonistic 4-1BB-specific antibodies enhance proliferation of T-cells, stimulate lymphokine secretion and decrease sensitivity of T-lymphocytes to activation-induced cells death (Snell L. M. et al. (2011) Immunol. Rev. 244, 197-217). In line with these co-stimulatory effects of 4-1BB antibodies on T-cells in vitro, their administration to tumor bearing mice leads to potent anti-tumor effects in many experimental tumor models (Melero I. et al. (1997), Nat. Med. 3, 682-685; Narazaki H. et al. (2010), Blood 115, 1941-1948). In vivo depletion experiments demonstrated that CD8⁺ T-cells play the most critical role in anti-tumoral effect of 4-1BB-specific antibodies. However, depending on the tumor model or combination therapy, which includes anti-4-1BB, contributions of other types of cells such as DCs, NK-cells or CD4⁺ T-cells have been reported (Murillo O. et al. (2009), Eur. J. Immunol. 39, 2424-2436; Stagg J. et al. (2011), Proc. Natl. Acad. Sci. USA 108, 7142-7147).

It appears that the immunomodulatory properties of 4-1BB agonistic antibodies in vivo require the presence of the wild type Fc-portion on the antibody molecule thereby implicating Fc-receptor binding as an important event required for the pharmacological activity of such reagents as has been described for agonistic antibodies specific to other apoptosis-inducing or immunomodulatory members of the TNFR-superfamily (Li F. and Ravetch J. V. (2011), Science 333, 1030-1034; Teng M. W. et al. (2009), J. Immunol. 183, 1911-1920). However, systemic administration of 4-1BB-specific agonistic antibodies with the functionally active Fc domain also induces expansion of CD8⁺ T-cells associated with liver toxicity (Dubrot J. et al. (2010), Cancer Immunol. Immunother. 59, 1223-1233) that is diminished or significantly ameliorated in the absence of functional Fc-receptors in mice.

Urelumab (BMS-666513, clone 10C7) is a fully human, agonistic non-ligand blocking monoclonal IgG4 antibody that binds to the 4-1BB extracellular domain. It is disclosed as 20H4.9-IgG4 in U.S. Pat. No. 7,288,638. In human clinical trials (ClinicalTrials.gov, NCT00309023 and NCT00612664), urelumab administered once every three weeks for 12 weeks induced stabilization of the disease in patients with melanoma, ovarian or renal cell carcinoma. However, the trials were terminated as the antibody caused grade 4 hepatitis leading to the occurrence of two hepatotoxicity-related deaths (Simeone E. and Ascierto P. A. (2012), J. Immunotoxicology 9, 241-247). Subsequent detailed analysis of the clinical safety data demonstrated that the development of severe transaminitis is mainly triggered by the dose of urelumab given. Grade 2+ neutropenia, leukopenia and thropmbocytopenia were also observed. Subsequent detailed analysis of the clinical safety data demonstrated that the development of severe transaminitis is mainly triggered by the dose of urelumab given. In 2012, urelumab re-entered clinical development, however under the condition that doses <1 mg/kg given every three weeks were used. The current recommended dose is 0.1 mg/kg given every three weeks (N. Segal et al. (2016), Results From an Integrated Safety Analysis of Urelumab, an Agonist Anti-CD137 Monoclonal Antibody, Clin. Cancer Res., published online Dec. 1, 2016). In view of the dose-limiting toxicity there is thus a need for improved antigen binding molecules specific for 4-1BB that should act only at the tumor-specific sites in order to avoid uncontrollable side effects. The bispecific antigen binding molecules of the invention combine a Fab fragment capable of preferred binding to tumor-specific or tumor-associated antigen (target cell antigen) with two Fab fragments capable of agonistic binding to 4-1BB. The bispecific antigen binding molecules of this invention in their specific format may be able to trigger 4-1BB not only effectively, but also very selectively at the desired site thereby reducing undesirable side effects that have been observed with conventional monospecific antibodies such as urelumab.

### SUMMARY OF THE INVENTION

The present invention relates to new bispecific antigen binding molecules, comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein the Fab fragments (a) and (b) are fused to each other, and to methods of producing these molecules and to methods of using the same. These bispecific antigen binding molecules are advantageous as they will preferably activate 4-1BB at the site where the target cell antigen is expressed, due to their binding capability towards a target cell antigen and reduce activation at other sites in the body, thereby avoiding side effects of an antibody specific for 4-1BB alone. They are further characterized by their specific structural features such as proximity of two Fab fragments capable of specific binding to 4-1BB and the target cell antigen, respectively, bivalent binding to 4-1BB and monovalent binding to the target cell antigen which makes these bispecific antigen binding molecules very potent.

In one aspect, the invention provides a bispecific antigen binding molecule, comprising
(a) a first Fab fragment capable of specific binding to 4-1BB,
(b) a second Fab fragment capable of specific binding to a target cell antigen,
(c) a third Fab fragment capable of specific binding to 4-1BB, and
(d) a Fc domain composed of a first and a second subunit capable of stable association, wherein the second Fab fragment (b) is fused at the C-terminus of the Fab heavy chain to the N-terminus of the Fab heavy chain of the first Fab fragment (a), which is in turn fused at its C-terminus to the N-terminus of the first Fc domain subunit, and the third Fab fragment (c) is fused at the C-terminus of the Fab heavy chain to the N-terminus of the second Fc domain subunit, and wherein in the second Fab fragment capable of specific binding to a target cell antigen (i) the variable regions VL and VH of the Fab light chain and Fab heavy chain are replaced by each other, or (ii) the constant regions CL and CH1 of the Fab light chain and Fab heavy chain are replaced by each other.

In a particular aspect, provided is a bispecific antigen binding molecule as described herein, wherein the bispecific antigen binding molecule provides bivalent binding to 4-1BB and monovalent binding to the target cell antigen.

In a further aspect, provided is a bispecific antigen binding molecule as described herein before, wherein the Fc domain composed of a first and a second subunit capable of stable association is an an IgG Fc domain, particularly an IgG1 Fc domain or an IgG4 Fc domain. In one aspect, in the CH3 domain of the first subunit of the Fc domain an amino acid residue is replaced with an amino acid residue having a larger side chain volume , thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. Thus, provided is a bispecific antigen binding molecule, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (numbering according to Kabat EU index) and the second subunit of the Fc domain comprises the amino acid substitutions Y349C, T366S and Y407V (numbering according to Kabat EU index).

In another aspect, provided is a bispecific antigen binding molecule as described herein before, wherein the Fc domain comprises one or more amino acid substitution that reduces binding to an Fc receptor and/or effector function. In one aspect, said one or more amino acid substitution is at one or more position selected from the group of L234, L235, and P329 (Kabat EU index numbering). In one aspect, the Fc domain comprises one or more amino acid substitution that reduces the binding affinity of the antigen binding molecule to an Fc receptor and/or effector function, particularly the amino acid mutations L234A, L235A and P329G (numbering according to Kabat EU index). In particular, the Fc receptor is an Fcγ receptor and/or the effector function is antibody-dependent cell-mediated cytotoxicity (ADCC).

In one aspect, provided is a bispecific antigen binding molecule as described herein before, wherein the first and the third Fab fragment capable of specific binding to 4-1BB (a) and (c) are identical. In a particular aspect, the invention provides a a bispecific antigen binding molecule as described herein before, wherein the first and the third Fab fragment capable of specific binding to 4-1BB each comprise a heavy chain variable region (V_{H}4-1BB) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a light chain variable region (V_{L}4-1BB) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6.

In a particular aspect, the bispecific antigen binding molecule comprises a first and a third Fab fragment capable of specific binding to 4-1BB each comprising a heavy chain variable region (V_{H}4-1BB) comprising an amino acid sequence that is at least about 95%, 96%, 97%, 98%, 99% or 100% identical to the amino acid sequence of SEQ ID NO:7 and a light chain variable region (V_{L}4-1BB) comprising an amino acid sequence that is at least about 95%, 96%, 97%, 98%, 99% or 100% identical to the amino acid sequence of SEQ ID NO:8. More particularly, the first and the third Fab fragment capable of specific binding to 4-1BB each comprise a heavy chain variable region (V_{H}4-1BB) comprising the amino acid sequence of SEQ ID NO:7 and a light chain variable region (V_{L}4-1BB) comprising the amino acid sequence of SEQ ID NO:8.

In one aspect, provided is a bispecific antigen binding molecule, comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein the second Fab fragment (b) is fused at the C-terminus of the Fab heavy chain to the N-terminus of the Fab heavy chain of the first Fab fragment (a), which is in turn fused at its C-terminus to the N-terminus of the first Fc domain subunit, and the third Fab fragment (c) is fused at the C-terminus of the Fab heavy chain to the N-terminus of the second Fc domain subunit, wherein in the third Fab fragment capable of specific binding to a target cell antigen (i) the variable domains VL and VH are replaced by each other, or (ii) the constant domains CL and CH1 are replaced by each other, and wherein in the constant domain CL of the first and the third Fab fragment capable of specific binding to 4-1BB the amino acid at position 124 is substituted by lysine (K) ((numbering according to Kabat EU index) and the amino acid at position 123 is substituted by arginine (R) or lysine (K) (numbering according to Kabat EU index), and wherein in the constant domain CH1 of the first and the third Fab fragment capable of specific binding to 4-1BB the amino acid at position 147 is substituted by glutamic acid (E) (numbering according to Kabat EU index) and the amino acid at position 213 is substituted by glutamic acid (E) (numbering according to Kabat EU index).

In a particular aspect, provided is a bispecific antigen binding molecule as described herein before, wherein in the second Fab fragment capable of specific binding to a target cell antigen variable regions VL and VH of the Fab light chain and the Fab heavy chain are replaced by each other.

In a further aspect, provided is a bispecific antigen binding molecule as described herein before, wherein the second Fab fragment capable of specific binding to a target cell antigen is selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20, CD33 and PD-L1. In one aspect, the target cell antigen is selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20 and CD33.In a particular aspect, the target cell antigen is selected from Fibroblast Activation Protein (FAP), Carcinoembryonic Antigen (CEA) and CD19. More particularly, the target cell antigen is selected from FAP and CEA. In another aspect, the target cell antigen is PD-L1.

In one aspect, the second Fab fragment capable of specific binding to a target cell antigen is a Fab fragment capable of specific binding to Fibroblast Activation Protein (FAP). Thus, provided is a bispecific antigen binding molecule as described herein before, wherein the second Fab fragment is capable of specific binding to FAP and comprises
(a) a heavy chain variable region (V_{H}FAP) comprising (i) CDR-H1 comprising the amino acid sequence of SEQ ID NO:9, (ii) CDR-H2 comprising the amino acid sequence of SEQ ID NO:10, and (iii) CDR-H3 comprising the amino acid sequence of SEQ ID NO:11, and a light chain variable region (V_{L}FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14, or
(b) a heavy chain variable region (V_{H}FAP) comprising (i) CDR-H1 comprising the amino acid sequence of SEQ ID NO:15, (ii) CDR-H2 comprising the amino acid sequence of SEQ ID NO:16, and (iii) CDR-H3 comprising the amino acid sequence of SEQ ID NO:17, and a a light chain variable region (V_{L}FAP) comprising (iv) CDR-L1 comprising the amino acid sequence of SEQ ID NO:18, (v) CDR-L2 comprising the amino acid sequence of SEQ ID NO:19, and (vi) CDR-L3 comprising the amino acid sequence of SEQ ID NO:20.

In a further aspect, the Fab fragment capable of specific binding to FAP comprises
(a) a heavy chain variable region (V_{H}FAP) comprising an amino acid sequence that is at least about 95%, 96%, 97%, 98%, 99% or 100% identical to the amino acid sequence of SEQ ID NO:21, and a light chain variable region (V_{L}FAP) comprising an amino acid sequence that is at least about 95%, 96%, 97%, 98%, 99% or 100% identical to the amino acid sequence of SEQ ID NO:22, or
(b) a heavy chain variable region (V_{H}FAP) comprising an amino acid sequence that is at least about 95%, 96%, 97%, 98%, 99% or 100% identical to the amino acid sequence of SEQ ID NO:23, and a light chain variable region (V_{L}FAP) comprising an amino acid sequence that is at least about 95%, 96%, 97%, 98%, 99% or 100% identical to the amino acid sequence of SEQ ID NO:24.

In particular, the Fab fragment capable of specific binding to FAP comprises a heavy chain variable region (V_{H}FAP) comprising the amino acid sequence of SEQ ID NO:21, and a light chain variable region (V_{L}FAP) comprising the amino acid sequence of SEQ ID NO:22, or a heavy chain variable region (V_{H}FAP) comprising the amino acid sequence of SEQ ID NO:23, and a light chain variable region (V_{L}FAP) comprising the amino acid sequence of SEQ ID NO:24. More particularly, the Fab fragment capable of specific binding to FAP comprises a heavy chain variable region (V_{H}FAP) comprising the amino acid sequence of SEQ ID NO:21 and a light chain variable region (V_{L}FAP) comprising the amino acid sequence of SEQ ID NO:22.

Furthermore, provided is a bispecific antigen binding molecule as described herein before, wherein
(i) the first and third Fab fragment capable of specific binding to 4-1BB each comprise a heavy chain variable region VH comprising the amino acid sequence of SEQ ID NO: 7 and a light chain variable region VL comprising an amino acid sequence of SEQ ID NO: 8 and
(ii) the second Fab fragment capable of specific binding to FAP comprises a heavy chain variable region VH comprising an amino acid sequence of SEQ ID NO:21 and a light chain variable region VL comprising an amino acid sequence of SEQ ID NO:22 or a heavy chain variable region VH comprising an amino acid sequence of SEQ ID NO:23 and a light chain variable region VL comprising an amino acid sequence of SEQ ID NO:24.

In another aspect, the invention provides a bispecific antigen binding molecule, wherein the second Fab fragment capable of specific binding to a target cell antigen is a Fab fragment capable of specific binding to Carcinoembryonic Antigen (CEA).

Thus, provided is a bispecific antigen binding molecule, wherein the second Fab fragment capable of specific binding to Carcinoembryonic Antigen (CEA) comprises
(a) a heavy chain variable region (V_{H}CEA) comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a light chain variable region (V_{L}CEA) comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30, or
(b) a heavy chain variable region (V_{H}CEA) comprising (i) CDR-H1 comprising the amino acid sequence of SEQ ID NO:33, (ii) CDR-H2 comprising the amino acid sequence of SEQ ID NO:34, and (iii) CDR-H3 comprising the amino acid sequence of SEQ ID NO:35, and a light chain variable region (V_{L}CEA) comprising (iv) CDR-L1 comprising the amino acid sequence of SEQ ID NO:36, (v) CDR-L2 comprising the amino acid sequence of SEQ ID NO:37, and (vi) CDR-L3 comprising the amino acid sequence of SEQ ID NO:38, or
(c) a heavy chain variable region (V_{H}CEA) comprising (i) CDR-H1 comprising the amino acid sequence of SEQ ID NO:41, (ii) CDR-H2 comprising the amino acid sequence of SEQ ID NO:42, and (iii) CDR-H3 comprising the amino acid sequence of SEQ ID NO:43, and a light chain variable region (V_{L}CEA) comprising (iv) CDR-L1 comprising the amino acid sequence of SEQ ID NO:44, (v) CDR-L2 comprising the amino acid sequence of SEQ ID NO:45, and (vi) CDR-L3 comprising the amino acid sequence of SEQ ID NO:46, or
(d) a heavy chain variable region (V_{H}CEA) comprising (i) CDR-H1 comprising the amino acid sequence of SEQ ID NO:49, (ii) CDR-H2 comprising the amino acid sequence of SEQ ID NO:50, and (iii) CDR-H3 comprising the amino acid sequence of SEQ ID NO:51, and a light chain variable region (V_{L}CEA) comprising (iv) CDR-L1 comprising the amino acid sequence of SEQ ID NO:52, (v) CDR-L2 comprising the amino acid sequence of SEQ ID NO:53, and (vi) CDR-L3 comprising the amino acid sequence of SEQ ID NO:54, or
(e) a heavy chain variable region (V_{H}CEA) comprising (i) CDR-H1 comprising the amino acid sequence of SEQ ID NO:115, (ii) CDR-H2 comprising the amino acid sequence of SEQ ID NO:116, and (iii) CDR-H3 comprising the amino acid sequence of SEQ ID NO:117, and a light chain variable region (V_{L}CEA) comprising (iv) CDR-L1 comprising the amino acid sequence of SEQ ID NO:118, (v) CDR-L2 comprising the amino acid sequence of SEQ ID NO:119, and (vi) CDR-L3 comprising the amino acid sequence of SEQ ID NO:120, or
(f) a heavy chain variable region (V_{H}CEA) comprising (i) CDR-H1 comprising the amino acid sequence of SEQ ID NO:123, (ii) CDR-H2 comprising the amino acid sequence of SEQ ID NO:124 or SEQ ID NO:125, and (iii) CDR-H3 comprising the amino acid sequence of SEQ ID NO:126, and a light chain variable region (V_{L}CEA) comprising (iv) CDR-L1 comprising the amino acid sequence of SEQ ID NO:127 or SEQ ID NO:128, (v) CDR-L2 comprising the amino acid sequence of SEQ ID NO:129 or SEQ ID NO:130 or SEQ ID NO:131, and (vi) CDR-L3 comprising the amino acid sequence of SEQ ID NO:132.

In one aspect, provided is a bispecific antigen binding molecule as described herein before, wherein the second Fab fragment is capable of specific binding to CEA and comprises
(a) a heavy chain variable region (V_{H}CEA) comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a light chain variable region (V_{L}CEA) comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30, or
(b) a heavy chain variable region (V_{H}CEA) comprising (i) CDR-H1 comprising the amino acid sequence of SEQ ID NO:33, (ii) CDR-H2 comprising the amino acid sequence of SEQ ID NO:34, and (iii) CDR-H3 comprising the amino acid sequence of SEQ ID NO:35, and a light chain variable region (V_{L}CEA) comprising (iv) CDR-L1 comprising the amino acid sequence of SEQ ID NO:36, (v) CDR-L2 comprising the amino acid sequence of SEQ ID NO:37, and (vi) CDR-L3 comprising the amino acid sequence of SEQ ID NO:38, or
(c) a heavy chain variable region (V_{H}CEA) comprising (i) CDR-H1 comprising the amino acid sequence of SEQ ID NO:41, (ii) CDR-H2 comprising the amino acid sequence of SEQ ID NO:42, and (iii) CDR-H3 comprising the amino acid sequence of SEQ ID NO:43, and a light chain variable region (V_{L}CEA) comprising (iv) CDR-L1 comprising the amino acid sequence of SEQ ID NO:44, (v) CDR-L2 comprising the amino acid sequence of SEQ ID NO:45, and (vi) CDR-L3 comprising the amino acid sequence of SEQ ID NO:46, or
(d) a heavy chain variable region (V_{H}CEA) comprising (i) CDR-H1 comprising the amino acid sequence of SEQ ID NO:49, (ii) CDR-H2 comprising the amino acid sequence of SEQ ID NO:50, and (iii) CDR-H3 comprising the amino acid sequence of SEQ ID NO:51, and a light chain variable region (V_{L}CEA) comprising (iv) CDR-L1 comprising the amino acid sequence of SEQ ID NO:52, (v) CDR-L2 comprising the amino acid sequence of SEQ ID NO:53, and (vi) CDR-L3 comprising the amino acid sequence of SEQ ID NO:54.

In another aspect, the Fab fragment capable of specific binding to Carcinoembryonic Antigen (CEA) comprises
(a) a heavy chain variable region (V_{H}CEA) comprising an amino acid sequence that is at least about 95%, 96%, 97%, 98%, 99% or 100% identical to the amino acid sequence of SEQ ID NO:31, and a light chain variable region (V_{L}CEA) comprising an amino acid sequence that is at least about 95%, 96%, 97%, 98%, 99% or 100% identical to the amino acid sequence of SEQ ID NO:32, or

(b) a heavy chain variable region (V_{H}CEA) comprising an amino acid sequence that is at least about 95%, 96%, 97%, 98%, 99% or 100% identical to the amino acid sequence of SEQ ID NO:39, and a light chain variable region (V_{L}CEA) comprising an amino acid sequence that is at least about 95%, 96%, 97%, 98%, 99% or 100% identical to the amino acid sequence of SEQ ID NO:40, or

(c) a heavy chain variable region (V_{H}CEA) comprising an amino acid sequence that is at least about 95%, 96%, 97%, 98%, 99% or 100% identical to the amino acid sequence of SEQ ID NO:47, and a light chain variable region (V_{L}CEA) comprising an amino acid sequence that is at least about 95%, 96%, 97%, 98%, 99% or 100% identical to the amino acid sequence of SEQ ID NO:48, or

(d) a heavy chain variable region (V_{H}CEA) comprising an amino acid sequence that is at least about 95%, 96%, 97%, 98%, 99% or 100% identical to the amino acid sequence of SEQ ID NO:55, and a light chain variable region (V_{L}CEA) comprising an amino acid sequence that is at least about 95%, 96%, 97%, 98%, 99% or 100% identical to the amino acid sequence of SEQ ID NO:56,

(d) a heavy chain variable region (V_{H}CEA) comprising an amino acid sequence that is at least about 95%, 96%, 97%, 98%, 99% or 100% identical to the amino acid sequence of SEQ ID NO:55, and a light chain variable region (V_{L}CEA) comprising an amino acid sequence that is at least about 95%, 96%, 97%, 98%, 99% or 100% identical to the amino acid sequence of SEQ ID NO:56, or

(e) a heavy chain variable region (V_{H}CEA) comprising an amino acid sequence that is at least about 95%, 96%, 97%, 98%, 99% or 100% identical to the amino acid sequence of SEQ ID NO:121, and a light chain variable region (V_{L}CEA) comprising an amino acid sequence that is at least about 95%, 96%, 97%, 98%, 99% or 100% identical to the amino acid sequence of SEQ ID NO:122, or

(f) a heavy chain variable region (V_{H}CEA) comprising an amino acid sequence that is at least about 95%, 96%, 97%, 98%, 99% or 100% identical to the amino acid sequence of SEQ ID NO:133, and a light chain variable region (V_{L}CEA) comprising an amino acid sequence that is at least about 95%, 96%, 97%, 98%, 99% or 100% identical to the amino acid sequence of SEQ ID NO:143, or

(g) a heavy chain variable region (V_{H}CEA) comprising an amino acid sequence that is at least about 95%, 96%, 97%, 98%, 99% or 100% identical to the amino acid sequence of SEQ ID NO:137, and a light chain variable region (V_{L}CEA) comprising an amino acid sequence that is at least about 95%, 96%, 97%, 98%, 99% or 100% identical to the amino acid sequence of SEQ ID NO:143.

More particularly, the Fab fragment capable of specific binding to Carcinoembryonic Antigen (CEA) comprises a heavy chain variable region (V_{H}CEA) comprising the amino acid sequence of SEQ ID NO:31 and a light chain variable region (V_{L}CEA) comprising to the amino acid sequence of SEQ ID NO:32, or a heavy chain variable region (V_{H}CEA) comprising the amino acid sequence of SEQ ID NO:39 and a light chain variable region (V_{L}CEA) comprising the amino acid sequence of SEQ ID NO:40, or a heavy chain variable region (V_{H}CEA) comprising the amino acid sequence of SEQ ID NO:47 and a light chain variable region (V_{L}CEA) comprising the amino acid sequence of SEQ ID NO:48, or a heavy chain variable region (V_{H}CEA) comprising the amino acid sequence of SEQ ID NO:55 and a light chain variable region (V_{L}CEA) comprising the amino acid sequence of SEQ ID NO:56. Furthermore, provided is a bispecific antigen binding molecule as described herein before, wherein the Fab fragment capable of specific binding to Carcinoembryonic Antigen (CEA) comprises a heavy chain variable region (V_{H}CEA) comprising the amino acid sequence of SEQ ID NO:121 and a light chain variable region (V_{L}CEA) comprising to the amino acid sequence of SEQ ID NO:122.

In one aspect, provided is a bispecific antigen binding molecule as described herein before, wherein the second Fab fragment is capable of specific binding to CEA and comprises comprises a heavy chain variable region (V_{H}CEA) comprising the amino acid sequence of SEQ ID NO:133, SEQ ID NO:134, SEQ ID NO:135, SEQ ID NO:136, SEQ ID NO:137 or SEQ ID NO:138, and a light chain variable region (V_{L}CEA) comprising the amino acid sequence of SEQ ID NO:139, SEQ ID NO:140, SEQ ID NO:141, SEQ ID NO:142, SEQ ID NO:143 or SEQ ID NO:144.

In particular, Fab fragment capable of specific binding to CEA comprises a heavy chain variable region (V_{H}CEA) comprising the amino acid sequence of SEQ ID NO:133 and a light chain variable region (V_{L}CEA) comprising to the amino acid sequence of SEQ ID NO:143, or a heavy chain variable region (V_{H}CEA) comprising the amino acid sequence of SEQ ID NO:137 and a light chain variable region (V_{L}CEA) comprising the amino acid sequence of SEQ ID NO:143, or a heavy chain variable region (V_{H}CEA) comprising the amino acid sequence of SEQ ID NO:134 and a light chain variable region (V_{L}CEA) comprising the amino acid sequence of SEQ ID NO:143, or a heavy chain variable region (V_{H}CEA) comprising the amino acid sequence of SEQ ID NO:138 and a light chain variable region (V_{L}CEA) comprising the amino acid sequence of SEQ ID NO:142, or a heavy chain variable region (V_{H}CEA) comprising the amino acid sequence of SEQ ID NO:137 and a light chain variable region (V_{L}CEA) comprising the amino acid sequence of SEQ ID NO:142, or a heavy chain variable region (V_{H}CEA) comprising the amino acid sequence of SEQ ID NO:135 and a light chain variable region (V_{L}CEA) comprising the amino acid sequence of SEQ ID NO:142, or a heavy chain variable region (V_{H}CEA) comprising the amino acid sequence of SEQ ID NO:133 and a light chain variable region (V_{L}CEA) comprising the amino acid sequence of SEQ ID NO:142.

Furthermore, provided is a bispecific antigen binding molecule as described herein before, wherein
(i) the first and third Fab fragment capable of specific binding to 4-1BB each comprise a heavy chain variable region VH comprising the amino acid sequence of SEQ ID NO: 7 and a light chain variable region VL comprising an amino acid sequence of SEQ ID NO: 8 and
(ii) the second Fab fragment capable of specific binding to CEA comprises a heavy chain variable region VH comprising an amino acid sequence of SEQ ID NO:31 and a light chain variable region VL comprising an amino acid sequence of SEQ ID NO:32, or a heavy chain variable region VH comprising an amino acid sequence of SEQ ID NO:39 and a light chain variable region VL comprising an amino acid sequence of SEQ ID NO:40, or a heavy chain variable region VH comprising an amino acid sequence of SEQ ID NO:47 and a light chain variable region VL comprising an amino acid sequence of SEQ ID NO:48, or a heavy chain variable region VH comprising an amino acid sequence of SEQ ID NO:55 and a light chain variable region VL comprising an amino acid sequence of SEQ ID NO:56.

Also provided is a bispecific antigen binding molecule as described herein before, wherein
(i) the first and third Fab fragment capable of specific binding to 4-1BB each comprise a heavy chain variable region VH comprising the amino acid sequence of SEQ ID NO: 7 and a light chain variable region VL comprising an amino acid sequence of SEQ ID NO: 8 and (ii) the second Fab fragment capable of specific binding to CEA comprises a heavy chain variable region VH comprising an amino acid sequence of SEQ ID NO:121 and a light chain variable region VL comprising an amino acid sequence of SEQ ID NO:122.

In a further aspect, provided is a bispecific antigen binding molecule as described herein before, wherein
(i) the first and third Fab fragment capable of specific binding to 4-1BB each comprise a heavy chain variable region VH comprising the amino acid sequence of SEQ ID NO: 7 and a light chain variable region VL comprising an amino acid sequence of SEQ ID NO: 8 and
(ii) the second Fab fragment capable of specific binding to CEA comprises a heavy chain variable region VH comprising an amino acid sequence of SEQ ID NO:133 and a light chain variable region VL comprising an amino acid sequence of SEQ ID NO:143, or a heavy chain variable region VH comprising an amino acid sequence of SEQ ID NO:137 and a light chain variable region VL comprising an amino acid sequence of SEQ ID NO:143, or a heavy chain variable region VH comprising an amino acid sequence of SEQ ID NO:134 and a light chain variable region VL comprising an amino acid sequence of SEQ ID NO:143, or a heavy chain variable region VH comprising an amino acid sequence of SEQ ID NO:138 and a light chain variable region VL comprising an amino acid sequence of SEQ ID NO:142, or a heavy chain variable region VH comprising an amino acid sequence of SEQ ID NO:137 and a light chain variable region VL comprising an amino acid sequence of SEQ ID NO:142, or a heavy chain variable region VH comprising an amino acid sequence of SEQ ID NO:135 and a light chain variable region VL comprising an amino acid sequence of SEQ ID NO:142, or a heavy chain variable region VH comprising an amino acid sequence of SEQ ID NO:135 and a light chain variable region VL comprising an amino acid sequence of SEQ ID NO:142.

In a particular aspect, provided is a bispecific antigen binding molecule, wherein (i) the first and third Fab fragment capable of specific binding to 4-1BB each comprise a heavy chain variable region VH comprising the amino acid sequence of SEQ ID NO: 7 and a light chain variable region comprising an amino acid sequence of SEQ ID NO: 8 and (ii) the second Fab fragment capable of specific binding to CEA comprises a heavy chain variable region VH comprising an amino acid sequence of SEQ ID NO:31 and a light chain variable region comprising an amino acid sequence of SEQ ID NO:32. More particularly, the second Fab fragment capable of specific binding to CEA comprises a heavy chain variable region VH comprising an amino acid sequence of SEQ ID NO:121 and a light chain variable region VL comprising an amino acid sequence of SEQ ID NO:122.

In a further particular aspect, provided is a bispecific antigen binding molecule, wherein (i) the first and third Fab fragment capable of specific binding to 4-1BB each comprise a heavy chain variable region VH comprising the amino acid sequence of SEQ ID NO: 7 and a light chain variable region comprising an amino acid sequence of SEQ ID NO: 8 and (ii) the Fab fragment capable of specific binding to CEA comprises a heavy chain variable region VH comprising an amino acid sequence of SEQ ID NO:39 and a light chain variable region comprising an amino acid sequence of SEQ ID NO:40. In another particular aspect, provided is a bispecific antigen binding molecule, wherein (i) the first and third Fab fragment capable of specific binding to 4-1BB each comprise a heavy chain variable region VH comprising the amino acid sequence of SEQ ID NO: 7 and a light chain variable region comprising an amino acid sequence of SEQ ID NO: 8 and (ii) the Fab fragment capable of specific binding to CEA comprises a heavy chain variable region VH comprising an amino acid sequence of SEQ ID NO:47 and a light chain variable region comprising an amino acid sequence of SEQ ID NO:48.

In another aspect, the invention provides a bispecific antigen binding molecule, wherein the second Fab fragment capable of specific binding to a target cell antigen is a Fab fragment capable of specific binding to CD19.

Particularly, a bispecific antigen binding molecule is provided, wherein the Fab fragment capable of specific binding to CD19 comprises
(a) a heavy chain variable region (V_{H}CD19) comprising (i) CDR-H1 comprising the amino acid sequence of SEQ ID NO:57, (ii) CDR-H2 comprising the amino acid sequence of SEQ ID NO:58, and (iii) CDR-H3 comprising the amino acid sequence of SEQ ID NO:59, and a light chain variable region (V_{L}CD19) comprising (iv) CDR-L1 comprising the amino acid sequence of SEQ ID NO:60, (v) CDR-L2 comprising the amino acid sequence of SEQ ID NO:61, and (vi) CDR-L3 comprising the amino acid sequence of SEQ ID NO:62.

Particularly, the Fab fragment capable of specific binding to CD19 comprises a heavy chain variable region (V_{H}CD19) comprising an amino acid sequence that is at least about 95%, 96%, 97%, 98%, 99% or 100% identical to the amino acid sequence of SEQ ID NO:63, and a light chain variable region (V_{L}CD19) comprising an amino acid sequence that is at least about 95%, 96%, 97%, 98%, 99% or 100% identical to the amino acid sequence of SEQ ID NO:64. More particularly, the Fab fragment capable of specific binding to CD19 comprises a heavy chain variable region (V_{H}CD19) comprising the amino acid sequence of SEQ ID NO:63 and a light chain variable region (V_{L}CD19) comprising the amino acid sequence of SEQ ID NO:64.

In a particular aspect, provided is a bispecific antigen binding molecule, wherein
(i) the first and third Fab fragment capable of specific binding to 4-1BB each comprise a heavy chain variable region VH comprising the amino acid sequence of SEQ ID NO: 7 and a light chain variable region comprising an amino acid sequence of SEQ ID NO: 8 and
(ii) the second Fab fragment capable of specific binding to CD19 comprises a heavy chain variable region VH comprising an amino acid sequence of SEQ ID NO:63 and a light chain variable region VL comprising an amino acid sequence of SEQ ID NO:64.

In yet another aspect, the invention provides a bispecific antigen binding molecule, wherein the second Fab fragment capable of specific binding to a target cell antigen is a Fab fragment capable of specific binding to PD-L1.

Particularly, a bispecific antigen binding molecule is provided, wherein the Fab fragment capable of specific binding to PD-L1 comprises a heavy chain variable region (V_{H}PD-L1) comprising (i) CDR-H1 comprising the amino acid sequence of SEQ ID NO:145, (ii) CDR-H2 comprising the amino acid sequence of SEQ ID NO:146, and (iii) CDR-H3 comprising the amino acid sequence of SEQ ID NO:147, and a light chain variable region (V_{L}PD-L1) comprising (iv) CDR-L1 comprising the amino acid sequence of SEQ ID NO:148, (v) CDR-L2 comprising the amino acid sequence of SEQ ID NO:149, and (vi) CDR-L3 comprising the amino acid sequence of SEQ ID NO:150.

Particularly, the Fab fragment capable of specific binding to PD-L1 comprises a heavy chain variable region (V_{H}PD-L1) comprising an amino acid sequence that is at least about 95%, 96%, 97%, 98%, 99% or 100% identical to the amino acid sequence of SEQ ID NO:152, and a light chain variable region (V_{L}PD-L1) comprising an amino acid sequence that is at least about 95%, 96%, 97%, 98%, 99% or 100% identical to the amino acid sequence of SEQ ID NO:153. More particularly, the Fab fragment capable of specific binding to PD-L1 comprises a heavy chain variable region (V_{H}PD-L1) comprising the amino acid sequence of SEQ ID NO:152 and a light chain variable region (V_{L}PD-L1) comprising the amino acid sequence of SEQ ID NO:153.

In a particular aspect, provided is a bispecific antigen binding molecule, wherein
(i) the first and third Fab fragment capable of specific binding to 4-1BB each comprise a heavy chain variable region VH comprising the amino acid sequence of SEQ ID NO: 7 and a light chain variable region comprising an amino acid sequence of SEQ ID NO: 8 and
(ii) the second Fab fragment capable of specific binding to PD-L1 comprises a heavy chain variable region VH comprising an amino acid sequence of SEQ ID NO:152 and a light chain variable region VL comprising an amino acid sequence of SEQ ID NO:153.

According to another aspect of the invention, there is provided an isolated polynucleotide encoding a bispecific antigen binding molecule as described herein before. The invention further provides a vector, particularly an expression vector, comprising the isolated polynucleotide of the invention and a host cell comprising the isolated polynucleotide or the vector of the invention. In some aspects the host cell is a eukaryotic cell, particularly a mammalian cell.

In another aspect, provided is a method for producing a bispecific antigen binding molecule as described herein before, comprising the steps of (i) culturing the host cell of the invention under conditions suitable for expression of the antigen binding molecule, and (ii) recovering the antigen binding molecule. The invention also encompasses the bispecific antigen binding molecule produced by the method of the invention.

The invention further provides a pharmaceutical composition comprising a bispecific antigen binding molecule as described herein before and at least one pharmaceutically acceptable excipient. In one aspect, the pharmaceutical composition is for use in the treatment of cancer.

Also encompassed by the invention is the bispecific antigen binding molecule as described herein before, or the pharmaceutical composition comprising the bispecific antigen binding molecule, for use as a medicament.

In one aspect, provided is a bispecific antigen binding molecule as described herein before or the pharmaceutical composition of the invention, for use
(i) in stimulating T cell response,
(ii) in supporting survival of activated T cells,
(iii) in the treatment of cancer,
(iv) in delaying progression of cancer, or
(v) in prolonging the survival of a patient suffering from cancer.

In one aspect, provided is the bispecific antigen binding molecule as described herein before, or the pharmaceutical composition of the invention, for use in the treatment of cancer. In another aspect, the invention provides the bispecific antigen binding molecule as described herein before for use in the treatment of cancer, wherein the bispecific antigen binding molecule is administered in combination with a chemotherapeutic agent, radiation therapy and/ or other agents for use in cancer immunotherapy.

In a further aspect, the invention provides a method of inhibiting the growth of tumor cells in an individual comprising administering to the individual an effective amount of the bispecific antigen binding molecule as described herein before, or the pharmaceutical composition of the invention, to inhibit the growth of the tumor cells.

Also provided is the use of the the bispecific antigen binding molecule as described herein before for the manufacture of a medicament for the treatment of a disease in an individual in need thereof, in particular for the manufacture of a medicament for the treatment of cancer or infectious diseases, as well as a method of treating a disease in an individual, comprising administering to said individual a therapeutically effective amount of a composition comprising the bispecific antigen binding molecule of the invention in a pharmaceutically acceptable form. In a specific aspect, the disease is cancer. In any of the above aspects the individual is a mammal, particularly a human.

### BRIEF DESCRIPTION OF THE DRAWINGS

FIG. 1A shows an example of the bispecific antigen binding molecules of the invention. The bispecific antigen binding molecule is in huIgG1 P329GLALA format comprising two anti-4-1BB Fab fragments (bivalent binding to 4-1BB) and one anti-FAP cross-Fab fragment (a Fab fragment, wherein the VH and VL region are exchanged) which is fused at the C-terminus of its heavy chain to the N-terminus of the heavy chain of one of the 4-1BB Fab fragments. This format is termed herein the Head to Head (H2H) 2+1 format. The big black dot symbolizes the knob-into-hole mutations, whereas the small black dots in the CH1/CL domains symbolize amino acid mutation that improve the correct pairing of the heavy chains with the anti-4-1BB light chains.

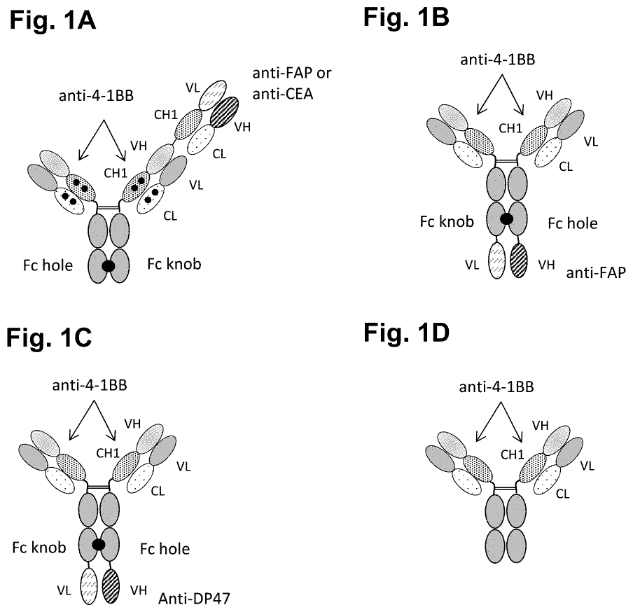

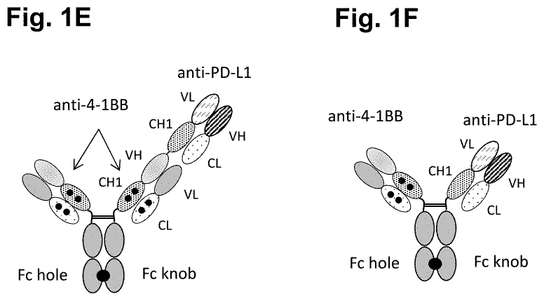

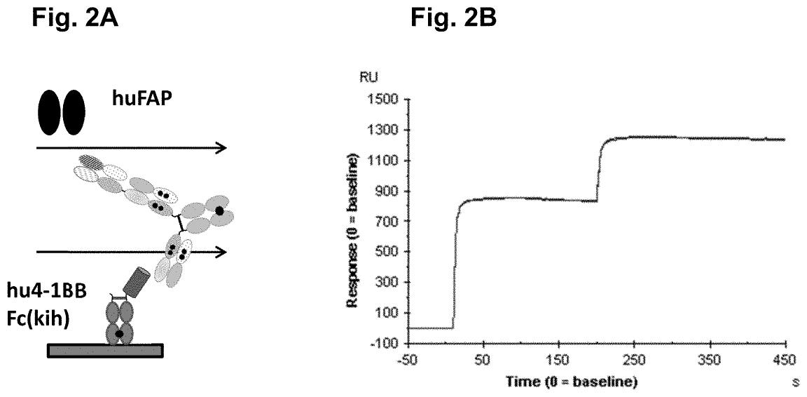

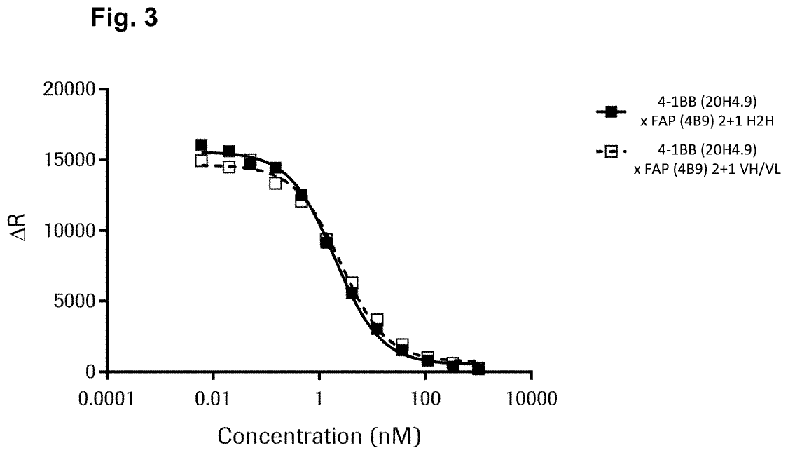

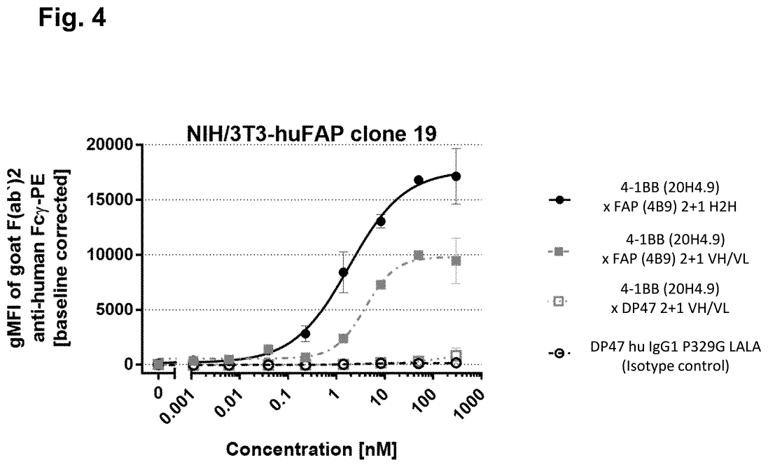

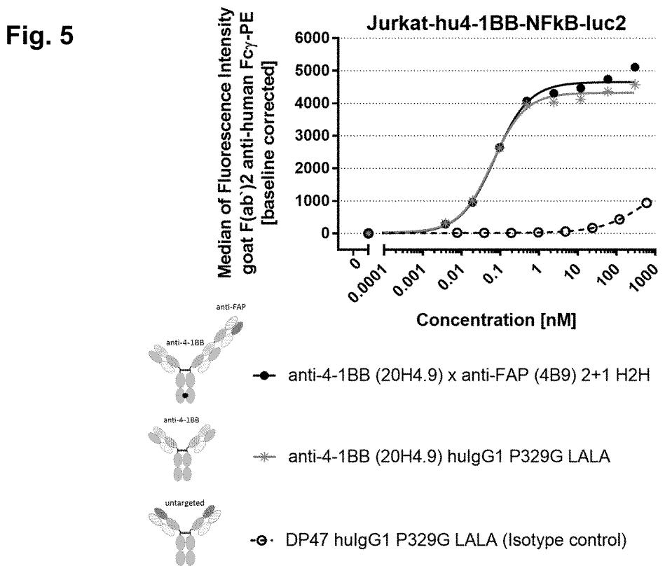

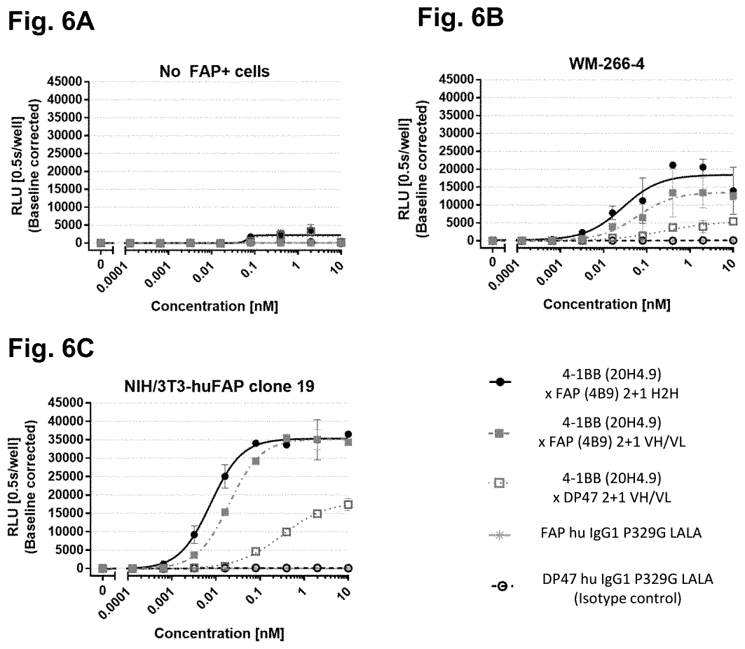

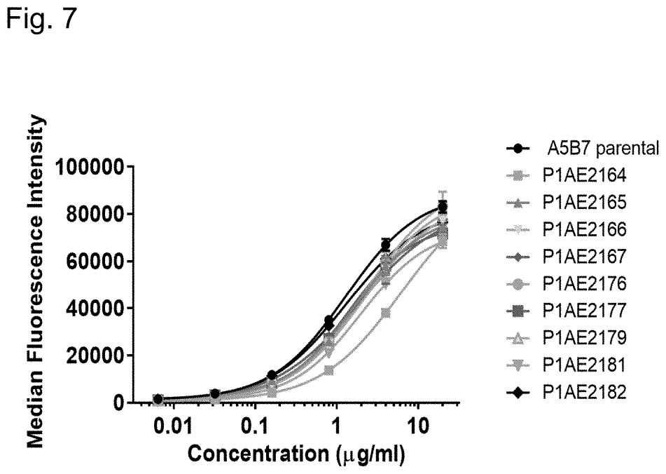

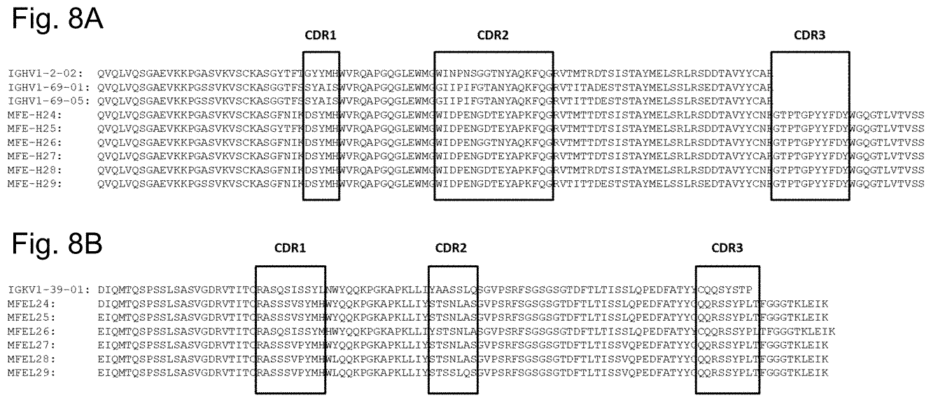

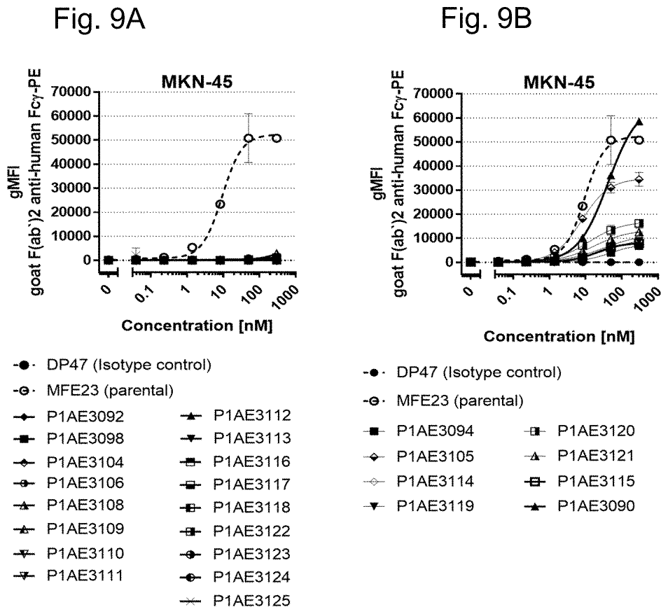

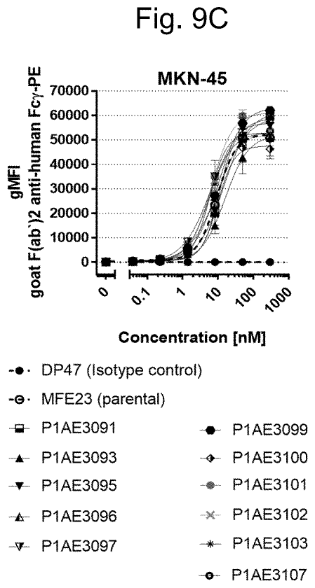

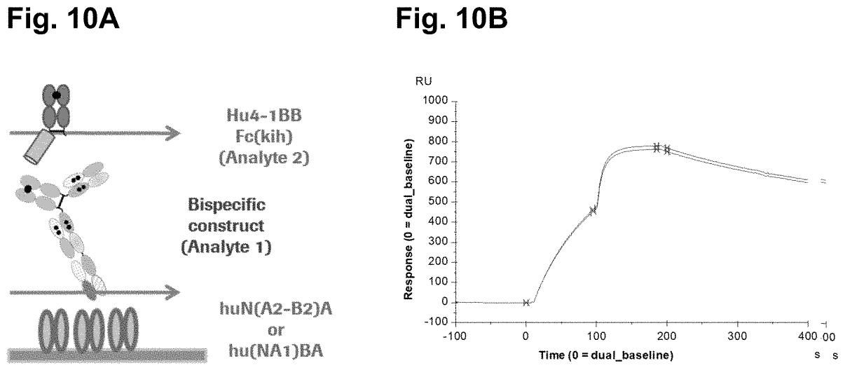

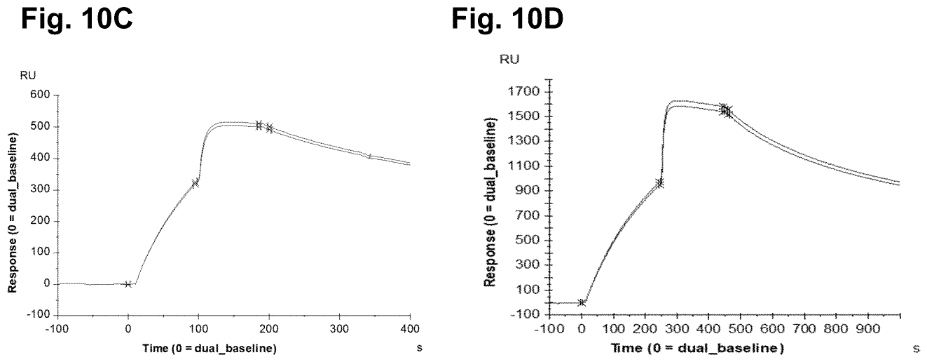

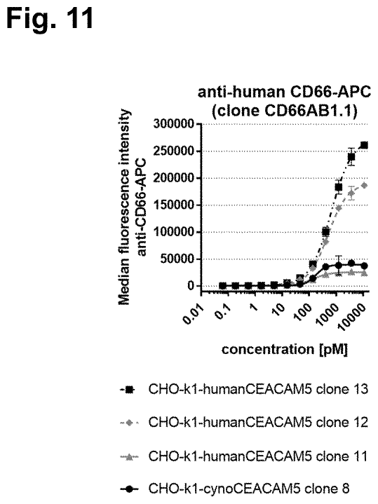

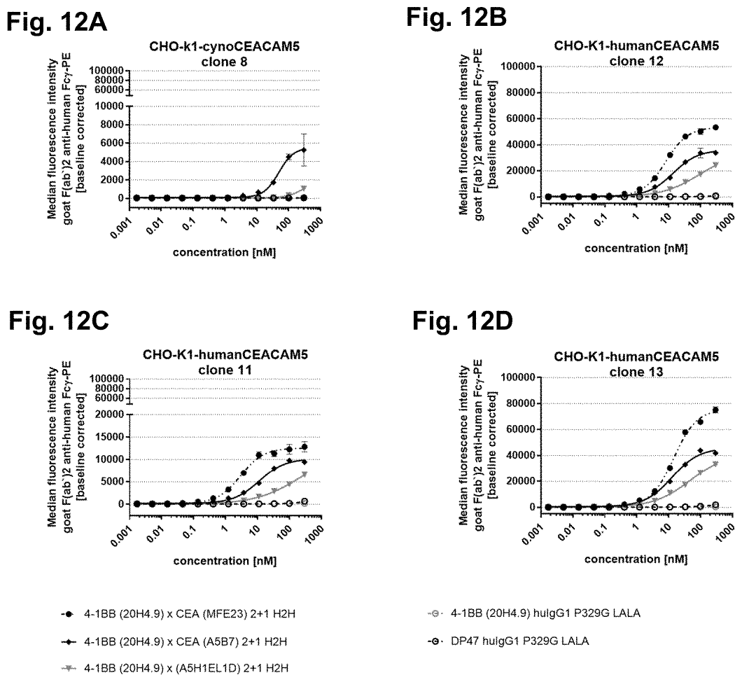

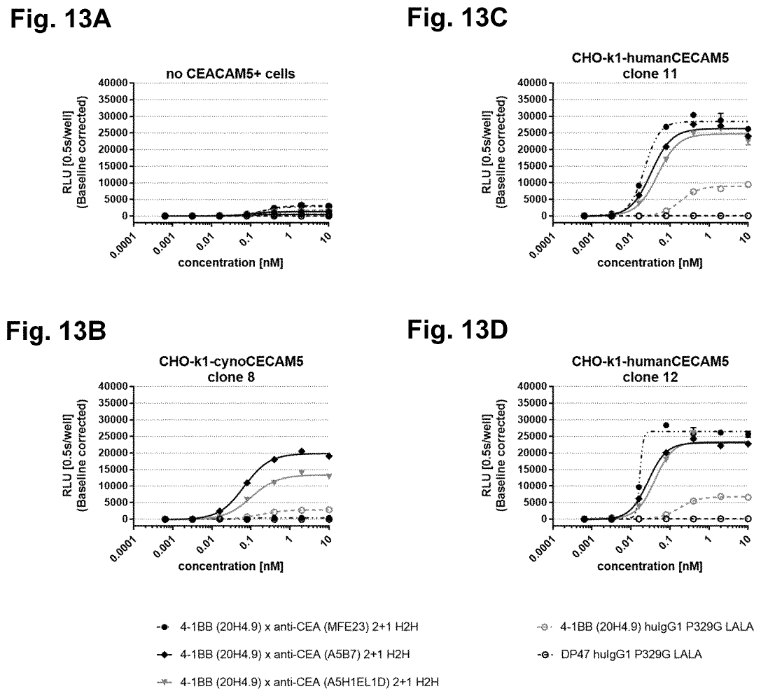

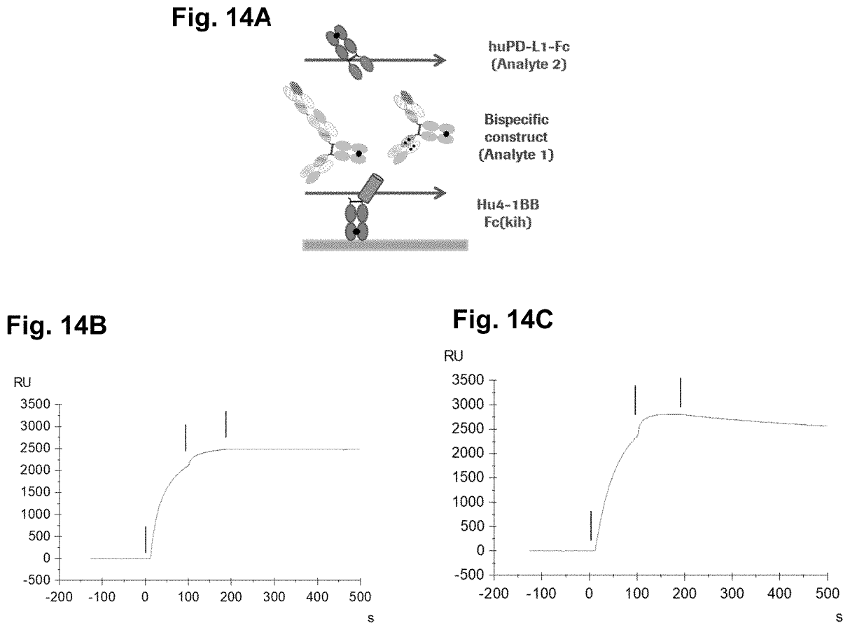

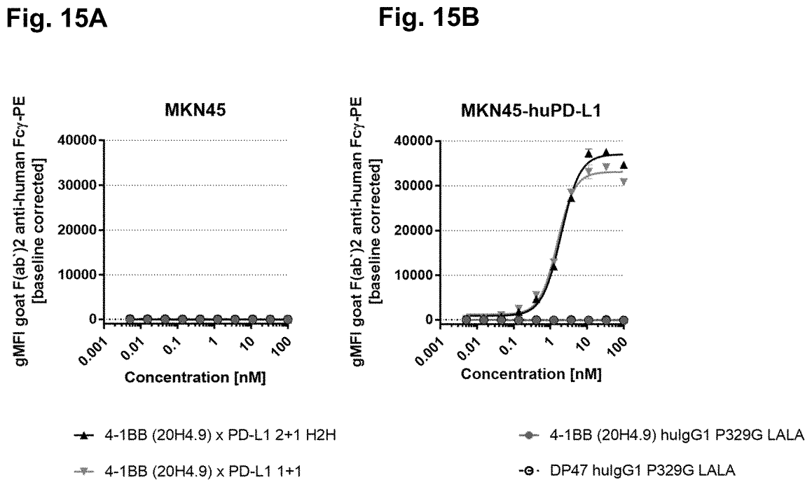

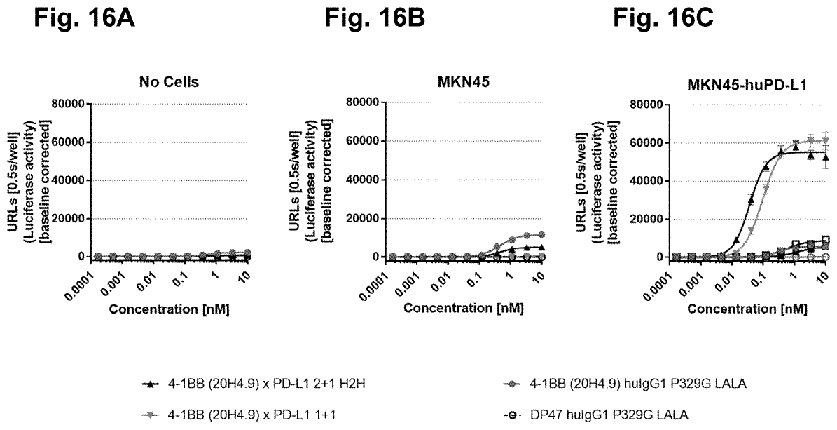

In FIG. 1B is shown another bispecific antigen binding molecule in huIgG1 P329GLALA format comprising two anti-4-1BB Fab fragments (bivalent binding to-4-1BB) and a VH and VL domain capable of specific binding to FAP fused at the C-terminus of the heavy chains, respectively. This format is termed herein 2+1 VH/VL format and is used herein as control molecule. In FIG. 1C a bispecific antigen binding molecule in huIgG1 P329GLALA format comprising two anti-4-1BB Fab fragments (bivalent binding to 4-1BB) and a VH and VL domain of germline control DP47 (untargeted control) is shown. FIG. 1D shows a standard antibody in huIgG1 P329GLALA format comprising two anti-4-1BB Fab fragments (bivalent binding to-4-1BB).

FIG. 1E shows another example of the bispecific antigen binding molecules of the invention. The bispecific antigen binding molecule is in huIgG1 P329GLALA format comprising two anti-4-1BB Fab fragments (bivalent binding to 4-1BB) and one anti-human PD-L1 (called anti-PD-L1) cross-Fab fragment (a Fab fragment, wherein the VH and VL region are exchanged) which is fused at the C-terminus of its heavy chain to the N-terminus of the heavy chain of one of the 4-1BB Fab fragments. This format is termed herein the Head to Head (H2H) 2+1 format. The big black dot symbolizes the knob-into-hole mutations, whereas the small black dots in the CH1/CL domains symbolize amino acid mutation that improve the correct pairing of the heavy chains with the anti-4-1BB light chains. FIG. 1F shows a bispecific antigen binding molecule in huIgG1 P329GLALA format comprising one anti-4-1BB Fab fragment (monovalent binding to 4-1BB) and one anti-human PD-L1 (called anti-PD-L1) cross-Fab fragment (a Fab fragment, wherein the VH and VL region are exchanged). This format is termed herein the 1+1 format. The big black dot symbolizes the knob-into-hole mutations, whereas the small black dots in the CH1/CL domains symbolize amino acid mutation that improve the correct pairing of the heavy chains with the anti-4-1BB light chains.

FIGS. 2A and 2B relate to the simultaneous binding of the bispecific 2+1 H2H anti-4-1BB×anti-FAP huIgG1 P329GLALA antigen binding molecules to 4-1BB and FAP. FIG. 2A is a pictogram of the setup of the assay. FIG. 2B shows the simultaneous binding of the bispecific H2H anti-4-1BB×anti-FAP antigen binding molecules (analyte 1) to immobilized human 4-1BB and human FAP (analyte 2).

FIG. 3 shows a FRET based competition assay to assess the bivalent binding of the 2+1 H2H anti-4-1BB (20H4.9)×anti-FAP (4B9) antigen binding molecules. The interaction between hu4-1BB-SNAP Tb-labeled expressed on transfected Hek cells and d2-labeled 4-1BB (clone 20H4.9) IgG was competed by addition of unlabeled 2+1 H2H anti-4-1BB (20H4.9)×anti-FAP (4B9) huIgG1 PGLALA construct (one of the Fabs against 4-1BB does not have a free N-terminus, filled square) or by unlabeled 2+1 VH/VL (C-terminal) 4-1BB (20H4.9)×FAP (4B9) bispecific construct (two “free Fabs” against 4-1BB, open square). Competition resulted in a decrease of the TR-FRET signal.

In FIG. 4 the binding to human FAP-expressing NIH/3T3-huFAP clone 19 cells is shown. The concentration of the 2+1 H2H anti-4-1BB (20H4.9)×anti-FAP(4B9) antigen binding molecules (black filled circle and line) or its control molecules is blotted against the geo mean of fluorescence intensity (gMFI) of the PE-conjugated secondary detection antibody. All values are baseline corrected by subtracting the baseline values of the blank control (e.g. no primary only secondary detection antibody). Only FAP antigen binding domain-containing constructs like anti-4-1BB (20H4.9)×anti-FAP (4B9) 2+1 H2H huIgG1 P329GLALA (black filled circle and line) and anti-4-1BB (20H4.9)×anti-FAP (4B9) 2+1 VH/VL huIgG1 P329GLALA (filled grey square and line) bind efficiently to FAP-expressing cells. It can be seen that the N-terminally fused anti-FAP cross-Fab fragment of anti-4-1BB (20H4.9)×anti-FAP (4B9) 2+1 H2H huIgG1 P329GLALA (black filled circle and line) showed a higher gMFI and lower EC₅₀ than the C-terminally fused VH/VL anti-FAP antigen binding domain of anti-4-1BB (20H4.9)×anti-FAP (4B9) VH/VL 2+1 huIgG1 P329GLALA (grey filled square and line).

FIG. 5 shows the binding to human 4-1BB (CD137) expressing reporter cell line Jurkat-hu4-1BB-NFkB-luc2. The concentration of 2+1 H2H anti-4-1BB (20H4.9)×anti-FAP(4B9) antigen binding molecules (black filled circle and line) or its controls is blotted against the geo mean of fluorescence intensity (gMFI) of the PE-conjugated secondary detection antibody. All values are baseline corrected by subtracting the baseline values of the blank control (e.g. no primary only secondary detection antibody). Anti-4-1BB (20H4.9)×anti-FAP (4B9) 2+1 H2H huIgG1 P329GLALA (black filled circle and line) binds similarly to 4-1BB as its control anti-4-1BB (20H4.9) huIgG1 P329GLALA (grey star and line).

FIGS. 6A to 6C show the NFκB-mediated luciferase expression activity in 4-1BB expressing reporter cell line Jurkat-hu4-1BB-NFκB-luc2. To test the functionality of 2+1 H2H anti-4-1BB (20H4.9)×anti-FAP(4B9) antigen binding molecules (black filled circle and line) versus 2+1 VH/VL anti-4-1BB (20H4.9)×anti-FAP (4B9) antigen binding molecules (grey filled square and line) versus controls, molecules were incubated with the reporter cell line Jurkat-hu4-1BB-NFkB-luc2 in the absence or presence of human-FAP expressing cell lines in a 1:5 ratio for 5 h. The concentration of 2+1 H2H anti-4-1BB (20H4.9)×anti-FAP(4B9) antigen binding molecules or its controls are blotted against the units of released light (RLU) measured after 5 h of incubation and addition of Luciferase detection solution. All values are baseline corrected by subtracting the baseline values of the blank control (e.g. no antibodies added). In FIG. 6A FAP-target-independent 4-1BB activation is shown, whereby 4-1BB-binding induces NFκB-controlled luciferase expression in the reporter cell line without any FAP-mediated crosslinking. In FIG. 6B FAP-expressing human melanoma cell line WM-266-4 human melanoma cell line (intermediate FAP-surface expression) was added. The FAP-expressing WM-266-4 cells lead to crosslinking of the bispecific 4-1BB (20H4.9)×FAP (4B9) antigen binding molecules. The bispecific FAP-targeted 2+1 H2H anti-4-1BB (20H4.9)×anti-FAP(4B9) antigen binding molecule (black filled circle and line) showed a slightly better activation (lower EC50 values), which may reflect the higher affinity to FAP. This can even be better seen in FIG. 6C which shows the NFκB-induced luciferase activation in the presence of high FAP-expressing cell line NIH/3T3-huFAP clone 19 (human-FAP-transgenic mouse fibroblast cell line).

FIG. 7 shows the binding of humanized A5B7 huIgG1 P329G LALA variants to MKN-45 as compared to the binding of the parental murine A5B7 antibody. Antibodies were detected with a fluorescently labeled secondary antibody and fluorescence was measured by flow cytometry.

FIGS. 8A and 8B show an alignment of the VH amino acid sequences (FIG. 8A) and VL amino acid sequences (FIG. 8B) of the humanized MFE23 antibody variants.

FIGS. 9A, 9B and 9C show the binding of humanized MFE23 huIgG1 P329G LALA variants to MKN-45 as compared to the binding of the parental murine MFE23 antibody. Antibodies were detected with a fluorescently labeled secondary antibody and fluorescence was measured by flow cytometry. The graph was split into three graphs displaying low binding, intermediate binding and similar binding to the parental MFE23 clone.

FIGS. 10A to 10D relate to simultaneous binding of CEA-targeting trimeric split 4-1BBL molecules to hu4-1BB and huN(A2-B2)A or hu(NA1)BA. FIG. 10A shows the assay setup. FIG. 10B shows the simultaneous binding of 2+1 H2H anti-human 4-1BB (20H4.9)×CEA (A5B7) huIgG1 P329GLALA (Analyte 1) to immobilized human N(A2-B2)A and human 4-1BB (Analyte 2). FIG. 10C shows the simultaneous binding of 2+1 H2H 4-1BB (20H4.9)×CEA (A5H1EL1D) huIgG1 P329GLALA (Analyte 1) to immobilized human N(A2-B2)A and human 4-1BB (Analyte 2). FIG. 10D shows the simultaneous binding of 2+1 H2H 4-1BB (20H4.9)×CEA (MFE23) huIgG1 P329GLALA (Analyte 1) to immobilized human (NA1)BA and human 4-1BB (Analyte 2). Duplicates are shown.

The cell surface CEACAM5 expression level of different CEACAM5 expressing clones used for the binding assays is shown in FIG. 11. Chinese hamster ovary cell line called CHO-k1 (ATCC CRL-9618) was transfected with cynomolgus monkey CEACAM5 (CHO-k1-cynoCEACAM5 clone 8) or human CEACAM5 (CHO-k1-huCEACAM5 clone 11, clone 12 and clone 13). The expression levels were determined using titrated APC-labeled anti-CD66 specific detection antibody (clone CD66AB.1.1) by flow cytometry. Shown is the median of fluorescence intensity versus the concentration of the detection antibody, whereby the median of fluorescence intensity correlates positively with the amount of bound detection antibody and therefore with the expression level of CEACAM5 molecules on the cell surface. CHO-k1-cynoCEACAM5 clone 8 and CHO-k1-huCEACAM5 clone 11 display a similar cell surface CEACAM5 expression, whereas CHO-k1-huCEACAM5 clone 12 and 13 show a high cell surface CEACAM5 expression level.

In FIGS. 12A to 12D, the binding to cynomolgus monkey CEACAM5 or human CEACAM5-expressing CHO-k1 cells is shown. The concentration of the 2+1 H2H anti-4-1BB (20H4.9)×anti-CEA(4B9) antigen binding molecules or control molecules is blotted against the median of fluorescence intensity of the PE-conjugated secondary detection antibody. All values are baseline corrected by subtracting the baseline values of the blank control (e.g. no primary only secondary detection antibody). Only CEACAM5 antigen binding domain-containing constructs, e.g. anti-4-1BB (20H4.9)×CEA (MFE23) huIgG1 P329GLALA 2+1 H2H (black filled circle, dotted line), anti-4-1BB (20H4.9)×CEA (A5B7) huIgG1 P329GLALA 2+1 H2H (black filled diamond, black line) and 4-1BB (20H4.9)×(A5H1EL1D) huIgG1 P329GLALA 2+1 H2H (grey down facing triangle, grey line) bind efficiently to humanCEACAM5-expressing cells (FIGS. 12B, 12C and 12D). In contrast, only 4-1BB (20H4.9)×CEA (A5B7) huIgG1 P329GLALA 2+1 H2H (black filled diamond, black line) bind detectable to cynomolgus monkey CEACAM5, 4-1BB (20H4.9)×(A5H1EL1D) huIgG1 P329GLALA 2+1 H2H (grey down facing triangle, grey line) show only very weak cynomolgus CEACAM5 and 4-1BB (20H4.9)×CEA (MFE23) huIgG1 P329GLALA 2+1 H2H (black filled circle, dotted line) shows no binding as the MFE23 is not human/cyno-crossreactive (FIG. 12A).

FIGS. 13A to 13D show the NFκB-mediated luciferase expression activity in 4-1BB expressing reporter cell line Jurkat-hu4-1BB-NFκB-luc2. To test the functionality of 2+1 H2H anti-4-1BB (20H4.9)×anti-CEA antigen binding molecules versus controls, molecules were incubated with the reporter cell line Jurkat-hu4-1BB-NFkB-luc2 in the absence or presence of cynomolgus monkey or human CEACAM5 expressing CHO-k1 cell lines in a 1:5 ratio for 5 h. The concentration of 2+1 H2H anti-4-1BB (20H4.9)×anti-CEA antigen binding molecules or its controls are blotted against the units of released light (RLU) measured after 5 h of incubation and addition of Luciferase detection solution. All values are baseline corrected by subtracting the baseline values of the blank control (e.g. no antibodies added). In correlation to the binding assay to CEACAM5 expressing CHO-k1 cells all CEACAM5 antigen binding domain-containing constructs, e.g. anti-4-1BB (20H4.9)×CEA (MFE23) huIgG1 P329GLALA 2+1 H2H (black filled circle, dotted line), anti-4-1BB (20H4.9)×CEA (A5B7) huIgG1 P329GLALA 2+1 H2H (black filled diamond, black line) and anti-4-1BB (20H4.9)×(A5H1EL1D) huIgG1 P329GLALA 2+1 H2H (grey down facing triangle, grey line) induce increased Jurkat-hu4-1BB-NFκB-luc2 reporter cell line activation in the presence of human CEACAM5 expression CHO-k1 cell lines (FIG. 13C and FIG. 13D). Whereas in the presence of cynomolgus monkey CEACAM5 only anti-4-1BB (20H4.9)×CEA (A5B7) huIgG1 P329GLALA 2+1 H2H (black filled diamond, black line) and anti-4-1BB (20H4.9)×(A5H1EL1D) huIgG1 P329GLALA 2+1 H2H (grey down facing triangle, grey line) but not anti-4-1BB (20H4.9)×CEA (MFE23) huIgG1 P329GLALA 2+1 H2H (black filled circle, dotted line) induced Jurkat-hu4-1BB-NFκB-luc2 reporter cell line activation as the MFE23 is not human/cynomolgus monkey-crossreactive (FIG. 13B). In the absence of CEACAM5-expressing cells no crosslink of 4-1BB (20H4.9)×CEA 2+1 H2H occurred and Jurkat-hu4-1BB-NFκB-luc2 reporter cell line were not activated.

FIG. 14A shows the setup to assess simultaneous binding of PD-L1 targeted 4-1BB agonist constructs to hu4-1BB and huPD-L1-Fc. FIG. 14B shows the simultaneous binding of 2+1 H2H 4-1BB (20H4.9)/PD-L1 human IgG1 PGLALA to huPD-L1-Fc and hu4-1BB-Fc(kih). FIG. 14C shows the simultaneous binding of 1+1 H2H 4-1BB (20H4.9)/PD-L1 human IgG1 PGLALAto huPD-L1-Fc and hu4-1BB-Fc(kih). Triplicates are shown.

In FIGS. 15A and 15B the binding to MKN45 and PD-L1 transfected MKN45 (MKN45-huPD-L1) cell are shown. The concentration of the 2+1 H2H anti-4-1BB (20H4.9)×anti-human PD-L1 antigen binding molecule, 1+1 anti-4-1BB (20H4.9)×anti-human PD-L1 antigen binding molecules or control molecules is blotted against the geo mean of fluorescence intensity of the PE-conjugated secondary detection antibody. All values are baseline corrected by subtracting the baseline values of the blank control (e.g. no primary only secondary detection antibody). Only PD-L1 antigen binding domain-containing constructs, e.g. 4-1BB (20H4.9)×PD-L1 huIgG1 P329GLALA 2+1 H2H (black upfacing triangle and line) and anti-4-1BB (20H4.9)×PD-L1 huIgG1 P329GLALA 1+1 (grey down-facing triangle and line line) bind efficiently to humanPD-L1-expressing MKN45-huPD-L1 cells (FIG. 15B) but not to the parental cell line MKN45 (FIG. 15A).

FIGS. 16A to 16C show the NFκB-mediated luciferase expression activity in 4-1BB expressing reporter cell line Jurkat-hu4-1BB-NFκB-luc2. To test the functionality of 2+1 H2H anti-4-1BB (20H4.9)×PD-L1 and 1+1 anti-4-1BB (20H4.9)×PD-L1 antigen binding molecules versus controls, molecules were incubated with the reporter cell line Jurkat-hu4-1BB-NFkB-luc2 in the absence or presence of human PD-L1 expressing MKN45 cell lines in a 1:5 ratio for 5 h. The concentration of 2+1 H2H anti-4-1BB (20H4.9)×PD-L1 and 1+1 anti4-1BB (20H4.9)×PD-L1 antigen binding molecules or its controls are blotted against the units of released light (RLU) measured after 5 h of incubation and addition of Luciferase detection solution. As shown in FIG. 16B, in the absence of PD-L1 expressing cells none of the molecules were able to induce strong human 4-1BB receptor activation in the Jurkat-hu4-1BB-NFkB-luc2 reporter cell line. As shown in FIG. 16C, in the presence of humanPD-L1-expressing MKN45 cells, crosslinking of bispecific 2+1 H2H anti-4-1BB, anti-PD-L1 huIgG1 PGLALA antibody (black triangle and line) or bispecific 1+1 anti-4-1BB, anti-PD-L1 huIgG1 PGLALA antibody (grey triangle and line) led to a strong increase of NFkB-activated Luciferase activity in the Jurkat-hu4-1BB-NFkB-luc2 reporter cell line, which was above the activation mediated by the untargeted control anti-4-1BB (20H4.9) huIgG1 P329GLALA (filled grey circle and line). All values are baseline corrected by subtracting the baseline values of the blank control (e.g. no antibodies added). Monovalent binding to 4-1BB as displayed by 1+1 anti4-1BB (20H4.9)×PD-L1 huIgG1 P329GLALA leads to a slightly lower EC₅₀ value.

### DETAILED DESCRIPTION OF THE INVENTION

Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, antibody fragments and scaffold antigen binding proteins.

As used herein, the term “antigen binding domain capable of specific binding to a target cell antigen” or “moiety capable of specific binding to a target cell antigen” refers to a polypeptide molecule that specifically binds to an antigenic determinant. In one aspect, the antigen binding domain is able to activate signaling through its target cell antigen. In a particular aspect, the antigen binding domain is able to direct the entity to which it is attached (e.g. the 4-1BB agonist) to a target site, for example to a specific type of tumor cell or tumor stroma bearing the antigenic determinant. Antigen binding domains capable of specific binding to a target cell antigen include antibodies and fragments thereof as further defined herein. In addition, antigen binding domains capable of specific binding to a target cell antigen include scaffold antigen binding proteins as further defined herein, e.g. binding domains which are based on designed repeat proteins or designed repeat domains (see e.g. WO 2002/020565).

In relation to an antibody or fragment thereof, the term “antigen binding domain capable of specific binding to a target cell antigen” refers to the part of the molecule that comprises the area which specifically binds to and is complementary to part or all of an antigen. A antigen binding domain capable of specific antigen binding may be provided, for example, by one or more antibody variable domains (also called antibody variable regions). Particularly, an antigen binding domain capable of specific antigen binding comprises an antibody light chain variable region (VL) and an antibody heavy chain variable region (VH).

In a particular aspect, the “antigen binding domain capable of specific binding to a target cell antigen ” is a Fab fragment or a cross-Fab fragment.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used within the current application denotes the presence of a specified number of binding sites specific for one distinct antigenic determinant in an antigen binding molecule that are specific for one distinct antigenic determinant. As such, the terms “bivalent”, “tetravalent”, and “hexavalent” denote the presence of two binding sites, four binding sites, and six binding sites specific for a certain antigenic determinant, respectively, in an antigen binding molecule. In particular aspects of the invention, the bispecific antigen binding molecules according to the invention can be monovalent for a certain antigenic determinant, meaning that they have only one binding site for said antigenic determinant or they can be bivalent or tetravalent for a certain antigenic determinant, meaning that they have two binding sites or four binding sites, respectively, for said antigenic determinant.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CHL CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, N.Y., pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” or “Fab molecule” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments wherein the cysteine residue(s) of the constant domains bear a free thiol group. Pepsin treatment yields an F(ab′)₂ fragment that has two antigen-combining sites (two Fab fragments) and a part of the Fc region. According to the present invention, the term “Fab fragment” also includes “cross-Fab fragments” or “crossover Fab fragments” as defined below.

The term “cross-Fab fragment” or “cross-Fab molecule” or “xFab fragment” or “crossover Fab fragment” refers to a Fab fragment, wherein either the variable regions or the constant regions of the heavy and light chain are exchanged. Two different chain compositions of a cross-Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab (VLVH). On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab (CLCH1).

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4⁺ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1. A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can beengineered to include upto 25 amino acids without affecting the overall fold of the microprotein. For further details of engineered knottin domains, see WO2008098796.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

The term “antigen binding domain” or “antigen-binding site” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

A “target cell antigen” as used herein refers to an antigenic determinant presented on the surface of a target cell, for example a cell in a tumor such as a cancer cell or a cell of the tumor stroma. In certain embodiments, the target cell antigen is a tumor-specific or a tumor-associated antigen (TAA). In one embodiment, the TAA is an antigen on the surface of a tumor cell. In one embodiment, the TAA is on a cell of the tumor stroma. In another aspect, the target cell antigen is a tumor-associated antigen on a T cell or B cell. In one embodiment, target cell antigen is selected from the group consisting of Fibroblast Activation Protein (FAP), Carcinoembryonic Antigen (CEA), Melanoma-associated Chondroitin Sulfate Proteoglycan (MCSP), Epidermal Growth Factor Receptor (EGFR), CD19, CD20, CD33 and PD-L1. In a particular aspect, the target cell antigen is Fibroblast Activation Protein (FAP), Carcinoembryonic Antigen (CEA) or CD19. More particularly, the target cell antigen is Fibroblast Activation Protein (FAP) or CEA. In another aspect, the target cell antigen is PD-L1.

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP that results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:86), or NCBI (www.ncbi.nlm.nih.gov/) RefSeq NP_004451.2. The extracellular domain (ECD) of human FAP extends from amino acid position 26 to 760. The amino acid sequence of a His-tagged human FAP ECD is shown in SEQ ID NO 87. The amino acid sequence of mouse FAP is shown in UniProt accession no. P97321 (version 126, SEQ ID NO:88), or NCBI RefSeq NP_032012.1. The extracellular domain (ECD) of mouse FAP extends from amino acid position 26 to 761. SEQ ID NO:89 shows the amino acid sequence of a His-tagged mouse FAP ECD. SEQ ID NO: 90 shows the amino acid sequence of a His-tagged cynomolgus FAP ECD. Preferably, an anti-FAP binding molecule of the invention binds to the extracellular domain of FAP.

The term “Carcinoembroynic antigen (CEA)”, also known as Carcinoembryonic antigen-related cell adhesion molecule 5 (CEACAM5), refers to any native CEA from any vertebrate source, including mammals such as primates (e.g. humans) non-human primates (e.g. cynomolgus monkeys) and rodents (e.g. mice and rats), unless otherwise indicated. The amino acid sequence of human CEA is shown in UniProt accession no. P06731 (version 151, SEQ ID NO:91). CEA has long been identified as a tumor-associated antigen (Gold and Freedman, J Exp Med., 121:439-462, 1965; Berinstein N. L., J Clin Oncol., 20:2197-2207, 2002). Originally classified as a protein expressed only in fetal tissue, CEA has now been identified in several normal adult tissues. These tissues are primarily epithelial in origin, including cells of the gastrointestinal, respiratory, and urogential tracts, and cells of colon, cervix, sweat glands, and prostate (Nap et al., Tumour Biol., 9(2-3):145-53, 1988; Nap et al., Cancer Res., 52(8):2329-23339, 1992). Tumors of epithelial origin, as well as their metastases, contain CEA as a tumor associated antigen. While the presence of CEA itself does not indicate transformation to a cancerous cell, the distribution of CEA is indicative. In normal tissue, CEA is generally expressed on the apical surface of the cell (Hammarström S., Semin Cancer Biol. 9(2):67-81 (1999)), making it inaccessible to antibody in the blood stream. In contrast to normal tissue, CEA tends to be expressed over the entire surface of cancerous cells (Hammarström S., Semin Cancer Biol. 9(2):67-81 (1999)). This change of expression pattern makes CEA accessible to antibody binding in cancerous cells. In addition, CEA expression increases in cancerous cells. Furthermore, increased CEA expression promotes increased intercellular adhesions, which may lead to metastasis (Marshall J., Semin Oncol., 30(a Suppl. 8):30-6, 2003). The prevalence of CEA expression in various tumor entities is generally very high. In concordance with published data, own analyses performed in tissue samples confirmed its high prevalence, with approximately 95% in colorectal carcinoma (CRC), 90% in pancreatic cancer, 80% in gastric cancer, 60% in non-small cell lung cancer (NSCLC, where it is co-expressed with HER3), and 40% in breast cancer; low expression was found in small cell lung cancer and glioblastoma.

CEA is readily cleaved from the cell surface and shed into the blood stream from tumors, either directly or via the lymphatics. Because of this property, the level of serum CEA has been used as a clinical marker for diagnosis of cancers and screening for recurrence of cancers, particularly colorectal cancer (Goldenberg D M., The International Journal of Biological Markers, 7:183-188, 1992; Chau I., et al., J Clin Oncol., 22:1420-1429, 2004; Flamini et al., Clin Cancer Res; 12(23):6985-6988, 2006).

The term “Melanoma-associated Chondroitin Sulfate Proteoglycan (MCSP)”, also known as Chondroitin Sulfate Proteoglycan 4 (CSPG4) refers to any native MCSP from any vertebrate source, including mammals such as primates (e.g. humans) non-human primates (e.g. cynomolgus monkeys) and rodents (e.g. mice and rats), unless otherwise indicated. The amino acid sequence of human MCSP is shown in UniProt accession no. Q6UVK1 (version 103, SEQ ID NO:92). The term “Epidermal Growth Factor Receptor (EGFR)”, also named Proto-oncogene c-ErbB-1 or Receptor tyrosine-protein kinase erbB-1, refers to any native EGFR from any vertebrate source, including mammals such as primates (e.g. humans) non-human primates (e.g. cynomolgus monkeys) and rodents (e.g. mice and rats), unless otherwise indicated. The amino acid sequence of human EGFR is shown in UniProt accession no. P00533 (version 211, SEQ ID NO:93). The term “CD19” refers to B-lymphocyte antigen CD19, also known as B-lymphocyte surface antigen B4 or T-cell surface antigen Leu-12 and includes any native CD19 from any vertebrate source, including mammals such as primates (e.g. humans) non-human primates (e.g. cynomolgus monkeys) and rodents (e.g. mice and rats), unless otherwise indicated. The amino acid sequence of human CD19 is shown in Uniprot accession no. P15391 (version 160, SEQ ID NO:94). “CD20” refers to B-lymphocyte antigen CD20, also known as membrane-spanning 4-domains subfamily A member 1 (MS4A1), B-lymphocyte surface antigen B1 or Leukocyte surface antigen Leu-16, and includes any native CD20 from any vertebrate source, including mammals such as primates (e.g. humans) non-human primates (e.g. cynomolgus monkeys) and rodents (e.g. mice and rats), unless otherwise indicated. The amino acid sequence of human CD20 is shown in Uniprot accession no. P11836 (version 149, SEQ ID NO:95). “CD33” refers to Myeloid cell surface antigen CD33, also known as SIGLEC3 or gp67, and includes any native CD33 from any vertebrate source, including mammals such as primates (e.g. humans) non-human primates (e.g. cynomolgus monkeys) and rodents (e.g. mice and rats), unless otherwise indicated. The amino acid sequence of human CD33 is shown in Uniprot accession no. P20138 (version 157, SEQ ID NO:96).

The term “PD-L1”, also known as CD274 or B7-H1, refers to any native PD-L1 from any vertebrate source, including mammals such as primates (e.g. humans) non-human primates (e.g. cynomolgus monkeys) and rodents (e.g. mice and rats), in particular to “human PD-L1”. The amino acid sequence of complete human PD-L1 is shown in UniProt (www.uniprot.org) accession no. Q9NZQ7 (SEQ ID NO:106). The term “anti-PD-L1 antibody” or “antibody binding to human PD-L1” or “antibody that specifically binds to human PD-L1” or “antagonistic anti-PD-L1” refers to an antibody specifically binding to the human PD-L1 antigen with a binding affinity of KD-value of 1.0×10⁻⁸ mol/l or lower, in one aspect of a KD-value of 1.0×10⁻⁹ mol/l or lower. The binding affinity is determined with a standard binding assay, such as surface plasmon resonance technique (BIAcore®, GE-Healthcare Uppsala, Sweden).

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W. H. Freeman and Co., page 91 (2007). A single VH or VL domain may be sufficient to confer antigen-binding specificity.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system. Kabat et al. defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” or “Kabat EU index” refers to the numbering system set forth by Kabat et al., Sequences of Proteins of Immunological Interest, 5th Ed. Public Health Service, National Institutes of Health, Bethesda, Md. (1991). Unless otherwise specified, references to the numbering of specific amino acid residue positions in an antibody variable region are according to the Kabat EU index numbering system.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “Fc domain” or “Fc region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (K447), or the C-terminal glycine (G446) and lysine (K447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, numbering according to EU index of Kabat). In one aspect, a heavy chain including an Fc region as specified herein, comprised in an antibody according to the invention, comprises an additional C-terminal glycine residue (G446, numbering according to EU index of Kabat). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The “knob-into-hole” technology is described e.g. in U.S. Pat. No. 5,731,168; U.S. Pat. No. 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. Fc γ RIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, 5239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “ADCC” or “antibody-dependent cellular cytotoxicity” is a function mediated by Fc receptor binding and refers to lysis of target cells by an antibody as reported herein in the presence of effector cells. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA) In particular, binding to FcγR on NK cells is measured.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The “Tumor Necrosis factor receptor superfamily” or “TNF receptor superfamily” currently consists of 27 receptors. It is a group of cytokine receptors characterized by the ability to bind tumor necrosis factors (TNFs) via an extracellular cysteine-rich domain (CRD). These pseudorepeats are defined by intrachain disulphides generated by highly conserved cysteine residues within the receptor chains.With the exception of nerve growth factor (NGF), all TNFs are homologous to the archetypal TNF-alpha. In their active form, the majority of TNF receptors form trimeric complexes in the plasma membrame. Accordingly, most TNF receptors contain transmembrane domains (TMDs). Several of these receptors also contain intracellular death domains (DDs) that recruit caspase-interacting proteins following ligand binding to initiate the extrinsic pathway of caspase activation. Other TNF superfamily receptors that lack death domains bind TNF receptor-associated factors and activate intracellular signaling pathways that can lead to proliferation or differentiation. These receptors can also initiate apoptosis, but they do so via indirect mechanisms. In addition to regulating apoptosis, several TNF superfamily receptors are involved in regulating immune cell functions such as B cell homeostasis and activation, natural killer cell activation, and T cell co-stimulation. Several others regulate cell type-specific responses such as hair follicle development and osteoclast development. Members of the TNF receptor superfamily include the following: Tumor necrosis factor receptor 1 (1A) (TNFRSF1A, CD120a), Tumor necrosis factor receptor 2 (1B) (TNFRSF1B, CD120b), Lymphotoxin beta receptor (LTBR, CD18), OX40 (TNFRSF4, CD134), CD40 (Bp50), Fas receptor (Apo-1, CD95, FAS), Decoy receptor 3 (TR6, M68, TNFRSF6B), CD27 (S152, Tp55), CD30 (Ki-1, TNFRSF8), 4-1BB (CD137, TNFRSF9), DR4 (TRAILR1, Apo-2, CD261, TNFRSF10A), DR5 (TRAILR2, CD262, TNFRSF10B), Decoy Receptor 1 (TRAILR3, CD263, TNFRSF10C), Decoy Receptor 2 (TRAILR4, CD264, TNFRSF10D), RANK (CD265, TNFRSF11A), Osteoprotegerin (OCIF, TR1, TNFRSF11B), TWEAK receptor (Fn14, CD266, TNFRSF12A), TACI (CD267, TNFRSF13B), BAFF receptor (CD268, TNFRSF13C), Herpesvirus entry mediator (HVEM, TR2, CD270, TNFRSF14), Nerve growth factor receptor (p75NTR, CD271, NGFR), B-cell maturation antigen (CD269, TNFRSF17), Glucocorticoid-induced TNFR-related (GITR, AITR, CD357, TNFRSF18), TROY (TNFRSF19), DR6 (CD358, TNFRSF21), DR3 (Apo-3, TRAMP, WS-1, TNFRSF25) and Ectodysplasin A2 receptor (XEDAR, EDA2R).

Several members of the tumor necrosis factor receptor (TNFR) family function after initial T cell activation to sustain T cell responses. The term “costimulatory TNF receptor family member” or “costimulatory TNF family receptor” refers to a subgroup of TNF receptor family members, which are able to costimulate proliferation and cytokine production of T-cells. The term refers to any native TNF family receptor from any vertebrate source, including mammals such as primates (e.g. humans), non-human primates (e.g. cynomolgus monkeys) and rodents (e.g. mice and rats), unless otherwise indicated. In specific embodiments of the invention, costimulatory TNF receptor family members are selected from the group consisting of OX40 (CD134), 4-1BB (CD137), CD27, HVEM (CD270), CD30, and GITR, all of which can have costimulatory effects on T cells. More particularly, the costimulatory TNF receptor family member is 4-1BB.

The term “4-1BB”, as used herein, refers to any native 4-1BB from any vertebrate source, including mammals such as primates (e.g. humans) and rodents (e.g., mice and rats), unless otherwise indicated. The term encompasses “full-length,” unprocessed 4-1BB as well as any form of 4-1BB that results from processing in the cell. The term also encompasses naturally occurring variants of 4-1BB, e.g., splice variants or allelic variants. The amino acid sequence of an exemplary human 4-1BB is shown in SEQ ID NO:97 (Uniprot accession no. Q07011), the amino acid sequence of an exemplary murine 4-1BB is shown in SEQ ID NO: 98 (Uniprot accession no. P20334) and the amino acid sequence of an exemplary cynomolgous 4-1BB (from Macaca mulatta) is shown in SEQ ID NO:99 (Uniprot accession no. F6W5G6).

The terms “anti-4-1BB antibody”, “anti-4-1BB”, “4-1BB antibody and “an antibody that specifically binds to 4-1BB” refer to an antibody that is capable of binding 4-1BB with sufficient affinity such that the antibody is useful as a diagnostic and/or therapeutic agent in targeting 4-1BB. In one embodiment, the extent of binding of an anti-4-1BB antibody to an unrelated, non-4-1BB protein is less than about 10% of the binding of the antibody to 4-1BB as measured, e.g., by a radioimmunoassay (RIA) or flow cytometry (FACS). In certain embodiments, an antibody that binds to 4-1BB has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁶ M or less, e.g. from 10⁻⁶⁸ M to 10⁻¹³ M, e.g., from 10⁻⁸ M to 10⁻¹⁰ M). In particular, the anti-4-1BB antibody is clone 20H4.9 as disclosed in U.S. Pat. No. 7,288,638.

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 2 and 4, in particular 2, i.e. the peptides selected from the group consisting of GGGGS (SEQ ID NO: 100) GGGGSGGGGS (SEQ ID NO:101), SGGGGSGGGG (SEQ ID NO:102) and GGGGSGGGGSGGGG (SEQ ID NO:103), but also include the sequences GSPGSSSSGS (SEQ ID NO:104), (G4S)₃ (SEQ ID NO:105), (G4S)₄ (SEQ ID NO:106), GSGSGSGS (SEQ ID NO:107), GSGSGNGS (SEQ ID NO:108), GGSGSGSG (SEQ ID NO:109), GGSGSG (SEQ ID NO:110), GGSG (SEQ ID NO:111), GGSGNGSG (SEQ ID NO:112), GGNGSGSG (SEQ ID NO:113) and GGNGSG (SEQ ID NO:114). Peptide linkers of particular interest are (G4S) (SEQ ID NO:100), (G₄S)₂ or GGGGSGGGGS (SEQ ID NO:101), (G4S)₃ (SEQ ID NO:105) and (G4S)₄ (SEQ ID NO:106), more particularly (G₄S)₂ or GGGGSGGGGS (SEQ ID NO:101).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

By “fused” or “connected” is meant that the components (e.g. a heavy chain of an antibody and a Fab fragment) are linked by peptide bonds, either directly or via one or more peptide linkers.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

In certain embodiments, amino acid sequence variants of the TNF ligand trimer-containing antigen binding molecules provided herein are contemplated. For example, it may be desirable to improve the binding affinity and/or other biological properties of the TNF ligand trimer-containing antigen binding molecules. Amino acid sequence variants of the TNF ligand trimer-containing antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table A under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE A

| | | Preferred |
|---|---|---|
| Original Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more HVR residues are mutated and the variant antigen binding molecules displayed on phage and screened for a particular biological activity (e.g. binding affinity). In certain embodiments, substitutions, insertions, or deletions may occur within one or more HVRs so long as such alterations do not substantially reduce the ability of the antigen binding molecule to bind antigen. For example, conservative alterations (e.g., conservative substitutions as provided herein) that do not substantially reduce binding affinity may be made in HVRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include bispecific antigen binding molecules of the invention with an N-terminal methionyl residue. Other insertional variants of the molecule include the fusion to the N- or C-terminus to a polypeptide which increases the serum half-life of the bispecific antigen binding molecules.

In certain embodiments, the bispecific antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the TNF ligand trimer-containing antigen binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in TNF family ligand trimer-containing antigen binding molecule may be made in order to create variants with certain improved properties. In one aspect, variants of bispecific antigen binding molecules or antibodies of the invention are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). In another aspect, variants of the bispecific antigen binding molecules or antibodies of the invention are provided with bisected oligosaccharides, e.g., in which a biantennary oligosaccharide attached to the Fc region is bisected by GlcNAc. Such variants may have reduced fucosylation and/or improved ADCC function., see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain aspects, it may be desirable to create cysteine engineered variants of the bispecific antigen binding molecules of the invention, e.g., “thioMAbs,” in which one or more residues of the molecule are substituted with cysteine residues. In particular aspects, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain aspects, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antigen binding molecules may be generated as described, e.g., in U.S. Pat. No. 7,521,541.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi:10.1038/nm.4356 or EP 2 101 823 B1).

An “isolated” polynucleotide refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated polynucleotide encoding a bispecific antigen binding molecule” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

Bispecific Antigen Binding Molecules of the Invention

The invention relates to novel bispecific agonistic 4-1BB antibodies with particularly advantageous properties such as producibility, stability, binding affinity, biological activity, targeting efficiency and reduced toxicity. They are further characterized by their specific structural features such as proximity of two Fab fragments capable of specific binding to 4-1BB and the target cell antigen, respectively, bivalent binding to 4-1BB and monovalent binding to the target cell antigen which makes these bispecific antigen binding molecules very potent without hampering the safety.

Exemplary Bispecific Antigen Binding Molecules

In one aspect, the invention provides a bispecific antigen binding molecule, comprising
(a) a first Fab fragment capable of specific binding to 4-1BB,
(b) a second Fab fragment capable of specific binding to a target cell antigen,
(c) a third Fab fragment capable of specific binding to 4-1BB, and
(d) a Fc domain composed of a first and a second subunit capable of stable association, wherein the second Fab fragment (b) is fused at the C-terminus of the Fab heavy chain to the N-terminus of the Fab heavy chain of the first Fab fragment (a), which is in turn fused at its C-terminus to the N-terminus of the first Fc domain subunit, and the third Fab fragment (c) is fused at the C-terminus of the Fab heavy chain to the N-terminus of the second Fc domain subunit, and wherein in the second Fab fragment capable of specific binding to a target cell antigen (i) the variable regions VL and VH of the Fab light chain and Fab heavy chain are replaced by each other, or (ii) the constant regions CL and CH1 of the Fab light chain and Fab heavy chain are replaced by each other.

In one aspect, the invention provides a bispecific antigen binding molecule, comprising
(a) a first Fab fragment capable of specific binding to 4-1BB,
(b) a second Fab fragment capable of specific binding to a target cell antigen,
(c) a third Fab fragment capable of specific binding to 4-1BB, and
(d) a Fc domain composed of a first and a second subunit capable of stable association, wherein the second Fab fragment (b) is fused at the C-terminus of the Fab heavy chain to the N-terminus of the Fab heavy chain of the first Fab fragment (a), which is in turn fused at its C-terminus to the N-terminus of the first Fc domain subunit, and the third Fab fragment (c) is fused at the C-terminus of the Fab heavy chain to the N-terminus of the second Fc domain subunit, and wherein in the second Fab fragment capable of specific binding to a target cell antigen the variable regions VL and VH of the Fab light chain and Fab heavy chain are replaced by each other.

In particular, the Fab fragment capable of specific binding to 4-1BB comprises a heavy chain variable region (V_{H}4-1BB) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a light chain variable region (V_{L}4-1BB) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6.

In a particular aspect, provided is a bispecific antigen binding molecule as described herein, wherein the bispecific antigen binding molecule provides bivalent binding to 4-1BB and monovalent binding to the target cell antigen. More particularly, the bispecific antigen binding molecule provides bivalent binding to 4-1BB and monovalent binding to a tumor-associated antigen (TAA).

The Fab fragments may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G4S)n, (SG4)n, (G4S)n or G4(SG4)n peptide linkers. “n” is generally an integer from 1 to 10, typically from 2 to 4. An exemplary peptide linker suitable for connecting the Fab heavy chains of the first and the second Fab fragments comprises the sequence (D)-(G₄S)₂. Another suitable such linker comprises the sequence (G₄S)₄. Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In one aspect, provided is a bispecific antigen binding molecule as described herein before, wherein the first and the third Fab fragment capable of specific binding to 4-1BB (a) and (c) are identical. In a particular aspect, the invention provides a a bispecific antigen binding molecule as described herein before, wherein the first and the third Fab fragment capable of specific binding to 4-1BB each comprise a heavy chain variable region (V_{H}4-1BB) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a light chain variable region (V_{L}4-1BB) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6.

Thus, in one aspect, provided is a bispecific antigen binding molecule, comprising
(a) a first Fab fragment capable of specific binding to 4-1BB,
(b) a second Fab fragment capable of specific binding to a target cell antigen,
(c) a third Fab fragment capable of specific binding to 4-1BB, and
(d) a Fc domain composed of a first and a second subunit capable of stable association, wherein the second Fab fragment (b) is fused at the C-terminus of the Fab heavy chain to the N-terminus of the Fab heavy chain of the first Fab fragment (a), which is in turn fused at its C-terminus to the N-terminus of the first Fc domain subunit, and the third Fab fragment (c) is fused at the C-terminus of the Fab heavy chain to the N-terminus of the second Fc domain subunit, and wherein in the second Fab fragment capable of specific binding to a target cell antigen (i) the variable regions VL and VH of the Fab light chain and Fab heavy chain are replaced by each other, or (ii) the constant regions CL and CH1 of the Fab light chain and Fab heavy chain are replaced by each other, and wherein the first and the third Fab fragment each comprises a heavy chain variable region (V_{H}4-1BB) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a light chain variable region (V_{L}4-1BB) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6.

In a particular aspect, the bispecific antigen binding molecule comprises a first and a third Fab fragment capable of specific binding to 4-1BB each comprising a heavy chain variable region (V_{H}4-1BB) comprising an amino acid sequence that is at least about 95%, 96%, 97%, 98%, 99% or 100% identical to the amino acid sequence of SEQ ID NO:7 and a light chain variable region (V_{L}4-1BB) comprising an amino acid sequence that is at least about 95%, 96%, 97%, 98%, 99% or 100% identical to the amino acid sequence of SEQ ID NO:8. More particularly, the first and the third Fab fragment capable of specific binding to 4-1BB each comprise a heavy chain variable region (V_{H}4-1BB) comprising the amino acid sequence of SEQ ID NO:7 and a light chain variable region (V_{L}4-1BB) comprising the amino acid sequence of SEQ ID NO:8.

In one aspect, the bispecific antigen binding molecule comprises a polypeptide (Fc hole heavy chain) comprising the amino acid sequence of SEQ ID NO:65 and two polypeptides (light chains) comprising the amino acid sequence of SEQ ID NO:67.

The bispecific antigen binding molecules of the invention are further characterized by comprising one Fab fragment capable of specific binding to a target cell antigen. The bispecific antigen binding molecules thus possess the advantage over conventional antibodies capable of specific binding to 4-1BB, that they selectively induce a costimulatory T cell response at the target cells, which are typically cancer cells. In one aspect, the target cell antigen is selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20, CD33 and PD-L1. In one aspect, the target cell antigen is selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20 and CD33. Particularly, the target cell antigen is selected from Fibroblast Activation Protein (FAP), Carcinoembryonic Antigen (CEA) and CD19. In one particular aspect, the target cell antigen is selected from Fibroblast Activation Protein (FAP) and Carcinoembryonic Antigen (CEA). More particularly, the target cell antigen is FAP. Alternatively, the target cell antigen is CEA. In another particular aspect, the target cell antigen is CD19. In a further aspect, the target cell antigen is PD-L1.

Bispecific Antigen Binding Molecules Wherein the Target Cell Antigen is FAP

In a particular aspect, the target cell antigen is Fibroblast Activation Protein (FAP). FAP binding moieties have been described in WO 2012/02006 which is included by reference in its entirety. FAP binding moieties of particular interest are described below.

In one aspect, the invention provides a bispecific antigen binding molecule, wherein the Fab fragment capable of specific binding to Fibroblast Activation Protein (FAP) comprises (a) a heavy chain variable region (V_{H}FAP) comprising (i) CDR-H1 comprising the amino acid sequence of SEQ ID NO:9, (ii) CDR-H2 comprising the amino acid sequence of SEQ ID NO:10, and (iii) CDR-H3 comprising the amino acid sequence of SEQ ID NO:11, and a light chain variable region (V_{L}FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14, or
(b) a heavy chain variable region (V_{H}FAP) comprising (i) CDR-H1 comprising the amino acid sequence of SEQ ID NO:15, (ii) CDR-H2 comprising the amino acid sequence of SEQ ID NO:16, and (iii) CDR-H3 comprising the amino acid sequence of SEQ ID NO:17, and a a light chain variable region (V_{L}FAP) comprising (iv) CDR-L1 comprising the amino acid sequence of SEQ ID NO:18, (v) CDR-L2 comprising the amino acid sequence of SEQ ID NO:19, and (vi) CDR-L3 comprising the amino acid sequence of SEQ ID NO:20.

In particular, provided is a a bispecific antigen binding molecule, wherein the Fab fragment capable of specific binding to FAP comprises a heavy chain variable region (V_{H}FAP) comprising (i) CDR-H1 comprising the amino acid sequence of SEQ ID NO:9, (ii) CDR-H2 comprising the amino acid sequence of SEQ ID NO:10, and (iii) CDR-H3 comprising the amino acid sequence of SEQ ID NO:11, and a a light chain variable region (V_{L}FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14.

In another aspect, the Fab fragment capable of specific binding to FAP comprises a heavy chain variable region (V_{H}FAP) comprising (i) CDR-H1 comprising the amino acid sequence of SEQ ID NO:15, (ii) CDR-H2 comprising the amino acid sequence of SEQ ID NO:16, and (iii) CDR-H3 comprising the amino acid sequence of SEQ ID NO:17, and a light chain variable region (V_{L}FAP) comprising (iv) CDR-L1 comprising the amino acid sequence of SEQ ID NO:18, (v) CDR-L2 comprising the amino acid sequence of SEQ ID NO:19, and (vi) CDR-L3 comprising the amino acid sequence of SEQ ID NO:20.

Particularly, provided is a bispecific antigen binding molecule, wherein the Fab fragment capable of specific binding to Fibroblast Activation Protein (FAP) comprises
(a) a heavy chain variable region (V_{H}FAP) comprising an amino acid sequence that is at least about 95%, 96%, 97%, 98%, 99% or 100% identical to the amino acid sequence of SEQ ID NO:21, and a light chain variable region (V_{L}FAP) comprising an amino acid sequence that is at least about 95%, 96%, 97%, 98%, 99% or 100% identical to the amino acid sequence of SEQ ID NO:22, or
(b) a heavy chain variable region (V_{H}FAP) comprising an amino acid sequence that is at least about 95%, 96%, 97%, 98%, 99% or 100% identical to the amino acid sequence of SEQ ID NO:23, and a light chain variable region (V_{L}FAP) comprising an amino acid sequence that is at least about 95%, 96%, 97%, 98%, 99% or 100% identical to the amino acid sequence of SEQ ID NO:24.

In particular, the Fab fragment capable of specific binding to FAP comprises a heavy chain variable region (V_{H}FAP) comprising the amino acid sequence of SEQ ID NO:21, and a light chain variable region (V_{L}FAP) comprising the amino acid sequence of SEQ ID NO:22, or a heavy chain variable region (V_{H}FAP) comprising the amino acid sequence of SEQ ID NO:23, and a light chain variable region (V_{L}FAP) comprising the amino acid sequence of SEQ ID NO:24. More particularly, the Fab fragment capable of specific binding to FAP comprises a heavy chain variable region (V_{H}FAP) comprising the amino acid sequence of SEQ ID NO:21 and a light chain variable region (V_{L}FAP) comprising the amino acid sequence of SEQ ID NO:22.

In another aspect, provided is a bispecific antigen binding molecule, wherein
(i) the first and third Fab fragment capable of specific binding to 4-1BB each comprise a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:21 and a light chain variable region comprising an amino acid sequence of SEQ ID NO:22.

In a further aspect, provided is a bispecific antigen binding molecule, wherein
(i) the first and third Fab fragment capable of specific binding to 4-1BB each comprise a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:23 and a light chain variable region comprising an amino acid sequence of SEQ ID NO:24.

In a particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO: 66, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:68. In a further particular embodiment, the bispecific antigen binding molecule comprises a polypeptide sequence of SEQ ID NO:65, a polypeptide sequence of SEQ ID NO:66, two polypeptide sequences of SEQ ID NO:67 and a polypeptide sequence of SEQ ID NO:68.

Bispecific Antigen Binding Molecules Wherein the Target Cell Antigen is CEA

In a particular aspect, the target cell antigen is Carcinoembryonic Antigen (CEA). CEA binding moieties have been described for example in WO 92/01059, WO 2007/071422, WO 2016/075278 A2 or WO 2007/071426 which are included by reference in its entirety. CEA binding moieties of particular interest are described below.

In one aspect, the invention provides a bispecific antigen binding molecule, wherein the Fab fragment capable of specific binding to Carcinoembryonic Antigen (CEA) comprises (a) a heavy chain variable region (V_{H}CEA) comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a light chain variable region (V_{L}CEA) comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30, or
(b) a heavy chain variable region (V_{H}CEA) comprising (i) CDR-H1 comprising the amino acid sequence of SEQ ID NO:33, (ii) CDR-H2 comprising the amino acid sequence of SEQ ID NO:34, and (iii) CDR-H3 comprising the amino acid sequence of SEQ ID NO:35, and a light chain variable region (V_{L}CEA) comprising (iv) CDR-L1 comprising the amino acid sequence of SEQ ID NO:36, (v) CDR-L2 comprising the amino acid sequence of SEQ ID NO:37, and (vi) CDR-L3 comprising the amino acid sequence of SEQ ID NO:38, or
(c) a heavy chain variable region (V_{H}CEA) comprising (i) CDR-H1 comprising the amino acid sequence of SEQ ID NO:41, (ii) CDR-H2 comprising the amino acid sequence of SEQ ID NO:42, and (iii) CDR-H3 comprising the amino acid sequence of SEQ ID NO:43, and a light chain variable region (V_{L}CEA) comprising (iv) CDR-L1 comprising the amino acid sequence of SEQ ID NO:44, (v) CDR-L2 comprising the amino acid sequence of SEQ ID NO:45, and (vi) CDR-L3 comprising the amino acid sequence of SEQ ID NO:46, or
(d) a heavy chain variable region (V_{H}CEA) comprising (i) CDR-H1 comprising the amino acid sequence of SEQ ID NO:49, (ii) CDR-H2 comprising the amino acid sequence of SEQ ID NO:50, and (iii) CDR-H3 comprising the amino acid sequence of SEQ ID NO:51, and a light chain variable region (V_{L}CEA) comprising (iv) CDR-L1 comprising the amino acid sequence of SEQ ID NO:52, (v) CDR-L2 comprising the amino acid sequence of SEQ ID NO:53, and (vi) CDR-L3 comprising the amino acid sequence of SEQ ID NO:54, or
(e) a heavy chain variable region (V_{H}CEA) comprising (i) CDR-H1 comprising the amino acid sequence of SEQ ID NO:115, (ii) CDR-H2 comprising the amino acid sequence of SEQ ID NO:116, and (iii) CDR-H3 comprising the amino acid sequence of SEQ ID NO:117, and a light chain variable region (V_{L}CEA) comprising (iv) CDR-L1 comprising the amino acid sequence of SEQ ID NO:118, (v) CDR-L2 comprising the amino acid sequence of SEQ ID NO:119, and (vi) CDR-L3 comprising the amino acid sequence of SEQ ID NO:120, or
(f) a heavy chain variable region (V_{H}CEA) comprising (i) CDR-H1 comprising the amino acid sequence of SEQ ID NO:123, (ii) CDR-H2 comprising the amino acid sequence of SEQ ID NO:124 or SEQ ID NO:125, and (iii) CDR-H3 comprising the amino acid sequence of SEQ ID NO:126, and a light chain variable region (V_{L}CEA) comprising (iv) CDR-L1 comprising the amino acid sequence of SEQ ID NO:127 or SEQ ID NO:128, (v) CDR-L2 comprising the amino acid sequence of SEQ ID NO:129 or SEQ ID NO:130 or SEQ ID NO:131, and (vi) CDR-L3 comprising the amino acid sequence of SEQ ID NO:132.

In particular, provided is a bispecific antigen binding molecule, wherein the Fab fragment capable of specific binding to CEA comprises a heavy chain variable region (V_{H}CEA) comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a light chain variable region (V_{L}CEA) comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30.

Particularly, provided is a bispecific antigen binding molecule, wherein the Fab fragment capable of specific binding to Carcinoembryonic Antigen (CEA) comprises (a) a heavy chain variable region (V_{H}CEA) comprising an amino acid sequence that is at least about 95%, 96%, 97%, 98%, 99% or 100% identical to the amino acid sequence of SEQ ID NO:31, and a light chain variable region (V_{L}CEA) comprising an amino acid sequence that is at least about 95%, 96%, 97%, 98%, 99% or 100% identical to the amino acid sequence of SEQ ID NO:32. In one aspect, the Fab fragment capable of specific binding to Carcinoembryonic Antigen (CEA) comprises (a) a heavy chain variable region (V_{H}CEA) comprising the amino acid sequence of SEQ ID NO:31, and a light chain variable region (V_{L}CEA) comprising the amino acid sequence of SEQ ID NO:32 (antibody A5B7).

In one aspect, the Fab fragment capable of specific binding to Carcinoembryonic Antigen (CEA) comprises a humanized heavy chain variable region (V_{H}CEA) that is based on human acceptor framework IGHV3-23-02 comprising an amino acid sequence of SEQ ID NO: 153 and a humanized light chain variable region (V_{L}CEA) that is based on human acceptor framework IGKV3-11 comprising an amino acid sequence of SEQ ID NO: 165.

In one particular aspect, the Fab fragment capable of specific binding to CEA comprises a heavy chain variable region (V_{H}CEA) comprising (i) CDR-H1 comprising the amino acid sequence of SEQ ID NO:115, (ii) CDR-H2 comprising the amino acid sequence of SEQ ID NO:116, and (iii) CDR-H3 comprising the amino acid sequence of SEQ ID NO:117, and a light chain variable region (V_{L}CEA) comprising (iv) CDR-L1 comprising the amino acid sequence of SEQ ID NO:118, (v) CDR-L2 comprising the amino acid sequence of SEQ ID NO:119, and (vi) CDR-L3 comprising the amino acid sequence of SEQ ID NO:120.

In one aspect, the Fab fragment capable of specific binding to CEA comprises a heavy chain variable region (V_{H}CEA) comprising an amino acid sequence that is at least about 95%, 96%, 97%, 98%, 99% or 100% identical to the amino acid sequence of SEQ ID NO:121, and a light chain variable region (V_{L}CEA) comprising an amino acid sequence that is at least about 95%, 96%, 97%, 98%, 99% or 100% identical to the amino acid sequence of SEQ ID NO:122. In particular, the Fab fragment capable of specific binding to CEA comprises a heavy chain variable region (V_{H}CEA) comprising an amino acid sequence of SEQ ID NO:121 and a light chain variable region (V_{L}CEA) comprising an amino acid sequence of SEQ ID NO:122 (antibody A5H1EL1D). In another aspect, the Fab fragment may comprise an antibody with higher affinity towards CEA compared to A5H1EL1D that comprises identical framework regions, but mutations in the CDR regions. Thus, a Fab fragment capable of specific binding to CEA comprising a heavy chain variable region (V_{H}CEA) comprising an amino acid sequence that is at least about 95% identical to the amino acid sequence of SEQ ID NO:121 and a light chain variable region (V_{L}CEA) comprising an amino acid sequence that is at least about 95%, identical to the amino acid sequence of SEQ ID NO:122 is also provided.

In another aspect, the Fab fragment capable of specific binding to CEA comprises a heavy chain variable region (V_{H}CEA) comprising (i) CDR-H1 comprising the amino acid sequence of SEQ ID NO:33, (ii) CDR-H2 comprising the amino acid sequence of SEQ ID NO:34, and (iii) CDR-H3 comprising the amino acid sequence of SEQ ID NO:35, and a light chain variable region (V_{L}CEA) comprising (iv) CDR-L1 comprising the amino acid sequence of SEQ ID NO:36, (v) CDR-L2 comprising the amino acid sequence of SEQ ID NO:37, and (vi) CDR-L3 comprising the amino acid sequence of SEQ ID NO:38.

In one aspect, the Fab fragment capable of specific binding to CEA a heavy chain variable region (V_{H}CEA) comprising an amino acid sequence that is at least about 95%, 96%, 97%, 98%, 99% or 100% identical to the amino acid sequence of SEQ ID NO:39, and a light chain variable region (V_{L}CEA) comprising an amino acid sequence that is at least about 95%, 96%, 97%, 98%, 99% or 100% identical to the amino acid sequence of SEQ ID NO:40. In one aspect, the Fab fragment capable of specific binding to CEA a heavy chain variable region (V_{H}CEA) comprising the amino acid sequence of SEQ ID NO:39 and a light chain variable region (V_{L}CEA) comprising the amino acid sequence of SEQ ID NO:40 (antibody MFE23).

In particular, provided is a bispecific antigen binding molecule, wherein wherein the Fab fragment capable of specific binding to Carcinoembryonic Antigen (CEA) comprises humanized heavy chain and light chain variable domains. In one aspect, the Fab fragment capable of specific binding to Carcinoembryonic Antigen (CEA) comprises a heavy chain variable region (V_{H}CEA) comprising the amino acid sequence of SEQ ID NO:133, SEQ ID NO:134, SEQ ID NO:135, SEQ ID NO:136, SEQ ID NO:137 or SEQ ID NO:138, and a light chain variable region (V_{L}CEA) comprising the amino acid sequence of SEQ ID NO:139, SEQ ID NO:140, SEQ ID NO:141, SEQ ID NO:142, SEQ ID NO:143 or SEQ ID NO:144.

In one aspect, the Fab fragment capable of specific binding to Carcinoembryonic Antigen (CEA) comprises

(a) a heavy chain variable region (V_{H}CEA) comprising an amino acid sequence of SEQ ID NO:133, and a light chain variable region (V_{L}CEA) comprising an amino acid sequence of SEQ ID NO:143, or

(b) a heavy chain variable region (V_{H}CEA) comprising an amino acid sequence of SEQ ID NO:137, and a light chain variable region (V_{L}CEA) comprising an amino acid sequence of SEQ ID NO:143, or

(c) a heavy chain variable region (V_{H}CEA) comprising an amino acid sequence of SEQ ID NO:134, and a light chain variable region (V_{L}CEA) comprising an amino acid sequence of SEQ ID NO:143, or

(d) a heavy chain variable region (V_{H}CEA) comprising an amino acid sequence of SEQ ID NO:138, and a light chain variable region (V_{L}CEA) comprising an amino acid sequence of SEQ ID NO:142, or

(e) a heavy chain variable region (V_{H}CEA) comprising an amino acid sequence of SEQ ID NO:137, and a light chain variable region (V_{L}CEA) comprising an amino acid sequence of SEQ ID NO:142, or

(f) a heavy chain variable region (V_{H}CEA) comprising an amino acid sequence of SEQ ID NO:135, and a light chain variable region (V_{L}CEA) comprising an amino acid sequence of SEQ ID NO:142, or

(g) a heavy chain variable region (V_{H}CEA) comprising an amino acid sequence of SEQ ID NO:133, and a light chain variable region (V_{L}CEA) comprising an amino acid sequence of SEQ ID NO:142.

In a further aspect, the Fab fragment capable of specific binding to CEA comprises a heavy chain variable region (V_{H}CEA) comprising (i) CDR-H1 comprising the amino acid sequence of SEQ ID NO:41, (ii) CDR-H2 comprising the amino acid sequence of SEQ ID NO:42, and (iii) CDR-H3 comprising the amino acid sequence of SEQ ID NO:43, and a light chain variable region (V_{L}CEA) comprising (iv) CDR-L1 comprising the amino acid sequence of SEQ ID NO:44, (v) CDR-L2 comprising the amino acid sequence of SEQ ID NO:45, and (vi) CDR-L3 comprising the amino acid sequence of SEQ ID NO:46.

In one aspect, the Fab fragment capable of specific binding to CEA comprises a heavy chain variable region (V_{H}CEA) comprising an amino acid sequence that is at least about 95%, 96%, 97%, 98%, 99% or 100% identical to the amino acid sequence of SEQ ID NO:47, and a light chain variable region (V_{L}CEA) comprising an amino acid sequence that is at least about 95%, 96%, 97%, 98%, 99% or 100% identical to the amino acid sequence of SEQ ID NO:48. Particularly, the Fab fragment capable of specific binding to CEA comprises a heavy chain variable region (V_{H}CEA) comprising the amino acid sequence of SEQ ID NO:47 and a light chain variable region (V_{L}CEA) comprising the amino acid sequence of SEQ ID NO:48 (antibody T84.66-LCHA).

In a further aspect, the Fab fragment capable of specific binding to CEA comprises a heavy chain variable region (V_{H}CEA) comprises (i) CDR-H1 comprising the amino acid sequence of SEQ ID NO:49, (ii) CDR-H2 comprising the amino acid sequence of SEQ ID NO:50, and (iii) CDR-H3 comprising the amino acid sequence of SEQ ID NO:51, and a light chain variable region (V_{L}CEA) comprising (iv) CDR-L1 comprising the amino acid sequence of SEQ ID NO:52, (v) CDR-L2 comprising the amino acid sequence of SEQ ID NO:53, and (vi) CDR-L3 comprising the amino acid sequence of SEQ ID NO:54.

In one aspect, the Fab fragment capable of specific binding to CEA comprises a heavy chain variable region (V_{H}CEA) comprises a heavy chain variable region (V_{H}CEA) comprising an amino acid sequence that is at least about 95%, 96%, 97%, 98%, 99% or 100% identical to the amino acid sequence of SEQ ID NO:55, and a light chain variable region (V_{L}CEA) comprising an amino acid sequence that is at least about 95%, 96%, 97%, 98%, 99% or 100% identical to the amino acid sequence of SEQ ID NO:56. In particular, the Fab fragment capable of specific binding to CEA comprises a heavy chain variable region (V_{H}CEA) comprises a heavy chain variable region (V_{H}CEA) comprising the amino acid sequence of SEQ ID NO:55 and a light chain variable region (V_{L}CEA) comprising the amino acid sequence of SEQ ID NO:56 (antibody CH1A1A 98/99/2F1).

In one aspect, the antigen binding domain capable of specific binding to CEA comprises a heavy chain variable region VH comprising the amino acid sequence of SEQ ID NO: 31 and a light chain variable region comprising an amino acid sequence of SEQ ID NO: 32 or the antigen binding domain capable of specific binding to FAP comprises a heavy chain variable region VH comprising an amino acid sequence of SEQ ID NO:39 and a light chain variable region comprising an amino acid sequence of SEQ ID NO:40.

In a further aspect, the antigen binding domain capable of specific binding to CEA comprises a heavy chain variable region VH comprising the amino acid sequence of SEQ ID NO:47 and a light chain variable region comprising an amino acid sequence of SEQ ID NO:48 or the antigen binding domain capable of specific binding to FAP comprises a heavy chain variable region VH comprising an amino acid sequence of SEQ ID NO:55 and a light chain variable region comprising an amino acid sequence of SEQ ID NO:56.

In another aspect, provided is a bispecific antigen binding molecule, wherein
(i) the first and third Fab fragment capable of specific binding to 4-1BB comprises a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:31 and a light chain variable region comprising an amino acid sequence of SEQ ID NO:32.

In a further aspect, provided is a bispecific antigen binding molecule, wherein
(i) the first and third Fab fragment capable of specific binding to 4-1BB comprises a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:39 and a light chain variable region comprising an amino acid sequence of SEQ ID NO:40.

In a further aspect, provided is a bispecific antigen binding molecule, wherein
(i) the first and third Fab fragment capable of specific binding to 4-1BB comprises a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:47 and a light chain variable region comprising an amino acid sequence of SEQ ID NO:48.

In another aspect, provided is a bispecific antigen binding molecule, wherein
(i) the first and third Fab fragment capable of specific binding to 4-1BB comprises a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:55 and a light chain variable region comprising an amino acid sequence of SEQ ID NO:56.

In another aspect, provided is a bispecific antigen binding molecule, wherein
(i) the first and third Fab fragment capable of specific binding to 4-1BB comprises a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:121 and a light chain variable region comprising an amino acid sequence of SEQ ID NO:122.

In a further aspect, provided is a bispecific antigen binding molecule, wherein
(i) the first and third Fab fragment capable of specific binding to 4-1BB comprises a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:133 and a light chain variable region comprising an amino acid sequence of SEQ ID NO:143.

In another aspect, provided is a bispecific antigen binding molecule, wherein
(i) the first and third Fab fragment capable of specific binding to 4-1BB comprises a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:137 and a light chain variable region comprising an amino acid sequence of SEQ ID NO:143.

In another aspect, provided is a bispecific antigen binding molecule, wherein
(i) the first and third Fab fragment capable of specific binding to 4-1BB comprises a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:134 and a light chain variable region comprising an amino acid sequence of SEQ ID NO:143.

In yet another aspect, provided is a bispecific antigen binding molecule, wherein
(i) the first and third Fab fragment capable of specific binding to 4-1BB comprises a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:138 and a light chain variable region comprising an amino acid sequence of SEQ ID NO:142.

In another aspect, provided is a bispecific antigen binding molecule, wherein
(i) the first and third Fab fragment capable of specific binding to 4-1BB comprises a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:137 and a light chain variable region comprising an amino acid sequence of SEQ ID NO:142.

In another aspect, provided is a bispecific antigen binding molecule, wherein
(i) the first and third Fab fragment capable of specific binding to 4-1BB comprises a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:135 and a light chain variable region comprising an amino acid sequence of SEQ ID NO:142.

In yet a further aspect, provided is a bispecific antigen binding molecule, wherein
(i) the first and third Fab fragment capable of specific binding to 4-1BB comprises a heavy chain variable region VH comprising the amino acid sequence of SEQ ID NO: 7 and a light chain variable region comprising an amino acid sequence of SEQ ID NO: 8 and
(ii) the second Fab fragment capable of specific binding to FAP comprises a heavy chain variable region VH comprising an amino acid sequence of SEQ ID NO:133 and a light chain variable region comprising an amino acid sequence of SEQ ID NO:142.

In a particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:76, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:77. In a further particular embodiment, the bispecific antigen binding molecule comprises a polypeptide sequence of SEQ ID NO:65, a polypeptide sequence of SEQ ID NO:76, two polypeptide sequences of SEQ ID NO:67 and a polypeptide sequence of SEQ ID NO:77.

In another particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:78, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:79. In a further particular embodiment, the bispecific antigen binding molecule comprises a polypeptide sequence of SEQ ID NO:65, a polypeptide sequence of SEQ ID NO:78, two polypeptide sequences of SEQ ID NO:67 and a polypeptide sequence of SEQ ID NO:79.

In a further particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:80, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:81. In a further particular embodiment, the bispecific antigen binding molecule comprises a polypeptide sequence of SEQ ID NO:65, a polypeptide sequence of SEQ ID NO:80, two polypeptide sequences of SEQ ID NO:67 and a polypeptide sequence of SEQ ID NO:81.

In yet another particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:82, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:83. In a further particular embodiment, the bispecific antigen binding molecule comprises a polypeptide sequence of SEQ ID NO:65, a polypeptide sequence of SEQ ID NO:82, two polypeptide sequences of SEQ ID NO:67 and a polypeptide sequence of SEQ ID NO:83.

In a further particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:173, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:174. In a further particular embodiment, the bispecific antigen binding molecule comprises a polypeptide sequence of SEQ ID NO:65, a polypeptide sequence of SEQ ID NO:173, two polypeptide sequences of SEQ ID NO:67 and a polypeptide sequence of SEQ ID NO:174.

In another particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:179, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:180. In a further particular embodiment, the bispecific antigen binding molecule comprises a polypeptide sequence of SEQ ID NO:65, a polypeptide sequence of SEQ ID NO:179, two polypeptide sequences of SEQ ID NO:67 and a polypeptide sequence of SEQ ID NO:180.

In another particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:181, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:182. In a further particular embodiment, the bispecific antigen binding molecule comprises a polypeptide sequence of SEQ ID NO:65, a polypeptide sequence of SEQ ID NO:181, two polypeptide sequences of SEQ ID NO:67 and a polypeptide sequence of SEQ ID NO:182.

In another particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:183, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:184. In a further particular embodiment, the bispecific antigen binding molecule comprises a polypeptide sequence of SEQ ID NO:65, a polypeptide sequence of SEQ ID NO:183, two polypeptide sequences of SEQ ID NO:67 and a polypeptide sequence of SEQ ID NO:184.

In another particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:185, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:186. In a further particular embodiment, the bispecific antigen binding molecule comprises a polypeptide sequence of SEQ ID NO:65, a polypeptide sequence of SEQ ID NO:185, two polypeptide sequences of SEQ ID NO:67 and a polypeptide sequence of SEQ ID NO:186.

In another particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:187, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:188. In a further particular embodiment, the bispecific antigen binding molecule comprises a polypeptide sequence of SEQ ID NO:65, a polypeptide sequence of SEQ ID NO:187, two polypeptide sequences of SEQ ID NO:67 and a polypeptide sequence of SEQ ID NO:188.

In another particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:189, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:190. In a further particular embodiment, the bispecific antigen binding molecule comprises a polypeptide sequence of SEQ ID NO:65, a polypeptide sequence of SEQ ID NO:189, two polypeptide sequences of SEQ ID NO:67 and a polypeptide sequence of SEQ ID NO:190.

In another particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:191, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:192. In a further particular embodiment, the bispecific antigen binding molecule comprises a polypeptide sequence of SEQ ID NO:65, a polypeptide sequence of SEQ ID NO:191, two polypeptide sequences of SEQ ID NO:67 and a polypeptide sequence of SEQ ID NO:192.

Bispecific Antigen Binding Molecules Wherein the Target Cell Antigen is CD19

In a particular aspect, the target cell antigen is CD19. CD19 binding moieties have been described for example in WO 2016/075278 A1 which is included by reference in its entirety. CD19 binding moieties of particular interest are described below.

In one aspect, the invention provides a bispecific antigen binding molecule, wherein the Fab fragment capable of specific binding to CD19 comprises
(a) a heavy chain variable region (V_{H}CD19) comprising (i) CDR-H1 comprising the amino acid sequence of SEQ ID NO:57, (ii) CDR-H2 comprising the amino acid sequence of SEQ ID NO:58, and (iii) CDR-H3 comprising the amino acid sequence of SEQ ID NO:59, and a light chain variable region (V_{L}CD19) comprising (iv) CDR-L1 comprising the amino acid sequence of SEQ ID NO:60, (v) CDR-L2 comprising the amino acid sequence of SEQ ID NO:61, and (vi) CDR-L3 comprising the amino acid sequence of SEQ ID NO:62.

Particularly, the Fab fragment capable of specific binding to CD19 comprises a heavy chain variable region (V_{H}CD19) comprising an amino acid sequence that is at least about 95%, 96%, 97%, 98%, 99% or 100% identical to the amino acid sequence of SEQ ID NO:63, and a light chain variable region (V_{L}CD19) comprising an amino acid sequence that is at least about 95%, 96%, 97%, 98%, 99% or 100% identical to the amino acid sequence of SEQ ID NO:64. More particularly, the Fab fragment capable of specific binding to CD19 comprises a heavy chain variable region (V_{H}CD19) comprising the amino acid sequence of SEQ ID NO:63 and a light chain variable region (V_{L}CD19) comprising the amino acid sequence of SEQ ID NO:64.

In a particular aspect, provided is a bispecific antigen binding molecule, wherein
(i) the first and third Fab fragment capable of specific binding to 4-1BB each comprise a heavy chain variable region VH comprising the amino acid sequence of SEQ ID NO: 7 and a light chain variable region comprising an amino acid sequence of SEQ ID NO: 8 and
(ii) the second Fab fragment capable of specific binding to CD19 comprises a heavy chain variable region VH comprising an amino acid sequence of SEQ ID NO:63 and a light chain variable region VL comprising an amino acid sequence of SEQ ID NO:64.

In a particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:84, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:85. In a further particular embodiment, the bispecific antigen binding molecule comprises a polypeptide sequence of SEQ ID NO:65, a polypeptide sequence of SEQ ID NO:84, two polypeptide sequences of SEQ ID NO:67 and a polypeptide sequence of SEQ ID NO:85.

Bispecific Antigen Binding Molecules Wherein the Target Cell Antigen is PD-L1

In a particular aspect, the target cell antigen is PD-L1. CD19 binding moieties have been described for example in WO 2010/077634 which is included by reference in its entirety. PD-L1 binding moieties of particular interest are described below.

In one aspect, the invention provides a bispecific antigen binding molecule, wherein the Fab fragment capable of specific binding to PD-L1 comprises a heavy chain variable region (V_{H}PD-L1) comprising (i) CDR-H1 comprising the amino acid sequence of SEQ ID NO:145, (ii) CDR-H2 comprising the amino acid sequence of SEQ ID NO:146, and (iii) CDR-H3 comprising the amino acid sequence of SEQ ID NO:147, and a light chain variable region (V_{L}PD-L1) comprising (iv) CDR-L1 comprising the amino acid sequence of SEQ ID NO:148, (v) CDR-L2 comprising the amino acid sequence of SEQ ID NO:149, and (vi) CDR-L3 comprising the amino acid sequence of SEQ ID NO:150.

Particularly, the Fab fragment capable of specific binding to PD-L1 comprises a heavy chain variable region (V_{H}PD-L1) comprising an amino acid sequence that is at least about 95%, 96%, 97%, 98%, 99% or 100% identical to the amino acid sequence of SEQ ID NO:152, and a light chain variable region (V_{L}PD-L1) comprising an amino acid sequence that is at least about 95%, 96%, 97%, 98%, 99% or 100% identical to the amino acid sequence of SEQ ID NO:153. More particularly, the Fab fragment capable of specific binding to PD-L1 comprises a heavy chain variable region (V_{H}PD-L1) comprising the amino acid sequence of SEQ ID NO:152 and a light chain variable region (V_{L}PD-L1) comprising the amino acid sequence of SEQ ID NO:153.

In a particular aspect, provided is a bispecific antigen binding molecule, wherein
(i) the first and third Fab fragment capable of specific binding to 4-1BB each comprise a heavy chain variable region VH comprising the amino acid sequence of SEQ ID NO: 7 and a light chain variable region comprising an amino acid sequence of SEQ ID NO: 8 and
(ii) the second Fab fragment capable of specific binding to CD19 comprises a heavy chain variable region VH comprising an amino acid sequence of SEQ ID NO:152 and a light chain variable region VL comprising an amino acid sequence of SEQ ID NO:153.

In a particular aspect, the bispecific antigen binding molecule comprises a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:193, two polypeptides that are at least 95%, 96%, 97%, 98%, or 99% identical to the sequence of SEQ ID NO:67, and a polypeptide that is at least 95%, 96%, 97%, 98%, or 99% identical to the sequence of SEQ ID NO:194. In a further particular embodiment, the bispecific antigen binding molecule comprises a polypeptide sequence of SEQ ID NO:65, a polypeptide sequence of SEQ ID NO:193, two polypeptide sequences of SEQ ID NO:67 and a polypeptide sequence of SEQ ID NO:194.

Also disclosed is an antigen binding molecule with monovalent binding to 4-1BB (1+1 format). Provided is thus a bispecific antigen binding molecule comprising a polypeptide that is at least 95%, 96%, 97%, 98%, or 99% identical to the sequence of SEQ ID NO: 65, a polypeptide that is at least 95%, 96%, 97%, 98%, or 99% identical to the sequence of SEQ ID NO:195, a polypeptides that is at least 95%, 96%, 97%, 98%, or 99% identical to the sequence of SEQ ID NO:67, and a polypeptide that is at least 95%, 96%, 97%, 98%, or 99% identical to the sequence of SEQ ID NO:194. In a further particular embodiment, the bispecific antigen binding molecule comprises a polypeptide sequence of SEQ ID NO:65, a polypeptide sequence of SEQ ID NO:195, a polypeptide sequence of SEQ ID NO:67 and a polypeptide sequence of SEQ ID NO:194.

Fc Domain Modifications Reducing Fc receptor Binding and/or Effector Function

The bispecific antigen binding molecules of the invention further comprise a Fc domain composed of a first and a second subunit capable of stable association.

In certain aspects, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

The Fc domain confers favorable pharmacokinetic properties to the bispecific antibodies of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular embodiments the Fc domain of the the bispecific antibodies of the invention exhibits reduced binding affinity to an Fc receptor and/or reduced effector function, as compared to a native IgG Fc domain, in particular an IgG1 Fc domain or an IgG4 Fc domain. More particularly, the Fc domain is an IgG1 Fc domain.

In one such aspect the Fc domain (or the bispecific antigen binding molecule of the invention comprising said Fc domain) exhibits less than 50%, preferably less than 20%, more preferably less than 10% and most preferably less than 5% of the binding affinity to an Fc receptor, as compared to a native IgG1 Fc domain (or the bispecific antigen binding molecule of the invention comprising a native IgG1 Fc domain), and/or less than 50%, preferably less than 20%, more preferably less than 10% and most preferably less than 5% of the effector function, as compared to a native IgG1 Fc domain (or the bispecific antigen binding molecule of the invention comprising a native IgG1 Fc domain). In one aspect, the Fc domain (or the bispecific antigen binding molecule of the invention comprising said Fc domain) does not substantially bind to an Fc receptor and/or induce effector function. In a particular aspect the Fc receptor is an Fcγ receptor. In one aspect, the Fc receptor is a human Fc receptor. In one aspect, the Fc receptor is an activating Fc receptor. In a specific aspect, the Fc receptor is an activating human Fcγ receptor, more specifically human FcγRIIIa, FcγRI or FcγRIIa, most specifically human FcγRIIIa. In one aspect, the Fc receptor is an inhibitory Fc receptor. In a specific aspect, the Fc receptor is an inhibitory human Fcγ receptor, more specifically human FcγRIIB. In one aspect the effector function is one or more of CDC, ADCC, ADCP, and cytokine secretion. In a particular aspect, the effector function is ADCC. In one aspect, the Fc domain domain exhibits substantially similar binding affinity to neonatal Fc receptor (FcRn), as compared to a native IgG1 Fc domain. Substantially similar binding to FcRn is achieved when the Fc domain (or the the bispecific antigen binding molecule of the invention comprising said Fc domain) exhibits greater than about 70%, particularly greater than about 80%, more particularly greater than about 90% of the binding affinity of a native IgG1 Fc domain (or the the bispecific antigen binding molecule of the invention comprising a native IgG1 Fc domain) to FcRn.

In a particular aspect, the Fc domain is engineered to have reduced binding affinity to an Fc receptor and/or reduced effector function, as compared to a non-engineered Fc domain. In a particular aspect, the Fc domain of the bispecific antigen binding molecule of the invention comprises one or more amino acid mutation that reduces the binding affinity of the Fc domain to an Fc receptor and/or effector function. Typically, the same one or more amino acid mutation is present in each of the two subunits of the Fc domain. In one aspect, the amino acid mutation reduces the binding affinity of the Fc domain to an Fc receptor. In another aspect, the amino acid mutation reduces the binding affinity of the Fc domain to an Fc receptor by at least 2-fold, at least 5-fold, or at least 10-fold. In one aspect, the bispecific antigen binding molecule of the invention comprising an engineered Fc domain exhibits less than 20%, particularly less than 10%, more particularly less than 5% of the binding affinity to an Fc receptor as compared to bispecific antibodies of the invention comprising a non-engineered Fc domain. In a particular aspect, the Fc receptor is an Fcγ receptor. In other aspects, the Fc receptor is a human Fc receptor. In one aspect, the Fc receptor is an inhibitory Fc receptor. In a specific aspect, the Fc receptor is an inhibitory human Fcγ receptor, more specifically human FcγRIIB. In some aspects the Fc receptor is an activating Fc receptor. In a specific aspect, the Fc receptor is an activating human Fcγ receptor, more specifically human FcγRIIIa, FcγRI or FcγRIIa, most specifically human FcγRIIIa. Preferably, binding to each of these receptors is reduced. In some aspects, binding affinity to a complement component, specifically binding affinity to C1q, is also reduced. In one aspect, binding affinity to neonatal Fc receptor (FcRn) is not reduced. Substantially similar binding to FcRn, i.e. preservation of the binding affinity of the Fc domain to said receptor, is achieved when the Fc domain (or the bispecific antigen binding molecule of the invention comprising said Fc domain) exhibits greater than about 70% of the binding affinity of a non-engineered form of the Fc domain (or the bispecific antigen binding molecule of the invention comprising said non-engineered form of the Fc domain) to FcRn. The Fc domain, or the the bispecific antigen binding molecule of the invention comprising said Fc domain, may exhibit greater than about 80% and even greater than about 90% of such affinity. In certain embodiments the Fc domain of the bispecific antigen binding molecule of the invention is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described. (e.g. U.S. Pat. No. 6,737,056; WO 2004/056312, and Shields, R. L. et al., J. Biol. Chem. 276 (2001) 6591-6604).

In one aspect of the invention, the Fc domain comprises an amino acid substitution at a position of E233, L234, L235, N297, P331 and P329. In some aspects, the Fc domain comprises the amino acid substitutions L234A and L235A (“LALA”). In one such embodiment, the Fc domain is an IgG1 Fc domain, particularly a human IgG1 Fc domain. In one aspect, the Fc domain comprises an amino acid substitution at position P329. In a more specific aspect, the amino acid substitution is P329A or P329G, particularly P329G. In one embodiment the Fc domain comprises an amino acid substitution at position P329 and a further amino acid substitution selected from the group consisting of E233P, L234A, L235A, L235E, N297A, N297D or P331S. In more particular embodiments the Fc domain comprises the amino acid mutations L234A, L235A and P329G (“P329G LALA”). The “P329G LALA” combination of amino acid substitutions almost completely abolishes Fcγ receptor binding of a human IgG1 Fc domain, as described in PCT Patent Application No. WO 2012/130831 A1. Said document also describes methods of preparing such mutant Fc domains and methods for determining its properties such as Fc receptor binding or effector functions.such antibody is an IgG1 with mutations L234A and L235A or with mutations L234A, L235A and P329G (numbering according to EU index of Kabat et al , Kabat et al., Sequences of Proteins of Immunological Interest, 5th Ed. Public Health Service, National Institutes of Health, Bethesda, MD, 1991).

In one aspect, the Fc domain is an IgG4 Fc domain. In a more specific embodiment, the Fc domain is an IgG4 Fc domain comprising an amino acid substitution at position 5228 (Kabat numbering), particularly the amino acid substitution S228P. In a more specific embodiment, the Fc domain is an IgG4 Fc domain comprising amino acid substitutions L235E and S228P and P329G. This amino acid substitution reduces in vivo Fab arm exchange of IgG4 antibodies (see Stubenrauch et al., Drug Metabolism and Disposition 38, 84-91 (2010)).

Antibodies with increased half lives and improved binding to the neonatal Fc receptor (FcRn), which is responsible for the transfer of maternal IgGs to the fetus (Guyer, R.L. et al., J. Immunol. 117 (1976) 587-593, and Kim, J. K. et al., J. Immunol. 24 (1994) 2429-2434), are described in US 2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan, A. R. and Winter, G., Nature 322 (1988) 738-740; U.S. Pat. No. 5,648,260; U.S. Pat. No. 5,624,821; and WO 94/29351 concerning other examples of Fc region variants.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor. Effector function of an Fc domain, or bispecific antigen binding molecules of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

The following section describes preferred aspects of the bispecific antigen binding molecules of the invention comprising Fc domain modifications reducing Fc receptor binding and/or effector function. In one aspect, the invention relates to a bispecific antigen binding molecule comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein the Fc domain comprises one or more amino acid substitution that reduces the binding affinity of the antibody to an Fc receptor, in particular towards Fcγ receptor. In another aspect, the invention relates to the bispecific antigen binding molecule comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein the Fc domain comprises one or more amino acid substitution that reduces effector function. In another aspect, the invention relates to the bispecific antigen binding molecule comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein the Fc domain exhibits reduced binding affinity to an Fc receptor, particularly an Fcγ receptor, and/or reduced effector function, particularly antibody-dependent cell-mediated cytotoxicity (ADCC), as compared to a native IgG₁ Fc domain. In another aspect, the invention relates to the bispecific antigen binding molecule comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein the Fc domain comprises one or more amino acid substitution that reduces binding to an Fc receptor and/or effector function, particularly wherein said one or more amino acid substitution is at one or more position selected from the group of L234, L235, and P329 (Kabat EU index numbering). In another aspect, the invention relates to the bispecific antigen binding molecule comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein each subunit of the Fc domain comprises three amino acid substitutions that reduce binding to an activating Fc receptor and/or effector function wherein said amino acid substitutions are L234A, L235A and P329G (Kabat EU index numbering). In particular aspect, the Fc domain is of human IgG1 subclass with the amino acid mutations L234A, L235A and P329G (numbering according to Kabat EU index).

Fc Domain Modifications Promoting Heterodimerization

The bispecific antigen binding molecules of the invention comprise different antigen-binding sites, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the bispecific antigen binding molecules of the invention in recombinant production, it will thus be advantageous to introduce in the Fc domain of the bispecific antigen binding molecules of the invention a modification promoting the association of the desired polypeptides.

Accordingly, the invention relates to the bispecific antigen binding molecule comprising (a) at least one antigen binding domain capable of specific binding to 4-1BB, (b) at least one antigen binding domain capable of specific binding to a target cell antigen, and (c) a Fc domain composed of a first and a second subunit capable of stable association, wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect said modification is a so-called “knob-into-hole” modification, comprising a “knob” modification in one of the two subunits of the Fc domain and a “hole” modification in the other one of the two subunits of the Fc domain. Thus, the invention relates to the bispecific antigen binding molecule, wherein in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. Provided is thus a bispecific antigen molecule comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein the first subunit of the Fc domain comprises knobs and the second subunit of the Fc domain comprises holes according to the knobs into holes method. In particular, said amino acid residue having a larger side chain volume is selected from the group consisting of arginine (R), phenylalanine (F), tyrosine (Y), and tryptophan (W), and said amino acid residue having a smaller side chain volume is selected from the group consisting of alanine (A), serine (S), threonine (T), and valine (V). In one aspect, in the first subunit of the Fc domain the threonine residue at position 366 is replaced with a tryptophan residue (T366W), and in the second subunit of the Fc domain the tyrosine residue at position 407 is replaced with a valine residue (Y407V), and optionally in the second subunit of the Fc domain additionally the threonine residue at position 366 is replaced with a serine residue (T366S) and the leucine residue at position 368 is replaced with an alanine residue (L368A) (all numberings according to Kabat EU index). In a particular aspect, the first subunit of the Fc domain comprises the amino acid substitutions S354C and T366W (numbering according to Kabat EU index) and the second subunit of the Fc domain comprises the amino acid substitutions Y349C, T366S and Y407V (numbering according to Kabat EU index).

The knob-into-hole technology is described e.g. in U.S. Pat. No. 5,731,168; U.S. Pat. No. 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in one aspect, in the CH3 domain of the first subunit of the Fc domain of the bispecific antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g. by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

The C-terminus of the heavy chain of the bispecific antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, a bispecific antibody comprising a heavy chain including a C-terminal CH3 domain as specified herein, comprises the C-terminal glycine-lysine dipeptide (G446 and K447, numbering according to Kabat EU index). In one embodiment of all aspects as reported herein, a bispecific antibody comprising a heavy chain including a C-terminal CH3 domain, as specified herein, comprises a C-terminal glycine residue (G446, numbering according to Kabat EU index).

Modifications in the CH1/CL Domains

To further improve correct pairing, the bispecific antigen binding molecules can contain different charged amino acid substitutions (so-called “charged residues”). These modifications are introduced in the crossed or non-crossed CH1 and CL domains. In a particular aspect, the invention relates to a bispecific antigen binding molecule, wherein in at least one of CL domains the amino acid at position 123 (EU numbering) has been replaced by arginine (R) and the amino acid at position 124 (EU numbering) has been substituted by lysine (K) and wherein in at least one of the CH1 domains the the amino acids at position 147 (EU numbering) and at position 213 (EU numbering) have been substituted by glutamic acid (E). More particularly, the invention relates to a bispecific antigen binding molecule, wherein in the CL domain of the Fab domain that binds to 4-1BB the amino acid at position 123 (EU numbering) has been replaced by arginine (R) and the amino acid at position 124 (EU numbering) has been substituted by lysine (K), and wherein in the CH1 domain of the Fab domain that binds to 4-1BB the amino acids at position 147 (EU numbering) and at position 213 (EU numbering) have been substituted by glutamic acid (E).

In one aspect, provided is a bispecific antigen binding molecule, comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein the second Fab fragment (b) is fused at the C-terminus of the Fab heavy chain to the N-terminus of the Fab heavy chain of the first Fab fragment (a), which is in turn fused at its C-terminus to the N-terminus of the first Fc domain subunit, and the third Fab fragment (c) is fused at the C-terminus of the Fab heavy chain to the N-terminus of the second Fc domain subunit, wherein in the third Fab fragment capable of specific binding to a target cell antigen (i) the variable domains VL and VH are replaced by each other, or (ii) the constant domains CL and CH1 are replaced by each other, and wherein in the constant domain CL of the first and the third Fab fragment capable of specific binding to 4-1BB the amino acid at position 124 is substituted by lysine (K) ((numbering according to Kabat EU index) and the amino acid at position 123 is substituted by arginine (R) or lysine (K) (numbering according to Kabat EU index), and wherein in the constant domain CH1 of the first and the third Fab fragment capable of specific binding to 4-1BB the amino acid at position 147 is substituted by glutamic acid (E) (numbering according to Kabat EU index) and the amino acid at position 213 is substituted by glutamic acid (E) (numbering according to Kabat EU index).

Modifications in the Fab Domains

The invention relates to a bispecific antigen binding molecule comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein in the second Fab fragment capable of specific binding to a target cell antigen (i) the variable regions VL and VH of the Fab light chain and Fab heavy chain are replaced by each other, or (ii) the constant regions CL and CH1 of the Fab light chain and Fab heavy chain are replaced by each other. The bispecific antibodies are thus prepared according to the Crossmab technology.

Multispecific antibodies with a domain replacement/exchange in one binding arm (CrossMab VH-VL or CrossMab CH-CL) are described in detail in WO2009/080252 and Schaefer, W. et al, PNAS, 108 (2011) 11187-1191. They clearly reduce the byproducts caused by the mismatch of a light chain against a first antigen with the wrong heavy chain against the second antigen (compared to approaches without such domain exchange).

In one aspect, the invention relates to a bispecific antigen binding molecule wherein in the second Fab fragment capable of specific binding to a target cell antigen (i) the variable regions VL and VH of the Fab light chain and Fab heavy chain are replaced by each other, or (ii) the constant regions CL and CH1 of the Fab light chain and Fab heavy chain are replaced by each other, wherein in the second Fab fragment the constant domains CL and CH1 are replaced by each other so that the CH1 domain is part of the light chain and the CL domain is part of the heavy chain (CH-CL crossmab). In another aspect, in the second Fab fragment the variable domains VL and VH are replaced by each other so that the VH domain is part of the light chain and the VL domain is part of the heavy chain (VH-VL crossmab). More particularly, in the second Fab fragment capable of specific binding to a target cell antigen variable regions VL and VH of the Fab light chain and the Fab heavy chain are replaced by each other so that the VH domain is part of the light chain and the VL domain is part of the heavy chain.

Polynucleotides

The invention further provides isolated polynucleotides encoding a bispecific antigen binding molecule as described herein or a fragment thereof.

The isolated polynucleotides encoding bispecific antigen binding molecules of the invention may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated polynucleotide encodes a polypeptide comprised in the bispecific molecule according to the invention as described herein.

In one aspect, the present invention is directed to an isolated polynucleotide encoding a bispecific antigen binding molecule comprising (a) a first Fab fragment capable of specific binding to 4-1BB, (b) a second Fab fragment capable of specific binding to a target cell antigen, (c) a third Fab fragment capable of specific binding to 4-1BB, and (d) a Fc domain composed of a first and a second subunit capable of stable association, wherein the first and third Fab fragment capable of specific binding to 4-1BB each comprise a heavy chain variable region (V_{H}4-1BB) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a light chain variable region (V_{L}4-1BB) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

Bispecific antigen binding molecules of the invention may be obtained, for example, by recombinant production. For recombinant production one or more polynucleotide encoding the bispecific antigen binding molecule or polypeptide fragments thereof are provided. The one or more polynucleotide encoding the bispecific antigen binding molecule are isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the bispecific antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the bispecific antigen binding molecule or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the bispecific antigen binding molecule of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the bispecific antigen binding molecule or polypeptide fragments thereof is desired, DNA encoding a signal sequence may be placed upstream of the nucleic acid encoding the bispecific antigen binding molecule of the invention or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the fusion protein may be included within or at the ends of the polynucleotide encoding a bispecific antigen binding molecule of the invention or polypeptide fragments thereof.

In a further aspect of the invention, a host cell comprising one or more polynucleotides of the invention is provided. In certain aspects, a host cell comprising one or more vectors of the invention is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) a bispecific antigen binding molecule of the invention of the invention. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr-CHO cells (Urlaub et al., Proc Natl Acad Sci USA 77, 4216 (1980)); and myeloma cell lines such as YO, NSO, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing a bispecific antigen binding molecule of the invention or polypeptide fragments thereof is provided, wherein the method comprises culturing a host cell comprising polynucleotides encoding the bispecific antigen binding molecule of the invention or polypeptide fragments thereof, as provided herein, under conditions suitable for expression of the bispecific antigen binding molecule of the invention or polypeptide fragments thereof, and recovering the bispecific antigen binding molecule of the invention or polypeptide fragments thereof from the host cell (or host cell culture medium).

Bispecific molecules of the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the bispecific antigen binding molecule binds. For example, for affinity chromatography purification of fusion proteins of the invention, a matrix with protein A or protein G may be used. Sequential Protein A or G affinity chromatography and size exclusion chromatography can be used to isolate an antigen binding molecule essentially as described in the examples. The purity of the bispecific antigen binding molecule or fragments thereof can be determined by any of a variety of well-known analytical methods including gel electrophoresis, high pressure liquid chromatography, and the like. For example, the bispecific antigen binding molecules expressed as described in the Examples were shown to be intact and properly assembled as demonstrated by reducing and non-reducing SDS-PAGE.

Assays

The bispecific antigen binding molecules provided herein may be identified, screened for, or characterized for their physical/chemical properties and/or biological activities by various assays known in the art.

1. Affinity Assays

The affinity of the bispecific antigen binding molecules, antibodies and antibody fragments provided herein for 4-1BB and the target cell antigen can be determined in accordance with the methods set forth in the examples by surface plasmon resonance (SPR), using standard instrumentation such as a BIAcore instrument (GE Healthcare), and receptors or target proteins such as may be obtained by recombinant expression. The affinity of the bispecific antigen binding molecule for the target cell antigen can also be determined by surface plasmon resonance (SPR), using standard instrumentation such as a BIAcore instrument (GE Healthcare), and receptors or target proteins such as may be obtained by recombinant expression. A specific illustrative and exemplary embodiment for measuring binding affinity is described in Example 1.2. According to one aspect, K_{D} is measured by surface plasmon resonance using a BIACORE® T100 machine (GE Healthcare) at 25° C.

2. Binding Assays and Other Assays

Binding of the bispecific antigen binding molecule provided herein to the corresponding receptor expressing cells may be evaluated using cell lines expressing the particular receptor or target antigen, for example by flow cytometry (FACS). In one aspect, a reporter cell line Jurkat-hu4-1BB-NFκB-luc2 expressing 4-1BB is used in the binding assay. In a further aspect, cancer cell lines expressing the target cell antigen, for example FAP or CEA, were used to demonstrate the binding of the antigen binding molecules to the target cell antigen.

In another aspect, competition assays may be used to identify an antigen binding molecule that competes with a specific antibody or antigen binding molecule for binding to the target or 4-1BB, respectively. In certain embodiments, such a competing antigen binding molecule binds to the same epitope (e.g., a linear or a conformational epitope) that is bound by a specific anti-target antibody or a specific anti-4-1BB antibody. Detailed exemplary methods for mapping an epitope to which an antibody binds are provided in Morris (1996) “Epitope Mapping Protocols,” in Methods in Molecular Biology vol. 66 (Humana Press, Totowa, N.J.).

3. Activity Assays

In one aspect, assays are provided for identifying bispecific antigen binding molecules that bind to a specific target cell antigen and to 4-1BB having biological activity. Biological activity may include, e.g., agonistic signalling through 4-1BB on cells expressing the target cell antigen. Bispecific antigen binding molecules identified by the assays as having such biological activity in vitro are also provided.

In certain aspects, a bispecific antigen binding molecule of the invention is tested for such biological activity. Furthermore, assays for detecting cell lysis (e.g. by measurement of LDH release), induced apoptosis kinetics (e.g. by measurement of Caspase 3/7 activity) or apoptosis (e.g. using the TUNEL assay) are well known in the art. In addition the biological activity of such complexes can be assessed by evaluating their effects on survival, proliferation and lymphokine secretion of various lymphocyte subsets such as NK cells, NKT-cells or γδ T-cells or assessing their capacity to modulate phenotype and function of antigen presenting cells such as dendritic cells, monocytes/macrophages or B-cells.

Pharmaceutical Compositions, Formulations and Routes of Administation

In a further aspect, the invention provides pharmaceutical compositions comprising one or more of the bispecific antigen binding molecules provided herein, e.g., for use in any of the below therapeutic methods. In one embodiment, a pharmaceutical composition comprises any of the bispecific antigen binding molecules provided herein and at least one pharmaceutically acceptable excipient. In another embodiment, a pharmaceutical composition comprises any of the bispecific antigen binding molecules provided herein and at least one additional therapeutic agent, e.g., as described below.

Pharmaceutical compositions of the present invention comprise a therapeutically effective amount of one or more bispecific antigen binding molecules dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one bispecific antigen binding molecule according to the invention and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the bispecific antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the bispecific antigen binding molecules may be in powder form for constitution with a suitable vehicle, e.g., sterile pyrogen-free water, before use. Sterile injectable solutions are prepared by incorporating the antigen binding molecules of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the antigen binding molecules may also be formulated as a depot preparation. Such long acting formulations may be administered by implantation (for example subcutaneously or intramuscularly) or by intramuscular injection. Thus, for example, the fusion proteins may be formulated with suitable polymeric or hydrophobic materials (for example as emulsion in an acceptable oil) or ion exchange resins, or as sparingly soluble derivatives, for example, as a sparingly soluble salt.

Pharmaceutical compositions comprising the bispecific antigen binding molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The bispecific antigen binding molecules may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

Therapeutic Methods and Compositions

Any of the bispecific antigen binding molecules provided herein may be used in therapeutic methods. For use in therapeutic methods, bispecific antigen binding molecules of the invention can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, bispecific antigen binding molecules of the invention for use as a medicament are provided.

In further aspects, bispecific antigen binding molecules of the invention for use (i) in stimulating or enhancing T cell response, (ii) for use in supporting survival of activated T cells, (iii) for use in the treatment of cancer, (iv) for use in delaying progression of cancer, or (v) for use in prolonging the survival of a patient suffering from cancer, are provided. In a particular aspect, bispecific antigen binding molecules of the invention for use in treating a disease, in particular for use in the treatment of cancer, are provided.

In certain aspects, bispecific antigen binding molecules of the invention for use in a method of treatment are provided. In one aspect, the invention provides a bispecific antigen binding molecule as described herein for use in the treatment of a disease in an individual in need thereof. In certain aspects, the invention provides a bispecific antigen binding molecule for use in a method of treating an individual having a disease comprising administering to the individual a therapeutically effective amount of the bispecific antigen binding molecule. In certain aspects the disease to be treated is cancer. The subject, patient, or “individual” in need of treatment is typically a mammal, more specifically a human.

In one aspect, provided is a method for (i) stimulating or enhancing T-cell response, (ii) supporting survival of activated T cells, (iii) treating cancer, (iv) delaying progression of cancer or (v) prolonging the survival of a patient suffering from cancer, wherein the method comprises administering a therapeutically effective amount of the bispecific antigen binding molecule of the invention to an individual in need thereof.

In a further aspect, the invention provides for the use of the bispecific antigen binding molecule of the invention in the manufacture or preparation of a medicament for the treatment of a disease in an individual in need thereof In one aspect, the medicament is for use in a method of treating a disease comprising administering to an individual having the disease a therapeutically effective amount of the medicament. In certain aspects, the disease to be treated is a proliferative disorder, particularly cancer. Examples of cancers include, but are not limited to, bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other examples of cancer include carcinoma, lymphoma (e.g., Hodgkin's and non-Hodgkin's lymphoma), blastoma, sarcoma, and leukemia. Other cell proliferation disorders that can be treated using the bispecific antigen binding molecule or antibody of the invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the the bispecific antigen binding molecule or antibody of the invention may not provide a cure but may provide a benefit. In some aspects, a physiological change having some benefit is also considered therapeutically beneficial. Thus, in some aspects, an amount of the bispecific antigen binding molecule or antibody of the invention that provides a physiological change is considered an “effective amount” or a “therapeutically effective amount”.

For the prevention or treatment of disease, the appropriate dosage of a bispecific antigen binding molecule of the invention (when used alone or in combination with one or more other additional therapeutic agents) will depend on the type of disease to be treated, the route of administration, the body weight of the patient, the specific molecule, the severity and course of the disease, whether the the bispecific antigen binding molecule or antibody of the invention is administered for preventive or therapeutic purposes, previous or concurrent therapeutic interventions, the patient's clinical history and response to the fusion protein, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The bispecific antigen binding molecule of the invention is suitably administered to the patient at one time or over a series of treatments. Depending on the type and severity of the disease, about 1 μg/kg to 15 mg/kg (e.g. 0.1 mg/kg-10 mg/kg) of the bispecific antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the the bispecific antigen binding molecule or antibody of the invention would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 0.1 mg/kg body weight to about 20 mg/kg body weight, about 5 μg/kg body weight to about 1 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the fusion protein). In a particular aspect, the bispecific antigen binding molecule will be administered every three weeks. An initial higher loading dose, followed by one or more lower doses may be administered. However, other dosage regimens may be useful. The progress of this therapy is easily monitored by conventional techniques and assays.

The bispecific antigen binding molecule of the invention will generally be used in an amount effective to achieve the intended purpose. For use to treat or prevent a disease condition, the bispecific antigen binding molecule or antibody of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the the bispecific antigen binding molecule or antibody of the invention which are sufficient to maintain therapeutic effect. Usual patient dosages for administration by injection range from about 0.1 to 50 mg/kg/day, typically from about 0.1 to 1 mg/kg/day. Therapeutically effective plasma levels may be achieved by administering multiple doses each day. Levels in plasma may be measured, for example, by HPLC.

In cases of local administration or selective uptake, the effective local concentration of the bispecific antigen binding molecule or antibody of the invention may not be related to plasma concentration. One skilled in the art will be able to optimize therapeutically effective local dosages without undue experimentation.

A therapeutically effective dose of the bispecific antigen binding molecule of the invention described herein will generally provide therapeutic benefit without causing substantial toxicity. Toxicity and therapeutic efficacy of a fusion protein can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD_{50/}ED₅₀. Bispecific antigen binding molecules that exhibit large therapeutic indices are preferred. In one aspect, the the bispecific antigen binding molecule or antibody of the invention exhibits a high therapeutic index. The data obtained from cell culture assays and animal studies can be used in formulating a range of dosages suitable for use in humans. The dosage lies preferably within a range of circulating concentrations that include the ED50 with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with bispecific antibodies of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Other Agents and Treatments

The bispecific antigen binding molecule of the invention may be administered either alone or in combination with one or more other agents in therapy. For instance, the bispecific antigen binding molecule or antibody of the invention of the invention may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is another anti-cancer agent or chemotherapeutic agent, for example a microtubule disruptor, an antimetabolite, a topoisomerase inhibitor, a DNA intercalator, an alkylating agent, an anthracycline, a hormonal therapy, a kinase inhibitor, a receptor antagonist, an activator of tumor cell apoptosis, or an antiangiogenic agent. In certain aspects, an additional therapeutic agent is an immunomodulatory agent, a cytostatic agent, an inhibitor of cell adhesion, a cytotoxic or cytostatic agent, an activator of cell apoptosis, or an agent that increases the sensitivity of cells to apoptotic inducers.

In one aspect, the bispecific antigen binding molecule of the invention is administered in combination with a chemotherapeutic agent, radiation therapy and/ or other agents for use in cancer immunotherapy. A chemotherapeutic agent is an anti-cancer agent as defined above. Alternatively, a chemotherapeutic agent is selected from the group consisting of nucleotide analogs (e.g. azacitidine, capecitabine, doxifluridine, fluorouracil, gemcitabine, hydroxyurea or methotrexate), platinum-based agents (e.g. carboplatin, cisplatin or oxaliplatin), taxanes (e.g. paclitaxel, docetaxel, abraxane or taxotere), alkylating agents (e.g. cyclophosphamide, chlorambucil, dacarbazine or temozolomide), anthracyclines (e.g. doxorubicin or idarubicin), topoisomerase I inhibitors (e.g. irinotecan or topotecan), topoisomerase II inhibitors (e.g. etoposide or teniposide), kinase inhibitors (e.g. erlotinib, imatinib, vemurafenib or vismodegib), retinoids, histone deacetylase inhibitors and vinca alkaloids. Other agents for use in cancer immunotherapy include for example agents blocking PD-L1/PD-1 interaction such as a PD1 antibody (e.g. pembrolizumab or nivolumab) or a PD-L1 antibody (e.g. atezolizumab). Bispecific antigen binding molecules of the invention may also be used in combination with radiation therapy.

Such other agents are suitably present in combination in amounts that are effective for the purpose intended. The effective amount of such other agents depends on the amount of fusion protein used, the type of disorder or treatment, and other factors discussed above. The the bispecific antigen binding molecule or antibody of the invention are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the bispecific antigen binding molecule or antibody of the invention can occur prior to, simultaneously, and/or following, administration of the additional therapeutic agent and/or adjuvant. In one aspect, administration of the bispecific antigen binding molecule and administration of an additional therapeutic agent occur within about one month, or within about one, two or three weeks, or within about one, two, three, four, five, or six days, of each other.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a bispecific antigen binding molecule of the invention.

The label or package insert indicates that the composition is used for treating the condition of choice. Moreover, the article of manufacture may comprise (a) a first container with a composition contained therein, wherein the composition comprises a bispecific antigen binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE B
(Sequences):

| SEQ | | |
|---|---|---|
| ID | | |
| NO: | Name | Sequence |
| 1 | 4-1BB (20H4.9) | GYYWS |
| | CDR-H1 | |
| 2 | 4-1BB (20H4.9) | EINHGGYVTYNPSLES |
| | CDR-H2 | |
| 3 | 4-1BB (20H4.9) | DYGPGNYDWYFDL |
| | CDR-H3 | |
| 4 | 4-1BB (20H4.9) | RASQSVSSYLA |
| | CDR-L1 | |
| 5 | 4-1BB (20H4.9) | DASNRAT |
| | CDR-L2 | |
| 6 | 4-1BB (20H4.9) | QQRSNWPPALT |
| | CDR-L3 | |
| 7 | 4-1BB (20H4.9) | QVQLQQWGAGLLKPSETLSLTCAVYGGSFSG |
| | VH | YYWSWIRQSPEKGLEWIGEINHGGYVTYNPS |
| | | LESRVTISVDTSKNQFSLKLSSVTAADTAVY |
| | | YCARDYGPGNYDWYFDLWGRGTLVTVSS |
| 8 | 4-1BB (20H4.9) | EIVLTQSPATLSLSPGERATLSCRASQSVSS |
| | VL | YLAWYQQKPGQAPRLLIYDASNRATGIPARF |
| | | SGSGSGTDFTLTISSLEPEDFAVYYCQQRSN |
| | | WPPALTFGGGTKVEIK |
| 9 | FAP(4B9) CDR-H1 | SYAMS |
| 10 | FAP(4B9) CDR-H2 | AIIGSGASTYYADSVKG |
| 11 | FAP(4B9) CDR-H3 | GWFGGFNY |
| 12 | FAP(4B9) CDR-L1 | RASQSVTSSYLA |
| 13 | FAP(4B9) CDR-L2 | VGSRRAT |
| 14 | FAP(4B9) CDR-L3 | QQGIMLPPT |
| 15 | FAP (28H1) CDR-H1 | SHAMS |
| 16 | FAP (28H1) CDR-H2 | AIWASGEQYYADSVKG |
| 17 | FAP (28H1) CDR-H3 | GWLGNFDY |
| 18 | FAP (28H1) CDR-L1 | RASQSVSRSYLA |
| 19 | FAP (28H1) CDR-L2 | GASTRAT |
| 20 | FAP (28H1) CDR-L3 | QQGQVIPPT |
| 21 | FAP(4B9) VH | EVQLLESGGGLVQPGGSLRLSCAASGFTFSS |
| | | YAMSWVRQAPGKGLEWVSAIIGSGASTYYAD |
| | | SVKGRFTISRDNSKNTLYLQMNSLRAEDTAV |
| | | YYCAKGWFGGFNYWGQGTLVTVSS |
| 22 | FAP(4B9) VL | EIVLTQSPGTLSLSPGERATLSCRASQSVTS |
| | | SYLAWYQQKPGQAPRLLINVGSRRATGIPDR |
| | | FSGSG |
| | | SGTDFTLTISRLEPEDFAVYYCQQGIMLPPT |
| | | FGQGTKVEIK |
| 23 | FAP(28H1) VH | EVQLLESGGGLVQPGGSLRLSCAASGFTFSS |
| | | HAMSWVRQAPGKGLEWVSAIWASGEQYYADS |
| | | VKGRFTISRDNSKNTLYLQMNSLRAEDTAVY |
| | | YCAKGWLGNFDYWGQGTLVTVSS |
| 24 | FAP(28H1) VL | EIVLTQSPGTLSLSPGERATLSCRASQSVSR |
| | | SYLAWYQQKPGQAPRLLIIGASTRATGIPDR |
| | | FSGSGSGTDFTLTISRLEPEDFAVYYCQQGQ |
| | | VIPPTFGQGTKVEIK |
| 25 | CEA (A5B7)-CDR-H1 | DYYMN |
| 26 | CEA (A5B7)-CDR-H2 | FIGNKANGYTTEYSASVKG |
| 27 | CEA (A5B7)-CDR-H3 | DRGLRFYFDY |
| 28 | CEA (A5B7)-CDR-L1 | RASSSVTYIH |
| 29 | CEA (A5B7)-CDR-L2 | ATSNLAS |
| 30 | CEA (A5B7)-CDR-L3 | QHWSSKPPT |
| 31 | CEA (A5B7) VH | EVKLVESGGGLVQPGGSLRLSCATSGFTFTD |
| | (parental) | YYMNWVRQPPGKALEWLGFIGNKANGYTTEY |
| | | SASVKGRFTISRDKSQSILYLQMNTLRAEDS |
| | | ATYYCTRDRGLRFYFDYWGQGTTLTVSS |
| 32 | CEA (A5B7) VL | QTVLSQSPAILSASPGEKVTMTCRASSSVTY |
| | (parental) | IHWYQQKPGSSPKSWIYATSNLASGVPARFS |
| | | GSGSGTSYSLTISRVEAEDAATYYCQHWSSK |
| | | PPTFGGGTKLEIK |
| 33 | CEA (MFE23)-CDR-H1 | DSYMH |
| 34 | CEA (MFE23)-CDR-H2 | WIDPENGDTEYAPKFQG |
| 35 | CEA (MFE23)-CDR-H3 | GTPTGPYYFDY |
| 36 | CEA (MFE23) CDR-L1 | SASSSVSYMH |
| 37 | CEA (MFE23)-CDR-L2 | STSNLAS |
| 38 | CEA (MFE23)-CDR-L3 | QQRSSYPLT |
| 39 | CEA (MFE23) VH | QVKLQQSGAELVRSGTSVKLSCTASGFNIKD |
| | | SYMHWLRQGPEQGLEWIGWIDPENGDTEYAP |
| | | KFQGKATFTTDTSSNTAYLQLSSLTSEDTAV |
| | | YYCNEGTPTGPYYFDYWGOGTTVTVSS |
| 40 | CEA (MFE23) VL | ENVLTQSPAIMSASPGEKVTITCSASSSVSY |
| | | MHWFQQKPGTSPKLWIYSTSNLASGVPARFS |
| | | GSGSGTSYSLTISRMEAEDAATYYCQQRSSY |
| | | PLTFGAGTKLELK |
| 41 | CEA (T84.66-LCHA)- | DTYMH |
| | CDR-H1 | |
| 42 | CEA (T84.66-LCH A)- | RIDPANGNSKYVPKFQG |
| | CDR-H2 | |
| 43 | CEA (T84.66-LCHA)- | FGYYVSDYAMAY |
| | CDR-H3 | |
| 44 | CEA (T84.66-LCHA)- | RAGESVDIFGVGFLH |
| | CDR-L1 | |
| 45 | CEA (T84.66-LCHA)- | RASNRAT |
| | CDR-L2 | |
| 46 | CEA (T84.66-LCHA)- | QQTNEDPYT |
| | CDR-L3 | |
| 47 | CEA (T84.66-LCHA) | QVQLVQSGAEVKKPGSSVKVSCKASGFNIKD |
| | VH | TYMHWVRQAPGQGLEWMGRIDPANGNSKYVP |
| | | KFQGRVTITADTSTSTAYMELSSLRSEDTAV |
| | | YYCAPFGYYVSDYAMAYWGQGTLVTVSS |
| 48 | CEA (T84.66-LCHA) | EIVLTQSPATLSLSPGERATLSCRAGESVDI |
| | VL | FGVGFLHWYQQKPGQAPRLLIYRASNRATGI |
| | | PARFSGSGSGTDFTLTISSLEPEDFAVYYCQ |
| | | QTNEDPYTFGQGTKLEIK |
| 49 | CEA (CH1A1A 98/99)- | EFGMN |
| | CDR-H1 | |
| 50 | CEA (CH1A1A 98/99)- | WINTKTGEATYVEEFKG |
| | CDR-H2 | |
| 51 | CEA (CH1A1A 98/99)- | WDFAYYVEAMDY |
| | CDR-H3 | |
| 52 | CEA (CEA 2F1)- | KASAAVGTYVA |
| | CDR-L1 | |
| 53 | CEA (CEA 2F1)- | SASYRKR |
| | CDR-L2 | |
| 54 | CEA (CEA 2F1)- | HQYYTYPLFT |
| | CDR-L3 | |
| 55 | CEA (CH1A1A 98/99) | QVQLVQSGAEVKKPGASVKVSCKASGYTFTE |
| | VH | FGMNWVRQAPGQGLEWMGWINTKTGEATYVE |
| | | EFKGRVTFTTDTSTSTAYMELRSLRSDDTAV |
| | | YYCARWDFAYYVEAMDYWGQGTTVTVSS |
| 56 | CEA (CEA 2F1) VL | DIQMTQSPSSLSASVGDRVTITCKASAAVGT |
| | | YVAWYQQKPGKAPKLLIYSASYRKRGVPSRF |
| | | SGSGSGTDFTLTISSLQPEDFATYYCHQYYT |
| | | YPLFTF |
| | | GQGTKLEIK |
| 57 | CD19 (8B8-2B11) | DYIMH |
| | CDR-H1 | |
| 58 | CD 19 (8B8-2B11) | YINPYNDGSKYTEKFQG |
| | CDR-H2 | |
| 59 | CD 19 (8B8-2B11) | GTYYYGPQLFDY |
| | CDR-H3 | |
| 60 | CD 19 (8B8-2B11) | KSSQSLETSTGTTYLN |
| | CDR-L1 | |
| 61 | CD 19 (8B8-2B11) | RVSKRFS |
| | CDR-L2 | |
| 62 | CD 19 (8B8-2B11) | LQLLEDPYT |
| | CDR-L3 | |
| 63 | CD 19 (8B8-2B11) | QVQLVQSGAEVKKPGASVKVSCKASGYTFTD |
| | VH | YIMHWVRQAPGQGLEWMGYINPYNDGSKYTE |
| | | KFQGRVTMTSDTSISTAYMELSRLRSDDTAV |
| | | YYCARGTYYYGPQLFDYWGQGTTVTVSS |
| 64 | CD 19 (8B8-2B11) | DIVMTQTPLSLSVTPGQPASISCKSSQSLET |
| | VL | STGTTYLNWYLQKPGQSPQLLIYRVSKRFSG |
| | | VPDRFSGSGSGTDFTLKISRVEAEDVGVYYC |
| | | LQLLEDPYTFGQGTKLEIK |
| 65 | VHCH1(EE) | see Table 1 |
| | (20H4.9)- | |
| | Heavy chain HC1 | |
| | (Fc hole) | |
| 66 | VLCH1 (4B9) | see Table 1 |
| | VHCH1(EE) | |
| | (20H4.9)-Heavy | |
| | chain HC2 | |
| | (Fc knob) | |
| 67 | VLCL(RK)-Light | see Table 1 |
| | chain | |
| | (20H4.9) | |
| 68 | VHCL-Light chain | see Table 1 |
| | (4B9) | |
| 69 | Fc hole chain | sec Table 3 |
| 70 | human 4-1BB | see Table 3 |
| | antigen Fc | |
| | knob chain | |
| 71 | VHCH1 (20H4.9)- | see Table 4 |
| | heavy | |
| | chain HC1 | |
| | (Fchole)-VH | |
| | (4B9) | |
| 72 | VHCH1 (20H4.9)- | see Table 4 |
| | Heavy | |
| | chain HC2 | |
| | (Fc knob)-VL | |
| | (4B9) | |
| 73 | VLCL-Light chain | sec Table 4 |
| | (20H4.9) | |
| 74 | VHCH1 (20H4.9)- | see Table 6 |
| | heavy | |
| | chain HC1 | |
| | (Fc hole)-VH | |
| | (DP47) | |
| 75 | VHCH1 (20H4.9)- | see Table 6 |
| | Heavy | |
| | chain HC2 | |
| | (Fc knob)-VL | |
| | (DP47) | |
| 76 | VLCH1 (A5B7) | see Table 7 |
| | VHCH1(EE) | |
| | (20H4.9)- | |
| | Heavy chain HC2 | |
| | (Fc knob) | |
| 77 | VHCL-Light chain | see Table 7 |
| | (A5B7) | |
| 78 | VLCH1 (MFE23) | sec Table 8 |
| | VHCH1(EE) | |
| | (20H4.9)- | |
| | Heavy chain | |
| | HC2 (Fc knob) | |
| 79 | VHCL-Light chain | see Table 8 |
| | (MFE23) | |
| 80 | VLCH1 (T84.66-LCHA) | see Table 9 |
| | VHCH1(EE) (20H4.9)- | |
| | Heavy chain HC2 | |
| | (Fc knob) | |
| 81 | VHCL-Light chain | sec Table 9 |
| | (T84.66- | |
| | LCHA) | |
| 82 | VLCH1 (CEA 2F1) | see Table 10 |
| | VHCH1(EE) (20H4.9)- | |
| | Heavy chain HC2 | |
| | (Fc knob) | |
| 83 | VHCL-Light chain | see Table 10 |
| | (CEA | |
| | CH1A1 98/99) | |
| 84 | VLCH1 (2B11) | see Table 30 |
| | VHCH1(EE) | |
| | (20H4.9)- | |
| | Heavy chain | |
| | HC2 (Fc knob) | |
| 85 | VHCL-Light | see Table 30 |
| | chain (2B11) | |
| 86 | Human (hu) | UniProt no. Q12884 |
| | FAP | |
| 87 | hu FAP | RPSRVHNSEENTMRALTLKDILNGTFSYKTF |
| | ectodomain + | FPNWISGQEYLHQSADNNIVLYNIETGQSYT |
| | poly-lys- | ILSNRTMKSVNASNYGLSPDRQFVYLESDYS |
| | tag + his₆- | KLWRYSYTATYYIYDLSNGEFVRGNELPRPI |
| | tag | QYLCWSPVGSKLAYVYQNNIYLKQRPGDPPF |
| | | QITFNGRENKIFNGIPDWVYEEEMLATKYAL |
| | | WWSPNGKFL |
| | | AYAEFNDTDIPVIAYSYYGDEQYPRTINIPY |
| | | PKAGAKNPVVRIFIIDTTYPAYVGPQEVPVP |
| | | AMIASSDYYFSWLTWVTDERVCLQWLKRVQN |
| | | VSVLSICDFREDWQTWDCPKTQEHIEESRTG |
| | | WAGGFFVSTPVFSYDAISYYKIFSDKDGYKH |
| | | IHYIKDTVENAIQITSGKWEAINIFRVTQDS |
| | | LFYSSNEFEEYPGRRNIYRISIGSYPPSKKC |
| | | VTCHLRKERCQYYTASFSDYAKYYALVCYGP |
| | | GIPISTLHDGRTDQEIKILEENKELENALKN |
| | | IQLPKEEIKKLEVDEITLWYKMILPPQFDRS |
| | | KKYPLLIQVYGGPCSQSVRSVFAVNWISYLA |
| | | SKEGMVIALVDGRGTAFQGDKLLYAVYRKLG |
| | | VYEVEDQITAVRKFIEMGFIDEKRIAIWGWS |
| | | YGGYVSSLALASGTGLFKCGIAVAPVSSWEY |
| | | YASVYTERFMGLPTKDDNLEHYKNSTVMARA |
| | | EYFRNVDYLLIHGTADDNVHFQNSAQIAKAL |
| | | VNAQVDFQAMWYSDQNHGLSGLSTNHLYTHM |
| | | THFLKQCFSLSDGKKKKKKGHHHHHH |
| 88 | mouse FAP | UniProt no. P97321 |
| 89 | Murine FAP | RPSRVYKPEGNTKRALTLKDILNGTFSYKTY |
| | ectodomain + | FPNWISEQEYLHQSEDDNIVFYNIETRESYI |
| | poly-lys- | ILSNSTMKSVNATDYGLSPDRQFVYLESDYS |
| | tag + his₆- | KLWRYSYTATYYIYDLQNGEFVRGYELPRPI |
| | tag | QYLCWSPVGSKLAYVYQNNIYLKQRPGDPPF |
| | | QITYTGRENRIFNGIPDWVYEEEMLATKYAL |
| | | WWSPDGKFLAYVEFNDSDIPIIAYSYYGDGQ |
| | | YPRTINIPYPKAGAKNPVVRVFIVDTTYPHH |
| | | VGPMEVPVPEMIASSDYYFSWLTWVSSERVC |
| | | LQWLKRVQNVSVLSICDFREDWHAWECPKNQ |
| | | EHVEESRTGWAGGFFVSTPAFSQDATSYYKI |
| | | FSDKDGYKHIHYIKDTVENAIQITSGKWEAI |
| | | YIFRVTQDSLFYSSNEFEGYPGRRNIYRISI |
| | | GNSPPSKKCVTCHLRKERCQYYTASFSYKAK |
| | | YYALVCYGPGLPISTLHDGRTDQEIQVLEEN |
| | | KELENSLRNIQLPKVEIKKLKDGGLTFWYKM |
| | | ILPPQFDRSKKYPLLIQVYGGPCSQSVKSVF |
| | | AVNWITYLASKEGIVIALVDGRGTAFQGDKF |
| | | LHAVYRKLGVYEVEDQLTAVRKFIEMGFIDE |
| | | ERIAIWGWSYGGYVSSLALASGTGLFKCGIA |
| | | VAPVSSWEYYASIYSERFMGLPTKDDNLEHY |
| | | KNSTVMARAEYFRNVDYLLIHGTADDNVHFQ |
| | | NSAQIAKALVNAQVDFQAMWYSDQNHGILSG |
| | | RSQNHLYTHMTHFLKQCFSLSDGKKKKKKGH |
| | | HHHFIH |
| 90 | Cynomolgus FAP | RPPRVHNSEENTMRALTLKDILNGTFSYKTF |
| | ectodomain + | FPNWISGQEYLHQSADNNIVLYNIETGQSYT |
| | poly-lys- | ILSNRTMKSVNASNYGLSPDRQFVYLESDYS |
| | tag + his₆- | KLWRYSYTATYYIYDLSNGEFVRGNELPRPI |
| | tag | QYLCWSPVGSKLAYVYQNNIYLKQRPGDPPF |
| | | QITFNGRENKIFNGIPDWVYEEEMLATKYAL |
| | | WWSPNGKFLAYAEFNDTDIPVIAYSYYGDEQ |
| | | YPRTINIPYPKAGAKNPFVRIFIIDTTYPAY |
| | | VGPQEVPVPAMIASSDYYFSWLTWVTDERVC |
| | | LQWLKRVQNVSVLSICDFREDWQTWDCPKTQ |
| | | EHIEESRTGWAGGFFVSTPVFSYDAISYYKI |
| | | FSDKDGYKHIHYIKDTVEN |
| | | AIQITSGKWEAINIFRVTQDSLFYSSNEFED |
| | | YPGRRNIYRISIGSYPPSKKCVTCHLRKERC |
| | | QYYTASFSDYAKYYALVCYGPGIPISTLHDG |
| | | RTDQEIKILEENKELENALKNIQLPKEEIKK |
| | | LEVDEITLWYKMILPPQFDRSKKYPLLIQVY |
| | | GGPCSQSVRSVFAVNWISYLASKEGMVIALV |
| | | DGRGTAFQGDKLLYAVYRKLGVYEVEDQITA |
| | | VRKFIEMGFIDEKRIAIWGWSYGGYVSSLAL |
| | | ASGTGLFKCGIAVAPVSSWEYYASVYTERFM |
| | | GLPTKDDNLEHYKNSTVMARAEYFRNVDYLL |
| | | IHGTADDNVHFQNSAQIAKALVNAQVDFQAM |
| | | WYSDQNHGLSGLSTNHLYTHMTHFLKQCFSL |
| | | SDGKKKKKKGHHHHHH |
| 91 | human CEA | UniProt no. P06731 |
| 92 | human MCSP | UniProt no. Q6UVK1 |
| 93 | human EGER | UniProt no. P00533 |
| 94 | human CD 19 | UniProt no. PI5391 |
| 95 | human CD20 | Uniprot no. P11836 |
| 96 | human CD33 | UniProt no. P20138 |
| 97 | human 4-1BB | UniProt no. Q07011 |
| 98 | murine 4-1BB | UniProt no. P20334 |
| 99 | cynomolgus | Uniprot no. F6W5G6 |
| | 4-1BB | |
| 100 | Peptide linker | GGGGS |
| | (G4S) | |
| 101 | Peptide linker | GGGGSGGGGS |
| | (G4S)₂ | |
| 102 | Peptide linker | SGGGGSGGGG |
| | (SG4)₂ | |
| 103 | Peptide linker | GGGGSGGGGSGGGG |
| | G4(SG4)₂ | |
| 104 | peptide linker | GSPGSSSSGS |
| 105 | (G4S)₃ peptide | GGGGSGGGGSGGGGS₃ |
| | linker | |
| 106 | (G4S)₄ peptide | GGGGSGGGGSGGGGSGGGGS |
| | linker | |
| 107 | peptide linker X | GSGSGSGS |
| 108 | peptide linker X1 | GSGSGNGS |
| 109 | peptide linker X2 | GGSGSGSG |
| 110 | peptide linker X3 | GGSGSG |
| 111 | peptide linker X4 | GGSG |
| 112 | peptide linker X5 | GGSGNGSG |
| 113 | peptide linker X6 | GGNGSGSG |
| 114 | peptide linker X7 | GGNGSG |
| 115 | CEA (A5H1EL1D)- | DYYMN |
| | CDR-H1 | |
| 116 | CEA (A5H1EL1D)- | FIGNKANAYTTEYSASVKG |
| | CDR-H2 | |
| 117 | CEA (A5H1EL1D)- | DRGLRFYFDY |
| | CDR-H3 | |
| 118 | CEA (A5H1EL1D)- | RASSSVTYIH |
| | CDR-L1 | |
| 119 | CEA (A5H1EL1D)- | ATSNLAS |
| | CDR-L2 | |
| 120 | CEA (A5H1EL1D)- | QHWSSKPPT |
| | CDR- | |
| | L3 | |
| 121 | CEA (A5H1EL1D) VH | EVQLLESGGGLVQPGGSLRLSCAASGFTFTD |
| | (3-23A5-1E) | YYMNWVRQAPGKGLEWLGFIGNKANAYTTEY |
| | | SASVKGRFTISRDKSKNTLYLQMNSLRAEDT |
| | | ATYYCTRDRGLRFYFDYWGQGTTVTVSS |
| 122 | CEA (A5H1EL1D) VL | EIVLTQSPATLSLSPGERATLSCRASSSVTY |
| | (A5-L1D) | IHWYQQKPGQAPRSWIYATSNLASGIPARFS |
| | | GSGSGTDFTLTISSLEPEDFAVYYCQHWSSK |
| | | PPTFGQGTKLEIK |
| 123 | CEA (MFE-H24 | DSYMH |
| | to H29)- | |
| | CDR-H1 | |
| 124 | CEA (MFE-H24, | WIDPENGDTEYAPKFQG |
| | H25, H27, | |
| | H28, H29)- | |
| | CDR-H2 | |
| 125 | CEA (MFE-H26)- | WIDPENGGTNYAQKFQG |
| | CDR-H2 | |
| 126 | CEA (MFE-H24 | GTPTGPYYFDY |
| | to H29)- | |
| | CDR-H3 | |
| 127 | CEA (MFE-L24, | RASSSVSYMH |
| | L25, L27, | |
| | L28, L29)- | |
| | CDR-L1 | |
| 128 | CEA (MFE-H26)- | RASQSISSYM |
| | CDR-L1 | |
| 129 | CEA (MFE-L24, | STSNLAS |
| | L25, L27, | |
| | L28)-CDR-L2 | |
| 130 | CEA (MFE-L26)- | YTSNLAS |
| | CDR-L2 | |
| 131 | CEA (MFE-L29)- | STSSLQS |
| | CDR-L2 | |
| 132 | CEA (MFE-L24, | QQRSSYPLT |
| | L25, L27, | |
| | L26, L28, L29)- | |
| | CDR-L3 | |
| 133 | MFE-H24 | QVQLVQSGAEVKKPGASVKVSCKASGFNIKD |
| | | SYMHWVRQAPGQGLEWMGWIDPENGDTEYAP |
| | | KFQGRVTMTTDTSISTAYMELSRLRSDDTAV |
| | | YYCNEGTPTGPYYFDYWGQGTLVTVSS |
| 134 | MFE-H25 | QVQLVQSGAEVKKPGASVKVSCKASGYTFKD |
| | | SYMHWVRQAPGQGLEWMGWIDPENGDTEYAP |
| | | KFQGRVTMTTDTSISTAYMELSRLRSDDTAV |
| | | YYCNEGTPTGPYYFDYWGQGTLVTVSS |
| 135 | MFE-H26 | QVQLVQSGAEVKKPGASVKVSCKASGFNIKD |
| | | SYMHWVRQAPGQGLEWMGWIDPENGGTNYAQ |
| | | KFQGRVTMTTDTSISTAYMELSRLRSDDTAV |
| | | YYCNEGTPTGPYYFDYWGQGTLVTVSS |
| 136 | MFE-H27 | QVQLVQSGAEVKKPGASVKVSCKASGFNIKD |
| | | SYMHWVRQAPGQGLEWMGWIDPENGDTEYAP |
| | | KFQGRVTMTTDTSISTAYMELSRLRSDDTAV |
| | | YYCARGTPTGPYYFDYWGQGTLVTVSS |
| 137 | MFE-H28 | QVQLVQSGAEVKKPGASVKVSCKASGFNIKD |
| | | SYMHWVRQAPGQGLEWMGWIDPENGDTEYAP |
| | | KFQGRVTMTRDTSISTAYMELSRLRSDDTAV |
| | | YYCNEGTPTGPYYFDYWGQGTLVTVSS |
| 138 | MFE-H29 | QVQLVQSGAEVKKPGSSVKVSCKASGFNIKD |
| | | SYMHWVRQAPGQGLEWMGWIDPENGDTEYAP |
| | | KFQGRVTITTDESTSTAYMELSSLRSEDTAV |
| | | YYCNEGTPTGPYYFDYWGQGTLVTVSS |
| 139 | MFE-L24 | DIQMTQSPSSLSASVGDRVTITCRASSSVSY |
| | | MHWYQQKPGKAPKLLIYSTSNLASGVPSRFS |
| | | GSGSGTDFTLTISSLQPEDFATYYCQQRSSY |
| | | PLTFGGGTKLEIK |
| 140 | MFE-L25 | EIQMTQSPSSLSASVGDRVTITCRASSSVSY |
| | | MHWYQQKPGKAPKLLIYSTSNLASGVPSRFS |
| | | GSGSGTDFTLTISSLQPEDFATYYCQQRSSY |
| | | PLTFGGGTKLEIK |
| 141 | MFE-L26 | EIQMTQSPSSLSASVGDRVTITCRASQSISS |
| | | YMHWYQQKPGKAPKLLIYSTSNLASGVPSRF |
| | | SGSGSGTDFTLTISSLQPEDFATYYCQQRSS |
| | | YPLTFGGGTKLEIK |
| 142 | MFE-L27 | EIQMTQSPSSLSASVGDRVTITCRASSSVPY |
| | | MHWYQQKPGKAPKLLIYSTSNLASGVPSRFS |
| | | GSGSGTDFTLTISSVQPEDFATYYCQQRSSY |
| | | PLTFGGGTKLEIK |
| 143 | MFE-L28 | EIQMTQSPSSLSASVGDRVTITCRASSSVPY |
| | | MHWLQQKPGKAPKLLIYSTSNLASGVPSRFS |
| | | GSGSGTDFTLTISSVQPEDFATYYCQQRSSY |
| | | PLTFGGGTKLEIK |
| 144 | MFE-L29 | EIQMTQSPSSLSASVGDRVTITCRASSSVPY |
| | | MHWLQQKPGKAPKLLIYSTSSLQSGVPSRFS |
| | | GSGSGTDFTLTISSVQPEDFATYYCQQRSSY |
| | | PLTFGGGTKLEIK |
| 145 | PD-L1 CDR-H1 | GFTFSDSWIH |
| 146 | PD-L1 CDR-H2 | AWISPYGGSTYYADSVKG |
| 147 | PD-L1 CDR-H3 | RHWPGGFDY |
| 148 | PD-L1 CDR-L1 | RASQDVSTAVA |
| 149 | PD-L1 CDR-L2 | SASFLYS |
| 150 | PD-L1 CDR-L3 | QQYLYHPAT |
| 151 | PD-L1 VH | EVQLVESGGGLVQPGGSLRLSCAASGFTFSD |
| | | SWIHWVRQAPGKGLEWVAWISPYGGSTYYAD |
| | | SVKGRFTISADTSKNTAYLQMNSLRAEDTAV |
| | | YYCARRHWPGGFDYWGQGTLVTVSS |
| 152 | PD-L1 VL | DIQMTQSPSSLSASVGDRVTITCRASQDVST |
| | | AVAWYQQKPGKAPKLLIYSASFLYSGVPSRF |
| | | SGSGSGTDFTLTISSLQPEDFATYYCQQYLY |
| | | HPATFGQGTKVEIK |
| 153 | IGHV3-23-02 | See Table 12 |
| 154 | IGHV3-15*01 | See Table 12 |
| 155 | 3-23A5-1 | See Table 12 |
| 156 | 3-23 A5-2 | See Table 12 |
| 157 | 3-23A5-3 | See Table 12 |
| 158 | 3-23 A5-4 | See Table 12 |
| 159 | 3-23A5-1A | See Table 12 |
| | (all | |
| | backmutations) | |
| 160 | 3-23A5-1C (A93T) | See Table 12 |
| 161 | 3-23A5-1D (K73) | See Table 12 |
| 162 | 3-15A5-1 | See Table 12 |
| 163 | 3-15A5-2 | See Table 12 |
| 164 | 3-15A5-3 | See Table 12 |
| 165 | IGKV3-11 | See Table 13 |
| 166 | A5-L1 | See Table 13 |
| 167 | A5-L2 | See Table 13 |
| 168 | A5-L3 | See Table 13 |
| 169 | A5-L4 | See Table 13 |
| 170 | A5-L1A(all | See Table 13 |
| | backmutations) | |
| 171 | A5-L1B (Q1T2) | See Table 13 |
| 172 | A5-L1C (FR2) | See Table 13 |
| 173 | VLCH1 | See Table 16 |
| | (CEA A5H1EL1D) | |
| | VHCH1(EE) | |
| | (20H4.9)- | |
| | Heavy chain | |
| | HC2 (Fc knob) | |
| 174 | VHCL-Light | See Table 16 |
| | chain (CEA | |
| | A5H1EL1D) | |
| 175 | IGHV1-2-02 | See Table 18 |
| 176 | IGHV1-69-01 | See Table 18 |
| 177 | IGHV1-69-05 | See Table 18 |
| 178 | IGKV1-39-01 | See Table 19 |
| 179 | VLCH1 (CEA | See Table 22 |
| | huMFE23- | |
| | L28-H24) | |
| | VHCH1(EE) | |
| | (20H4.9)- | |
| | Heavy chain | |
| | HC2 | |
| | (Fc knob) | |
| 180 | VHCL-Light | See Table 22 |
| | chain (CEA | |
| | huMFE23- | |
| | L28-H24) | |
| 181 | VLCH1 | See Table 23 |
| | (CEA huMFE23- | |
| | L28-H28) | |
| | VHCH1(EE) | |
| | (20H4.9)- | |
| | Heavy chain | |
| | HC2 | |
| | (Fc knob) | |
| 182 | VHCL-Light | See Table 23 |
| | chain (CEA | |
| | huMFE23- | |
| | L28-H28) | |
| 183 | VLCH1 (CEA | See Table 24 |
| | huMFE23- | |
| | L28-H25) | |
| | VHCH1(EE) | |
| | (20H4.9)- | |
| | Heavy chain HC2 | |
| | (Fc knob) | |
| 184 | VHCL-Light | See Table 24 |
| | chain (CEA | |
| | huMFE23-L28-H25) | |
| 185 | VLCH1 | See Table 25 |
| | (CEA huMFE23- | |
| | L27-H29) | |
| | VHCH1(EE) | |
| | (20H4.9)- | |
| | Heavy chain HC2 | |
| | (Fc knob) | |
| 186 | VHCL-Light | See Table 25 |
| | chain (CEA | |
| | huMFE23- | |
| | L27-H29) | |
| 187 | VLCH1 | See Table 26 |
| | (CEA huMFE23- | |
| | L27-H28) | |
| | VHCH1(EE) | |
| | (20H4.9)- | |
| | Heavy chain HC2 | |
| | (Fc knob) | |
| 188 | VHCL- | See Table 26 |
| | Light chain (CEA | |
| | huMFE23-L27-H28) | |
| 189 | VHCH1 | See Table 27 |
| | (CEA huMFE23- | |
| | L27-H26) | |
| | VHCH1(EE) | |
| | (20H4.9)- | |
| | Heavy chain HC2 | |
| | (Fc knob) | |
| 190 | VHCL-Light | See Table 27 |
| | chain (CEA | |
| | huMFE23- | |
| | L27-H26) | |
| 191 | VHCH1 | See Table 28 |
| | (CEA huMFE23- | |
| | L27-H24) | |
| | VHCH1(EE) | |
| | (20H4.9)- | |
| | Heavy chain HC2 | |
| | (Fc knob) | |
| 192 | VHCL-Light | See Table 28 |
| | chain (CEA | |
| | huMFE23- | |
| | L27-H24) | |
| 193 | VHCH1 (PD-L1) | See Table 43 |
| | VHCH1(EE) | |
| | (20H4.9)- | |
| | Heavy chain | |
| | HC2 (Fc knob) | |
| 194 | VHCL-Light | See Table 43 |
| | chain (PD-L1) | |
| 195 | VHCH1 | See Table 44 |
| | (PD-L1)-Heavy | |
| | chain HC2 | |
| | (Fc knob) | |
| 196 | Human PD-L1 | UniProt no. Q9NZQ7 |

All nucleotide sequences are presented without the respective stop codon sequences.

Aspects of the Invention

The following numbered paragraphs (paras) describe aspects of the present invention:

1. A bispecific antigen binding molecule, comprising
(a) a first Fab fragment capable of specific binding to 4-1BB,
(b) a second Fab fragment capable of specific binding to a target cell antigen,
(c) a third Fab fragment capable of specific binding to 4-1BB, and
(d) a Fc domain composed of a first and a second subunit capable of stable association, wherein the second Fab fragment (b) is fused at the C-terminus of the Fab heavy chain to the N-terminus of the Fab heavy chain of the first Fab fragment (a), which is in turn fused at its C-terminus to the N-terminus of the first Fc domain subunit, and the third Fab fragment (c) is fused at the C-terminus of the Fab heavy chain to the N-terminus of the second Fc domain subunit, and wherein in the second Fab fragment capable of specific binding to a target cell antigen (i) the variable domains VL and VH are replaced by each other, or (ii) the constant domains CL and CH1 are replaced by each other.

2. The bispecific antigen binding molecule of para 1, wherein the bispecific antigen binding molecule provides bivalent binding to 4-1BB and monovalent binding to the target cell antigen.

3. The bispecific antigen binding molecule of para 1 or 2, wherein the Fc domain composed of a first and a second subunit capable of stable association is an an IgG Fc domain, particularly an IgG1 Fc domain or an IgG4 Fc domain.

4. The bispecific antigen binding molecule of any one of paras 1 to 3, wherein the first subunit of the Fc domain comprises knobs and the second subunit of the Fc domain comprises holes according to the knobs into holes method.

5. The bispecific antigen binding molecule of any one of paras 1 to 4, wherein the Fc domain comprises one or more amino acid substitution that reduces the binding affinity of the antigen binding molecule to an Fc receptor and/or effector function, particularly the amino acid mutations L234A, L235A and P329G (numbering according to Kabat EU index).

6. The bispecific antigen binding molecule of any one of paras 1 to 5, wherein the first and the third Fab fragment capable of specific binding to 4-1BB are identical.

7. The bispecific antigen binding molecule of any one of paras 1 to 6, wherein the first and the third Fab fragment capable of specific binding to 4-1BB each comprise a heavy chain variable region (V_{H}4-1BB) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and a light chain variable region (V_{L}4-1BB) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6.

8. The bispecific antigen binding molecule of any one of paras 1 to 7, wherein the first and the third Fab fragment capable of specific binding to 4-1BB each comprise a heavy chain variable region (V_{H}4-1BB) comprising an amino acid sequence that is at least about 95%, 96%, 97%, 98%, 99% or 100% identical to the amino acid sequence of SEQ ID NO:7 and a light chain variable region (V_{L}4-1BB) comprising an amino acid sequence that is at least about 95%, 96%, 97%, 98%, 99% or 100% identical to the amino acid sequence of SEQ ID NO:8.

9. The bispecific antigen binding molecule of any one of paras 1 to 8, wherein in the constant domain CL of the first and the third Fab fragment capable of specific binding to 4-1BB the amino acid at position 124 is substituted by lysine (K) (numbering according to Kabat EU index) and the amino acid at position 123 is substituted by arginine (R) or lysine (K) (numbering according to Kabat EU index), and wherein in the constant domain CH1 of the first and the third Fab fragment capable of specific binding to 4-1BB the amino acid at position 147 is substituted by glutamic acid (E) (numbering according to Kabat EU index) and the amino acid at position 213 is substituted by glutamic acid (E) (numbering according to Kabat EU index).

10. The bispecific antigen binding molecule of any one of paras 1 to 9, wherein in the second Fab fragment capable of specific binding to a target cell antigen the variable domains VL and VH of the Fab light chain and the Fab heavy chain are replaced by each other.

11. The bispecific antigen binding molecule of any one of paras 1 to 10, wherein the second Fab fragment capable of specific binding to a target cell antigen selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20 and CD33.

12. The bispecific antigen binding molecule of any one of paras 1 to 11, wherein the second Fab fragment capable of specific binding to a target cell antigen is a Fab fragment capable of specific binding to Fibroblast Activation Protein (FAP).

13. The bispecific antigen binding molecule of any one of paras 1 to 12, wherein the Fab fragment capable of specific binding to Fibroblast Activation Protein (FAP) comprises (a) a heavy chain variable region (V_{H}FAP) comprising (i) CDR-H1 comprising the amino acid sequence of SEQ ID NO:9, (ii) CDR-H2 comprising the amino acid sequence of SEQ ID NO:10, and (iii) CDR-H3 comprising the amino acid sequence of SEQ ID NO:11, and a light chain variable region (V_{L}FAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14, or (b) a heavy chain variable region (V_{H}FAP) comprising (i) CDR-H1 comprising the amino acid sequence of SEQ ID NO:15, (ii) CDR-H2 comprising the amino acid sequence of SEQ ID NO:16, and (iii) CDR-H3 comprising the amino acid sequence of SEQ ID NO:17, and a a light chain variable region (V_{L}FAP) comprising (iv) CDR-L1 comprising the amino acid sequence of SEQ ID NO:18, (v) CDR-L2 comprising the amino acid sequence of SEQ ID NO:19, and (vi) CDR-L3 comprising the amino acid sequence of SEQ ID NO:20.

14. The bispecific antigen binding molecule of any one of paras 1 to 13, wherein the Fab fragment capable of specific binding to Fibroblast Activation Protein (FAP) comprises
(a) a heavy chain variable region (V_{H}FAP) comprising an amino acid sequence that is at least about 95%, 96%, 97%, 98%, 99% or 100% identical to the amino acid sequence of SEQ ID NO:21, and a light chain variable region (V_{L}FAP) comprising an amino acid sequence that is at least about 95%, 96%, 97%, 98%, 99% or 100% identical to the amino acid sequence of SEQ ID NO:22, or
(b) a heavy chain variable region (V_{H}FAP) comprising an amino acid sequence that is at least about 95%, 96%, 97%, 98%, 99% or 100% identical to the amino acid sequence of SEQ ID NO:23, and a light chain variable region (V_{L}FAP) comprising an amino acid sequence that is at least about 95%, 96%, 97%, 98%, 99% or 100% identical to the amino acid sequence of SEQ ID NO:24.

15. The bispecific antigen binding molecule of any one of paras 1 to 11, wherein the Fab fragment capable of specific binding to a target cell antigen is a Fab fragment capable of specific binding to Carcinoembryonic Antigen (CEA).

16. The bispecific antigen binding molecule of any one of paras 1 to 11 or 15, wherein the Fab fragment capable of specific binding to Carcinoembryonic Antigen (CEA) comprises
(a) a heavy chain variable region (V_{H}CEA) comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a light chain variable region (V_{L}CEA) comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30, or
(b) a heavy chain variable region (V_{H}CEA) comprising (i) CDR-H1 comprising the amino acid sequence of SEQ ID NO:33, (ii) CDR-H2 comprising the amino acid sequence of SEQ ID NO:34, and (iii) CDR-H3 comprising the amino acid sequence of SEQ ID NO:35, and a light chain variable region (V_{L}CEA) comprising (iv) CDR-L1 comprising the amino acid sequence of SEQ ID NO:36, (v) CDR-L2 comprising the amino acid sequence of SEQ ID NO:37, and (vi) CDR-L3 comprising the amino acid sequence of SEQ ID NO:38, or
(c) a heavy chain variable region (V_{H}CEA) comprising (i) CDR-H1 comprising the amino acid sequence of SEQ ID NO:41, (ii) CDR-H2 comprising the amino acid sequence of SEQ ID NO:42, and (iii) CDR-H3 comprising the amino acid sequence of SEQ ID NO:43, and a light chain variable region (V_{L}CEA) comprising (iv) CDR-L1 comprising the amino acid sequence of SEQ ID NO:44, (v) CDR-L2 comprising the amino acid sequence of SEQ ID NO:45, and (vi) CDR-L3 comprising the amino acid sequence of SEQ ID NO:46, or
(d) a heavy chain variable region (V_{H}CEA) comprising (i) CDR-H1 comprising the amino acid sequence of SEQ ID NO:49, (ii) CDR-H2 comprising the amino acid sequence of SEQ ID NO:50, and (iii) CDR-H3 comprising the amino acid sequence of SEQ ID NO:51, and a light chain variable region (V_{L}CEA) comprising (iv) CDR-L1 comprising the amino acid sequence of SEQ ID NO:52, (v) CDR-L2 comprising the amino acid sequence of SEQ ID NO:53, and (vi) CDR-L3 comprising the amino acid sequence of SEQ ID NO:54.

17. The bispecific antigen binding molecule of any one of paras 1 to 11 or 15 or 16, wherein the Fab fragment capable of specific binding to Carcinoembryonic Antigen (CEA) comprises

(a) a heavy chain variable region (V_{H}CEA) comprising an amino acid sequence that is at least about 95%, 96%, 97%, 98%, 99% or 100% identical to the amino acid sequence of SEQ ID NO:31, and a light chain variable region (V_{L}CEA) comprising an amino acid sequence that is at least about 95%, 96%, 97%, 98%, 99% or 100% identical to the amino acid sequence of SEQ ID NO:32, or

(b) a heavy chain variable region (V_{H}CEA) comprising an amino acid sequence that is at least about 95%, 96%, 97%, 98%, 99% or 100% identical to the amino acid sequence of SEQ ID NO:39, and a light chain variable region (V_{L}CEA) comprising an amino acid sequence that is at least about 95%, 96%, 97%, 98%, 99% or 100% identical to the amino acid sequence of SEQ ID NO:40, or

(c) a heavy chain variable region (V_{H}CEA) comprising an amino acid sequence that is at least about 95%, 96%, 97%, 98%, 99% or 100% identical to the amino acid sequence of SEQ ID NO:47, and a light chain variable region (V_{L}CEA) comprising an amino acid sequence that is at least about 95%, 96%, 97%, 98%, 99% or 100% identical to the amino acid sequence of SEQ ID NO:48, or

(d) a heavy chain variable region (V_{H}CEA) comprising an amino acid sequence that is at least about 95%, 96%, 97%, 98%, 99% or 100% identical to the amino acid sequence of SEQ ID NO:55, and a light chain variable region (V_{L}CEA) comprising an amino acid sequence that is at least about 95%, 96%, 97%, 98%, 99% or 100% identical to the amino acid sequence of SEQ ID NO:56.

18. The bispecific antigen binding molecule of any one of paras 1 to 11, wherein the Fab fragment capable of specific binding to a target cell antigen is a Fab fragment capable of specific binding to CD19.

19. The bispecific antigen binding molecule of any one of paras 1 to 11 or 18, wherein the Fab fragment capable of specific binding to CD19 comprises
(a) a heavy chain variable region (V_{H}CD19) comprising (i) CDR-H1 comprising the amino acid sequence of SEQ ID NO:57, (ii) CDR-H2 comprising the amino acid sequence of SEQ ID NO:58, and (iii) CDR-H3 comprising the amino acid sequence of SEQ ID NO:59, and a light chain variable region (V_{L}CD19) comprising (iv) CDR-L1 comprising the amino acid sequence of SEQ ID NO:60, (v) CDR-L2 comprising the amino acid sequence of SEQ ID NO:61, and (vi) CDR-L3 comprising the amino acid sequence of SEQ ID NO:62.

20. The bispecific antigen binding molecule of any one of paras 1 to 11 or 18 or 19, wherein the Fab fragment capable of specific binding to CD19 comprises a heavy chain variable region (V_{H}CD19) comprising an amino acid sequence that is at least about 95%, 96%, 97%, 98%, 99% or 100% identical to the amino acid sequence of SEQ ID NO:63, and a light chain variable region (V_{L}CD19) comprising an amino acid sequence that is at least about 95%, 96%, 97%, 98%, 99% or 100% identical to the amino acid sequence of SEQ ID NO:64.

21. A polynucleotide encoding the bispecific antigen binding molecule of any one of paras 1 to 20.

22. A host cell comprising the polynucleotide of para 21.

23. A method of producing the bispecific antigen binding molecule of any one of paras 1 to 20 comprising culturing the host cell of para 22 under conditions suitable for the expression of the bispecific antigen binding molecule.

24. A pharmaceutical composition comprising a bispecific antigen binding molecule of any one of paras 1 to 20 and at least one pharmaceutically acceptable excipient.

25. The pharmaceutical composition of para 24 for use in the treatment of cancer.

26. The bispecific antigen binding molecule of any one of paras 1 to 20, or the pharmaceutical composition of para 24, for use as a medicament.

27. The bispecific antigen binding molecule of any one of paras 1 to 20 for use
(i) in stimulating T cell response,
(ii) in supporting survival of activated T cells,
(iii) in the treatment of cancer,
(iv) in delaying progression of cancer, or
(v) in prolonging the survival of a patient suffering from cancer.

28. The bispecific antigen binding molecule of any one of paras 1 to 20, or the pharmaceutical composition of para 24, for use in the treatment of cancer.

29. The bispecific antigen binding molecule of any one of paras 1 to 20 for use in the treatment of cancer, wherein the bispecific antigen binding molecule is administered in combination with a chemotherapeutic agent, radiation therapy and/ or other agents for use in cancer immunotherapy.

30. Use of the bispecific antigen binding molecule of any one of paras 1 to 20, or the pharmaceutical composition of para 24, in the manufacture of a medicament for the treatment of cancer or infectious diseases.

31. A method of inhibiting the growth of tumor cells in an individual comprising administering to the individual an effective amount of the bispecific antigen binding molecule of any one of paras 1 to 20, or the pharmaceutical composition of para 24, to inhibit the growth of the tumor cells.

32. A method of treating cancer or an infectious disease comprising administering to the individual a therapeutically effective amount of the bispecific antigen binding molecule of any one of paras 1 to 20, or the pharmaceutical composition of para 24.

### EXAMPLE S

The following are examples of methods and compositions of the invention. It is understood that various other embodiments may be practiced, given the general description provided above.

### Recombinant DNA Techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

### DNA Sequencing

DNA sequences were determined by double strand sequencing.

### Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

### Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, size exclusion chromatography (SEC) or mass spectrometry.

### SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

### Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

### Mass Spectrometry

This section describes the characterization of the multispecific antibodies with VH/VL or CH/CL exchange (CrossMabs) with emphasis on their correct assembly. The expected primary structures were analyzed by electrospray ionization mass spectrometry (ESI-MS) of the deglycosylated intact CrossMabs and deglycosylated/plasmin digested or alternatively deglycosylated/limited LysC digested CrossMabs.

The CrossMabs were deglycosylated with N-Glycosidase F in a phosphate or Tris buffer at 37° C. for up to 17 h at a protein concentration of 1 mg/ml. The plasmin or limited LysC (Roche) digestions were performed with 100 μg deglycosylated VH/VL CrossMabs in a Tris buffer pH 8 at room temperature for 120 hours and at 37° C. for 40 min, respectively. Prior to mass spectrometry the samples were desalted via HPLC on a Sephadex G25 column (GE Healthcare). The total mass was determined via ESI-MS on a maXis 4G UHR-QTOF MS system (Bruker Daltonik) equipped with a TriVersa NanoMate source (Advion).

### Example 1

### Preparation, Purification and Characterization of Bispecific Antibodies With a Bivalent Binding to 4-1BB and a Monovalent Binding to FAP

### 1.1 Generation of Bispecific Antibodies With a Bivalent Binding to 4-1BB and a Monovalent Binding to FAP

Bispecific agonistic 4-1BB antibodies with bivalent binding for 4-1BB and monovalent binding for FAP were prepared as depicted in FIG. 1. This construct is also termed Head to Head (H2H) 2+1 format or 2+1 H2H 4-1BB (20H4.9)/FAP (4B9) P329GLALA IgG1 or 4-1BB (20H.9)×FAP 2+1 H2H.

The first heavy chain HC1 of the construct was comprised of the following components: VHCH1 of anti-4-1BB binder (clone 20H4.9), followed by Fc hole. The second heavy chain HC2 was comprised of VLCH1 of anti-FAP binder (clone 4B9 in cross Fab format) followed by VHCH1 of an anti-4-1BB (clone 20H4.9) and by Fc knob. The generation and preparation of FAP binder 4B9 is described in WO 2012/020006 A2, which is incorporated herein by reference. For the 4-1BB binder, the VH and VL sequences of clone 20H4.9 were obtained in accordance with U.S. Pat. No. 7,288,638 B2 or U.S. Pat. No. 7,659,384 B2. Combination of the two heavy chains allows generation of a heterodimer, which includes a FAP binding cross Fab and two 4-1BB binding Fabs (FIG. 1).

To improve correct pairing, the following mutations have been introduced in the CH-CL of the anti-4-1BB Fab molecules: E123R and Q124K in CL and K147E and K213E in CH1. The second light chain LC2 of the anti-FAP binder (clone 4B9) was composed of VHCL (cross Fab).

The knobs into hole technology was applied by introducing the Y349C/T366S/L368A/Y407V mutations in the first heavy chain HC1 (Fc hole heavy chain) and by introducing the S354C/T366W mutations in the second heavy chain HC2 (Fc knob heavy chain) to allow generation of a heterodimer.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO2012/130831A1.

The amino acid sequences for the bispecific antibody 4-1BB (20H4.9)/FAP (4B9) P329GLALA IgG1 2+1 (H2H) can be found in Table 1.

TABLE 1
Amino acid sequences of bispecific,
bivalent anti-4-1BB /monovalent anti-FAP
human IgG1 P329GLALA antigen binding
molecules (2 + 1 H2H 4-1BB
(20H4.9)/FAP (4B9) P329GLALA IgG1)

| SEQ | | |
|---|---|---|
| ID | Descrip- | |
| NO: | tion | Sequence |
| 65 | VHCH1(EE) | QVQLQQWGAGLLKPSE |
| | (20H4.9)- | TLSLTCAVYGGSFSGY |
| | Heavy | YWSWIRQSPEKGLEWI |
| | chain HC1 | GEINHGGYVTYNPSLE |
| | (Fc | SRVTISVDTSKNQFSL |
| | hole) | KLSSVTAADTAVYYCA |
| | | RDYGPGNYDWYFDLWG |
| | | RGTLVTVSSASTKGPS |
| | | VFPLAPSSKSTSGGTA |
| | | ALGCLVEDYFPEPVTV |
| | | SWNSGALTSGVHTFPA |
| | | VLQSSGLYSLSSVVTV |
| | | PSSSLGTQTYICNVNH |
| | | KPSNTKVDEKVEPKSC |
| | | DKTHTCPPCPAPEAAG |
| | | GPSVFLFPPKPKDTLM |
| | | ISRTPEVTCWVDVSHE |
| | | DPEVKFNWYVDGVEVH |
| | | NAKTKPREEQYNSTYR |
| | | VVSVLTVLHQDWLNGK |
| | | EYKCKVSNKALGAPIE |
| | | KTISKAKGQPREPQVC |
| | | TLPPSRDELTKNQVSL |
| | | SCAVKGFYPSDIAVEW |
| | | ESNGQPENNYKTTPPV |
| | | LDSDGSFFLVSKLTVD |
| | | KSRWQQGNVFSCSVMH |
| | | EALHNHYTQKSLSLSP |
| 66 | VLCH1 (4B9) | EIVLTQSPGTLSLSPG |
| | VHCH1(EE) | ERATLSCRASQSVTSS |
| | (20H4.9)- | YLAWYQQKPGQAPRLL |
| | Heavy | INVGSRRATGIPDRFS |
| | chain HC2 | GSGSGTDFTLTISRLE |
| | (Fc | PEDFAVYYCQQGIMLP |
| | knob) | PTFGQGTKVEIKSSAS |
| | | TKGPSVFPLAPSSKST |
| | | SGGTAALGCLVKDYFP |
| | | EPVTVSWNSGALTSGV |
| | | HTFPAVLQSSGLYSLS |
| | | SVVTVPSSSLGTQTYI |
| | | CNVNHKPSNTKVDKKV |
| | | EPKSCDGGGGSGGGGS |
| | | QVQLQQWGAGLLKPSE |
| | | TLSLTCAVYGGSFSGY |
| | | YWSWIRQSPEKGLEWI |
| | | GEINHGGYVTYNPSLE |
| | | SRVTISVDTSKNQFSL |
| | | KLSSVTAADTAVYYCA |
| | | RDYGPGNYDWYFDLWG |
| | | RGTLVTVSSASTKGPS |
| | | VFPLAPSSKSTSGGTA |
| | | ALGCLVEDYFPEPVTV |
| | | SWNSGALTSGVHTFPA |
| | | VLQSSGLYSLSSVVTV |
| | | PSSSLGTQTYICNVNH |
| | | KPSNTKVDEKVEPKSC |
| | | DKTHTCPPCPAPEAAG |
| | | GPSVFLFPPKPKDTLM |
| | | ISRTPEVTCVVVDVSH |
| | | EDPEVKFNWYVDGVEV |
| | | HNAKTKPREEQYNSTY |
| | | RVVSVLTVLHQDWLNG |
| | | KEYKCKVSNKALGAPI |
| | | EKTISKAKGQPREPQV |
| | | YTLPPCRDELTKNQVS |
| | | LWCLVKGFYPSDIAVE |
| | | WESNGQPENNYKTTPP |
| | | VLDSDGSFFLYSKLTV |
| | | DKSRWQQGNVFSCSVM |
| | | HEALHNHYTQKSLSLS |
| | | P |
| 67 | VLCL(RK)- | EIVLTQSPATLSLSPG |
| | Light | ERATLSCRASQSVSSY |
| | chain | LAWYQQKPGQAPRLLI |
| | (20H4.9) | YDASNRATGIPARFSG |
| | | SGSGTDFTLTISSLEP |
| | | EDFAVYYCQQRSNWPP |
| | | ALTFGGGTKVEIKRTV |
| | | AAPSVFIFPPSDRKLK |
| | | SGTASVVCLLNNFYPR |
| | | EAKVQWKVDNALQSGN |
| | | SQESVTEQDSKDSTYS |
| | | LSSTLTLSKADYEKHK |
| | | VYACEVTHQGLSSPVT |
| | | KSFNRGEC |
| 68 | VHCL-Light | EVQLLESGGGLVQPGG |
| | chain | SLRLSCAASGFTFSSY |
| | (4B9) | AMSWVRQAPGKGLEWV |
| | | SAIIGSGASTYYADSV |
| | | KGRFTISRDNSKNTLY |
| | | LQMNSLRAEDTAVYYC |
| | | AKGWFGGFNYWGQGTL |
| | | VTVSSASVAAPSVFIF |
| | | PPSDEQLKSGTASVVC |
| | | LLNNFYPREAKVQWKV |
| | | DNALQSGNSQESVTEQ |
| | | DSKDSTYSLSSTLTLS |
| | | KADYEKHKVYACEVTH |
| | | QGLSSPVTKSFNRGEC |

### 1.2 Production of Bispecific Antibodies With a Bivalent Binding to 4-1BB and a Monovalent Binding to FAP

Production of IgG-like proteins in HEK293 EBNA or CHO EBNA cells

Antibodies and bispecific antibodies were generated by transient transfection of HEK293 EBNA cells or CHO EBNA cells. Cells were centrifuged and, medium was replaced by pre-warmed CD CHO medium (Thermo Fisher, Cat N° 10743029). Expression vectors were mixed in CD CHO medium, PEI (Polyethylenimine, Polysciences, Inc, Cat N° 23966-1) was added, the solution vortexed and incubated for 10 minutes at room temperature. Afterwards, cells (2 Mio/ml) were mixed with the vector/PEI solution, transferred to a flask and incubated for 3 hours at 37° C. in a shaking incubator with a 5% CO₂ atmosphere. After the incubation, Excell medium with supplements (80% of total volume) was added (W. Zhou and A. Kantardjieff, Mammalian Cell Cultures for Biologics Manufacturing, DOI: 10.1007/978-3-642-54050-9; 2014). One day after transfection, supplements (Feed, 12% of total volume) were added. Cell supernatants were harvested after 7 days by centrifugation and subsequent filtration (0.2 μm filter), and proteins were purified from the harvested supernatant by standard methods as indicated below.

### Production of IgG-Like Proteins in CHO K1 Cells

Alternatively, the antibodies and bispecific antibodies described herein were prepared by Evitria using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). Supernatant was harvested by centrifugation and subsequent filtration (0.2 μm filter) and, proteins were purified from the harvested supernatant by standard methods.

### Purification of IgG-like Proteins

Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15, Cat N°: UFC903096), and aggregated protein was separated from monomeric protein by size exclusion chromatography in 20 mM histidine, 140 mM sodium chloride, pH 6.0.

### Analytics of IgG-Like Proteins

The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII (Perkin Elmer). Determination of the aggregate content was performed by HPLC chromatography at 25° C. using analytical size-exclusion column (TSKgel G3000 SW XL or UP-SW3000) equilibrated in running buffer (25 mM K₂HPO₄, 125 mM NaCl, 200 mM L-Arginine Monohydrocloride, pH 6.7 or 200 mM KH₂PO₄, 250 mM KCl pH 6.2, respectively).

TABLE 2
Biochemical analysis of 2 + 1 H2H anti-4-1BB,
anti-FAP huIgG1 P329GLALA antigen binding molecules

| | Monomer | Yield | CE-SDS |
|---|---|---|---|
| Molecule | [%] | [mg/l] | (non-red) |
| 2 + 1 H2H | 100 | 4.7 | 98 |
| 4-1BB (20H4.9)/FAP (4B9) | | | |
| P329GLALA IgG1 | | | |

Preparation of Antigen and Screening Tool Human 4-1BB Fc (kih):

The DNA sequences encoding the ectodomain of human 4-1BB (synthetized according to Q07011) was subcloned in frame with the human IgG1 heavy chain CH2 and CH3 domains on the knob. An AcTEV protease cleavage site was introduced between an antigen ectodomain and the Fc of human IgG1. An Avi tag for directed biotinylation was introduced at the C-terminus of the antigen-Fc knob. Combination of the antigen-Fc knob chain containing the S354C/T366W mutations, with a Fc hole chain containing the Y349C/T366S/L368A/Y407V mutations allows generation of a heterodimer which includes a single copy of 4-1BB ectodomain containing chain, thus creating a monomeric form of Fc-linked antigen. Table 3 shows the amino acid sequences of the antigen Fc-fusion constructs.

TABLE 3
Amino acid sequences of
monomeric antigen Fc(kih) fusion
molecules (produced by combination
of one Fc hole chain with one
antigen Fc knob chain)

| SEQ | | |
|---|---|---|
| ID | | |
| NO: | Antigen | Sequence |
| 69 | Fc hole | DKTHTCPPCPAPELLGGPSVF |
| | chain | LFPPKPKDTLMISRTPEVTCV |
| | | VVDVSHEDPEVKFNWYVDGVE |
| | | VHNAKTKPREEQYNSTYRVVS |
| | | VLTVLHQDWLNGKEYKCKVSN |
| | | KALPAPIEKTISKAKGQPREP |
| | | QVCTLPPSRDELTKNQVSLSC |
| | | AVKGFYPSDIAVEWESNGQPE |
| | | NNYKTTPPVLDSDGSFFLVSK |
| | | LTVDKSRWQQGNVFSCSVMHE |
| | | ALHNHYTQKSLSLSP |
| 70 | human | LQDPCSNCPAGTFCDNNRNQI |
| | 4-1BB | CSPCPPNSFSSAGGQRTCDIC |
| | antigen | RQCKGVFRTRKECSSTSNAEC |
| | Fc knob | DCTPGFHCLGAGCSMCEQDCK |
| | chain | QGQELTKKGCKDCCFGTFNDQ |
| | | KRGICRPWTNCSLDGKSVLVN |
| | | GTKERDVVCGPSPADLSPGAS |
| | | SVTPPAPAREPGHSPQVDEQL |
| | | YFQGGSPKSADKTHTCPPCPA |
| | | PELLGGPSVFLFPPKPKDTLM |
| | | ISRTPEVTCVVVDVSHEDPEV |
| | | KFNWYVDGVEYHNAKTKPREE |
| | | QYNSTYRVVSYLTVLIIQDWL |
| | | NGKEYKCKVSNKALPAPIEKT |
| | | ISKAKGQPREPQVYTLPPCRD |
| | | ELTKNQVSLWCLVKGFYPSDI |
| | | AVEWESNGQPENNYKTTPPVL |
| | | DSDGSFFLYSKLTVDKSRWQQ |
| | | GNVFSCSVMHEALHNHYTQKS |
| | | LSLSPGKSGGLNDIFEAQKIE |
| | | WHE |

1.3 Preparation of Bispecific Antibodies With a Bivalent Binding to 4-1BB and a Monovalent Binding to FAP, Wherein VH and VL of the FAP are Fused at the C-Terminus of the Heavy Chains (Control)

Bispecific agonistic 4-1BB antibodies with bivalent binding for 4-1BB and monovalent binding for FAP, also termed 4-1BB (20H4.9)/FAP (4B9) P329GLALA IgG1 2+1 VH/VL (C-terminal), were prepared wherein VH and VL of the FAP(4B9) binder are fused at the C-terminus of each of the heavy chains, respectively. The knobs into hole technology was applied by introducing the S354C/T366W mutations in the first heavy chain HC1 (Fc knob heavy chain) and introducing the Y349C/T366S/L368A/Y407V mutations in the second heavy chain HC2 (Fc hole heavy chain) to allow generation of a heterodimer.

In this example, the first heavy chain HC1 of the construct was comprised of the following components: VHCH1 of anti-4-1BB binder (clone 20H4.9), followed by Fc knob, at which C-terminus a VL of the anti-FAP binder was fused. The second heavy chain HC2 was comprised of VHCH1 of anti-4-1BB followed by Fc hole, at which C-terminus a VH of the anti-FAP binder (clone 4B9) was fused. Combination of the two heavy chains allows generation of a heterodimer, which includes a FAP binding moiety and two 4-1BB binding Fabs.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO2012/130831A1.

The amino acid sequences for 2+1 anti-4-1BB, anti-FAP constructs with a-FAP VH fused to the Fc knob heavy chain and VL fused to Fc hole heavy chain can be found in Table 4.

TABLE 4
Sequences of bispecific, bivalent
anti-4-1BB/monovalent anti-FAP human
IgG1 P329GLALA antigen binding molecules
(Constructs with a-FAP VH fused
to Fc hole chain and VL fused to
Fc knob chain, termed 2 + 1 VH/VL)

| SEQ | | |
|---|---|---|
| ID | | |
| NO: | Description | Sequence |
| 71 | VHCH1 | QVQLQQWGAGLLKPSETL |
| | (20H4.9)- | SLTCAVYGGSFSGYYWSW |
| | heavy | IRQSPEKGLEWIGEINHG |
| | chain HC1 | GYVTYNPSLESRVTISVD |
| | (Fc hole)- | TSKNQFSLKLSSVTAADT |
| | VH (4B9) | AVYYCARDYGPGNYDWYF |
| | | DLWGRGTLVTVSSASTKG |
| | | PSVFPLAPSSKSTSGGTA |
| | | ALGCLVKDYFPEPVTVSW |
| | | NSGALTSGVHTFPAVLQS |
| | | SGLYSLSSVVTVPSSSLG |
| | | TQTYICNV |
| | | NHKPSNTKVDKKVEPKSC |
| | | DKTHTCPPCPAPEAAGGP |
| | | SVFLFPPKPKDTLMISRT |
| | | PEVTCVVVDVSHEDPEVK |
| | | FNWYVDGVEVHNAKTKPR |
| | | EEQYNSTYRVVSVLTVLH |
| | | QDWLNGKEYKCKVSNKAL |
| | | GAPIEKTISKAKGQPREP |
| | | QVCTLPPSRDELTKNQVS |
| | | LSCAVKGFYPSDIAVEWE |
| | | SNGQPENNYKTTPPVLDS |
| | | DGSFFLVSKLTVDKSRWQ |
| | | QGNVFSCSVMHEALHNHY |
| | | TQKSLSLSPGGGGGSGGG |
| | | GSGGGGSGGGGSEVQLLE |
| | | SGGGLVQPGGSLRLSCAA |
| | | SGFTFSSYAMSWVRQAPG |
| | | KGLEWVSAIIGSGASTYY |
| | | ADSVKGRFTISRDNSKNT |
| | | LYLQMNSLRAEDTAVYYC |
| | | AKGWFGGFNYWGQGTLVT |
| | | VSS |
| 72 | VHCH1 | QVQLQQWGAGLLKPSETLS |
| | (20H4.9)- | LTCAVYGGSFSGYYWSWIR |
| | Heavy | QSPEKGLEWIGEINHGGYV |
| | chain HC2 | TYNPSLESRVTISVDTSKN |
| | (Fc knob)- | QFSLKLSSVTAADTAVYYC |
| | VL (4B9) | ARDYGPGNYDWYFDLWGRG |
| | | TLVTVSSASTKGPSVFPLA |
| | | PSSKSTSGGTAALGCLVKD |
| | | YFPEPVTVSWNSGALTSGV |
| | | HTFPAVLQSSGLYSLSSVV |
| | | TVPSSSLGTQTYICNVNHK |
| | | PSNTKVDKKVEPKSCDKTH |
| | | TCPPCPAPEAAGGPSVFLF |
| | | PPKPKDTLMISRTPEVTCV |
| | | VVDVSHEDPEVKFNWYVDG |
| | | VEVHNAKTKPREEQYNSTY |
| | | RVVSVLTVLHQDWLNGKEY |
| | | KCKVSNKALGAPIEKTISK |
| | | AKGQPREPQVYTLPPCRDE |
| | | LTKNQVSLWCLVKGFYPSD |
| | | IAVEWESNGQPENNYKTTP |
| | | PVLDSDGSFFLYSKLTVDK |
| | | SRWQQGNVFSCSVMHEALH |
| | | NHYTQKSLSLSPGGGGGSG |
| | | GGGSGGGGSGGGGSEIVLT |
| | | QSPGTLSLSPGERATLSCR |
| | | ASQSVTSSYLAWYQQKPGQ |
| | | APRLLINVGSRRATGIPDR |
| | | FSGSGSGTDFTLTISRLEP |
| | | EDFAVYYCQQGIMLPPTFG |
| | | QGTKVEIK |
| 73 | VLCL-Light | EIVLTQSPATLSLSPGERA |
| | chain(20H4.9) | TLSCRASQSVSSYLAWYQQ |
| | | KPGQAPRLLIYDASNRATG |
| | | IPARFSGSGSGTDFTLTIS |
| | | SLEPEDFAVYYCQQRSNWP |
| | | PALTFGGGTKVEIKRTVAA |
| | | PSVFIFPPSDEQLKSGTAS |
| | | VVCLLNNFYPREAKVQWKV |
| | | DNALQSGNSQESVTEQDSK |
| | | DSTYSLSSTLTLSKADYEK |
| | | HKVYACEVTHQGLSSPVTK |
| | | SFNRGEC |

TABLE 5
Biochemical analysis of bispecific antigen binding molecules
with a bivalent binding to 4-1BB and a monovalent binding
to FAP (2 + 1 VH/VL 4-1BB/FAP human IgG1 P329GLALA)

| | Monomer | Yield | CE-SDS |
|---|---|---|---|
| Molecule | [%] | [mg/l] | (non-red) |
| 4-1BB (20H4.9)/FAP (4B9) | 97.7 | 14 | 90 |
| P329GLALA IgG1 2 + 1 | | | |
| VH/VL | | | |

1.4 Preparation of Bispecific Antibodies With a Bivalent Binding to 4-1BB and an Untargeted VH and VL Moiety (DP47 Germline Control), Wherein VH and VL of DP47 are Fused at the C-Terminus of the Heavy Chains (Control)

Bispecific agonistic 4-1BB antibodies with bivalent binding for 4-1BB and monovalent untargeted DP47 germline control , also termed 4-1BB (20H4.9)/untargeted (DP47) P329GLALA IgG1 2+1 VH/VL (C-terminal), were prepared wherein VH and VL of of a non binding clone (DP47) are fused at the C-terminus of each of the heavy chains, respectively. The knobs into hole technology was applied by introducing the S354C/T366W mutations in the first heavy chain HC1 (Fc knob heavy chain) and introducing the Y349C/T366S/L368A/Y407V mutations in the second heavy chain HC2 (Fc hole heavy chain) to allow generation of a heterodimer.

In this example, the first heavy chain HC1 of the construct was comprised of the following components: VHCH1 of anti-4-1BB binder (clone 20H4.9), followed by Fc knob, at which C-terminus a VL of DP47 was fused. The second heavy chain HC2 was comprised of VHCH1 of anti-4-1BB followed by Fc hole, at which C-terminus a VH of DP47 was fused. Combination of the two heavy chains allows generation of a heterodimer, which includes a DP47 instead of the FAP binding moiety and two 4-1BB binding Fabs.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO2012/130831A1.

The amino acid sequences for 2+1 anti-4-1BB, untargeted (DP47) constructs with a-DP47VH fused to the Fc knob heavy chain and VL fused to Fc hole heavy chain can be found in Table 6.

TABLE 6
Sequences of bispecific, bivalent anti-
4-1BB /untargeted (DP47) human IgG1
P329GLALA antigen binding molecules
(Constructs with DP47 VH fused to
Fc hole chain and DP47 VL fused to
Fc knob chain, termed C-terminal)

| SEQ | | |
|---|---|---|
| ID | | |
| NO: | Description | Sequence |
| 74 | VHCH1 | QVQLQQWGAGLLKPSETLSLT |
| | (20H4.9)- | CAVYGGSFSGYYWSWIRQSPE |
| | heavy | KGLEWIGEINHGGYVTYNPSL |
| | chain HC1 | ESRVTISVDTSKNQFSLKLSS |
| | (Fc hole)- | VTAADTAVYYCARDYGPGNYD |
| | VH (DP47) | WYFDLWGRGTLVTVSSASTKG |
| | | PSVFPLAPSSKSTSGGTAALG |
| | | CLVKDYFPEPVTVSWNSGALT |
| | | SGVHTFPAVLQSSGLYSLSSV |
| | | VTVPSSSLGTQTYICNVNHKP |
| | | SNTKVDKKVEPKSCDKTHTCP |
| | | PCPAPEAAGGPSVFLFPPKPK |
| | | DTLMISRTPEVTCWVDVSHED |
| | | PEVKFNWYVDGVEVHNAKTKP |
| | | REEQYNSTYRVVSVLTVLHQD |
| | | WLNGKEYKCKVSNKALGAPIE |
| | | KTISKAKGQPREPQVCTLPPS |
| | | RDELTKNQVSLSCAVKGFYPS |
| | | DIAVEWESNGQPENNYKTTPP |
| | | VLDSDGSFFLVSKLTVDKSRW |
| | | QQGNVFSCSVMHEALHNHYTQ |
| | | KSLSLSPGGGGGSGGGGSEVQ |
| | | LLESGGGLVQPGGSLRLSCAA |
| | | SGFTFSSYAMSWVRQAPGKGL |
| | | EWVSAISGSGGSTYYADSVKG |
| | | RFTISRDNSKNTLYLQMNSLR |
| | | AEDTAVYYCAKGSGFDYWGQG |
| | | TLVTVSS |
| 75 | VHCH1 | QVQLQQWGAGLLKPSETLSLT |
| | (20H4.9)- | CAVYGGSFSGYYWSWIRQSPE |
| | Heavy | KGLEWIGEINHGGYVTYNPSL |
| | chain HC2 | ESRVTISVDTSKNQFSLKLSS |
| | (Fc | VTAADTAVYYCARDYGPGNYD |
| | knob)-VL | WYFDLWGRGTLVTVSSASTKG |
| | (DP47) | PSVFPLAPSSKSTSGGTAALG |
| | | CLVKDYFPEPVTVSWNSGALT |
| | | SGVHTFPAVLQSSGLYSLSSV |
| | | VTVPSSSLGTQTYICNVNHKP |
| | | SNTKVDKKVEPKSCDKTHTCP |
| | | PCPAPEAAGGPSVFLFPPKPK |
| | | DTLMISRTPEVTCVVVDVSHE |
| | | DPEVKFNWYVDGVEV |
| | | HNAKTKPREEQYNSTYRVVSV |
| | | LTVLHQDWLNGKEYKCKVSNK |
| | | ALGAPIEKTISKAKGQPREPQ |
| | | VYTLPPCRDELTKNQVSLWCL |
| | | VKGFYPSDIAVEWESNGQPEN |
| | | NYKTTPPVLDSDGSFFLYSKL |
| | | TVDKSRWQQGNVFSCSVMHEA |
| | | LHNHYTQKSLSLSPGGGGGSG |
| | | GGGSGGGGSGGGGSEIVLTQS |
| | | PGTLSLSPGERATLSCRASQS |
| | | VSSSYLAWYQQKPGQAPRLLI |
| | | YGASSRATGIPDRFSGSGSGT |
| | | DFTLTISRLEPEDFAVYYCQQ |
| | | YGSSPLTFGQGTKVEIK |
| 73 | VLCL-Light | See Table 4 |
| | chain (20H4.9) | |

### Example 2

### Preparation, Purification and Characterization of Bispecific Antibodies With a Bivalent Binding to 4-1BB and a Monovalent Binding to CEA

2.1 Generation and Production of Bispecific antibodies With a Bivalent Binding to 4-1BB and a Monovalent Binding to CEA

Bispecific agonistic 4-1BB antibodies with bivalent binding for 4-1BB and monovalent binding for CEA can also be prepared, by replacing the anti-FAP cross Fab with an anti-CEA cross Fab. This construct is also termed Head to Head (H2H) 2+1 format.

The first heavy chain HC1 of the construct is comprised of the following components: VHCH1 of anti-4-1BB binder (clone 20H4.9), followed by Fc hole. The second heavy chain HC2 was comprised of VLCH1 of anti-CEA binder in cross Fab format followed by VHCH1 of an anti-4-1BB (clone 20H4.9) and by Fc knob. Parental CEA binder A5B7 is described in WO 92/01059. The sequences of CEA binder MFE23 are described in WO 2007/071422. For the 4-1BB binder, the VH and VL sequences of clone 20H4.9 were obtained in accordance with U.S. Pat. No. 7,288,638 B2 or U.S. Pat. No. 7,659,384 B2. Combination of the two heavy chains allows generation of a heterodimer, which includes a CEA binding cross Fab and two 4-1BB binding Fabs (FIG. 1).

To improve correct pairing, the following mutations have been introduced in the CH-CL of the anti-4-1BB Fab molecules: E123R and Q124K in CL and K147E and K213E in CH1. The second light chain LC2 of the anti-CEA binder is composed of VHCL (cross Fab).

The knobs into hole technology is applied by introducing the Y349C/T366S/L368A/Y407V mutations in the first heavy chain HC1 (Fc hole heavy chain) and by introducing the S354C/T366W mutations in the second heavy chain HC2 (Fc knob heavy chain) to allow generation of a heterodimer.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO2012/130831A1.

The bispecific 2+1 H2H anti-4-1BB anti-CEA huIgG1 P329GLALA antibodies are produced as described in Example 1.2 for the 2+1 anti-4-1BB anti-FAP huIgG1 P329GLALA antibodies.

The amino acid sequences for the bispecific 4-1BB (20H4.9)/CEA (A5B7) P329GLALA IgG1 2+1 (H2H) antibody can be found in Table 7, whereas the amino acid sequences of the bispecific 4-1BB (20H4.9)/CEA (MFE23) P329GLALA IgG1 2+1 (H2H) antibody can be found in Table 8.

The proteins are produced and purified as described in Example 1.2.

TABLE 7
Amino acid sequences of bispecific,
bivalent anti-4-1BB /monovalent anti-
CEA(A5B7) human IgG1 P329GLALA
antibody (2 + 1 H2H)

| SEQ | | |
|---|---|---|
| ID | | |
| NO: | Description | Sequence |
| 65 | VHCH1(EE) | See Table 1 |
| | (20H4.9)- | |
| | Heavy | |
| | chain HC1 | |
| | (Fc | |
| | hole) | |
| 76 | VLCH1 (A5B7) | QTVLSQSPAILSASPGEKV |
| | VHCH1(EE) | TMTCRASSSVTYIHWYQQK |
| | (20H4.9)- | PGSSPKSWIYATSNLASGV |
| | Heavy | PARFSGSGSGTSYSLTISR |
| | chain HC2 | VEAEDAATYYCQHWSSKPP |
| | (Fc | TFGGGTKLEIKSSASTKGP |
| | knob) | SVFPLAPSSKSTSGGTAAL |
| | | GCLVKDYFPEPVTVSWNSG |
| | | ALTSGVHTFPAVLQSSGLY |
| | | SLSSVVTVPSSSLGTQTYI |
| | | CNVNHKPSNTKVDKKVEPK |
| | | SCDGGGGSGGGGSQVQLQQ |
| | | WGAGLLKPSETLSLTCAVY |
| | | GGSFSGYYWSWIRQSPEKG |
| | | LEWIGEINHGGYVTYNPSL |
| | | ESRVTISVDTSKNQFSLKL |
| | | SSVTAADTAVYYCARDYGP |
| | | GNYDWYFDLWGRGTLVTVS |
| | | SASTKGPSVFPLAPSSKST |
| | | SGGTAALGCLVEDYFPEPV |
| | | TVSWNSGALTSGVHTFPAV |
| | | LQSSGLYSLSSVVTVPSSS |
| | | LGTQTYICNVNHKPSNTKV |
| | | DEKVEPKSCDKTHTCPPCP |
| | | APEAAGGPSVFLFPPKPKD |
| | | TLMISRTPEVTCWVDVSHE |
| | | DPEVKFNWYVDGVEVHNAK |
| | | TKPREEQYNSTYRVVSVLT |
| | | VLHQDWLNGKEYKCKVSNK |
| | | ALGAPIEKTISKAKGQPRE |
| | | PQVYTLPPCRDELTKNQVS |
| | | LWCLVKGFYPSDIAVEWES |
| | | NGQPENNYKTTPPVLDSDG |
| | | SFFLYSKLTVDKSRWQQGN |
| | | VFSCSVMHEALHNHYTQKS |
| | | LSLSP |
| 67 | VLCL(RK)- | See Table 1 |
| | Light | |
| | chain | |
| | (20H4.9) | |
| 77 | VHCL-Light | EVKLVESGGGLVQPGGSLR |
| | chain | LSCATSGFTFTDYYMNWVR |
| | (A5B7) | QPPGKALEWLGFIGNKANG |
| | | YTTEYSASVKGRFTISRDK |
| | | SQSILYLQMNTLRAEDSAT |
| | | YYCTRDRGLRFYFDYWGQG |
| | | TTLTVSSASVAAPSVFIFP |
| | | PSDEQLKSGTASWCLLNNF |
| | | YPREAKVQWKVDNALQSGN |
| | | SQESVTEQDSKDSTYSLSS |
| | | TLTLSKADYEKHKVYACEV |
| | | THQGLSSPVTKSFNRGEC |

TABLE 8
Amino acid sequences of bispecific,
bivalent anti-4-lBB /monovalent anti-
CEA(MFE23) human IgGl P329GLALA antigen
binding molecules (2 + 1 H2H)

| SEQ ID | | |
|---|---|---|
| NO: | Description | Sequence |
| 65 | VHCH1(EE) | See Table 1 |
| | (20H4.9)-Heavy | |
| | chain HC1 (Fc | |
| | hole) | |
| 78 | VLCH1 (MFE23) | ENVLTQSPAIMSASPGEKV |
| | VHCH1(EE) | TITCSASSSVSYMHWFQQK |
| | (20H4.9)-Heavy | PGTSPKLWIYSTSNLASGV |
| | chain HC2 (Fc | PARFSGSGSGTSYSLTISR |
| | knob) | MEAEDAATYYCQQRSSYPL |
| | | TFGAGTKLELKSSASTKGP |
| | | SVFPLAPSSKSTSGGTAAL |
| | | GCLVKDYFPEPVTVSWNSG |
| | | ALTSGVHTFPAVLQSSGLY |
| | | SLSSVVTVPSSSLGTQTYI |
| | | CNVNHKPSNTKVDKKVEPK |
| | | SCDGGGGSGGGGSQVQLQQ |
| | | WGAGLLKPSETLSLTCAVY |
| | | GGSFSGYYWSWIRQSPEKG |
| | | LEWIGEINHGGYVTYNPSL |
| | | ESRVTISVDTSKNQFSLKL |
| | | SSVTAADTAVYYCARDYGP |
| | | GNYDWYFDLWGRGTLVTVS |
| | | SASTKGPSVFPLAPSSKST |
| | | SGGTAALGCLVEDYFPEPV |
| | | TVSWNSGALTSGVHTFPAV |
| | | LQSSGLYSLSSVVTVPSSS |
| | | LGTQTYICNVNHKPSNTKV |
| | | DEKVEPKSCDKTHTCPPCP |
| | | APEAAGGPSVFLFPPKPKD |
| | | TLMISRTPEVTCVVVDVSH |
| | | EDPEVKFNWYVDGVEVHNA |
| | | KTKPREEQYNSTYRVVSVL |
| | | TVLHQDWLNGKEYKCKVSN |
| | | KALGAPIEKTISKAKGQPR |
| | | EPQVYTLPPCRDELTKNQV |
| | | SLWCLVKGFYPSDIAVEWE |
| | | SNGQPENNYKTTPPVLDSD |
| | | GSFFLYSKLTVDKSRWQQG |
| | | NVFSCSVMHEALHNHYTQK |
| | | SLSLSP |
| 67 | VLCL(RK)-Light | See Table 1 |
| | chain (20H4.9) | |
| 79 | VHCL-Light | QVKLQQSGAELVRSGTSVKL |
| | chain (MFE23) | SCTASGFNIKDSYMHWLRQG |
| | | PEQGLEWIGWIDPENGDTEY |
| | | APKFQGKATFTTDTSSNTAY |
| | | LQLSSLTSEDTAVYYCNEGT |
| | | PTGPYYFDYWGQGTTVTVSS |
| | | ASVAAPSVFIFPPSDEQLKS |
| | | GTASVVCLLNNFYPREAKVQ |
| | | WKVDNALQSGNSQESVTEQD |
| | | SKDSTYSLSSTLTLSKADYE |
| | | KHKVYACEVTHQGLSSPVTK |
| | | SFNRGEC |

Further bispecific agonistic 4-1BB antibodies with bivalent binding for 4-1BB and monovalent binding for CEA can be prepared with anti-CEA binders clone anti-CEA (T84.66-LCHA) or clone anti-CEA(CH1A1A 98/99 SF1). The amino acid sequences for the bispecific 4-1BB (20H4.9)×CEA (T84.66-LCHA) P329GLALA IgG1 2+1 (H2H) antibody can be found in Table 9, whereas the amino acid sequences of the bispecific 4-1BB (20H4.9)×CEA (CH1A1A 98/99 SF1) P329GLALA IgG1 2+1 (H2H) antibody can be found in Table 10.

TABLE 9
Amino acid sequences of bispecific,
bivalent anti-4-1BB /monovalent anti-
CEA(T84.66-LCHA) human IgG1
P329GLALA antibody (2 + 1 H2H)

| SEQ | | |
|---|---|---|
| ID | | |
| NO: | Description | Sequence |
| 65 | VHCH1(EE) | See Table 1 |
| | (20H4.9)- | |
| | Heavy | |
| | chain HC1 | |
| | (Fc | |
| | hole) | |
| 80 | VLCH1 | EIVLTQSPATLSLSPGERA |
| | (T84.66- | TLSCRAGESVDIFGVGFLH |
| | LCHA) | WYQQKPGQAPRLLIYRASN |
| | VHCH1(EE) | RATGIPARFSGSGSGTDFT |
| | (20H4.9)- | LTISSLEPEDFAVYYCQQT |
| | Heavy | NEDPYTFGQGTKLEIKSSA |
| | chain | STKGPSVFPLAPSSKSTSG |
| | HC2 (Fc | GTAALGCLVKDYFPEPVTV |
| | knob) | SWNSGALTSGVHTFPAVLQ |
| | | SSGLYSLSSVVTVPSSSLG |
| | | TQTYICNVNHKPSNTKVDK |
| | | KVEPKSCDGGGGSGGGGSQ |
| | | VQLQQWGAGLLKPSETLSL |
| | | TCAVYGGSFSGYYWSWIRQ |
| | | SPEKGLEWIGEINHGGYVT |
| | | YNPSLESRVTISVDTSKNQ |
| | | FSLKLSSVTAADTAVYYCA |
| | | RDYGPGNYDWYFDLWGRGT |
| | | LVTVSSASTKGPSVFPLAP |
| | | SSKSTSGGTAALGCLVEDY |
| | | FPEPVTVSWNSGALTSGVH |
| | | TFPAVLQSSGLYSLSSVVT |
| | | VPSSSLGTQTYICNVNHKP |
| | | SNTKVDEKVEPKSCDKTHT |
| | | CPPCPAPEAAGGPSVFLFP |
| | | PKPKDTLMISRTPEVTCVV |
| | | VDVSHEDPEVKFNWYVDGV |
| | | EVHNAKTKPREEQYNSTYR |
| | | VVSVLTVLHQDWLNGKEYK |
| | | CKVSNKALGAPIEKTISKA |
| | | KGQPREPQVYTLPPCRDEL |
| | | TKNQVSLWCLVKGFYPSDI |
| | | AVEWESNGQPENNYKTTPP |
| | | VLDSDGSFFLYSKLTVDKS |
| | | RWQQGNVFSCSVMHEALHN |
| | | HYTQKSLSLSP |
| 67 | VLCL(RK)- | See Table 1 |
| | Light | |
| | chain | |
| | (20H4.9) | |
| 81 | VHCL-Light | QVQLVQSGAEVKKPGSSVK |
| | chain | VSCKASGFNIKDTYMHWVR |
| | (T84.66- | QAPGQGLEWMGRIDPANGN |
| | LCHA) | SKYVPKFQGRVTITADTST |
| | | STAYMELSSLRSEDTAVYY |
| | | CAPFGYYVSDYAMAYWGQG |
| | | TLVTVSSASVAAPSVFIFP |
| | | PSDEQLKSGTASWCLLNNF |
| | | YPREAKVQWKVDNALQSGN |
| | | SQESVTEQDSKDSTYSLSS |
| | | TLTLSKADYEKHKVYACEV |
| | | THQGLSSPVTKSFNRGEC |

TABLE 10
Amino acid sequences of bispecific,
bivalent anti-4-1BB /monovalent anti-
CEA(CH1A1A 98/99 2F1) human IgG1 P329GLALA
antigen binding molecules (2 + 1 H2H)

| SEQ | | |
|---|---|---|
| ID | | |
| NO: | Description | Sequence |
| 65 | VHCH1(EE) | See Table 1 |
| | (20H4.9)- | |
| | Heavy | |
| | chain HC1 | |
| | (Fc | |
| | hole) | |
| 82 | VLCH1 | DIQMTQSPSSLSASVGDR |
| | (CEA2F1) | VTITCKASAAVGTYVAWY |
| | VHCH1 | QQKPGKAPKLLIYSASYR |
| | (EE) | KRGVPSRFSGSGSGTDFT |
| | (20H4.9)- | LTISSLQPEDFATYYCHQ |
| | Heavy | YYTYPLFTFGQGTKLEIK |
| | chain | SSASTKGPSVFPLAPSSK |
| | HC2 | STSGGTAALGCLVKDYFP |
| | (Fcknob) | EPVTVSWNSGALTSGVHT |
| | | FPAVLQSSGLYSLSSVVT |
| | | VPSSSLGTQTYICNVNHK |
| | | PSNTKVDKKVEPKSCDGG |
| | | GGSGGGGSQVQLQQWGAG |
| | | LLKPSETLSLTCAVYGGS |
| | | FSGYYWSWIRQSPEKGLE |
| | | WIGEINHGGYVTYNPSLE |
| | | SRVTISVDTSKNQFSLKL |
| | | SSVTAADTAVYYCARDYG |
| | | PGNYDWYFDLWGRGTLVT |
| | | VSSASTKGPSVFPLAPSS |
| | | KSTSGGTAALGCLVEDYF |
| | | PEPVTVSWNSGALTSGVH |
| | | TFPAVLQSSGLYSLSSVV |
| | | TVPSSSLGTQTYICNVNH |
| | | KPSNTKVDEKVEPKSCDK |
| | | THTCPPCPAPEAAGGPSV |
| | | FLFPPKPKDTLMISRTPE |
| | | VTCVVVDVSHEDPEVKFN |
| | | WYVDGVEVHNAKTKPREE |
| | | QYNSTYRVVSVLTVLHQD |
| | | WLNGKEYKCKVSNKALGA |
| | | PIEKTISKAKGQPREPQV |
| | | YTLPPCRDELTKNQVSLW |
| | | CLVKGFYPSDIAVEWESN |
| | | GQPENNYKTTPPVLDSDG |
| | | SFFLYSKLTVDKSRWQQG |
| | | NVFSCSVMHEALHNHYTQ |
| | | KSLSLSP |
| 67 | VLCL(RK)- | See Table 1 |
| | Light | |
| | chain | |
| | (20H4.9) | |
| 83 | VHCL- | QVQLVQSGAEVKKPGASVK |
| | Light | VSCKASGYTFTEFGMNWVR |
| | chain | QAPGQGLEWMGWINTKTGE |
| | (CEA | ATYVEEFKGRVTFTTDTST |
| | CH1A1A | STAYMELRSLRSDDTAVYY |
| | 98/99) | CARWDFAYYVEAMDYWGQG |
| | | TTVTVSSASVAAPSVFIFP |
| | | PSDEQLKSGTASWCLLNNF |
| | | YPREAKVQWKVDNALQSGN |
| | | SQESVTEQDSKDSTYSLSS |
| | | TLTLSKADYEKHKVYACEV |
| | | THQGLSSPVTKSFNRGEC |

### 2.2 Generation of Humanized Variants Anti-CEA Antibody A5B7

### 2.2.1 Methodology

Anti-CEA antibody A5B7 is for example disclosed by M. J. Banfield et al, Proteins 1997, 29(2), 161-171 and its structure can be found as PDB ID:1CLO in the Protein structural database PDB (www.rcsb.org, H. M. Berman et al, The Protein Data Bank, Nucleic Acids Research, 2000, 28, 235-242). This entry includes the heavy and the light chain variable domain sequence. For the identification of a suitable human acceptor framework during the humanization of the anti-CEA binder A5B7, a classical approach was taken by searching for an acceptor framework with high sequence homology, grafting of the CDRs on this framework, and evaluating which back-mutations can be envisaged. More explicitly, each amino acid difference of the identified frameworks to the parental antibody was judged for impact on the structural integrity of the binder, and back mutations towards the parental sequence were introduced whenever appropriate. The structural assessment was based on Fv region homology models of both the parental antibody and its humanized versions created with an in-house antibody structure homology modeling tool implemented using the Biovia Discovery Studio Environment, version 4.5.

### 2.2.2 Choice of Acceptor Framework and Adaptations Thereof

The acceptor framework was chosen as described in Table 11 below:

TABLE 11
Acceptor framework

| | | Choice of human |
|---|---|---|
| | Closest murine | acceptor V-region |
| | V-region germline | germline |
| A5B7 VH | mu-IGHV7-3-02 | IGHV3-23-01 or |
| | | IGHV3-15-01 |
| A5B7 VL | mu-IGKV4-72-01 | IGKV3-11-01 |

Post-CDR3 framework regions were adapted from human J-element germline IGJH6 for the heavy chain, and a sequence similar to the kappa J-element IGKJ2, for the light chain.

Based on structural considerations, back mutations from the human acceptor framework to the amino acid in the parental binder were introduced at positions 93 and 94 of the heavy chain.

### 2.2.3 VH and VL Regions of the Resulting Humanized CEA Antibodies

The resulting VH domains of humanized CEA antibodies can be found in Table 12 below and the resulting VL domains of humanized CEA antibodies are listed in Table 13 below.

TABLE 12
Amino acid sequences of the VH domains
of humanized CEA antibodies, based
on human acceptor framework IGHV3-23
or IGHV3-15

| | | Seq |
|---|---|---|
| | | ID |
| Description | Sequence | No |
| A5B7 VH | EVKLVESGGGLVQPGGSLRLSCATSG | 31 |
| murine | FTFTDYYMNWVRQPPGKALEWLGFIG | |
| donor | NKANGYTTEYSASVKGRFTISRDKSQ | |
| sequence | SILYLQMNTLRAEDSATYYCTRDRGL | |
| | RFYFDYWGQGTTLTVSS | |
| IGHV3- | EVQLLESGGGLVQPGGSLRLSCAASG | 153 |
| 23-02 | FTFSSYAMSWVRQAPGKGLEWVSAIS | |
| human | GSGGSTYYGDSVKGRFTISRDNSKNT | |
| acceptor | LYLQMNSLRAEDTAVYYCAK | |
| sequence | | |
| Humanized | | |
| variants | | |
| 3-23A5-1 | EVQLLESGGGLVQPGGSLRLSCAASG | 155 |
| | FTFTDYYMNWVRQAPGKGLEWVGFIG | |
| | NKANGYTTEYSASVKGRFTISRDNSK | |
| | NTLYLQMNSLRAEDTAVYYCARDRGL | |
| | RFYFDYWGQGTTVTVSS | |
| 3-23A5-2 | EVQLLESGGGLVQPGGSLRLSCAASG | 156 |
| | FTFTDYYMNWVRQAPGKGLEWVGFIG | |
| | NKANGYTTYYGDSVKGRFTISRDNSK | |
| | NTLYLQMNSLRAEDTAVYYCARDRGL | |
| | RFYFDYWGQGTTVTVSS | |
| 3-23A5-3 | EVQLLESGGGLVQPGGSLRLSCAASG | 157 |
| | FTFTDYYMNWVRQAPGKGLEWVGFIG | |
| | NKGYTTEYSASVKGRFTISRDNSKNT | |
| | LYLQMNSLRAEDTAVYYCARDRGLRF | |
| | YFDYWGQGTTVTVSS | |
| 3-23A5-4 | EVQLLESGGGLVQPGGSLRLSCAASG | 158 |
| | FTFTDYYMSWVRQAPGKGLEWVGFIG | |
| | NKANGYTTEYSASVKGRFTISRDNSK | |
| | NTLYLQMNSLRAEDTAVYYCARDRGL | |
| | RFYFDYWGQGTTVTVSS | |
| 3-23A5-1A | EVQLLESGGGLVQPGGSLRLSCAASG | 159 |
| (all_ | FTFTDYYMNWVRQAPGKGLEWLGFIG | |
| back | NKANGYTTEYSASVKGRFTISRDKSK | |
| mutations) | NTLYLQMNSLRAEDTATYYCTRDRGL | |
| | RFYFDYWGQGTTVTVSS | |
| 3-23A5-1C | EVQLLESGGGLVQPGGSLRLSCAASG | 160 |
| (A93T) | FTFTDYYMNWVRQAPGKGLEWVGFIG | |
| | NKANGYTTEYSASVKGRFTISRDNSK | |
| | NTLYLQMNSLRAEDTAVYYCTRDRGL | |
| | RFYFDYWGQGTTVTVSS | |
| 3-23A5-1D | EVQLLESGGGLVQPGGSLRLSCAASG | 161 |
| (K73) | FTFTDYYMNWVRQAPGKGLEWVGFIG | |
| | NKANGYTTEYSASVKGRFTISRDKSK | |
| | NTLYLQMNSLRAEDTAVYYCARDRGL | |
| | RFYFDYWGQGTTVTVSS | |
| 3-23A5-1E | EVQLLESGGGLVQPGGSLRLSCAASG | 121 |
| (G54A) | FTFTDYYMNWVRQAPGKGLEWLGFIG | |
| | NKANAYTTEYSASVKGRFTISRDKSK | |
| | NTLYLQMNSLRAEDTATYYCTRDRGL | |
| | RFYFDYWGQGTTVTVSS | |
| IGHV3- | EVQLVESGGGLVKPGGSLRLSCAASG | 154 |
| 15*01 | FTFSNAWMSWVRQAPGKGLEWVGRIK | |
| human | SKTDGGTTDYAAPVKGRFTISRDDSK | |
| acceptor | NTLYLQMNSLKTEDTAVYYCTT | |
| sequence | | |
| Humanized | | |
| variants | | |
| 3-15A5-1 | EVQLVESGGGLVKPGGSLRLSCAASG | 162 |
| | FTFTDYYMNWVRQAPGKGLEWVGFIG | |
| | NKANGYTTEYSASVKGRFTISRDDSK | |
| | NTLYLQMNSLKTEDTAVYYCTRDRGL | |
| | RFYFDYWGQGTTVTVSS | |
| 3-15A5-2 | EVQLVESGGGLVKPGGSLRLSCAASG | 163 |
| | FTFTDYYMNWVRQAPGKGLEWVGFIG | |
| | NKANGYTTEYAAPVKGRFTISRDDSK | |
| | NTLYLQMNSLKTEDTAVYYCTRDRGL | |
| | RFYFDYWGQGTTVTVSS | |
| 3-15A5-3 | EVQLVESGGGLVKPGGSLRLSCAASG | 164 |
| | FTFTDYYMNWVRQAPGKGLEWVGFIG | |
| | NKANGGTTDYAAPVKGRFTISRDDSK | |
| | NTLYLQMNSLKTEDTAVYYCTRDRGL | |
| | RFYFDYWGQGTTVTVSS | |

For the heavy chain, the initial variant 3-23A5-1 was found suitable in binding assays (but showed slightly less binding than the parental murine antibody) and was chosen as starting point for further modifications. The variants based on IGHV3-15 showed less binding activity compared to humanized variant 3-23A5-1.

In order to restore the full binding activity of the parental chimeric antibody, variants 3-23A5-1A, 3-23A5-1C and 3-23A5-1D were created. It was also tested for variant 3-23A5-1 whether the length of CDR-H2 could be adapted to the human acceptor sequence, but this construct completely lost binding activity. Since a putative deamidation hotspot was present in CDR-H2 (Asn53-Gly54), we changed that motif to Asn53-Ala54. Another possible hotspot Asn73-Ser74 was backmutated to Lys73-Ser74. Thus, variant 3-23A5-1E was created.

TABLE 13
Amino acid sequences of the VL domains
of humanized CEA antibodies, based
on human acceptor framework IGKV3-11.

| | | Seq |
|---|---|---|
| Descrip- | | ID |
| tion | Sequence | No |
| A5B7 VL | QTVLSQSPAILSASPGEKVTMTCRASS | 32 |
| murine | SVTYIHWYQQKPGSSPKSWIYATSNLA | |
| donor | SGVPARFSGSGSGTSYSLTISRVEAED | |
| sequence | AATYYCQHWSSKPPTFGGGTKLEIK | |
| IGKV3-11 | EIVLTQSPATLSLSPGERATLSCRASQ | 165 |
| human | SVSSYLAWYQQKPGQAPRLLIYDASNR | |
| acceptor | ATGIPARFSGSGSGTDFTLTISSLEPE | |
| sequence | DFAVYYCQQRSNWP | |
| humanized | | |
| variants | | |
| A5-L1 | ElVLTQSPATLSLSPGERATLSCRASS | 166 |
| | SVTYIHWYQQKPGQAPRLLIYATSNLA | |
| | SGIPARFSGSGSGTDFTLTISSLEPED | |
| | FAVYYCQHWSSKPPTFGQGTKLEIK | |
| A5-L2 | EIVLTQSPATLSLSPGERATLSCRASQ | 167 |
| | SVSSYIHWYQQKPGQAPRLLIYATSNL | |
| | ASGIPARFSGSGSGTDFTLTISSLEPE | |
| | DFAVYYCQHWSSKPPTFGQGTKLEIK | |
| A5-L3 | EIVLTQSPATLSLSPGERATLSCRASS | 168 |
| | SVTYIHWYQQKPGQAPRLLIYDASNRA | |
| | TGIPARFSGSGSGTDFTLTISSLEPED | |
| | FAVYYCQHWSSKPPTFGQGTKLEIK | |
| A5-L4 | ElVLTQSPATLSLSPGERATLSCRASS | 169 |
| | SVTYIHWYQQKPGQAPRLLIYATSNLA | |
| | SGIPARFSGSGSGTDFTLTISSLEPED | |
| | FAVYYCQQWSSKPPTFGQGTKLEIK | |
| A5-L1A | QTVLTQSPATLSLSPGERATLSCRASS | 170 |
| (all_back | SVTYIHWYQQKPGSSPKSWIYATSNLA | |
| mutations) | SGIPARFSGSGSGTDYTLTISSLEPED | |
| | FAVYYCQHWSSKPPTFGQGTKLEIK | |
| A5-L1B | QTVLTQSPATLSLSPGERATLSCRASS | 171 |
| (Q1T2) | SVTYIHWYQQKPGQAPRLLIYATSNLA | |
| | SGIPARFSGSGSGTDFTLTISSLEPED | |
| | FAVYYCQHWSSKPPTFGQGTKLEIK | |
| A5-L1C | ElVLTQSPATLSLSPGERATLSCRASS | 172 |
| (FR2) | SVTYIHWYQQKPGSSPKSWIYATSNLA | |
| | SGIPARFSGSGSGTDFTLTISSLEPED | |
| | FAVYYCQHWSSKPPTFGQGTKLEIK | |
| A5-L1D | EIVLTQSPATLSLSPGERATLSCRASS | 122 |
| (46, 47) | SVTYIHWYQQKPGQAPRSWIYATSNLA | |
| | SGIPARFSGSGSGTDFTLTISSLEPED | |
| | FAVYYCQHWSSKPPTFGQGTKLEIK | |

The light chain was humanized based on the human IGKV3-11 acceptor framework. In the series A5-L1 to A5-L4, it was learned that variant A5-L1 shows good binding activity (but slightly less than the parental antibody). Partial humanization of CDR-L1 (variant A5-L2; Kabat positions 30 and 31) fully abrogates the binding. Likewise, humanization of CDR-H2 (variant A5-L3; Kabat positions 50 to 56) also fully abrogates the binding. The position 90 (variant A5-L4) shows significant contribution to the binding properties. The Histidine at this position is important for binding. Thus, variant A5-L1 was chosen for further modification.

The series A5-L1A to A5-L1D addressed the question which backmutations are required to restore the full binding potential of the parental chimeric antibody. Variant A5-L1A showed that backmutations at Kabat positions 1, 2, the entire framework 2, and Kabat position 71 do not add any further binding activity. Variants A5-L1B, and A5-L1C addressed subsets of those positions and confirm that they do not alter the binding properties. Variant A5l -L1D with back mutations at Kabat positions 46 and 47 showed the best binding activity.

### 2.2.4 Selection of Humanized A5B7 Antibodies

Based on the new humanization variants of VH and VL new CEA antibodies were expressed as huIgG1 antibodies with an effector silent Fc (P329G; L234, L235A) to abrogate binding to Fcγ receptors according to the method described in WO 2012/130831 A1 and their binding to CEA expressed on MKN45 cells was tested and compared to the respective parental murine A5B7 antibody.

TABLE 14
VH/VL combinations expressed as huIgG1_LALA_PG antibodies

| | A5-L1A | A5-L1B | A5-L1C | A5-L1D |
|---|---|---|---|---|
| 3-23A5-1A | P1AE2164 | P1AE2165 | P1AE2166 | P1AE2167 |
| 3-23A5-1C | — | — | P1AE2176 | P1AE2177 |
| 3-23A5-1D | P1AE2179 | — | P1AE2181 | P1AE2182 |

MKN45 (DSMZ ACC 409) is a human gastric adenocarcinoma cell line expressing CEA. The cells were cultured in advanced RPMI+2% FCS+1% Glutamax. Viability of MKN-45 cells was checked and cells were re-suspended and adjusted to a density of 1 Mio cells/ml. 100 μl of this cell suspension (containing 0.1 Mio cells) were seeded into a 96 well round bottom plate. The plate was centrifuged for 4 min at 400×g and the supernatant was removed. Then 40 μl of the diluted antibodies or FACS buffer were added to the cells and incubated for 30 min at 4° C. After the incubation the cells were washed twice with 150 μl FACS buffer per well. Then 20 μl of the diluted secondary PE anti-human Fc specific secondary antibody (109-116-170, Jackson ImmunoResearch) was added to the cells. The cells were incubated for an additional 30 min at 4° C. To remove unbound antibody, the cells were washed again twice with 150 μl per well FACS buffer. To fix the cells 100 μl of FACS buffer containing 1% PFA were added to the wells. Before measuring the cells were re-suspended in 150 μl FACS buffer. The fluorescence was measured using a BD flow cytometer.

In FIG. 7 binding curves of the humanized A5B7 variants are shown. All tested binders were able to bind to MKN45 cells but binding capacity was slightly reduced compared to the parental A5B7 antibody. The clone P1AE2167 had the best binding of all tested variants and was selected for further development.

2.2.5 Determination of affinities of Fab fragments of humanized variants of murine CEA-antibody A5B7 to human CEA using surface plasmon resonance (BIACORE)

The affinities of Fab fragments of the humanized variants of murine CEA antibody A5B7 to human CEA were assessed by surface plasmon resonance using a BIACORE T200 instrument. On a CM5 chip, human CEA (hu N(A2-B2)A-avi-His B) was immobilized at a 40 nM concentration by standard amine coupling on flow cell 2 for 30s to about 100RU. The Fab fragments of the humanized variants of murine CEA antibody A5B7 were subsequently injected as analytes in 3-fold dilutions ranging from 500-0.656nM for a contact time of 120s, a dissociation time of 250 or 1000s and at a flow rate of 30 μl/min. Regeneration at the level of human CEA (hu N(A2-B2)A-avi-His B) was achieved by 2 pulses of 10 mM glycine/HCl pH2.0 for 60s. Data were double-referenced against the unimmobilized flow cell 1 and a zero concentration of the analyte. The sensorgrams of the analytes were fitted to a simple 1:1 Langmuir interaction model. Affinity constants [K_{D}] for human CEA (A2 domain) are summarized in Table 15 below.

TABLE 15
Affinity constants of Fab fragments representing
different humanized variants of murine CEA
antibody A5B7 to human CEA (A2 domain).

| | | Affinity to human |
|---|---|---|
| | | N(A2-B2)A-avi-His B |
| Tapir ID | Name | [M] |
| P1AE0289 | CEA (A5B7) Fab (parental | 5.59E−10 |
| | murine antibody) | |
| P1AE4135 | Fab derived from P1AE2164 | 1.70E−09 |
| P1AE4136 | Fab derived from P1AE2165 | 1.25E−09 |
| P1AE4137 | Fab derived from P1AE2166 | 1.13E−08 |
| P1AE4138 | Fab derived from P1AE2167 | 1.47E−09 |
| P1AE4139 | Fab derived from P1AE2176 | 7.58E−09 |
| P1AE4140 | Fab derived from P1AE2177 | 7.62E−09 |
| P1AE4141 | Fab derived from P1AE2179 | 1.83E−09 |
| P1AE4142 | Fab derived from P1AE2181 | 2.64E−09 |
| P1AE4143 | Fab derived from P1AE2182 | 2.92E−09 |

The humanized variants of the murine CEA antibody A5B7 are of lower affinities than the parental murine antibody. The fab fragment P1AE4138, derived from P1AE2167 (heavy chain with VH variant 3-23A5-1A and Ckappa light chain with VL variant A5-L1D) was chosen as final humanized variant. Moreover, a glycine to alanine mutation at Kabat position 54 (G54A) was introduced into the VH domain in order to remove a deamidation site, leading to VL variant 3-23A5-1E. The final humanized antibody (heavy chain with VH variant 3-23A5-1E and Ckappa light chain with VL variant A5-L1D) has been named A5H1EL1D or huA5B7.

### 2.2.6 Generation and Production of Bispecific Antibodies With a Bivalent Binding to 4-1BB and a Monovalent Binding to CEA (A5H1EL1D)

The bispecific 2+1 H2H anti-4-1BB anti-CEA huIgG1 P329GLALA antibodies are produced as described in Example 2.1 for the 2+1 anti-4-1BB anti-CEA (A5B7) huIgG1 P329GLALA antibodies.

The amino acid sequences for the bispecific 4-1BB (20H4.9)/CEA (A5H1EL1D) P329GLALA IgG1 2+1 (H2H) antibody can be found in Table 16. The protein is produced and purified as described in Example 1.2.

TABLE 16
Amino acid sequences of bispecific,
bivalent anti-4-1BB/monovalent anti-
CEA(A5H1EL1D) human IgG1 P329GLALA
antigen binding molecules (2 + 1 H2H)

| SEQ | | |
|---|---|---|
| ID | | |
| NO: | Description | Sequence |
| 65 | VHCH1(EE) | Sec Table 1 |
| | (20H4.9)-Heavy | |
| | chain HC1 (Fc | |
| | hole) | |
| 173 | VLCH1 (CEA | EIVLTQSPATLSLSPGERATL |
| | A5H1EL1D) | SCRASSSVTYIHWYQ |
| | VHCH1(EE) | QKPGQAPRSWIYATSNLASGI |
| | (20H4.9)-Heavy | PARFSGSGSGTDFTLTISSLE |
| | chain HC2 (Fc | PEDFAVYYCQHWSSKPPTFGQ |
| | knob) | GTKLEIKSSASTKGPSVFPLA |
| | | PSSKSTSGGTAALGCLVKDYF |
| | | PEPVTVSWNSGALTSGVHTFP |
| | | AVLQSSGLYSLSSVVTVPSSS |
| | | LGTQTYICNVNHKPSNTKVDK |
| | | KVEPKSCDGGGGSGGGGSQVQ |
| | | LQQWGAGLLKPSETLSLTCAV |
| | | YGGSFSGYYWSWIRQSPEKGL |
| | | EWIGEINHGGYVTYNPSLESR |
| | | VTISVDTSKNQFSLKLSSVTA |
| | | ADTAVYYCARDYGPGNYDWYF |
| | | DLWGRGTLVTVSSASTKGPSV |
| | | FPLAPSSKSTSGGTAALGCLV |
| | | EDYFPEPVTVSWNSGALTSGV |
| | | HTFPAVLQSSGLYSLSSVVTV |
| | | PSSSLGTQTYICNVNHKPSNT |
| | | KVDEKVEPKSCDKTHTCPPCP |
| | | APEAAGGPSVFLFPPKPKDTL |
| | | MISRTPEVTCVVVDVSHEDPE |
| | | VKFNWYVDGVEVHNAKTKPRE |
| | | EQYNSTYRVVSVLTVLHQDWL |
| | | NGKEYKCKVSNKALGAPIEKT |
| | | ISKAKGQPREPQVYTLPPCRD |
| | | ELTKNQVSLWCLVKGFYPSDI |
| | | AVEWESNGQPENNYKTTPPVL |
| | | DSDGSFFLYSKLTVDKSRWQQ |
| | | GNVFSCSVMHEALHNHYTQKS |
| | | LSLSP |
| 67 | VLCL(RK)-Light | See Table 1 |
| | chain (20H4.9) | |
| 174 | VHCL-Light | EVQLLESGGGLVQPGGSLRL |
| | chain (CEA | SCAASGFTFTDYYMNWVRQA |
| | A5H1EL1D) | PGKGLEWLGFIGNKANAYTT |
| | | EYSASVKGRFTISRDKSKNT |
| | | LYLQMNSLRAEDTATYYCTR |
| | | DRGLRFYFDYWGQGTTVTVS |
| | | SASVAAPSVFIFPPSDEQLK |
| | | SGTASVVCLLNNFYPREAKV |
| | | QWKVDNALQSGNSQESVTEQ |
| | | DSKDSTYSLSSTLTLSKADY |
| | | EKHKVYACEVTHQGLSSPVT |
| | | KSFNRGEC |

### 2.3 Generation of Humanized Variants of Anti-CEA Antibody MFE23

### 2.3.1 Methodology

Anti-CEA antibody MFE23 is for example disclosed by M. K. Boehm et al, Biochem. J. 2000, 346, 519-528 and its structure can be found as PDB ID:11QOK in the Protein structural database PDB (www.rcsb.org, H. M. Berman et al, The Protein Data Bank, Nucleic Acids Research, 2000, 28, 235-242). This entry includes the heavy and the light chain variable domain sequence. For the identification of a suitable human acceptor framework during the humanization of the anti-CEA binder MFE23, a classical approach was taken by searching for an acceptor framework with high sequence homology, grafting of the CDRs on this framework, and evaluating which back-mutations can be envisaged. More explicitly, each amino acid difference of the identified frameworks to the parental antibody was judged for impact on the structural integrity of the binder, and back mutations towards the parental sequence were introduced whenever appropriate. The structural assessment was based on Fv region homology models of both the parental antibody and its humanized versions created with an in-house antibody structure homology modeling tool implemented using the Biovia Discovery Studio Environment, version 4.5.

In order to increase confidence in the choice of back mutations, we identified the closest murine homologous sequence, from which this antibody might have derived. There we looked for positions that have undergone extensive somatic hypermutation during the maturation of this antibody in the murine B-cell. Those mutations would be of potential importance to be incorporated in the humanized construct.

### 2.3.2 Choice of Acceptor Framework and Adaptations Thereof

The acceptor framework was chosen as described in Table 17 below:

TABLE 17
Acceptor framework

| | | Choice of human |
|---|---|---|
| | Closest murine | acceptor V-region |
| | V-region germline | germline |
| MFE23 VH | m-IGHV14-4-02 | IGHV1-2-02 |
| MFE23 VL | m-IGKV4-57-01 | IGKV1-39-01 |

Post-CDR3 framework regions were adapted from human J-element germline IGHJ4-01 for the heavy chain, and a sequence similar to the kappa J-element IGKJ4-01, for the light chain. Based on structural considerations, back mutations from the human acceptor framework to the amino acid in the parental binder were introduced at Kabat positions 71 and 93 of the heavy chain. Based on considerations that framework mutations in the murine germline, leading to the final matured MFE23 sequence, would be of importance, the residues at Kabat position 94 of VH was changed back to the murine sequence.

In order to evaluate further affinity and/or stability improvements on the MFE23 sequence, we incorporated the following mutations in the light chain sequence: Phe26Leu, Ser30Pro, or Tyr, Leu78Val as described by C. P. Graff et al., Protein Engineering, Design & Selection 2004, 17(4), 293-304.

### 2.3.3 VH and VL Domains of the Resulting Humanized CEA Antibodies

The resulting VH domains of humanized CEA antibodies can be found in Table 18 below and the resulting VL domains of humanized CEA antibodies are listed in Table 19 below.

TABLE 18
Amino acid sequences of the VH domains
of humanized CEA antibodies, based
on human acceptor framework IGHV1-2-02

| | | Seq |
|---|---|---|
| | | ID |
| Description | Sequence | No |
| MFE23 VH | QVKLQQSGAELVRSGTSVKLSCTASG | 39 |
| murine | FNIKDSYMHWLRQGPEQGLEWIGWID | |
| donor | PENGDTEYAPKFQGKATFTTDTSSNT | |
| sequence | AYLQLSSLTSEDTAVYYCNEGTPTGP | |
| | YYFDYWGQGTTVTVSS | |
| IGHV1-2-02 | QLVQSGAEVKKPGASVKVSCKASGYT | 175 |
| human | FTGYYMHWVRQAPGQGLEWMGWINPN | |
| acceptor | SGGTNYAQKFQGRVTMTRDTSISTAY | |
| sequence | MELSRLRSDDTAVYYCAR | |
| IGHV1-69-01 | QVQLVQSGAEVKKPGSSVKVSCKASG | 176 |
| human | GTFSSYAISWVRQAPGQGLEWMGGII | |
| acceptor | PIFGTANYAQKFQGRVTITADESTST | |
| sequence | AYMELSSLRSEDTAVYYCAR | |
| IGHV1-69-05 | QVQLVQSGAEVKKPGSSVKVSCKASG | 177 |
| human | GTFSSYAISWVRQAPGQGLEWMGGII | |
| acceptor | PIFGTANYAQKFQGRVTITTDESTST | |
| sequence | AYMELSSLRSEDTAVYYCAR | |
| Humanized | | |
| variants | | |
| MFE-H24 | QVQLVQSGAEVKKPGASVKVSCKASG | 133 |
| | FNIKDSYMHWVRQAPGQGLEWMGWID | |
| | PENGDTEYAPKFQGRVTMTTDTSIST | |
| | AYMELSRLRSDDTAVYYCNEGTPTGP | |
| | YYFDYWGQGTLVTVSS | |
| MFE-H25 | QVQLVQSGAEVKKPGASVKVSCKASG | 134 |
| | YTFKDSYMHWVRQAPGQGLEWMGWID | |
| | PENGDTEYAPKFQGRVTMTTDTSIST | |
| | AYMELSRLRSDDTAVYYCNEGTPTGP | |
| | YYFDYWGQGTLVTVSS | |
| MFE-H26 | QVQLVQSGAEVKKPGASVKVSCKASG | 135 |
| | FNIKDSYMHWVRQAPGQGLEWMGWID | |
| | PENGGTNYAQKFQGRVTMTTDTSIST | |
| | AYMELSRLRSDDTAVYYCNEGTPTGP | |
| | YYFDYWGQGTLVTVSS | |
| MFE-H27 | QVQLVQSGAEVKKPGASVKVSCKASG | 136 |
| | FNIKDSYMHWVRQAPGQGLEWMGWID | |
| | PENGDTEYAPKFQGRVTMTTDTSIST | |
| | AYMELSRLRSDDTAVYYCARGTPTGP | |
| | YYFDYWGQGTLVTVSS | |
| MFE-H28 | QVQLVQSGAEVKKPGASVKVSCKASG | 137 |
| | FNIKDSYMHWVRQAPGQGLEWMGWID | |
| | PENGDTEYAPKFQGRVTMTRDTSIST | |
| | AYMELSRLRSDDTAVYYCNEGTPTGP | |
| | YYFDYWGQGTLVTVSS | |
| MFE-H29 | QVQLVQSGAEVKKPGSSVKVSCKASG | 138 |
| | FNIKDSYMHWVRQAPGQGLEWMGWID | |
| | PENGDTEYAPKFQGRVTITTDESTST | |
| | AYMELSSLRSEDTAVYYCNEGTPTGP | |
| | YYFDYWGQGTLVTVSS | |

TABLE 19
Amino acid sequences of the VL domains
of humanized CEA antibodies, based
on human acceptor framework IGKV1-39-01

| | | Seq |
|---|---|---|
| | | ID |
| Description | Sequence | No |
| MFE23 VL | ENVLTQSPAIMSASPGEKVTITCSAS | 40 |
| murine donor | SSVSYMHWFQQKPGTSPKLWIYSTSN | |
| sequence | LASGVPARFSGSGSGTSYSLTISRME | |
| | AEDAATYYCQQRSSYPLTFGAGTKLE | |
| | LK | |
| IGKV1-39-01 | DIQMTQSPSSLSASVGDRVTITCRAS | 178 |
| human | QSISSYLNWYQQKPGKAPKLLIYAAS | |
| acceptor | SLQSGVPSRFSGSGSGTDFTLTISSL | |
| sequence | QPEDFATYYCQQSYSTP | |
| Humanized | | |
| variants | | |
| MFE-L24 | DIQMTQSPSSLSASVGDRVTITCRAS | 139 |
| | SSVSYMHWYQQKPGKAPKLLIYSTSN | |
| | LASGVPSRFSGSGSGTDFTLTISSLQ | |
| | PEDFATYYCQQRSSYPLTFGGGTKLE | |
| | IK | |
| MFE-L25 | EIQMTQSPSSLSASVGDRVTITCRAS | 140 |
| | SSVSYMHWYQQKPGKAPKLLIYSTSN | |
| | LASGVPSRFSGSGSGTDFTLTISSLQ | |
| | PEDFATYYCQQRSSYPLTFGGGTKLE | |
| | IK | |
| MFE-L26 | EIQMTQSPSSLSASVGDRVTITCRAS | 141 |
| | QSISSYMHWYQQKPGKAPKLLIYSTS | |
| | NLASGVPSRFSGSGSGTDFTLTISSL | |
| | QPEDFATYYCQQRSSYPLTFGGGTKL | |
| | EIK | |
| MFE-L27 | EIQMTQSPSSLSASVGDRVTITCRAS | 142 |
| | SSVPYMHWYQQKPGKAPKLLIYSTSN | |
| | LASGVPSRFSGSGSGTDFTLTISSVQ | |
| | PEDFATYYCQQRSSYPLTFGGGTKLE | |
| | IK | |
| MFE-L28 | EIQMTQSPSSLSASVGDRVTITCRAS | 143 |
| | SSVPYMHWLQQKPGKAPKLLIYSTSN | |
| | LASGVPSRFSGSGSGTDFTLTISSVQ | |
| | PEDFATYYCQQRSSYPLTFGGGTKLE | |
| | IK | |
| MFE-L29 | EIQMTQSPSSLSASVGDRVTITCRAS | 144 |
| | SSVPYMHWLQQKPGKAPKLLIYSTSS | |
| | LQSGVPSRFSGSGSGTDFTLTISSVQ | |
| | PEDFATYYCQQRSSYPLTFGGGTKLE | |
| | IK | |

FIG. 8 shows an alignment of the sequences as listed in Table 18 and 19, respectively.

The variable region of six heavy and six light chain DNA sequences, encoding the humanized CEA binder, were subcloned in frame with either the constant heavy chain or the constant light chain of human IgG1 containing P239G, L234A and L235A mutations to abrogate binding to Fcγ receptors (WO 2012/130831 Al). The antibodies were produced as described below. The resulting 36 variants (Table 20) were tested for binding on MKN45 cells; and 7 variants were selected for further development.

TABLE 20
Nomenclature for VH/VL combinations expressed
as huIgG1_LALA_PG antibodies

| | MFE-L24 | MFE-L25 | MFE-L26 | MFE-L27 | MFE-L28 | MFE-L29 |
|---|---|---|---|---|---|---|
| MFE-H24 | P1AE3125 | P1AE3119 | P1AE3113 | P1AE3107 | P1AE3101 | P1AE3095 |
| MFE-H25 | P1AE3124 | P1AE3118 | P1AE3112 | P1AE3106 | P1AE3100 | P1AE3094 |
| MFE-H26 | P1AE3123 | P1AE3117 | P1AE3111 | P1AE3105 | P1AE3099 | P1AE3093 |
| MFE-H27 | P1AE3122 | P1AE3116 | P1AE3110 | P1AE3104 | P1AE3098 | P1AE3092 |
| MFE-H28 | P1AE3121 | P1AE3115 | P1AE3109 | P1AE3103 | P1AE3097 | P1AE3091 |
| MFE-H29 | P1AE3120 | P1AE3114 | P1AE3108 | P1AE3102 | P1AE3096 | P1AE3090 |

### 2.3.4 Selection of Humanized MFE23 Antibodies

Binding of the 36 humanized MFE23 huIgG1 P329G LALA variants to CEA expressed on MKN45 cells was compared to the respective parental murine MFE23 huIgG1 P329G LALA antibody. 17 clones lost their binding capacity to human CEACAM5 expressing MKN45 cells (FIG. 9A). Eight clones showed reduced binding if compared to the parental clone 1VIFE23 (FIG. 9B). Eleven clones showed similar binding if compared to the parental clone MFE23 (FIG. 9C). The fitting EC₅₀ values and area under the curve values (AUC) of these binding curves are displayed in Table 21.

TABLE 21
EC₅₀ values and area under the curve (AUC) of binding
curves of different humanized MFE23 huIgG1 P329G
LALA antibodies displayed in FIGS. 8A, 8B and 8C

| | EC₅₀ | |
|---|---|---|
| binding to MKN45 | [nM] | AUC |
| P1AD5108-002 DP47 huIgG1 PG LALA (Isotype | n.d. | 420 |
| control) | | |
| P1AE0096-001 MFE23 huIgG1 PG LALA (parental) | 9.36 | 83489 |
| P1AE3104 | n.d. | 414 |
| P1AE3122 | n.d. | 422 |
| P1AE3116 | n.d. | 443 |
| P1AE3092 | n.d. | 482 |
| P1AE3110 | n.d. | 547 |
| P1AE3112 | n.d. | 551 |
| P1AE3118 | n.d. | 559 |
| P1AE3111 | n.d. | 578 |
| P1AE3109 | n.d. | 695 |
| P1AE3113 | n.d. | 757 |
| P1AE3124 | n.d. | 847 |
| P1AE3123 | n.d. | 1090 |
| P1AE3106 | n.d. | 1126 |
| P1AE3117 | n.d. | 1181 |
| P1AE3098 | n.d. | 1191 |
| P1AE3108 | n.d. | 2086 |
| P1AE3125 | n.d. | 3145 |
| P1AE3094 | 47.57 | 6908 |
| P1AE3115 | 20.68 | 10177 |
| P1AE3119 | 41.90 | 10327 |
| P1AE3114 | 30.60 | 12769 |
| P1AE3121 | 17.13 | 17464 |
| P1AE3120 | 12.37 | 24181 |
| P1AE3105 | 7.92 | 55868 |
| P1AE3090 | 42.00 | 61809 |
| P1AE3093 | 16.14 | 68082 |
| P1AE3100 | 9.89 | 74137 |
| P1AE3091 | 14.60 | 83061 |
| P1AE3095 | 11.12 | 84917 |
| P1AE3096 | 10.46 | 87775 |
| P1AE3107 | 5.83 | 91203 |
| P1AE3102 | 5.41 | 91481 |
| P1AE3103 | 5.88 | 92448 |
| P1AE3099 | 10.21 | 95311 |
| P1AE3097 | 6.45 | 98656 |
| P1AE3101 | 6.58 | 103966 |

### 2.3.5 Generation and Production of Bispecific Antibodies With a Bivalent Binding to 4-1BB and a Monovalent Binding to Humanized CEA (MFE23) Binders

The bispecific 2+1 H2H anti-4-1BB anti-CEA huIgG1 P329GLALA antibodies are produced as described in Example 2.1 for the 2+1 anti-4-1BB anti-CEA (MFE23) huIgG1 P329GLALA antibodies.

The amino acid sequences for the bispecific 4-1BB (20H4.9)/CEA (huMFE23-L28-H24) P329GLALA IgG1 2+1 (H2H) antibody can be found in Table 22 and of 4-1BB (20H4.9)/CEA (huMFE23-L28-H28) P329GLALA IgG1 2+1 (H2H) antibody in Table 23. The protein is produced and purified as described in Example 1.2. Further bispecific constructs are shown in Tables 24 to 28.

TABLE 22
Amino acid sequences of bispecific,
bivalent anti-4-1BB/monovalent anti-
CEA(huMFE23-L28-H24) human IgG1 P329GLALA
antigen binding molecules (2 + 1 H2H)

| SEQ | | |
|---|---|---|
| ID | Descrip- | |
| NO: | tion | Sequence |
| 65 | VHCH1(EE) | See Table 1 |
| | (20H4.9)- | |
| | Heavy | |
| | chain | |
| | HC1 | |
| | (Fc | |
| | hole) | |
| 179 | VLCH1 | EIQMTQSPSSLSASVGDRVTITCRASSSVPYM |
| | (CEA | HWLQQKPGKAPKLLIYSTSNLASGVPSRFSGS |
| | huMFE23- | GSGTDFTLTISSVQPEDFATYYCQQRSSYPL |
| | L28-H24) | TFGGGTKLEIKSSASTKGPSVFPLAPSSKST |
| | VHCH1(EE) | SGGTAALGCLVKDYFPEPVTVSWNSGALTSG |
| | (20H4.9)- | VHTFPAVLQSSGLYSLSSVVTVPSSSLGTQT |
| | Heavy | YICNVNHKPSNTKVDKKVEPKSCDGGGGSGG |
| | chain | GGSQVQLQQWGAGLLKPSETLSLTCAVYGGS |
| | HC2 | FSGYYWSWIRQSPEKGLEWIGEINHGGYVTY |
| | (Fc knob) | NPSLESRVTISVDTSKNQFSLKLSSVTAADT |
| | | AVYYCARDYGPGNYDWYFDLWGRGTLVTVSS |
| | | ASTKGPSVFPLAPSSKSTSGGTAALGCLVED |
| | | YFPEPVTVSWNSGALTSGVHTFPAVLQSSGL |
| | | YSLSSVVTVPSSSLGTQTYICNVNHKPSNTK |
| | | VDEKVEPKSCDKTHTCPPCPAPEAAGGPSVF |
| | | LFPPKPKDTLMISRTPEVTCVVVDVSHEDPE |
| | | VKFNWYVDGVEVHNAKTKPREEQYNSTYRVV |
| | | SVLTVLHQDWLNGKEYKCKVSNKALGAPIEK |
| | | TISKAKGQPREPQVYTLPPCRDELTKNQVSL |
| | | WCLVKGFYPSDIAVEWESNGQPENNYKTTPP |
| | | VLDSDGSFFLYSKLTVDKSRWQQGNVFSCSV |
| | | MHEALHNHYTQKSLSLSP |
| 67 | VLCL | See Table 1 |
| | (RK)- | |
| | Light | |
| | chain | |
| | (20H4.9) | |
| 180 | VHCL- | QVQLVQSGAEVKKPGASVKVSCKASGFNIK |
| | Light | DSYMHWVRQAPGQGLEWMGWIDPENGDTEY |
| | chain | APKFQGRVTMTTDTSISTAYMELSRLRSDD |
| | (CEA | TAVYYCNEGTPTGPYYFDYWGQGTLVTVSS |
| | huMFE23- | ASVAAPSVFIFPPSDEQLKSGTASVVCLLN |
| | L28-H24) | NFYPREAKVQWKVDNALQSGNSQESVTEQD |
| | | SKDSTYSLSSTLTLSKADYEKHKVYACEVT |
| | | HQGLSSPVTKSFNRGEC |

TABLE 23
Amino acid sequences of bispecific,
bivalent anti-4-1BB/monovalent anti-
CEA(huMFE23-L28-H28) human IgG1 P329GLALA
antigen binding molecules (2+1 H2H)

| SEQ | | |
|---|---|---|
| ID | | |
| NO: | Description | Sequence |
| 65 | VHCH1(EE) | See Table 1 |
| | (20H4.9)-Heavy | |
| | chain HC1 (Fc | |
| | hole) | |
| 181 | VLCH1 (CEA | EIQMTQSPSSLSASVGDRV |
| | huMFE23- | TITCRASSSVPYMHWLQQK |
| | L28-H28) | PGKAPKLLIYSTSNLASGV |
| | VHCH1(EE) | PSRFSGSGSGTDFTLTISS |
| | (20H4.9)- | VQPEDFATYYCQQRSSYPL |
| | Heavy | TFGGGTKLEIKSSASTKGP |
| | chain | SVFPLAPSSKSTSGGTAAL |
| | HC2 (Fc | GCLVKDYFPEPVTVSWNSG |
| | knob) | ALTSGVHTFPAVLQSSGLY |
| | | SLSSVVTVPSSSLGTQTYI |
| | | CNVNHKPSNTKVDKKVEPK |
| | | SCDGGGGSGGGGSQVQLQQ |
| | | WGAGLLKPSETLSLTCAVY |
| | | GGSFSGYYWSWIRQSPEKG |
| | | LEWIGEINHGGYVTYNPSL |
| | | ESRVTISVDTSKNQFSLKL |
| | | SSVTAADTAVYYCARDYGP |
| | | GNYDWYFDLWGRGTLVTVS |
| | | SASTKGPSVFPLAPSSKST |
| | | SGGTAALGCLVEDYFPEPV |
| | | TVSWNSGALTSGVHTFPAV |
| | | LQSSGLYSLSSVVTVPSSS |
| | | LGTQTYICNVNHKPSNTKV |
| | | DEKVEPKSCDKTHTCPPCP |
| | | APEAAGGPSVFLFPPKPKD |
| | | TLMISRTPEVTCVWDVSHE |
| | | DPEVKFNWYVDGVEVHNAK |
| | | TKPREEQYNSTYRVVSVLT |
| | | VLHQDWLNGKEYKCKVSNK |
| | | ALGAPIEKTISKAKGQPRE |
| | | PQVYTLPPCRDELTKNQVS |
| | | LWCLVKGFYPSDIAVEWES |
| | | NGQPENNYKTTPPVLDSDG |
| | | SFFLYSKLTVDKSRWQQGN |
| | | VFSCSVMHEALHNHYTQKS |
| | | LSLSP |
| 67 | VLCL(RK)- | See Table 1 |
| | Light | |
| | chain | |
| | (20H4.9) | |
| 182 | VHCL-Light | QVQLVQSGAEVKKPGASVK |
| | chain | VSCKASGFNIKDSYMHWVR |
| | (CEA | QAPGQGLEWMGWIDPENGD |
| | huMFE23- | TEYAPKFQGRVTMTRDTSI |
| | L28-H28) | STAYMELSRLRSDDTAVYY |
| | | CNEGTPTGPYYFDYWGQGT |
| | | LVTVSSASVAAPSVFIFPP |
| | | SDEQLKSGTASWCLLNNFY |
| | | PREAKVQWKVDNALQSGNS |
| | | QESVTEQDSKDSTYSLSST |
| | | LTLSKADYEKHKVYACEVT |
| | | HQGLSSPVTKSFNRGEC |

TABLE 24
Amino acid sequences of bispecific,
bivalent anti-4-1BB/monovalent anti-
CEA(huMFE23-L28-H25) human IgG1
P329GLALA antigen binding
molecules (2 + 1 H2H)

| SEQ | | |
|---|---|---|
| ID | | |
| NO: | Description | Sequence |
| 65 | VHCH1(EE) | See Table 1 |
| | (20H4.9) | |
| | Heavy | |
| | chain | |
| | HC (Fc | |
| | hole) | |
| 183 | VLCH1(CEA | EIQMTQSPSSLSASVGDRVT |
| | huMFE23- | ITCRASSSVPYMHWLQQKPG |
| | L28-H25) | KAPKLLIYSTSNLASGVPSR |
| | VHCH1(EE) | FSGSGSGTDFTLTISSVQPE |
| | (20H4.9)- | DFATYYCQQRSSYPLTFGGG |
| | Heavy | TKLEIKSSASTKGPSVFPLA |
| | chain | PSSKSTSGGTAALGCLVKDY |
| | HC2 | FPEPVTVSWNSGALTSGVHT |
| | (Fc | FPAVLQSSGLYSLSSVVTVP |
| | knob) | SSSLGTQTYICNVNHKPSNT |
| | | KVDKKVEPKSCDGGGGSGGG |
| | | GSQVQLQQWGAGLLKPSETL |
| | | SLTCAVYGGSFSGYYWSWIR |
| | | QSPEKGLEWIGEINHGGYVT |
| | | YNPSLESRVTISVDTSKNQF |
| | | SLKLSSVTAADTAVYYCARD |
| | | YGPGNYDWYFDLWGRGTLVT |
| | | VSSASTKGPSVFPLAPSSKS |
| | | TSGGTAALGCLVEDYFPEPV |
| | | TVSWNSG |

TABLE 25
Amino acid sequences of bispecific,
bivalent anti-4-1BB/monovalent anti-
CEA(huMFE23-L27-H29) human IgG1
P329GLALA antigen binding
molecules (2 + 1 H2H)

| SEQ | | |
|---|---|---|
| ID | | |
| NO: | Description | Sequence |
| 65 | VHCH1(EE) | See Table 1 |
| | (20H4.9)- | |
| | Heavy | |
| | chain | |
| | HC1 (Fc | |
| | hole) | |
| 185 | VLCH1 | EIQMTQSPSSLSASVGDRVTI |
| | (CEA | TCRASSSVPYMHWYQQKPGKA |
| | huMFE23- | PKLLIYSTSNLASGVPSRFSG |
| | L27- | SGSGTDFTLTISSVQPEDFAT |
| | H29) | YYCQQRSSYPLTFGGGTKLEI |
| | VHCH1 | KSSASTKGPSVFPLAPSSKST |
| | (EE) | SGGTAALGCLVKDYFPEPVTV |
| | (20H4.9)- | SWNSGALTSGVHTFPAVLQSS |
| | Heavy | GLYSLSSVVTVPSSSLGTQTY |
| | chain | ICNVNHKPSNTKVDKKVEPKS |
| | HC2 (Fc | CDGGGGSGGGGSQVQLQQWGA |
| | knob) | GLLKPSETLSLTCAVYGGSFS |
| | | GYYWSWIRQSPEKGLEWIGEI |
| | | NHGGYVTYNPSLESRVTISVD |
| | | TSKNQFSLKLSSVTAADTAVY |
| | | YCARDYGPGNYDWYFDLWGRG |
| | | TLVTVSSASTKGPSVFPLAPS |
| | | SKSTSGGTAALGCLVEDYFPE |
| | | PVTVSWNSGALTSGVHTFPAV |
| | | LQSSGLYSLSSVVTVPSSSLG |
| | | TQTYICNVNHKPSNTKVDEKV |
| | | EPKSCDKTHTCPPCPAPEAAG |
| | | GPSVFLFPPKPKDTLMISRTP |
| | | EVTCVVVDVSHEDPEVKFNWY |
| | | VDGVEVHNAKTKPREEQYNST |
| | | YRVVSVLTVLHQDWLNGKEYK |
| | | CKVSNKALGAPIEKTISKAKG |
| | | QPREPQVYTLPPCRDELTKNQ |
| | | VSLWCLVKGFYPSDIAVEWES |
| | | NGQPENNYKTTPPVLDSDGSF |
| | | FLYSKLTVDKSRWQQGNVFSC |
| | | SVMHEALHNHYTQKSLSLSP |
| 67 | VLCL(RK)- | See Table 1 |
| | Light | |
| | chain | |
| | (20H4.9) | |
| 186 | VHCL- | QVQLVQSGAEVKKPGSSVKVSCKA |
| | Light | SGFNIKDSYMHWVRQAPGQGLEWM |
| | chain | GWIDPENGDTEYAPKFQGRVTITT |
| | (CEA | DESTSTAYMELSSLRSEDTAVYYC |
| | huMFE23- | NEGTPTGPYYFDYWGQGTLVTVSS |
| | L27-H29) | ASVAAPSVFIFPPSDEQLKSGTAS |
| | | VVCLLNNFYPREAKVQWKVDNALQ |
| | | SGNSQESVTEQDSKDSTYSLSSTL |
| | | TLSKADYEKHKVYACEVTHQGLSS |
| | | PVTKSFNRGEC |

TABLE 26
Amino acid sequences of bispecific,
bivalent anti-4-1BB/monovalent anti-
CEA(huMFE23-L27-H28) human IgG1 P329GLALA
antigen binding molecules (2 + 1 H2H)

| SEQ | | |
|---|---|---|
| ID | | |
| NO: | Description | Sequence |
| 65 | VHCH1(EE) | See Table 1 |
| | (20H4.9)- | |
| | Heavy | |
| | chain | |
| | HC1 (Fc | |
| | hole) | |
| 187 | VLCH1 | EIQMTQSPSSLSASVGDRVTIT |
| | (CEA | CRASSSVPYMHWYQQKPGKAPK |
| | huMFE23- | LLIYSTSNLASGVPSRFSGSGS |
| | L27- | GTDFTLTISSVQPEDFATYYCQ |
| | H28) | QRSSYPLTFGGGTKLEIKSSAS |
| | VHCH1 | TKGPSVFPLAPSSKSTSGGTAA |
| | (EE) | LGCLVKDYFPEPVTVSWNSGAL |
| | (20H4.9)- | TSGVHTFPAVLQSSGLYSLSSV |
| | Heavy | VTVPSSSLGTQTYICNVNHKPS |
| | chain | NTKVDKKVEPKSCDGGGGSGGG |
| | HC2 | GSQVQLQQWGAGLLKPSETLSL |
| | (Fc | TCAVYGGSFSGYYWSWIRQSPE |
| | knob) | KGLEWIGEINHGGYVTYNPSLE |
| | | SRVTISVDTSKNQFSLKLSSVT |
| | | AADTAVYYCARDYGPGNYDWYF |
| | | DLWGRGTLVTVSSASTKGPSVF |
| | | PLAPSSKSTSGGTAALGCLVED |
| | | YFPEPVTVSWNSGALTSGVHTF |
| | | PAVLQSSGLYSLSSVVTVPSSS |
| | | LGTQTYICNVNHKPSNTKVDEK |
| | | VEPKSCDKTHTCPPCPAPEAAG |
| | | GPSVFLFPPKPKDTLMISRTPE |
| | | VTCVWDVSHEDPEVKFNWYVDG |
| | | VEVHNAKTKPREEQYNSTYRVV |
| | | SVLTVLHQDWLNGKEYKCKVSN |
| | | KALGAPIEKTISKAKGQPREPQ |
| | | VYTLPPCRDELTKNQVSLWCLV |
| | | KGFYPSDIAVEWESNGQPENNY |
| | | KTTPPVLDSDGSFFLYSKLTVD |
| | | KSRWQQGNVFSCSVMHEALHNH |
| | | YTQKSLSLSP |
| 67 | VLCL(RK)- | See Table 1 |
| | Light | |
| | chain | |
| | (20H4.9) | |
| 188 | VHCL- | QVQLVQSGAEVKKPGAS |
| | Light | VKVSCKASGFNIKDSYM |
| | chain | HWVRQAPGQGLEWMGWI |
| | (CEA | DPENGDTEYAPKFQGRV |
| | huMFE23- | TMTRDTSISTAYMELSR |
| | L27- | LRSDDTAVYYCNEGTPT |
| | H28) | GPYYFDYWGQGTLVTVS |
| | | SASVAAPSVFIFPPSDE |
| | | QLKSGTASWCLLNNFYP |
| | | REAKVQWKVDNALQSGN |
| | | SQESVTEQDSKDSTYSL |
| | | SSTLTLSKADYEKHKVY |
| | | ACEVTHQGLSSPVTKSF |
| | | NRGEC |

TABLE 27
Amino acid sequences of bispecific,
bivalent anti-4-1BB/monovalent anti-
CEA(huMFE23- L27-H26) human IgG1 P329GLALA
antigen binding molecules (2 + 1 H2H)

| SEQ | | |
|---|---|---|
| ID | | |
| NO: | Description | Sequence |
| 65 | VHCH1 | See Table 1 |
| | (EE) | |
| | (20H4.9)- | |
| | Heavy | |
| | chain | |
| | HC1 | |
| | (Fc | |
| | hole) | |
| 189 | VLCH1 | EIQMTQSPSSLSASVGDRVTI |
| | (CEA | TCRASSSVPYMHWYQQKPGKA |
| | huMFE23- | PKLLIYSTSNLASGVPSRFSG |
| | L27- | SGSGTDFTLTISSVQPEDFAT |
| | H26) | YYCQQRSSYPLTFGGGTKLEI |
| | VHCH1 | KSSASTKGPSVFPLAPSSKST |
| | (EE) | SGGTAALGCLVKDYFPEPVTV |
| | (20H4.9)- | SWNSGALTSGVHTFPAVLQSS |
| | Heavy | GLYSLSSVVTVPSSSLGTQTY |
| | chain | ICNVNHKPSNTKVDKKVEPKS |
| | HC2 | CDGGGGSGGGGSQVQLQQWGA |
| | (Fc | GLLKPSETLSLTCAVYGGSFS |
| | knob) | GYYWSWIRQSPEKGLEWIGEI |
| | | NHGGYVTYNPSLESRVTISVD |
| | | TSKNQFSLKLSSVTAADTAVY |
| | | YCARDYGPGNYDWYFDLWGRG |
| | | TLVTVSSASTKGPSVFPLAPS |
| | | SKSTSGGTAALGCLVEDYFPE |
| | | PVTVSWNSGALTSGVHTFPAV |
| | | LQSSGLYSLSSVVTVPSSSLG |
| | | TQTYICNVNHKPSNTKVDEKV |
| | | EPKSCDKTHTCPPCPAPEAAG |
| | | GPSVFLFPPKPKDTLMISRTP |
| | | EVTCVVVDVSHEDPEVKFNWY |
| | | VDGVEVHNAKTKPREEQYNST |
| | | YRVVSVLTVLHQDWLNGKEYK |
| | | CKVSNKALGAPIEKTISKAKG |
| | | QPREPQVYTLPPCRDELTKNQ |
| | | VSLWCLVKGFYPSDIAVEWES |
| | | NGQPENNYKTTPPVLDSDGSF |
| | | FLYSKLTVDKSRWQQGNVFSC |
| | | SVMHEALHNHYTQKSLSLSP |
| 67 | VLCL(RK)- | See Table 1 |
| | Light | |
| | chain | |
| | (20H4.9) | |
| 190 | VHCL-Light | QVQLVQSGAEVKKPGASVKVS |
| | chain | CKASGFNIKDSYMHWVRQAPG |
| | (CEA | QGLEWMGWIDPENGGTNYAQK |
| | huMFE23- | FQGRVTMTTDTSISTAYMELS |
| | L27-H26) | RLRSDDTAVYYCNEGTPTGPY |
| | | YFDYWGQGTLVTVSSASVAAP |
| | | SVFIFPPSDEQLKSGTASVVC |
| | | LLNNFYPREAKVQWKVDNALQ |
| | | SGNSQESVTEQDSKDSTYSLS |
| | | STLTLSKADYEKHKVYACEVT |
| | | HQGLSSPVTKSFNRGEC |

TABLE 28
Amino acid sequences of bispecific,
bivalent anti-4-1BB/monovalent anti-
CEA(huMFE23-L27-H24) human IgG1 P329GLALA
antigen binding molecules (2 + 1 H2H)

| SEQ ID | | |
|---|---|---|
| NO: | Description | Sequence |
| 65 | VHCH1(EE) | See Table 1 |
| | (20H4.9)- | |
| | Heavy | |
| | chain | |
| | HC1 (Fc | |
| | hole) | |
| 191 | VLCH1 | EIQMTQSPSSLSASVGDRVTI |
| | (CEA | TCRASSSVPYMHWYQQKPGKA |
| | huMFE23- | PKLLIYSTSNLASGVPSRFSG |
| | L27-H24) | SGSGTDFTLTISSVQPEDFAT |
| | VHCH1 | YYCQQRSSYPLTFGGGTKLEI |
| | (EE) | KSSASTKGPSVFPLAPSSKST |
| | (20H4.9)- | SGGTAALGCLVKDYFPEPVTV |
| | Heavy | SWNSGALTSGVHTFPAVLQSS |
| | chain | GLYSLSSVVTVPSSSLGTQTY |
| | HC2 (Fc | ICNVNHKPSNTKVDKKVEPKS |
| | knob) | CDGGGGSGGGGSQVQLQQWGA |
| | | GLLKPSETLSLTCAVYGGSFS |
| | | GYYWSWIRQSPEKGLEWIGEI |
| | | NHGGYVTYNPSLESRVTISVD |
| | | TSKNQFSLKLSSVTAADTAVY |
| | | YCARDYGPGNYDWYFDLWGRG |
| | | TLVTVSSASTKGPSVFPLAPS |
| | | SKSTSGGTAALGCLVEDYFPE |
| | | PVTVSWNSGALTSGVHTFPAV |
| | | LQSSGLYSLSSVVTVPSSSLG |
| | | TQTYICNVNHKPSNTKVDEKV |
| | | EPKSCDKTHTCPPCPAPEAAG |
| | | GPSVFLFPPKPKDTLMISRTP |
| | | EVTCVVVDVSHEDPEVKFNWY |
| | | VDGVEVHNAKTKPREEQYNST |
| | | YRVVSVLTVLHQDWLNGKEYK |
| | | CKVSNKALGAPIEKTISKAKG |
| | | QPREPQVYTLPPCRDELTKNQ |
| | | VSLWCLVKGFYPSDIAVEWES |
| | | NGQPENNYKTTPPVLDSDGSF |
| | | FLYSKLTVDKSRWQQGNVFSC |
| | | SVMHEALHNHYTQKSLSLSP |
| 67 | VLCL(RK)- | See Table 1 |
| | Light | |
| | chain | |
| | (20H4.9) | |
| 192 | VHCL- | QVQLVQSGAEVKKPGASVKVS |
| | Light | CKASGFNIKDSYMHWVRQAPG |
| | chain | QGLEWMGWIDPENGDTEYAPK |
| | (CEA | FQGRVTMTTDTSISTAYMELS |
| | huMFE23- | RLRSDDTAVYYCNEGTPTGPY |
| | L27-H24) | YFDYWGQGTLVTVSSASVAAP |
| | | SVFIFPPSDEQLKSGTASVVC |
| | | LLNNFYPREAKVQWKVDNALQ |
| | | SGNSQESVTEQDSKDSTYSLS |
| | | STLTLSKADYEKHKVYACEVT |
| | | HQGLSSPVTKSFNRGEC |

The production of the bispecific antibodies was carried out as described in Example 1.2. Exemplary analysis of obtained products is shown in Table 29 below.

TABLE 29
Biochemical analysis of 2 + 1 H2H anti-4-
1BB, anti-CEA huIgG1 PGLALA bispecific antibodies

| | | | CE-SDS |
|---|---|---|---|
| | Monomer | Yield | (non-red, |
| Molecule | [%] | [mg/l] | main peak) |
| 2 + 1 H2H 4-1BB (20H4.9)/ | 98 | 7.4 | 95 |
| CEA(A5B7) human IgG1 | | | |
| PGLALA | | | |
| 2 + 1 H2H 4-1BB (20H4.9)/ | 98 | 7 | 94 |
| CEA(MFE23) human IgG1 | | | |
| PGLALA | | | |
| 2 + 1 H2H 4-1BB (20H4.9)/ | 97 | 6.6 | 97 |
| CEA(A5H1EL1D) human | | | |
| IgG1 PGLALA | | | |

### Example 3

### Preparation, Purification and Characterization of Bispecific Antibodies With a Bivalent Binding to 4-1BB and a Monovalent Binding to CD19

### 2.1 Generation and Production of Bispecific Antibodies With a Bivalent Binding to 4-1BB and a Monovalent Binding to CD19

Bispecific agonistic 4-1BB antibodies with bivalent binding for 4-1BB and monovalent binding for CEA can also be prepared, by replacing the anti-FAP cross Fab with an anti-CD19 cross Fab. This construct is also termed Head to Head (H2H) 2+1 format.

The generation and preparation of the CD19 binder clone 2B11 is described in WO 2017/055328 A1. The amino acid sequences for 2+1 H2H bivalent bispecific 4-1BB (20H4.9)×CD19(2B11) P329GLALA IgG1 2+1 (H2H) antibody huIgG1 PGLALA can be found in Table 30 below.

TABLE 30
Amino acid sequences of bispecific,
bivalent anti-4-1BB/monovalent anti-
CD19 (2B11) human IgG1 P329GLALA antigen
binding molecules (2 +1 H2H)

| SEQ | | |
|---|---|---|
| ID | | |
| NO: | Description | Sequence |
| 65 | VHCH1(EE) | See Table 1 |
| | (20H4.9)- | |
| | Heavy | |
| | chain | |
| | HC1 (Fc | |
| | hole) | |
| 84 | VLCH1 | DIVMTQTPLSLSVTPGQPA |
| | (2B11) | SISCKSSQSLETSTGTTYL |
| | VHCH1(EE) | NWYLQKPGQSPQLLIYRVS |
| | (20H4.9)- | KRFSGVPDRFSGSGSGTDF |
| | Heavy | TLKISRVEAEDVGVYYCLQ |
| | chain | LLEDPYTFGQGTKLEIKSS |
| | HC2 (Fc | ASTKGPSVFPLAPSSKSTS |
| | knob) | GGTAALGCLVKDYFPEPVT |
| | | VSWNSGALTSGVHTFPAVL |
| | | QSSGLYSLSSVVTVPSSSL |
| | | GTQTYICNVNHKPSNTKVD |
| | | KKVEPKSCDGGGGSGGGGS |
| | | QVQLQQWGAGLLKPSETLS |
| | | LTCAVYGGSFSGYYWSWIR |
| | | QSPEKGLEWIGEINHGGYV |
| | | TYNPSLESRVTISVDTSKN |
| | | QFSLKLSSVTAADTAVYYC |
| | | ARDYGPGNYDWYFDLWGRG |
| | | TLVTVSSASTKGPSVFPLA |
| | | PSSKSTSGGTAALGCLVED |
| | | YFPEPVTVSWNSGALTSGV |
| | | HTFPAVLQSSGLYSLSSVV |
| | | TVPSSSLGTQTYICNVNHK |
| | | PSNTKVDEKVEPKSCDKTH |
| | | TCPPCPAPEAAGGPSVFLF |
| | | PPKPKDTLMISRTPEVTCV |
| | | VVDVSHEDPEVKFNWYVDG |
| | | VEVHNAKTKPREEQYNSTY |
| | | RVVSVLTVLHQDWLNGKEY |
| | | KCKVSNKALGAPIEKTISK |
| | | AKGQPREPQVYTLPPCRDE |
| | | LTKNQVSLWCLVKGFYPSD |
| | | LAVEWESNGQPENNYKTTP |
| | | PVLDSDGSFFLYSKLTVDK |
| | | SRWQQGNVFSCSVMHEALH |
| | | NHYTQKSLSLSP |
| 67 | VLCL(RK)- | See Table 1 |
| | Light | |
| | chain | |
| | (20H4.9) | |
| 85 | VHCL- | QVQLVQSGAEVKKPGASVKVS |
| | Light | CKASGYTFTDYIMHWVRQAPG |
| | chain | QGLEWMGYINPYNDGSKYTEK |
| | (2B11) | FQGRVTMTSDTS1STAYMELS |
| | | RLRSDDTAVYYCARGTYYYGP |
| | | QLFDYWGQGTTVTVSSASVAA |
| | | PSVFIFPPSDEQLKSGTASVV |
| | | CLLNNFYPREAKVQWKVDNAL |
| | | QSGNSQESVTEQDSKDSTYSL |
| | | SSTLTLSKADYEKHKVYACEV |
| | | THQGLSSPVTKSFNRGEC |

### Example 4

### Functional Characterization of 2+1 H2H Bispecific Agonistic 4-1BB Antigen Binding Molecules With Monovalent Binding for FAP

### 4.1 Surface Plasmon Resonance (Simultaneous Binding)

The capacity of binding simultaneously to human 4-1BB Fc (kih) and human FAP was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany). Human 4-1BB Fc(kih) was directly coupled to a flow cell by amine coupling (CM5 sensor chip). Immobilization levels of 710 resonance units (RU) were used.

The 2+1 H2H anti-4-1BB/anti-FAP huIgG1 PGLALA construct was passed at a concentration range of 200 nM with a flow of 30 μL/minute through the flow cells over 90 seconds and dissociation was set to zero sec. Human FAP was injected as second analyte with a flow of 30 μL/minute through the flow cells over 90 sec at a concentration of 500 nM (see setup of the assay in FIG. 2A). The dissociation was monitored for 120 sec. Bulk refractive index differences were corrected for by subtracting the response obtained in a reference flow cell, where no protein was immobilized.

As can be seen in the graphs of FIG. 2B, the 2+1 H2H 4-1BB (20H4.9)/FAP (4B9) P329GLALA IgG1 antibody construct can bind simultaneously human 4-1BB and human FAP.

4.2 Competition Assay by TagLite to Confirm Bivalent Binding to hu4-1BB

To confirm the bivalent binding of the bispecific 2+1 H2H anti-4-1BB, anti-FAP huIgG1 P329GLALA antibody to hu 4-1BB, a competition assay using time-resolved fluorescence resonance energy transfer (TR-FRET) (referred to as TagLite) was performed.

Binding of a d2-labeled 4-1BB(clone 20H4.9) IgG1 to hu4-1BB-SNAP Tb-labeled expressed on transfected Hek cells led to a TR-FRET signal. For the competition assay the bound d2-labeled 4-1BB(clone 20H4.9) IgG1 was displaced by unlabeled 2+1 H2H 4-1BB (20H4.9)/FAP (4B9) P329GLALA IgG1 antibody (one of the Fabs against 4-1BB does not have a free N-terminus) or by unlabeled 2+1 VH/VL (C-terminal) 4-1BB (20H4.9)/FAP (4B9) P329GLALA IgG1 antibody (two “free Fabs” against 4-1BB) resulting in a decrease of the TR-FRET signal (Table 31).

TABLE 31
Samples used in the competition assay

| | | |
|---|---|---|
| Sample | conc. (mg/ml) | Format |

Binding antibody labeled with donor

| | | | |
|---|---|---|---|
| 4-1BB(clone 20H4.9) huIgG1 - d2 | 0.515 | 2 + 0 | IgG |

Unlabeled constructs used for competition

| | | | |
|---|---|---|---|
| 2 + 1 H2H 4-1BB (20H4.9)/FAP (4B9) | 1.92 | 2 + 1 | Bsp |
| P329GLALA IgG1 | | | |
| 2 + 1 VH/VL (C-terminal) 4-1BB | 1.27 | 2 + 1 | Bsp |
| (20H4.9)/FAP (4B9) P329GLALA IgG1 | | | |

Briefly, pre-labeled Tb hu4-1BB-SNAP expressing cells were thawed, washed and 5000 cells per well in 10 μl were mixed with 5 μl of acceptor (d2) labeled 4-1BB (clone 20H4.9) IgG1 at 0.6 nM and 5 μl of unlabeled competitor constructs in a 1:3 concentration dilution ranging from 0.006-1000 nM; final volume of 20 μl in a 384 well plate. The fluorescent signal was measured at 620 nm for the fluorescent donor (Tb) and at 665 nm for the fluorescent acceptor (d2) dye after 0 h, 2 h and 4 h at RT (M1000 Pro, Tecan). The ratio of 665/620*10000 (R) was calculated, and the ratio of the reference (cells only) was subtracted, resulting in the plotted ΔR value. For IC₅₀ determination the results were analyzed using the one site—fit log IC₅₀ in Graph Pad Prism6 (Table 32). The assay was performed in duplicates.

TABLE 32
Kᵢ values after 4 hours with 95% confidence interval
Unlabeled constructs used for competition

| | IC₅₀ nM | |
|---|---|---|
| Sample | (95% CI) | Format |
| 4-1BB (20H4.9)/FAP (4B9) P329GLALA IgG1 | 1.9 (1.6-2.4) | 2 + 1 Bsp |
| 2 + 1 (H2H) | | |
| 4-1BB (20H4.9)/FAP (4B9) P329GLALA IgG1 | 2.5 (1.9-3.3) | 2 + 1 Bsp |
| 2 + 1 (C-terminal) | | |

The result indicates that both 4-1BB FAP bispecific constructs can compete similarly (similar IC₅₀) with 4-1BB (clone 20H4.9) IgG for binding to hu4-1BB. This suggests that both Fab arms against 4-1BB of the bispecific 2+1 H2H anti-4-1BB, anti-FAP huIgG1 P329GLALA antibody can bind to 4-1BB. 2+1 H2H 4-1BB (20H4.9)/FAP (4B9) huIgG1 P329GLALA antibody can therefore bind bivalently to 4-1BB (FIG. 3).

### 4.3 Binding to Human FAP-Expressing Cell Lines

For testing the binding to cell-surface-expressed human Fibroblast Activation Protein (FAP) NIH/3T3-huFAP clone 19 cells were used. NIH/3T3-huFAP clone 19 was generated by transfection of mouse embryonic fibroblast NIH/3T3 cells (ATCC CRL-1658) with the expression pETR4921 plasmid encoding human FAP under a CMV promoter. Cells were maintained in DMEM (GIBCO by life technologies, Cat.-No.: 42340-025) supplied with fetal bovine serum (FBS, GIBCO by Life Technologies, Cat.-No. 16000-044, Lot 941273, gamma irradiated mycoplasma free, heat inactivated), 2 mM L-alanyl-L-glutamine dipeptide (Gluta-MAX-I, GIBCO by Life Technologies, Cat.-No. 35050-038) and 1.5 μg/mL puromycin (InvivoGen, Cat.-No.: ant-pr-5). For the binding assay, 2×10⁵ of NIH/3T3-huFAP clone 19 cells were added to each well of a round-bottom suspension cell 96-well plate (Greiner bio-one, cellstar, Cat.-No. 650185). Cells were washed once with 200 μL DPBS and pellets were resuspended in 100 μL/well of 4° C. cold DPBS buffer containing 1:5000 diluted Fixable Viability Dye eFluor 450 (eBioscience, Cat. No. 65 0863 18). Plates were incubated for 30 minutes at 4° C. and washed once with 200 μL 4° C. cold DPBS buffer. Afterwards cells were resuspended in 50 μL/well of 4° C. cold FACS buffer containing different titrated concentrations of 2+1 H2H bispecific agonistic 4-1BB (20H4.9)/FAP (4B9) P329GLALA antibody with monovalent binding for FAP (also called anti-4-1BB (20H4.9)×anti-FAP (4B9) 2+1 H2H) or control molecules followed by an incubation for 1 hour at 4° C. in the dark. After washing four times with with 200 μL DPBS/well, cells were stained with 50 μL/well of 4° C. cold FACS buffer containing 2.5 μg/mL PE-conjugated AffiniPure anti-human IgG Fcγ-fragment-speciic goat F(ab′)2 fragment (Jackson ImmunoResearch, Cat.-No. 109-116-098) for 30 minutes at 4° C. Cells were washed twice with 200 μL 4° C. DPBS buffer and then resuspended in 50 μL/well DPBS containing 1% Formaldehyde for fixation. The same or the next day cells were resuspended in 100 μL FACS-buffer and acquired using MACSQuant Analyzer 10 (Miltenyi Biotec).

As shown in FIG. 4, the FAP-targeted molecules, but not the non-FAP-targeted hu IgG1 P293G LALA formats bind efficiently to human FAP-expressing NIH/3T3-huFAP clone 19 cells. Thereby the N-terminal fused anti-FAP cross-Fab (black filled circle and line) binds with a higher affinity to the FAP than the C-terminal fused anti-FAP VH/VL binding domain (grey filled squares and dotted line). This is reflected by a lower EC₅₀ value for the 2+1 H2H anti-4-1BB (20H4.9)×anti-FAP (4B9) huIgG1 P329GLALA antibody but also in a higher gMFI at saturation resulting into a higher area under the curve (AUC) value. The fitting EC₅₀ values and the values of area under the curve are listed in Table 33 and the fitting AUC values in Table 34.

TABLE 33
EC₅₀ values of binding to FAP expressing cell line NIH/3T3-huFAP clone 19

| | 2+1 H2H anti- | 2+1 VH/VL anti- | 2+1 VH/VL anti- | Untargeted |
|---|---|---|---|---|
| | 4-1BB (20H4.9) × | 4-1BB (20H4.9) × | 4-1BB (20H4.9) × | (DP47) huIgG1 |
| EC₅₀ [nM] | anti-FAP (4B9) | anti-FAP (4B9) | untargeted (DP47) | P329GLALA |
| NIH/3T3-huFAP | 1.813 | 3.944 | n.d. | 2.292 |
| clone 19 | | | | |

TABLE 34
Area under the curve (AUC) values of binding to
FAP expressing cell line NIH/3T3-huFAP clone 19

| | 2+1 H2H anti- | 2+1 VH/VL anti- | 2+1 VH/VL anti- | Untargeted |
|---|---|---|---|---|
| | 4-1BB (20H4.9) × | 4-1BB (20H4.9) × | 4-1BB (20H4.9) × | (DP47) huIgG1 |
| AUC | anti-FAP (4B9) | anti-FAP (4B9) | untargeted (DP47) | P329GLALA |
| NIH/3T3-huFAP | 40184 | 21211 | 799 | 362 |
| clone 19 | | | | |

### 4.4 Binding to Human 4-1BB Expressing Reporter Cell Line Jurkat-hu4-1BB-NFκB-luc2

For determining the binding to cell-surface-expressed human 4-1BB (CD137) Jurkat-hu4-1BB-NFκB-luc2 reporter cell line (Promega, Germany) was used. Cells were maintained as suspension cells in RPMI 1640 medium (GIBCO by Life Technologies, Cat No 42401-042) supplied with 10% (v/v) fetal bovine serum (FBS, GIBCO by Life Technologies, Cat.-No. 16000-044, Lot 941273, gamma irradiated mycoplasma free, heat inactivated), 2 mM L-alanyl-L-glutamine dipeptide (Gluta-MAX-I, GIBCO by Life Technologies, Cat.-No. 35050-038), 1 mM Sodium Pyruvate (SIGMA-Aldrich Cat.-No. S8636), 1% (v/v) MEM-Non essential Aminoacid Solution 100× (SIGMA-Aldrich, Cat.-No. M7145), 600 μg/ml G-418 (Roche, Cat.-No. 04727894001), 400 μg/ml Hygromycin B (Roche, Cat.-No.: 10843555001) and 25 mM HEPES (Sigma Life Sience, Cat.-No.: H0887-100 mL). For the binding assay 2×10⁵ of Jurkat-hu4-1BB-NFkB-luc2 were added to each well of a round-bottom suspension cell 96-well plates (Greiner bio-one, cellstar, Cat.-No. 650185). Cells were washed once with 200 μL DPBS and pellets were resuspended in 100 μL/well of 4° C. cold DPBS buffer containing 1:5000 diluted Fixable Viability Dye eFluor 450 (eBioscience, Cat. No. 65 0863 18). Plates were incubated for 30 minutes at 4° C. and washed once with 200 μL 4° C. cold DPBS buffer. Afterwards cells were resuspended in 50 μL/well of 4° C. cold FACS buffer containing different titrated concentrations of 2+1 H2H agonistic anti-4-1BB (20H4.9)×anti-FAP (4B9) huIgG1 P329GLALA antibody—or control molecules followed by an incubation for 1 hour at 4° C. in the dark. After washing four times with with 200 μL DPBS/well, cells were stained with 50 μL/well of 4° C. cold FACS buffer containing 2.5 μg/mL PE-conjugated AffiniPure anti-human IgG Fcγ-fragment-specific goat F(ab′)2 fragment (Jackson ImmunoResearch, Cat.-No. 109-116-098) for 30 minutes at 4° C. Cells were washed twice with 200 μL 4° C. DPBS buffer and then resuspended in 50 μL/well DPBS containing 1% Formaldehyde for fixation. The same or the next day cells were resuspended in 100 μL FACS-buffer and acquired using MACSQuant Analyzer X (Miltenyi Biotec).

As shown in FIG. 5, 2+1 H2H agonistic anti-4-1BB (20H4.9)×anti-FAP (4B9) huIgG1 P329GLALA antibody binds similar to 4-1BB as its control anti-4-1BB (20H4.9) huIgG1 P329G LALA. Therefore N-terminal head to head fusion of the anti-FAP cross-Fab did not influence the binding to 4-1BB. EC₅₀ values and AUC of the binding curves are listed in Table 35 and Table 36, respectively.

TABLE 35
Summary of EC₅₀ values of binding curves to
cell-expressed human 4-1BB as shown in FIG. 5

| EC₅₀ [nM] | Jurkat-hu4-1BB-NFκB-luc2 |
|---|---|
| 2 + 1 H2H anti-4-1BB (20H4.9) × anti-FAP | 0.062 |
| (4B9) huIgG1 P329GLALA | |
| anti-4-1BB (20H4.9) huIgG1 | 0.073 |
| P329GLALA | |

TABLE 36
Summary of AUC values of binding curves to cell-
expressed human 4-1BB as shown in FIG. 5

| AUC | Jurkat-hu4-1BB-NFκB-luc2 |
|---|---|
| 2 + 1 H2H anti-4-1BB (20H4.9) × anti-FAP | 16912 |
| (4B9) huIgG1 P329GLALA | |
| anti-4-1BB (20H4.9) huIgG1 P329GLALA | 16011 |

### 4.5 NF-κB Activation in Human 4-1BB and NFκB-Luciferase Reporter Gene Expressing Reporter Cell Line Jurkat-hu4-1BB-NFκB-luc2

Agonistic binding of the 4-1BB (CD137) receptor to its ligand (4-1BBL) induces 4-1BB-downstream signaling via activation of nuclear factor kappa B (NFkB) and promotes survival and activity of CD8 T cells (Lee H W, Park S J, Choi B K, Kim H H, Nam K O, Kwon B S. 4-1BB promotes the survival of CD8 (+) T lymphocytes by increasing expression of Bcl-x(L) and Bfl-1. J Immunol 2002; 169:4882-4888). To monitor this NFκB-activation mediated by 2+1 H2H anti-4-1BB, anti-FAP huIgG1 PGLALA bispecific antibody, Jurkat-hu4-1BB-NFκB-luc2 reporter cell line was purchased from Promega (Germany). The cells were cultured as described above (Binding to human 4-1BB expressing reporter cell line Jurkat-hu4-1BB-NFkB-luc2). For the assay, cells were harvested and resuspended in assay medium RPMI 1640 medium supplied with 10% (v/v) FBS and 1% (v/v) GlutaMAX-I. 10 μl containing 2×10³ Jurkat-hu4-1BB-NFκB-luc2 reporter cells were transferred to each well of a sterile white 384-well flat bottom tissue culture plate with lid (Corning, Cat.-No.:3826). 10 μL of assay medium containing titrated concentrations of 2+1 H2H bispecific agonistic anti-4-1BB (20H4.9)×anti-FAP (4B9) huIgG1 P329GLALA antibody with monovalent binding for FAP (also called anti-4-1BB (20H4.9)×anti-FAP (4B9) 2+1 H2H) or control molecules were added. Finally, 10 μL of assay medium alone or containing 1×10⁴ cells FAP-expressing cells, human melanoma cell line WM-266-4 (ATCC CRL-1676) or NIH/3T3-huFAP clone 19 (as described above) were supplied and plates were incubated for 6 hours at 37° C. and 5% CO₂ in a cell incubator. 6 μl freshly thawed One-Glo Luciferase assay detection solution (Promega, Cat.-No.: E6110) were added to each well and Luminescence light emission were measured immediately using Tecan microplate reader (500 ms integration time, no filter collecting all wavelength).

As shown in FIG. 6, in the absence of FAP expressing cells none of the molecules was able to induce strong human 4-1BB receptor activation in the Jurkat-hu4-1BB-NFkB-luc2 reporter cell line, leading to NFkB-activation and therefore Luciferase expression. In the presence of FAP-expressing cells like WM-266-4 (human melanoma cell line, intermediate FAP-expression) or NIH/3T3-huFAP clone 19 (human-FAP-transgenic mouse fibroblast cell line) crosslinking of bispecific 2+1 anti-4-1BB, anti-FAP huIgG1 PGLALA antibodies (2+1 H2H anti-4-1BB (20H4.9)×anti-FAP (4B9) antibody, black filled circle and line, or 2+1 VH/VL anti-4-1BB (20H4.9)×anti-FAP (4B9) antibody, grey filled square and dotted line) led to a strong increase of NFkB-activated Luciferase activity in the Jurkat-hu4-1BB-NFkB-luc2 reporter cell line, which was above the activation mediated by the untargeted control anti-4-1BB (20H4.9)×untargeted (DP47) 2+1 VH/VL (open grey square and small dotted line). Thereby the bispecific 2+1 H2H anti-4-1BB×anti-FAP huIgG1 P329GLALA antibody (anti-4-1BB (20H4.9)×anti-FAP (4B9) 2+1 H2H, black filled circle and line) showed a slightly better activation (lower EC₅₀ values), which may reflect the higher affinity to FAP. EC₅₀ values and area under the curve (AUC) of activation curves are listed in Table 37 and Table 38.

TABLE 37
EC₅₀ values of activation curves shown in FIG. 6

| | | NIH/3T3-huFAP |
|---|---|---|
| EC₅₀ [nM] | WM-266-4 | clone 19 |
| 2 + 1 H2H anti-4-1BB (20H4.9) × anti- | 0.060 | 0.020 |
| FAP (4B9) huIgG1 P329GLALA | | |
| 2 + 1 VH/VL (C-terminal) anti-4-1BB | 0.028 | 0.008 |
| (20H4.9) × anti-FAP (4B9) huIgG1 | | |
| P329GLALA | | |
| 2 + 1 VH/VL (C-terminal) anti-4-1BB | 0.253 | 0.313 |
| (20H4.9) × untargeted (DP47) huIgG1 | | |
| P329GLALA | | |
| Anti-FAP (4B9) huIgG1 P329GLALA | n.d. | n.d. |
| Untargeted (DP47) huIgG1 | n.d. | n.d. |
| P329GLALA | | |

TABLE 38
Values of area under the curve (AUC)
of activation curves shown in FIG. 6

| | No FAP⁺ | | NIH/3T3-huFAP |
|---|---|---|---|
| AUC | cells | WM-266-4 | clone 19 |
| 2 + 1 H2H anti-4-1BB | 715 | 49406 | 109410 |
| (20H4.9) × anti-FAP (4B9) | | | |
| huIgG1 P329GLALA | | | |
| 2 + 1 VH/VL (C-terminal) | 4363 | 30910 | 95104 |
| anti-4-1BB (20H4.9) × | | | |
| anti-FAP (4B9) huIgG1 | | | |
| P329GLALA | | | |
| 2 + 1 VH/VL (C-terminal) | 5778 | 8840 | 27218 |
| anti-4-1BB (20H4.9) × | | | |
| untargeted (DP47) huIgG1 | | | |
| P329GLALA | | | |
| Anti-FAP (4B9) huIgG1 | 181 | 191 | 195 |
| P329GLALA | | | |
| Untargeted (DP47) huIgG1 | 286 | 384 | 521 |
| P329G LALA | | | |

### Example 5

### Functional Characterization of 2+1 H2H Bispecific Agonistic 4-1BB Antigen Binding Molecules With Monovalent Binding for CEA

### 5.1 Surface Plasmon Resonance (Simultaneous Binding)

The capacity to bind simultaneously human 4-1BB Fc(kih) and human CEA, in form of NABA construct, was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany). Human N(A2B2)A or (NA1)BA protein was directly coupled to a flow cell of a CM5 chip by amine coupling. Immobilization level of approx. 600 RU was used.

The CEA targeted 4-1BB agonist construct was passed at a concentration range of 200 nM with a flow of 30 μL/minute through the flow cells over 90 seconds and dissociation was set to zero sec. Human 4-1BB Fc(kih) was injected as second analyte with a flow of 30 μL/minute through the flow cells over 90 seconds at a concentration of 500 nM (FIG. 10A). The dissociation was monitored for 120 sec. Bulk refractive index differences were corrected for by subtracting the response obtained in a reference flow cell, where no protein was immobilized.

As can be seen in FIGS. 10B and 10C, 2+1 H2H 4-1BB (20H4.9)/CEA (A5B7) P329GLALA IgG1 and 2+1 H2H 4-1BB (20H4.9)/CEA (A5H1EL1D) huIgG1 P329GLALA can bind simultaneously human CEA (in form of N(A2B2)A construct) and human 4-1BB. FIG. 10D shows 2+1 H2H 4-1BB (20H4.9)/CEA (MFE23) huIgG1 P329GLALA binding simultaneously human CEA (in form of (NA1)BA construct) and human 4-1BB.

### 5.2 Binding to Cynomolgus Monkey and Human CEACAM5-Expressing Cell Lines

First cell lines expressing cynomolgus monkey CEACAM5 or human CEACAM5 were generated. Full-length cDNAs encoding human and cynomolgus CEACAM5 were subcloned into mammalian expression vector. The plasmids were transfected into CHO-K1 (ATCC CRL-9618) cells using Lipofectamine LTX Reagent (Invitrogen, #15338100) according to the manufacturer's protocol. Stably transfected CEACAM5-positive CHO cells were maintained in DMEM/F-12 medium (GIBCO by Lifetechnologies, #11320033) supplemented with 10% fetal bovine serum (FBS, GIBCO by Life Technologies, Cat.-No. 16000-044, Lot 941273, gamma irradiated mycoplasma free, heat inactivated) and 2 mM L-alanyl-L-glutamine dipeptide (Gluta-MAX-I, GIBCO by Life Technologies, Cat.-No. 35050-038). Two days after transfection, puromycin (Invivogen; #ant-pr-1) was added to 6 μg/mL and the cells were cultured for several passages. After initial selection, the cells with high cell surface expression of human and cynomolgus CEACAM5 (detection antibody anti-CD66 clone CD66AB.1.1) were sorted by BD FACSAria II cell sorter (BD Biosciences) and cultured to establish stable cell clones. The expression level and stability was confirmed by flow cytometry analysis over a period of 4 weeks. For the binding assay CHO-k1-cynoCEACAM5 clone 8, CHO-k1-huCEACAM5 clone 11, CHO-k1-huCEACAM5 clone 12 or CHO-k1-huCEACAM5 clone 13 were harvested, washed with DPBS (GIBCO by life technologies, #14190-136) stained in DPBS containing fixable viability dye eF450 (eBioscience #65-0863-18) for 30 min at 4° C. Cells were washed and seeded to 384 well plates (Corning #3830) to 3×10⁴ cells/well. Cells were centrifuged (350×g, 5 min), supernatant was removed and cells were resuspended in 10 μL/well FACS-buffer (DPBS supplied with 2% FBS, 5 nM EDTA, 7.5 mM sodium azide) containing titrated concentrations of 2+1 H2H bispecific agonistic 4-1BB (20H4.9)×CEA huIgG1 P329GLALA antibodies or controls (start concentration 300 nM). Cells were incubated for 30 min at 4° C. and then washed twice with 80 μL/well DPBS. Cells were resuspended in 10 μL/well FACS-buffer containing 2.5 μg/mL PE-conjugated AffiniPure anti-human IgG Fcγ-fragment-specific goat F(ab′)2 fragment (Jackson ImmunoResearch, Cat.-No. 109-116-098) for 30 minutes at 4° C. Cells were washed twice with 80 μL/well DPBS and then fixed in 30 μL/well DPBS containing 1% Formaldehyde for at least 15 minutes. The same or the next day cells were resuspended in 50 μL/well FACS-buffer and acquired using MACSQuant Analyzer X (Miltenyi Biotec).

As shown in FIGS. 12A to 12D, the 2+1 H2H bispecific agonistic 4-1BB (20H4.9)×CEA huIgG1 P329GLALA antibodies, but not the non-CEA-targeted huIgG1 P293G LALA formats bind efficiently to human CEACAM5-expressing CHO-k1 clone 12 and clone 13 cells. In contrast only 2+1 H2H bispecific agonistic 4-1BB (20H4.9)×CEA (A5B7) huIgG1 P329GLALA antibody bound well to cynomolgus monkey CEACAM5 expressing CHO-k1-cynoCEACAM5 cell line. The binding of 2+1 H2H bispecific agonistic 4-1BB (20H4.9)×CEA (A5H1EL1D) huIgG1 P329GLALA antibody to cynomolgus monkey CECAM5 expressing CHO-k1-cynoCEACAM5 is very weak, whereas 2+1 H2H bispecific agonistic 4-1BB (20H4.9)×CEA (MFE23) huIgG1 P329GLALA antibody does not bind to CHO-k1-cynoCEACAM5, as the MFE23 clone is not human/cynomolgus monkey crossreactive. The fitting EC₅₀ values and the values of area under the curve are listed in Table 39 and the fitting AUC values in Table 40.

TABLE 39
EC₅₀ values of binding to CEACAM5 expressing cell lines shown in FIG. 9

| | CHO-k1- | CHO-k1- | CHO-k1- | CHO-k1- |
|---|---|---|---|---|
| | cynoCEACAM5 | humanCEACAM5 | humanCEACAM5 | humanCEACAM5 |
| EC₅₀ [nM] | clone 8 | clone 11 | clone 12 | clone 13 |
| 2 + 1 H2H anti-4-1BB | n.d. | 3 | 8.1 | 14.6 |
| (20H4.9) × anti-CEA | | | | |
| (MFE23) huIgG1 | | | | |
| P329GLALA | | | | |
| 2 + 1 H2H anti-4-1BB | 48.8 | 11.4 | 13.9 | 12.9 |
| (20H4.9) × anti-CEA | | | | |
| (A5B7) huIgG1 | | | | |
| P329GLALA | | | | |
| 2 + 1 H2H anti-4-1BB | n.d. | n.d. | n.d. | n.d. |
| (20H4.9) × anti-CEA | | | | |
| (A5H1EL1D) huIgG1 | | | | |
| P329GLALA | | | | |
| Anti-4-1BB (20H4.9) | n.d. | n.d. | n.d. | n.d. |
| huIgG1 P329G LALA | | | | |
| DP47 huIgG1 | n.d. | n.d. | n.d. | n.d. |
| P329GLALA | | | | |

TABLE 40
Area under the curve (AUC) values of binding to CEACAM5 expressing cell line shown in FIG. 9

| | CHO-k1- | CHO-k1- | CHO-k1- | CHO-k1- |
|---|---|---|---|---|
| | cynoCEACAM5 | humanCEACAM5 | humanCEACAM5 | humanCEACAM5 |
| AUC | clone 8 | clone 11 | clone 12 | clone 13 |
| 2 + 1 H2H anti-4-1BB | 169 | 25740 | 85983 | 102116 |
| (20H4.9) × anti-CEA | | | | |
| (MFE23) huIgG1 | | | | |
| P329GLALA | | | | |
| 2 + 1 H2H anti-4-1BB | 4850 | 15517 | 50894 | 66183 |
| (20H4.9) × anti-CEA | | | | |
| (A5B7) huIgG1 | | | | |
| P329GLALA | | | | |
| 2 + 1 H2H anti-4-1BB | 614 | 6797 | 25024 | 38663 |
| (20H4.9) × anti-CEA | | | | |
| (A5H1EL1D) huIgG1 | | | | |
| P329GLALA | | | | |
| Anti-4-1BB (20H4.9) | 136 | 474 | 754 | 842 |
| huIgG1 P329GLALA | | | | |
| DP47 huIgG1 | 219 | 746 | 1187 | 1866 |
| P329GLALA | | | | |

### 5.3 NFκB Activation in Human 4-1BB and NFκB-Luciferase Reporter Gene Expressing Reporter Cell Line Jurkat-hu4-1BB-NFκB-luc2

Agonistic binding of the 4-1BB (CD137) receptor to its ligand (4-1BBL) induces 4-1BB-downstream signaling via activation of nuclear factor kappa B (NFκB) and promotes survival and activity of CD8 T cells (Lee H W, Park S J, Choi B K, Kim H H, Nam K O, Kwon B S. 4-1BB promotes the survival of CD8 (+) T lymphocytes by increasing expression of Bcl-x(L) and Bfl-1. J Immunol 2002; 169:4882-4888). To monitor this NFκB-activation mediated by 2+1 H2H anti-4-1BB, anti-CEA huIgG1 PGLALA bispecific antibody, Jurkat-hu4-1BB-NFκB-luc2 reporter cell line was purchased from Promega (Germany). The cells were cultured as described above. For the assay, cells were harvested and resuspended in assay medium RPMI 1640 medium supplied with 10% (v/v) FBS and 1% (v/v) GlutaMAX-I. 10 μl containing 2×10³ Jurkat-hu4-1BB-NFκB-luc2 reporter cells were transferred to each well of a sterile white 384-well flat bottom tissue culture plate with lid (Corning, Cat.-No.:3826). 10 μL of assay medium containing titrated concentrations of 2+1 H2H bispecific agonistic anti-4-1BB (20H4.9)×anti-CEA huIgG1 P329GLALA antibody with monovalent binding for CEA (different clones, e.g. A5B7, A5H1EL1D or MFE23) or control molecules were added. Finally, 10 μL of assay medium alone or containing 1×10⁴ cells of different CHO-k1 cell transfected with cynomolgus monkey or human CEACAM5 were supplied and plates were incubated for 6 hours at 37° C. and 5% CO₂ in a cell incubator. 6 μl freshly thawed One-Glo Luciferase assay detection solution (Promega, Cat.-No.: E6110) were added to each well and Luminescence light emission were measured immediately using Tecan microplate reader (500 ms integration time, no filter collecting all wavelength).

As shown in FIGS. 13A to 13D, in the absence of CEACAM5 expressing cells none of the molecules was able to induce strong human 4-1BB receptor activation in the Jurkat-hu4-1BB-NFkB-luc2 reporter cell line, leading to NFkB-activation and therefore Luciferase expression. In the presence of humanCEACAM5-expressing cells like CHO-k1-humanCEACAM5 clone 11 and CHO-k1-humanCEACAM5 clone 12 crosslinking of bispecific 2+1 anti-4-1BB, anti-CEA huIgG1 PGLALA antibodies (2+1 H2H anti-4-1BB (20H4.9)×anti-CEA (MFE23) antibody, black filled circle and dotted line, or 2+1 H2H anti-4-1BB (20H4.9)×anti-CEA (A5B7) antibody, black filled diamond and line or 2+1 H2H anti-4-1BB (20H4.9)×anti-CEA (A5H1EL1D) antibody, grey facing-down triangle triangle) led to a strong increase of NFkB-activated Luciferase activity in the Jurkat-hu4-1BB-NFkB-luc2 reporter cell line, which was above the activation mediated by the untargeted control anti-4-1BB (20H4.9) huIgG1 P329GLALA (open grey square and dotted line). In the presence of CHO-k1-cynoCEACAM5 clone 8 only 2+1 H2H anti-4-1BB (20H4.9)×anti-CEA (A5B7) huIgG1 P329GLALA antibody (black filled diamond and line) and 2+1 H2H anti-4-1BB (20H4.9)×anti-CEA (A5H1EL1D) huIgG1 P329GLALA antibody (grey facing-down triangle triangle) induced strong increase of NFkB-activated Luciferase activity in the Jurkat-hu4-1BB-NFkB-luc2 reporter cell line but not 2+1 H2H anti-4-1BB (20H4.9)×anti-CEA (MFE23) huIgG1 P329GLALA antibody (black filled circle and dotted line), as the MFE23 binder is not human/cynomolgus monkey cross-reactive.

EC₅₀ values and area under the curve (AUC) of activation curves are listed in Table 41 and Table 42.

TABLE 41
EC₅₀ values of activation curves shown in FIG. 10

| | | CHO-k1- | CHO-k1- | CHO-k1- |
|---|---|---|---|---|
| | no CEA+ | cynoCECAM5 | humanCECAM5 | humanCECAM5 |
| EC₅₀ [nM] | cells | clone 8 | clone 11 | clone 12 |
| 2 + 1 H2H anti-4-1BB | 0.1 | 0.14 | 0.02 | 0.02 |
| (20H4.9) × anti-CEA | | | | |
| (MFE23) huIgG1 | | | | |
| P329GLALA | | | | |
| 2 + 1 H2H anti-4-1BB | 0.04 | 0.07 | 0.04 | 0.03 |
| (20H4.9) × anti-CEA | | | | |
| (A5B7) huIgG1 | | | | |
| P329GLALA | | | | |
| 2 + 1 H2H anti-4-1BB | 0.09 | 0.1 | 0.05 | 0.04 |
| (20H4.9) × anti-CEA | | | | |
| (A5H1EL1D) huIgG1 | | | | |
| P329GLALA | | | | |
| Anti-4-1BB (20H4.9) | 0.19 | 0.17 | 0.18 | 0.19 |
| huIgG1 P329GLALA | | | | |
| DP47 huIgG1 | n.d. | n.d. | n.d. | n.d. |
| P329GLALA | | | | |

TABLE 42
Values of area under the curve (AUC) of activation curves shown in FIG. 10

| | | CHO-k1- | CHO-k1- | CHO-k1- |
|---|---|---|---|---|
| | no CEA+ | cynoCECAM5 | humanCECAM5 | humanCECAM5 |
| AUC | cells | clone 8 | clone 11 | clone 12 |
| 2 + 1 H2H anti-4-1BB | 774 | 802 | 76184 | 72254 |
| (20H4.9) × anti-CEA | | | | |
| (MFE23) huIgG1 | | | | |
| P329GLALA | | | | |
| 2 + 1 H2H anti-4-1BB | 1325 | 43107 | 65721 | 58883 |
| (20H4.9) × anti-CEA | | | | |
| (A5B7) huIgG1 | | | | |
| P329GLALA | | | | |
| 2 + 1 H2H anti-4-1BB | 1131 | 26476 | 58133 | 56614 |
| (20H4.9) × anti-CEA | | | | |
| (A5H1EL1D) huIgG1 | | | | |
| P329GLALA | | | | |
| Anti-4-1BB (20H4.9) | 5067 | 4918 | 15362 | 11937 |
| huIgG1 P329GLALA | | | | |
| DP47 huIgG1 | 47 | 77 | 264 | 313 |
| P329GLALA | | | | |

### Example 6

### Preparation, Purification and Characterization of Bispecific Antibodies With a Bivalent Binding to 4-1BB and a Monovalent Binding to PD-L1

### 2.1 Generation and Production of Bispecific Antibodies With a Bivalent Binding to 4-1BB and a Monovalent Binding to PD-L1

Bispecific agonistic 4-1BB antibodies with bivalent binding for 4-1BB and monovalent binding for PD-L1 can also be prepared, by replacing the anti-FAP cross Fab with an anti-PD-L1 cross Fab. This construct is also termed Head to Head (H2H) 2+1 format.

The first heavy chain HC1 of the construct is comprised of the following components: VHCH1 of anti-4-1BB binder (clone 20H4.9), followed by Fc hole. The second heavy chain HC2 was comprised of VLCH1 of anti-PD-L1 binder (clone YW243.55.S70 in cross Fab format) followed by VHCH1 of an anti-4-1BB (clone 20H4.9) and by Fc knob. PD-L1 binder YW243.55.S70 is described in WO 2010/077634. For the 4-1BB binder, the VH and VL sequences of clone 20H4.9 were obtained in accordance with U.S. Pat. No. 7,288,638 B2 or U.S. Pat. No. 7,659,384 B2. Combination of the two heavy chains allows generation of a heterodimer, which includes a PD-L1 binding cross Fab and two 4-1BB binding Fabs (FIG. 1E). Another heterodimer with monovalent binding to 4-1BB was construed from a first heavy chain HC1 comprising VHCH1 of anti-4-1BB binder (clone 20H4.9) followed by Fc hole and a second heavy chain HC2 comprising VLCH1 of anti-PD-L1 binder (clone YW243.55.S70 in cross Fab format) followed by Fc knob (FIG. 1F).

To improve correct pairing, the following mutations were introduced in the CH-CL of the anti-4-1BB Fab molecules: E123R and Q124K in CL and K147E and K213E in CH1. The second light chain LC2 of the anti-PD-L1 binder is composed of VHCL (cross Fab). The knobs into hole technology was applied by introducing the Y349C/T366S/L368A/Y407V mutations in the first heavy chain HC1 (Fc hole heavy chain) and by introducing the S354C/T366W mutations in the second heavy chain HC2 (Fc knob heavy chain) to allow generation of a heterodimer.

Furthermore, the Pro329Gly, Leu234Ala and Leu235Ala mutations have been introduced in the constant region of the knob and hole heavy chains to abrogate binding to Fc gamma receptors according to the method described in International Patent Appl. Publ. No. WO2012/130831A1.

The bispecific 2+1 H2H anti-4-1BB anti-PD-L1 huIgG1 P329GLALA antibodies are produced as described in Example 1.2 for the 2+1 anti-4-1BB anti-FAP huIgG1 P329GLALA antibodies.

The amino acid sequences for the bispecific 4-1BB (20H4.9)/PD-L1 P329GLALA IgG1 2+1 (H2H) antibody can be found in Table 43, whereas the amino acid sequences of the bispecific 4-1BB (20H4.9)/PD-L1 P329GLALA IgG1 1+1 antibody can be found in Table 44.

The proteins are produced and purified as described in Example 1.2.

TABLE 43
Amino acid sequences of bispecific,
bivalent anti-4-1BB/monovalent anti-PD-
L1 human IgG1 P329GLALA
antibody (2 + 1 H2H)

| SEQ | | |
|---|---|---|
| ID | Descrip- | |
| NO: | tion | Sequence |
| 65 | VHCH1(EE) | See Table 1 |
| | (20H4.9)- | |
| | Heavy | |
| | chain | |
| | HC1 (Fc | |
| | hole) | |
| 193 | VLCH1 | DIQMTQSPSSLSASVGDR |
| | (PD-L1) | VTITCRASQDVSTAVAWY |
| | VHCH1(EE) | QQKPGKAPKLLIYSASFL |
| | (20H4.9)- | YSGVPSRFSGSGSGTDFT |
| | Heavy | LTISSLQPEDFATYYCQQ |
| | chain | YLYHPATFGQGTKVEIKS |
| | HC2 (Fc | SASTKGPSVFPLAPSSKS |
| | knob) | TSGGTAALGCLVKDYFPE |
| | | PVTVSWNSGALTSGVHTF |
| | | PAVLQSSGLYSLSSVVTV |
| | | PSSSLGTQTYICNVNHKP |
| | | SNTKVDKKVEPKSCDGGG |
| | | GSGGGGSQVQLQQWGAGL |
| | | LKPSETLSLTCAVYGGSF |
| | | SGYYWSWIRQSPEKGLEW |
| | | IGEINHGGYVTYNPSLES |
| | | RVTISVDTSKNQFSLKLS |
| | | SVTAADTAVYYCARDYGP |
| | | GNYDWYFDLWGRGTLVTV |
| | | SSASTKGPSVFPLAPSSK |
| | | STSGGTAALGCLVEDYFP |
| | | EPVTVSWNSGALTSGVHT |
| | | FPAVLQSSGLYSLSSVVT |
| | | VPSSSLGTQTYICNVNHK |
| | | PSNTKVDEKVEPKSCDKT |
| | | HTCPPCPAPEAAGGPSVF |
| | | LFPPKPKDTLMISRTPEV |
| | | TCVVVDVSHEDPEVKFNW |
| | | YVDGVEVHNAKTKPREEQ |
| | | YNSTYRVVSVLTVLHQDW |
| | | LNGKEYKCKVSNKALGAP |
| | | IEKTISKAKGQPREPQVY |
| | | TLPPCRDELTKNQVSLWC |
| | | LVKGFYPSDIAVEWESNG |
| | | QPENNYKTTPPVLDSDGS |
| | | FFLYSKLTVDKSRWQQGN |
| | | VFSCSVMHEALHNHYTQK |
| | | SLSLSP |
| 67 | VLCL(RK)-Light | See Table 1 |
| | chain (20H4.9) | |
| 194 | VHCL-Light | EVQLVESGGGLVQPGGSL |
| | chain (PD-L1) | RLSCAASGFTFSDSWIHW |
| | | VRQAPGKGLEWVAWISPY |
| | | GGSTYYADSVKGRFTISA |
| | | DTSKNTAYLQMNSLRAED |
| | | TAVYYCARRHWPGGFDYW |
| | | GQGTLVTVSSASVAAPSV |
| | | FIFPPSDEQLKSGTASVV |
| | | CLLNNFYPREAKVQWKVD |
| | | NALQSGNSQESVTEQDSK |
| | | DSTYSLSSTLTLSKADYE |
| | | KHKVYACEVTHQGLSSPV |
| | | TKSFNRGEC |

TABLE 44
Amino acid sequences of bispecific,
monovalent anti-4-1BB/monovalent anti-
PD-L1 human IgG1 P329GLALA antigen
binding molecules (1 + 1)

| SEQ ID | | |
|---|---|---|
| NO: | Description | Sequence |
| 65 | VHCH1(EE) | See Table 1 |
| | (20H4.9)-Heavy | |
| | chain HC1 (Fc | |
| | hole) | |
| 195 | VLCH1 (PD-L1) | DIQMTQSPSSLSASVG |
| | -Heavy chain | DRVTITCRASQDVSTA |
| | HC2 (Fc knob) | VAWYQQKPGKAPKLLI |
| | | YSASFLYSGVPSRFSG |
| | | SGSGTDFTLTISSLQP |
| | | EDFATYYCQQYLYHPA |
| | | TFGQGTKVEIKSSAST |
| | | KGPSVFPLAPSSKSTS |
| | | GGTAALGCLVKDYFPE |
| | | PVTVSWNSGALTSGVH |
| | | TFPAVLQSSGLYSLSS |
| | | VVTVPSSSLGTQTYIC |
| | | NVNHKPSNTKVDKKVE |
| | | PKSCDKTHTCPPCPAP |
| | | EAAGGPSVFLFPPKPK |
| | | DTLMISRTPEVTCVVV |
| | | DVSHEDPEVKFNWYVD |
| | | GVEVHNAKTKPREEQY |
| | | NSTYRVVSVLTVLHQD |
| | | WLNGKEYKCKVSNKAL |
| | | GAPIEKTISKAKGQPR |
| | | EPQVYTLPPCRDELTK |
| | | NQVSLWCLVKGFYPSD |
| | | IAVEWESNGQPENNYK |
| | | TTPPVLDSDGSFFLYS |
| | | KLTVDKSRWQQGNVFS |
| | | CSVMHEALHNHYTQKS |
| | | LSLSP |
| 67 | VLCL(RK)-Light | See Table 1 |
| | chain (20H4.9) | |
| 194 | VHCL-Light | see Table 43 |
| | chain (PD-L1) | |

The production of the bispecific antibodies was carried out as described in Example 1.2. Exemplary analysis of obtained products is shown in Table 45 below.

TABLE 45
Biochemical analysis of anti-4-1BB, anti-PD-L1 huIgG1 PGLALA

| | | | CE-SDS |
|---|---|---|---|
| | Monomer | Yield | (non-red, |
| Molecule | [%] | [mg/l] | main peak) |
| 2 + 1 H2H 4-1BB (20H4.9)/ | 100 | 13.8 | 90 |
| PD-L1 human IgG1 | | | |
| PGLALA | | | |
| 1 + 1 H2H 4-1BB (20H4.9)/ | 100 | 14.3 | 84 |
| PD-L1 human IgG1 | | | |
| PGLALA | | | |

### Example 7

### Functional Characterization of 2+1 11211 Bispecific Agonistic 4-1BB Antigen Binding Molecules With Monovalent Binding for PD-L1

### 7.1 Surface Plasmon Resonance (Simultaneous Binding)

The capacity to bind simultaneously human 4-1BB Fc(kih) and human PD-L1, was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany). Human 4-1BB-Fc(kih) protein was directly coupled to a flow cell of a CM5 chip by amine coupling. Immobilization level of approx. 900 RU was used.

The PD-L1 targeted 4-1BB agonist construct was passed at a concentration range of 150 nM with a flow of 10 μL/minute through the flow cells over 90 seconds and dissociation was set to zero sec. Human PD-L1-Fc (recombinant Human PD-L1/B7-H1 Fc Chimera Protein, 156-B7-100: R&D Systems) was injected as second analyte with a flow of 30 μL/minute through the flow cells over 90 seconds at a concentration of 200 nM (FIG. 14A). The dissociation was monitored for 240 sec. Bulk refractive index differences were corrected for by subtracting the response obtained in a reference flow cell, where no protein was immobilized.

As can be seen in FIGS. 14B and 14C, both the 2+1H2H and the 1+1 4-1BB (20H4.9)/PD-L1 human IgG1 PGLALA antibody can bind simultaneously human PD-L1 and human 4-1BB.

### 7.2 Binding to Human PD-L1 Expressing Cell Lines

First a cell line expressing human PD-L1 was generated. Full-length cDNAs encoding human PD-L1 were subcloned into mammalian expression vector. The plasmids were transfected into MKN45 (DSMZ 409) cells using Lipofectamine LTX Reagent (Invitrogen, #15338100) according to the manufacturer's protocol. Stably transfected PD-L1-positive PD-L1 cells were maintained in RPMI 1640 medium (GIBCO by Life Technologies, Cat No 42401-042) supplemented with 10% fetal bovine serum (FBS, GIBCO by Life Technologies, Cat.-No. 16000-044, Lot 941273, gamma irradiated mycoplasma free, heat inactivated) and 2 mM L-alanyl-L-glutamine dipeptide (Gluta-MAX-I, GIBCO by Life Technologies, Cat.-No. 35050-038) and under selection of 200 μg/mL Hygromycin B (Roche, Cat.-No. 10843555001) and 1.5 μg/mL Puromycin (Gibco by Life Technologies, Cat.-No. A11138-02). For the binding assay MKN45 cells and MKN45-huPD-L1 were harvested, washed with DPBS (GIBCO by life technologies, #14190-136) stained in DPBS containing fixable viability dye eF450 (eBioscience #65-0863-18) for 30 min at 4° C. Cells were washed and seeded to 384 well plates (Corning #3830) to 3×10⁴ cells/well. Cells were centrifuged (350×g, 5 min), supernatant was removed and cells were resuspended in 10 μL/well FACS-buffer (DPBS supplied with 2% FBS, 5 nM EDTA, 7.5 mM sodium azide) containing titrated concentrations of 2+1 H2H bispecific agonistic 4-1BB (20H4.9)×PD-L1 huIgG1 P329GLALA, 1+1 bispecific agonistic 4-1BB (20H4.9)×PD-L1 huIgG1 P329GLALA antibodies or controls (start concentration 300 nM). Cells were incubated for 30 min at 4° C. and then washed twice with 80 μL/well DPBS. Cells were resuspended in 10 μL/well FACS-buffer containing 2.5 μg/mL PE-conjugated AffiniPure anti-human IgG Fcγ-fragment-specific goat F(ab′)2 fragment (Jackson ImmunoResearch, Cat.-No. 109-116-098) for 30 minutes at 4° C. Cells were washed twice with 80 μL/well DPBS and then fixed in 30 μL/well DPBS containing 1% Formaldehyde for at least 15 minutes. The same or the next day cells were resuspended in 50 μL/well FACS-buffer and acquired using MACSQuant Analyzer X (Miltenyi Biotec).

As shown in FIG. 15B, the 2+1 H2H bispecific agonistic 4-1BB (20H4.9)×PD-L1 huIgG1 P329GLALA antibody (black triangle and line) and the 1+1 bispecific agonistic 4-1BB (20H4.9)×PD-L1 (grey triangle and line), but not the non-PD-L1-targeted huIgG1 P293GLALA formats bind efficiently to human PD-L1-expressing MKN45-huPD-L1 cells but not to the parental cell line MKN45. The fitting EC₅₀ values and the values of area under the curve are listed in Table 46 and the fitting AUC values in Table 47.

TABLE 46
EC₅₀ values of binding to PD-L1 expressing
cell line shown in FIG. 15B

| | MKN45- |
|---|---|
| EC₅₀ [nM] | huPD-L1 |
| 2 + 1 H2H anti-4-1BB | 1.95 |
| (20H4.9) × anti-PD-L1 | |
| huIgG1 P329GLALA | |
| 1 + 1 anti-4-1BB | 1.54 |
| (20H4.9) × anti-PD-L1) | |
| huIgG1 P329GLALA | |

TABLE 47
Area under the curve (AUC) values of binding to
PD-L1 expressing cell line shown in FIG. 15B

| | MKN45- |
|---|---|
| AUC | huPD-L1 |
| 2 + 1 H2H anti-4-1BB | 66296 |
| (20H4.9) × anti-PD-L1 | |
| huIgG1 P329GLALA | |
| 1 + 1 anti-4-1BB | 63744 |
| (20H4.9) × anti-PD-L1) | |
| huIgG1 P329GLALA | |

### 7.3 NF-κB Activation in Human 4-1BB and NFκB-Luciferase Reporter Gene Expressing Reporter Cell Line Jurkat-hu4-1BB-NFκB-luc2

Agonistic binding of the 4-1BB (CD137) receptor to its ligand (4-1BBL) induces 4-1BB-downstream signaling via activation of nuclear factor kappa B (NFkB) and promotes survival and activity of CD8 T cells (Lee H W, Park S J, Choi B K, Kim H H, Nam K O, Kwon B S. 4-1BB promotes the survival of CD8 (+) T lymphocytes by increasing expression of Bcl-x(L) and Bfl-1. J Immunol 2002; 169:4882-4888). To monitor this NFκB-activation mediated by 2+1 H2H anti-4-1BB×anti-PD-L1 huIgG1 PGLALA bispecific antibody, Jurkat-hu4-1BB-NFκB-luc2 reporter cell line was purchased from Promega (Germany). The cells were cultured as described above. For the assay, cells were harvested and resuspended in assay medium RPMI 1640 medium supplied with 10% (v/v) FBS and 1% (v/v) GlutaMAX-I. 10 μl containing 2×10³ Jurkat-hu4-1BB-NFκB-luc2 reporter cells were transferred to each well of a sterile white 384-well flat bottom tissue culture plate with lid (Corning, Cat.-No.:3826). 10 μL of assay medium containing titrated concentrations of 2+1 H2H bispecific agonistic anti-4-1BB (20H4.9)×anti-PD-L1 huIgG1 P329GLALA antibody, 1+1 bispecific agonistic anti-4-1BB (20H4.9)×anti-PD-L1 huIgG1 P329GLALA antibody or control molecules were added. Finally, 10 μL of assay medium alone or containing 1×10⁴ cells of parental MKN45 or MKN45 cells transfected with human PD-L1 were supplied and plates were incubated for 6 hours at 37° C. and 5% CO₂ in a cell incubator. 6 μl freshly thawed One-Glo Luciferase assay detection solution (Promega, Cat.-No.: E6110) were added to each well and Luminescence light emission were measured immediately using Tecan microplate reader (500 ms integration time, no filter collecting all wavelength).

As shown in FIGS. 16A to 16C, in the absence of PD-L1 expressing cells none of the molecules was able to induce strong human 4-1BB receptor activation in the Jurkat-hu4-1BB-NFkB-luc2 reporter cell line, leading to NFkB-activation and therefore Luciferase expression. In the presence of humanPD-L1-expressing MKN45 cells crosslinking of bispecific 2+1 H2H anti-4-1BB, anti-PD-L1 huIgG1 PGLALA antibody (black triangle and line) or bispecific 1+1 anti-4-1BB, anti-PD-L1 huIgG1 PGLALA antibody (grey triangle and line) led to a strong increase of NFkB-activated Luciferase activity in the Jurkat-hu4-1BB-NFkB-luc2 reporter cell line, which was above the activation mediated by the untargeted control anti-4-1BB (20H4.9) huIgG1 P329GLALA (filled grey circle and line). EC₅₀ values and area under the curve (AUC) of activation curves are listed in Table 48 and Table 49.

TABLE 48
EC₅₀ values of activation curves shown in FIG. 16B

| | MKN45- |
|---|---|
| EC₅₀ [nM] | huPD-L1 |
| 2 + 1 H2H anti-4-1BB | 0.04 |
| (20H4.9) × anti-PD-L1 | |
| huIgG1 P329GLALA | |
| 1 + 1 anti-4-1BB | 0.10 |
| (20H4.9) × anti-PD-L1) | |
| huIgG1 P329GLALA | |

TABLE 49
Values of area under the curve (AUC) of
activation curves shown in FIG. 16C

| | MKN45- |
|---|---|
| AUC | huPD-L1 |
| 2 + 1 H2H anti-4-1BB | 134607 |
| (20H4.9) × anti-PD-L1 | |
| huIgG1 P329GLALA | |
| 1 + 1 anti-4-1BB | 122416 |
| (20H4.9) × anti-PD-L1) | |
| huIgG1 P329GLALA | |

1. A bispecific antigen binding molecule, comprising
(a) a first Fab fragment capable of specific binding to 4-1BB,
(b) a second Fab fragment capable of specific binding to a target cell antigen,
(c) a third Fab fragment capable of specific binding to 4-1BB, and
(d) a Fc domain composed of a first and a second subunit capable of stable association, wherein the second Fab fragment (b) is fused at the C-terminus of the Fab heavy chain to the N-terminus of the Fab heavy chain of the first Fab fragment (a), which is in turn fused at its C-terminus to the N-terminus of the first Fc domain subunit, and the third Fab fragment (c) is fused at the C-terminus of the Fab heavy chain to the N-terminus of the second Fc domain subunit, and
wherein in the second Fab fragment capable of specific binding to a target cell antigen (i) the variable domains VL and VH are replaced by each other, or (ii) the constant domains CL and CH1 are replaced by each other.

2. The bispecific antigen binding molecule of claim 1, wherein the bispecific antigen binding molecule provides bivalent binding to 4-1BB and monovalent binding to the target cell antigen.

3. The bispecific antigen binding molecule of claim 1, wherein the Fc domain composed of a first and a second subunit capable of stable association is an IgG Fc domain.

4. The bispecific antigen binding molecule of claim 1, wherein the first subunit of the Fc domain comprises knobs and the second subunit of the Fc domain comprises holes.

5. The bispecific antigen binding molecule of claim 1, wherein the Fc domain comprises one or more amino acid mutations that reduces the binding affinity of the antigen binding molecule to an Fc receptor and/or effector function, and wherein the one or more amino acid mutations are selected from the group consisting of L234A, L235A and P329G (numbering according to Kabat EU index).

6. The bispecific antigen binding molecule of claim 1, wherein the first and the third Fab fragment capable of specific binding to 4-1BB each comprise
a heavy chain variable region (VH4-1BB) comprising (i) CDR-H1 comprising the amino acid sequence of SEQ ID NO:1, (ii) CDR-H2 comprising the amino acid sequence of SEQ ID NO:2, and (iii) CDR-H3 comprising the amino acid sequence of SEQ ID NO:3, and
a light chain variable region (VL4-1BB) comprising (iv) CDR-L1 comprising the amino acid sequence of SEQ ID NO:4, (v) CDR-L2 comprising the amino acid sequence of SEQ ID NO:5, and (vi) CDR-L3 comprising the amino acid sequence of SEQ ID NO:6.

7. The bispecific antigen binding molecule of claim 1, wherein the first and the third Fab fragment capable of specific binding to 4-1BB each comprise
a heavy chain variable region (VH4-1BB) comprising an amino acid sequence that is at least about 95%, 96%, 97%, 98%, 99% or 100% identical to the amino acid sequence of SEQ ID NO:7 and
a light chain variable region (VL4-1BB) comprising an amino acid sequence that is at least about 95%, 96%, 97%, 98%, 99% or 100% identical to the amino acid sequence of SEQ ID NO:8.

8. The bispecific antigen binding molecule of claim 1, wherein in the constant domain CL of the first and the third Fab fragment capable of specific binding to 4-1BB the amino acid at position 124 is substituted by lysine (K) (numbering according to Kabat EU index) and the amino acid at position 123 is substituted by arginine (R) or lysine (K) (numbering according to Kabat EU index), and wherein in the constant domain CH1 of the first and the third Fab fragment capable of specific binding to 4-1BB the amino acid at position 147 is substituted by glutamic acid (E) (numbering according to Kabat EU index) and the amino acid at position 213 is substituted by glutamic acid (E) (numbering according to Kabat EU index).

9. The bispecific antigen binding molecule of claim 1, wherein the second Fab fragment is capable of specific binding to a target cell antigen selected from the group consisting of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20, CD33 and PD-L1.

10. The bispecific antigen binding molecule of claim 9, wherein the second Fab fragment is capable of specific binding to Fibroblast Activation Protein (FAP).

11. The bispecific antigen binding molecule of claim 10, wherein the second Fab fragment capable of specific binding to Fibroblast Activation Protein (FAP) comprises
(a) a heavy chain variable region (VHFAP) comprising (i) CDR-H1 comprising the amino acid sequence of SEQ ID NO:9, (ii) CDR-H2 comprising the amino acid sequence of SEQ ID NO:10, and (iii) CDR-H3 comprising the amino acid sequence of SEQ ID NO:11, and a light chain variable region (VLFAP) comprising (iv) CDR-L1 comprising the amino acid sequence of SEQ ID NO:12, (v) CDR-L2 comprising the amino acid sequence of SEQ ID NO:13, and (vi) CDR-L3 comprising the amino acid sequence of SEQ ID NO:14, or
(b) a heavy chain variable region (VHFAP) comprising (i) CDR-H1 comprising the amino acid sequence of SEQ ID NO:15, (ii) CDR-H2 comprising the amino acid sequence of SEQ ID NO:16, and (iii) CDR-H3 comprising the amino acid sequence of SEQ ID NO:17, and a light chain variable region (VLFAP) comprising (iv) CDR-L1 comprising the amino acid sequence of SEQ ID NO:18, (v) CDR-L2 comprising the amino acid sequence of SEQ ID NO:19, and (vi) CDR-L3 comprising the amino acid sequence of SEQ ID NO:20.

12. The bispecific antigen binding molecule of claim 10, wherein the second Fab fragment capable of specific binding to Fibroblast Activation Protein (FAP) comprises
(a) a heavy chain variable region (VHFAP) comprising an amino acid sequence that is at least about 95%, 96%, 97%, 98%, 99% or 100% identical to the amino acid sequence of SEQ ID NO:21, and a light chain variable region (VLFAP) comprising an amino acid sequence that is at least about 95%, 96%, 97%, 98%, 99% or 100% identical to the amino acid sequence of SEQ ID NO:22, or
(b) a heavy chain variable region (VHFAP) comprising an amino acid sequence that is at least about 95%, 96%, 97%, 98%, 99% or 100% identical to the amino acid sequence of SEQ ID NO:23, and a light chain variable region (VLFAP) comprising an amino acid sequence that is at least about 95%, 96%, 97%, 98%, 99% or 100% identical to the amino acid sequence of SEQ ID NO:24.

13. The bispecific antigen binding molecule of claim 9, wherein the second Fab fragment is capable of specific binding to Carcinoembryonic Antigen (CEA).

14. The bispecific antigen binding molecule of claim 13, wherein the second Fab fragment capable of specific binding to Carcinoembryonic Antigen (CEA) comprises
(a) a heavy chain variable region (VHCEA) comprising (i) CDR-H1 comprising the amino acid sequence of SEQ ID NO:25, (ii) CDR-H2 comprising the amino acid sequence of SEQ ID NO:26, and (iii) CDR-H3 comprising the amino acid sequence of SEQ ID NO:27, and a light chain variable region (VLCEA) comprising (iv) CDR-L1 comprising the amino acid sequence of SEQ ID NO:28, (v) CDR-L2 comprising the amino acid sequence of SEQ ID NO:29, and (vi) CDR-L3 comprising the amino acid sequence of SEQ ID NO:30, or
(b) a heavy chain variable region (VHCEA) comprising (i) CDR-H1 comprising the amino acid sequence of SEQ ID NO:33, (ii) CDR-H2 comprising the amino acid sequence of SEQ ID NO:34, and (iii) CDR-H3 comprising the amino acid sequence of SEQ ID NO:35, and a light chain variable region (VLCEA) comprising (iv) CDR-L1 comprising the amino acid sequence of SEQ ID NO:36, (v) CDR-L2 comprising the amino acid sequence of SEQ ID NO:37, and (vi) CDR-L3 comprising the amino acid sequence of SEQ ID NO:38, or
(c) a heavy chain variable region (VHCEA) comprising (i) CDR-H1 comprising the amino acid sequence of SEQ ID NO:41, (ii) CDR-H2 comprising the amino acid sequence of SEQ ID NO:42, and (iii) CDR-H3 comprising the amino acid sequence of SEQ ID NO:43, and a light chain variable region (VLCEA) comprising (iv) CDR-L1 comprising the amino acid sequence of SEQ ID NO:44, (v) CDR-L2 comprising the amino acid sequence of SEQ ID NO:45, and (vi) CDR-L3 comprising the amino acid sequence of SEQ ID NO:46, or
(d) a heavy chain variable region (VHCEA) comprising (i) CDR-H1 comprising the amino acid sequence of SEQ ID NO:49, (ii) CDR-H2 comprising the amino acid sequence of SEQ ID NO:50, and (iii) CDR-H3 comprising the amino acid sequence of SEQ ID NO:51, and a light chain variable region (VLCEA) comprising (iv) CDR-L1 comprising the amino acid sequence of SEQ ID NO:52, (v) CDR-L2 comprising the amino acid sequence of SEQ ID NO:53, and (vi) CDR-L3 comprising the amino acid sequence of SEQ ID NO:54, or
(e) a heavy chain variable region (VHCEA) comprising (i) CDR-H1 comprising the amino acid sequence of SEQ ID NO:115, (ii) CDR-H2 comprising the amino acid sequence of SEQ ID NO:116, and (iii) CDR-H3 comprising the amino acid sequence of SEQ ID NO:117, and a light chain variable region (VLCEA) comprising (iv) CDR-L1 comprising the amino acid sequence of SEQ ID NO:118, (v) CDR-L2 comprising the amino acid sequence of SEQ ID NO:119, and (vi) CDR-L3 comprising the amino acid sequence of SEQ ID NO:120, or
(f) a heavy chain variable region (VHCEA) comprising (i) CDR-H1 comprising the amino acid sequence of SEQ ID NO:123, (ii) CDR-H2 comprising the amino acid sequence of SEQ ID NO:124 or SEQ ID NO:125, and (iii) CDR-H3 comprising the amino acid sequence of SEQ ID NO:126, and a light chain variable region (VLCEA) comprising (iv) CDR-L1 comprising the amino acid sequence of SEQ ID NO:127 or SEQ ID NO:128, (v) CDR-L2 comprising the amino acid sequence of SEQ ID NO:129 or SEQ ID NO:130 or SEQ ID NO:131, and (vi) CDR-L3 comprising the amino acid sequence of SEQ ID NO:132.

15. The bispecific antigen binding molecule of claim 13, wherein the second Fab fragment capable of specific binding to Carcinoembryonic Antigen (CEA) comprises
(a) a heavy chain variable region (VHCEA) comprising an amino acid sequence that is at least about 95%, 96%, 97%, 98%, 99% or 100% identical to the amino acid sequence of SEQ ID NO:31, and a light chain variable region (VLCEA) comprising an amino acid sequence that is at least about 95%, 96%, 97%, 98%, 99% or 100% identical to the amino acid sequence of SEQ ID NO:32, or
(b) a heavy chain variable region (VHCEA) comprising an amino acid sequence that is at least about 95%, 96%, 97%, 98%, 99% or 100% identical to the amino acid sequence of SEQ ID NO:39, and a light chain variable region (VLCEA) comprising an amino acid sequence that is at least about 95%, 96%, 97%, 98%, 99% or 100% identical to the amino acid sequence of SEQ ID NO:40, or
(c) a heavy chain variable region (VHCEA) comprising an amino acid sequence that is at least about 95%, 96%, 97%, 98%, 99% or 100% identical to the amino acid sequence of SEQ ID NO:47, and a light chain variable region (VLCEA) comprising an amino acid sequence that is at least about 95%, 96%, 97%, 98%, 99% or 100% identical to the amino acid sequence of SEQ ID NO:48, or
(d) a heavy chain variable region (VHCEA) comprising an amino acid sequence that is at least about 95%, 96%, 97%, 98%, 99% or 100% identical to the amino acid sequence of SEQ ID NO:55, and a light chain variable region (VLCEA) comprising an amino acid sequence that is at least about 95%, 96%, 97%, 98%, 99% or 100% identical to the amino acid sequence of SEQ ID NO:56, or
(e) a heavy chain variable region (VHCEA) comprising an amino acid sequence that is at least about 95%, 96%, 97%, 98%, 99% or 100% identical to the amino acid sequence of SEQ ID NO:121, and a light chain variable region (VLCEA) comprising an amino acid sequence that is at least about 95%, 96%, 97%, 98%, 99% or 100% identical to the amino acid sequence of SEQ ID NO:122, or
(f) a heavy chain variable region (VHCEA) comprising an amino acid sequence that is at least about 95%, 96%, 97%, 98%, 99% or 100% identical to the amino acid sequence of SEQ ID NO:55, and a light chain variable region (VLCEA) comprising an amino acid sequence that is at least about 95%, 96%, 97%, 98%, 99% or 100% identical to the amino acid sequence of SEQ ID NO:56, or
(g) a heavy chain variable region (VHCEA) comprising an amino acid sequence that is at least about 95%, 96%, 97%, 98%, 99% or 100% identical to the amino acid sequence of SEQ ID NO:133, and a light chain variable region (VLCEA) comprising an amino acid sequence that is at least about 95%, 96%, 97%, 98%, 99% or 100% identical to the amino acid sequence of SEQ ID NO:143, or
(h) a heavy chain variable region (VHCEA) comprising an amino acid sequence that is at least about 95%, 96%, 97%, 98%, 99% or 100% identical to the amino acid sequence of SEQ ID NO:137, and a light chain variable region (VLCEA) comprising an amino acid sequence that is at least about 95%, 96%, 97%, 98%, 99% or 100% identical to the amino acid sequence of SEQ ID NO:143.

16. The bispecific antigen binding molecule of claim 13, wherein the Fab fragment capable of specific binding to Carcinoembryonic Antigen (CEA) comprises
a heavy chain variable region (VHCEA) comprising the amino acid sequence of SEQ ID NO:133, SEQ ID NO:134, SEQ ID NO:135, SEQ ID NO:136, SEQ ID NO:137 or SEQ ID NO:138, and
a light chain variable region (VLCEA) comprising the amino acid sequence of SEQ ID NO:139, SEQ ID NO:140, SEQ ID NO:141, SEQ ID NO:142, SEQ ID NO:143 or SEQ ID NO:144.

17. The bispecific antigen binding molecule of claim 9, wherein the second Fab fragment is capable of specific binding to CD19.

18. The bispecific antigen binding molecule of claim 17, wherein the second Fab fragment capable of specific binding to CD19 comprises
a heavy chain variable region (VHCD19) comprising (i) CDR-H1 comprising the amino acid sequence of SEQ ID NO:57, (ii) CDR-H2 comprising the amino acid sequence of SEQ ID NO:58, and (iii) CDR-H3 comprising the amino acid sequence of SEQ ID NO:59, and
a light chain variable region (VLCD19) comprising (iv) CDR-L1 comprising the amino acid sequence of SEQ ID NO:60, (v) CDR-L2 comprising the amino acid sequence of SEQ ID NO:61, and (vi) CDR-L3 comprising the amino acid sequence of SEQ ID NO:62.

19. The bispecific antigen binding molecule of claim 17, wherein the second Fab fragment capable of specific binding to CD19 comprises
a heavy chain variable region (VHCD19) comprising an amino acid sequence that is at least about 95%, 96%, 97%, 98%, 99% or 100% identical to the amino acid sequence of SEQ ID NO:63, and
a light chain variable region (VLCD19) comprising an amino acid sequence that is at least about 95%, 96%, 97%, 98%, 99% or 100% identical to the amino acid sequence of SEQ ID NO:64.

20. The bispecific antigen binding molecule of claim 9, wherein the second Fab fragment is capable of specific binding to PD-L1.

21. The bispecific antigen binding molecule of claim 20, wherein the second Fab fragment capable of specific binding to PD-L1 comprises
a heavy chain variable region (VHPD-L1) comprising (i) CDR-H1 comprising the amino acid sequence of SEQ ID NO:145, (ii) CDR-H2 comprising the amino acid sequence of SEQ ID NO:146, and (iii) CDR-H3 comprising the amino acid sequence of SEQ ID NO:147, and
a light chain variable region (VLPD-L1) comprising (iv) CDR-L1 comprising the amino acid sequence of SEQ ID NO:148, (v) CDR-L2 comprising the amino acid sequence of SEQ ID NO:149, and (vi) CDR-L3 comprising the amino acid sequence of SEQ ID NO:150.

22. The bispecific antigen binding molecule of claim 20, wherein the Fab fragment capable of specific binding to PD-L1 comprises
a heavy chain variable region (VHPD-L1) comprising an amino acid sequence that is at least about 95%, 96%, 97%, 98%, 99% or 100% identical to the amino acid sequence of SEQ ID NO:152, and
a light chain variable region (VLPD-L1) comprising an amino acid sequence that is at least about 95%, 96%, 97%, 98%, 99% or 100% identical to the amino acid sequence of SEQ ID NO:153.

23. A polynucleotide encoding the bispecific antigen binding molecule of claim 1.

24. A vector comprising the polynucleotide of claim 23.

25. A host cell comprising the vector of claim 24.

26. A method of producing a bispecific antigen binding molecule comprising culturing the host cell of claim 25 under conditions suitable for the expression of the bispecific antigen binding molecule.

27. A pharmaceutical composition comprising a bispecific antigen binding molecule of claim 1 and at least one pharmaceutically acceptable excipient.

28-32. (canceled)

33. A method of treating cancer or an infectious disease, or inhibiting the growth of tumor cells, in an individual comprising administering to the individual a therapeutically effective amount of a pharmaceutical composition comprising the bispecific antigen binding molecule of claim 1 and at least one pharmaceutically acceptable excipient.

34. The method of claim 33, for treating cancer, wherein the bispecific antigen binding molecule is administered in combination with a chemotherapeutic agent, radiation therapy and/or other agent used in cancer immunotherapy.

35. The bispecific antigen binding molecule of claim 3, wherein the Fc domain composed of a first and a second subunit capable of stable association is an IgG1 Fc domain or an IgG4 Fc domain.


SEQUENCE LISTING (196 sequences)

SEQ ID NO: 1 - protein, 5 aa, Artificial Sequence
  4-1BB (20H4.9) CDR-H1
GYYWS

SEQ ID NO: 2 - protein, 16 aa, Artificial Sequence
  4-1BB (20H4.9) CDR-H2
EINHGGYVTYNPSLES

SEQ ID NO: 3 - protein, 13 aa, Artificial Sequence
  4-1BB (20H4.9) CDR-H3
DYGPGNYDWYFDL

SEQ ID NO: 4 - protein, 11 aa, Artificial Sequence
  4-1BB (20H4.9) CDR-L1
RASQSVSSYLA

SEQ ID NO: 5 - protein, 7 aa, Artificial Sequence
  4-1BB (20H4.9) CDR-L2
DASNRAT

SEQ ID NO: 6 - protein, 11 aa, Artificial Sequence
  4-1BB (20H4.9) CDR-L3
QQRSNWPPALT

SEQ ID NO: 7 - protein, 121 aa, Artificial Sequence [sequence omitted]

SEQ ID NO: 8 - protein, 109 aa, Artificial Sequence [sequence omitted]

SEQ ID NO: 9 - protein, 5 aa, Artificial Sequence
  FAP(4B9)  CDR-H1
SYAMS

SEQ ID NO: 10 - protein, 17 aa, Artificial Sequence
  FAP(4B9)  CDR-H2
AIIGSGASTYYADSVKG

SEQ ID NO: 11 - protein, 8 aa, Artificial Sequence
  FAP(4B9)  CDR-H3
GWFGGFNY

SEQ ID NO: 12 - protein, 12 aa, Artificial Sequence
  FAP(4B9)  CDR-L1
RASQSVTSSYLA

SEQ ID NO: 13 - protein, 7 aa, Artificial Sequence
  FAP(4B9)  CDR-L2
VGSRRAT

SEQ ID NO: 14 - protein, 9 aa, Artificial Sequence
  FAP(4B9)  CDR-L3
QQGIMLPPT

SEQ ID NO: 15 - protein, 5 aa, Artificial Sequence
  FAP(28H1)  CDR-H1
SHAMS

SEQ ID NO: 16 - protein, 16 aa, Artificial Sequence
  FAP(28H1)  CDR-H2
AIWASGEQYYADSVKG

SEQ ID NO: 17 - protein, 8 aa, Artificial Sequence
  FAP(28H1)  CDR-H3
GWLGNFDY

SEQ ID NO: 18 - protein, 12 aa, Artificial Sequence
  FAP(28H1)  CDR-L1
RASQSVSRSYLA

SEQ ID NO: 19 - protein, 7 aa, Artificial Sequence
  FAP(28H1)  CDR-L2
GASTRAT

SEQ ID NO: 20 - protein, 9 aa, Artificial Sequence
  FAP(28H1)  CDR-L3
QQGQVIPPT

SEQ ID NO: 21 - protein, 117 aa, Artificial Sequence
  FAP(4B9) VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 22 - protein, 108 aa, Artificial Sequence
  FAP(4B9) VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 23 - protein, 116 aa, Artificial Sequence
  FAP(28H1) VH
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 24 - protein, 108 aa, Artificial Sequence
  FAP(28H1) VL
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 25 - protein, 5 aa, Artificial Sequence
  CEA (A5B7)- CDR-H1
DYYMN

SEQ ID NO: 26 - protein, 19 aa, Artificial Sequence
  CEA (A5B7)- CDR-H2
FIGNKANGYTTEYSASVKG

SEQ ID NO: 27 - protein, 10 aa, Artificial Sequence
  CEA (A5B7)- CDR-H3
DRGLRFYFDY

SEQ ID NO: 28 - protein, 10 aa, Artificial Sequence
  CEA (A5B7)- CDR-L1
RASSSVTYIH

SEQ ID NO: 29 - protein, 7 aa, Artificial Sequence
  CEA (A5B7)- CDR-L2
ATSNLAS

SEQ ID NO: 30 - protein, 9 aa, Artificial Sequence
  CEA (A5B7)- CDR-L3
QHWSSKPPT

SEQ ID NO: 31 - protein, 121 aa, Artificial Sequence
  CEA (A5B7) VH (parental)
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSS

SEQ ID NO: 32 - protein, 106 aa, Artificial Sequence
  CEA (A5B7) VL (parental)
QTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIK

SEQ ID NO: 33 - protein, 5 aa, Artificial Sequence
  CEA (MFE23)- CDR-H1
DSYMH

SEQ ID NO: 34 - protein, 17 aa, Artificial Sequence
  CEA (MFE23)- CDR-H2
WIDPENGDTEYAPKFQG

SEQ ID NO: 35 - protein, 11 aa, Artificial Sequence
  CEA (MFE23)- CDR-H3
GTPTGPYYFDY

SEQ ID NO: 36 - protein, 10 aa, Artificial Sequence
  CEA (MFE23)- CDR-L1
SASSSVSYMH

SEQ ID NO: 37 - protein, 7 aa, Artificial Sequence
  CEA (MFE23)- CDR-L2
STSNLAS

SEQ ID NO: 38 - protein, 9 aa, Artificial Sequence
  CEA (MFE23)- CDR-L3
QQRSSYPLT

SEQ ID NO: 39 - protein, 120 aa, Artificial Sequence
  CEA (MFE23) VH
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSS

SEQ ID NO: 40 - protein, 106 aa, Artificial Sequence
  CEA (MFE23) VL
ENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELK

SEQ ID NO: 41 - protein, 5 aa, Artificial Sequence
  CEA (T84.66-LCHA)- CDR-H1
DTYMH

SEQ ID NO: 42 - protein, 17 aa, Artificial Sequence
  CEA (T84.66-LCHA)- CDR-H2
RIDPANGNSKYVPKFQG

SEQ ID NO: 43 - protein, 12 aa, Artificial Sequence
  CEA (T84.66-LCHA)- CDR-H3
FGYYVSDYAMAY

SEQ ID NO: 44 - protein, 15 aa, Artificial Sequence
  CEA (T84.66-LCHA)- CDR-L1
RAGESVDIFGVGFLH

SEQ ID NO: 45 - protein, 7 aa, Artificial Sequence
  CEA (T84.66-LCHA)- CDR-L2
RASNRAT

SEQ ID NO: 46 - protein, 9 aa, Artificial Sequence
  CEA (T84.66-LCHA)- CDR-L3
QQTNEDPYT

SEQ ID NO: 47 - protein, 121 aa, Artificial Sequence
  CEA (T84.66-LCHA) VH
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSS

SEQ ID NO: 48 - protein, 111 aa, Artificial Sequence
  CEA (T84.66-LCHA) VL
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIK

SEQ ID NO: 49 - protein, 5 aa, Artificial Sequence
  CEA (CH1A1A 98/99)- CDR-H1
EFGMN

SEQ ID NO: 50 - protein, 17 aa, Artificial Sequence
  CEA (CH1A1A 98/99)- CDR-H2
WINTKTGEATYVEEFKG

SEQ ID NO: 51 - protein, 12 aa, Artificial Sequence
  CEA (CH1A1A 98/99)- CDR-H3
WDFAYYVEAMDY

SEQ ID NO: 52 - protein, 11 aa, Artificial Sequence
  CEA (CEA 2F1)- CDR-L1
KASAAVGTYVA

SEQ ID NO: 53 - protein, 7 aa, Artificial Sequence
  CEA (CEA 2F1)- CDR-L2
SASYRKR

SEQ ID NO: 54 - protein, 10 aa, Artificial Sequence
  CEA (CEA 2F1)- CDR-L3
HQYYTYPLFT

SEQ ID NO: 55 - protein, 121 aa, Artificial Sequence
  CEA (CH1A1A 98/99) VH
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 56 - protein, 108 aa, Artificial Sequence
  CEA (CEA 2F1) VL
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 57 - protein, 5 aa, Artificial Sequence
  CD19 (8B8-2B11) CDR-H1
DYIMH

SEQ ID NO: 58 - protein, 17 aa, Artificial Sequence
  CD19 (8B8-2B11) CDR-H2
YINPYNDGSKYTEKFQG

SEQ ID NO: 59 - protein, 12 aa, Artificial Sequence
  CD19 (8B8-2B11) CDR-H3
GTYYYGPQLFDY

SEQ ID NO: 60 - protein, 16 aa, Artificial Sequence
  CD19 (8B8-2B11) CDR-L1
KSSQSLETSTGTTYLN

SEQ ID NO: 61 - protein, 7 aa, Artificial Sequence
  CD19 (8B8-2B11) CDR-L2
RVSKRFS

SEQ ID NO: 62 - protein, 9 aa, Artificial Sequence
  CD19 (8B8-2B11) CDR-L3
LQLLEDPYT

SEQ ID NO: 63 - protein, 121 aa, Artificial Sequence
  CD19 (8B8-2B11) VH
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSS

SEQ ID NO: 64 - protein, 112 aa, Artificial Sequence
  CD19 (8B8-2B11) VL
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIK

SEQ ID NO: 65 - protein, 449 aa, Artificial Sequence
  VHCH1(EE) (20H4.9)-Heavy chain HC1 (Fc hole)
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 66 - protein, 673 aa, Artificial Sequence
  VLCH1 (4B9) VHCH1(EE) (20H4.9) -Heavy chain HC2 (Fc knob)
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 67 - protein, 216 aa, Artificial Sequence
  VLCL(RK)-Light chain (20H4.9)
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 224 aa, Artificial Sequence
  VHCL-Light chain (4B9)
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 225 aa, Artificial Sequence
  Fc hole chain
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 70 - protein, 422 aa, Artificial Sequence
  human 4-1BB antigen Fc knob chain
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQVDEQLYFQGGSPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 71 - protein, 587 aa, Artificial Sequence
  VHCH1 (20H4.9)- heavy chain HC1 (Fc hole) -VH (4B9)
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 72 - protein, 578 aa, Artificial Sequence
  VHCH1 (20H4.9)-Heavy chain HC2 (Fc knob) -VL (4B9)
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 73 - protein, 216 aa, Artificial Sequence
  VLCL-Light chain (20H4.9)
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 575 aa, Artificial Sequence
  VHCH1 (20H4.9)- heavy chain HC1 (Fc hole) -VH (DP47)
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSS

SEQ ID NO: 75 - protein, 578 aa, Artificial Sequence
  VHCH1 (20H4.9)-Heavy chain HC2 (Fc knob) -VL (DP47)
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 76 - protein, 671 aa, Artificial Sequence
  VLCH1 (A5B7) VHCH1(EE) (20H4.9) -Heavy chain HC2 (Fc knob)
QTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 77 - protein, 228 aa, Artificial Sequence
  VHCL-Light chain (A5B7)
EVKLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTLTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 671 aa, Artificial Sequence
  VLCH1 (MFE23) VHCH1(EE) (20H4.9) -Heavy chain HC2 (Fc knob)
ENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 79 - protein, 227 aa, Artificial Sequence
  VHCL-Light chain (MFE23)
QVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 676 aa, Artificial Sequence
  VLCH1 (T84.66-LCHA) VHCH1(EE) (20H4.9) -Heavy chain HC2 (Fc knob)
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 81 - protein, 228 aa, Artificial Sequence
  VHCL-Light chain (T84.66-LCHA)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 673 aa, Artificial Sequence
  VLCH1 (CEA 2F1) VHCH1(EE) (20H4.9) -Heavy chain HC2 (Fc knob)
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 83 - protein, 228 aa, Artificial Sequence
  VHCL-Light chain (CEA CH1A1A 98/99)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 677 aa, Artificial Sequence
  VLCH1 (2B11) VHCH1(EE) (20H4.9) -Heavy chain HC2 (Fc knob)
DIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 85 - protein, 228 aa, Artificial Sequence
  VHCL-Light chain (2B11)
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 87 - protein, 748 aa, Artificial Sequence
  hu FAP ectodomain+poly-lys-tag+his6-tag
RPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 88 - protein, 761 aa, Mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 89 - protein, 749 aa, Artificial Sequence
  Murine FAP ectodomain+poly-lys-tag+his6-tag
RPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSDGKKKKKKGHHHHHH

SEQ ID NO: 90 - protein, 748 aa, Artificial Sequence
  Cynomolgus FAP ectodomain+poly-lys-tag+his6-tag
RPPRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPFVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEDYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSDGKKKKKKGHHHHHH

SEQ ID NO: 91 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 92 - protein, 2322 aa, Homo sapiens
MQSGPRPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHRGGPAQDLTFRVSDGLQASPPATLKVVAIRPAIQIHRSTGLRLAQGSAMPILPANLSVETNAVGQDVSVLFRVTGALQFGELQKQGAGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRHGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPRDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGALQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV

SEQ ID NO: 93 - protein, 1210 aa, Homo sapiens [sequence omitted]

SEQ ID NO: 94 - protein, 556 aa, Homo sapiens [sequence omitted]

SEQ ID NO: 95 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 96 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 97 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 98 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 99 - protein, 254 aa, Macaca fascicularis
MGNSCYNIVATLLLVLNFERTRSLQDLCSNCPAGTFCDNNRSQICSPCPPNSFSSAGGQRTCDICRQCKGVFKTRKECSSTSNAECDCISGYHCLGAECSMCEQDCKQGQELTKKGCKDCCFGTFNDQKRGICRPWTNCSLDGKSVLVNGTKERDVVCGPSPADLSPGASSATPPAPAREPGHSPQIIFFLALTSTVVLFLLFFLVLRFSVVKRSRKKLLYIFKQPFMRPVQTTQEEDGCSCRFPEEEEGGCEL

SEQ ID NO: 100 - protein, 5 aa, Artificial sequence
  Peptide linker
GGGGS

SEQ ID NO: 101 - protein, 10 aa, Artificial sequence
  Peptide linker
GGGGSGGGGS

SEQ ID NO: 102 - protein, 10 aa, Artificial sequence
  Peptide linker
SGGGGSGGGG

SEQ ID NO: 103 - protein, 14 aa, Artificial sequence
  Peptide linker
GGGGSGGGGSGGGG

SEQ ID NO: 104 - protein, 10 aa, Artificial sequence
  Peptide linker
GSPGSSSSGS

SEQ ID NO: 105 - protein, 15 aa, Artificial sequence
  Peptide linker
GGGGSGGGGSGGGGS

SEQ ID NO: 106 - protein, 20 aa, Artificial sequence
  Peptide linker
GGGGSGGGGSGGGGSGGGGS

SEQ ID NO: 107 - protein, 8 aa, Artificial sequence
  Peptide linker
GSGSGSGS

SEQ ID NO: 108 - protein, 8 aa, Artificial sequence
  Peptide linker
GSGSGNGS

SEQ ID NO: 109 - protein, 8 aa, Artificial sequence
  Peptide linker
GGSGSGSG

SEQ ID NO: 110 - protein, 6 aa, Artificial sequence
  Peptide linker
GGSGSG

SEQ ID NO: 111 - protein, 4 aa, Artificial sequence
  Peptide linker
GGSG

SEQ ID NO: 112 - protein, 8 aa, Artificial sequence
  Peptide linker
GGSGNGSG

SEQ ID NO: 113 - protein, 8 aa, Artificial sequence
  Peptide linker
GGNGSGSG

SEQ ID NO: 114 - protein, 6 aa, Artificial sequence
  Peptide linker
GGNGSG

SEQ ID NO: 115 - protein, 5 aa, Artificial Sequence
  CEA (A5H1EL1D)- CDR-H1
DYYMN

SEQ ID NO: 116 - protein, 19 aa, Artificial Sequence
  CEA (A5H1EL1D)- CDR-H2
FIGNKANAYTTEYSASVKG

SEQ ID NO: 117 - protein, 10 aa, Artificial Sequence
  CEA (A5H1EL1D)- CDR-H3
DRGLRFYFDY

SEQ ID NO: 118 - protein, 10 aa, Artificial Sequence
  CEA (A5H1EL1D)- CDR-L1
RASSSVTYIH

SEQ ID NO: 119 - protein, 7 aa, Artificial Sequence
  CEA (A5H1EL1D)- CDR-L2
ATSNLAS

SEQ ID NO: 120 - protein, 9 aa, Artificial Sequence
  CEA (A5H1EL1D)- CDR-L3
QHWSSKPPT

SEQ ID NO: 121 - protein, 121 aa, Artificial Sequence
  CEA (A5H1EL1D) VH (3-23A5-1E)
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSS

SEQ ID NO: 122 - protein, 106 aa, Artificial Sequence
  CEA (A5H1EL1D) VL (A5-L1D)
EIVLTQSPATLSLSPGERATLSCRASSSVTYIHWYQQKPGQAPRSWIYATSNLASGIPARFSGSGSGTDFTLTISSLEPEDFAVYYCQHWSSKPPTFGQGTKLEIK

SEQ ID NO: 123 - protein, 5 aa, Artificial Sequence
  CEA (MFE-H24 to H29)- CDR-H1
DSYMH

SEQ ID NO: 124 - protein, 17 aa, Artificial Sequence
  CEA (MFE-H24, H25, H27, H28, H29)- CDR-H2
WIDPENGDTEYAPKFQG

SEQ ID NO: 125 - protein, 17 aa, Artificial Sequence
  CEA (MFE-H26)- CDR-H2
WIDPENGGTNYAQKFQG

SEQ ID NO: 126 - protein, 11 aa, Artificial Sequence
  CEA (MFE-H24 to H29)- CDR-H3
GTPTGPYYFDY

SEQ ID NO: 127 - protein, 10 aa, Artificial Sequence
  CEA (MFE-L24, L25, L27, L28, L29)- CDR-L1
RASSSVSYMH

SEQ ID NO: 128 - protein, 10 aa, Artificial Sequence
  CEA (MFE-H26)- CDR-L1
RASQSISSYM

SEQ ID NO: 129 - protein, 7 aa, Artificial Sequence
  CEA (MFE-L24, L25, L27, L28)- CDR-L2
STSNLAS

SEQ ID NO: 130 - protein, 7 aa, Artificial Sequence
  CEA (MFE-L26)- CDR-L2
YTSNLAS

SEQ ID NO: 131 - protein, 7 aa, Artificial Sequence
  CEA (MFE-L29)- CDR-L2
STSSLQS

SEQ ID NO: 132 - protein, 9 aa, Artificial Sequence
  CEA (MFE-L24, L25, L27, L26, L28, L29)- CDR-L3
QQRSSYPLT

SEQ ID NO: 133 - protein, 120 aa, Artificial Sequence
  MFE-H24
QVQLVQSGAEVKKPGASVKVSCKASGFNIKDSYMHWVRQAPGQGLEWMGWIDPENGDTEYAPKFQGRVTMTTDTSISTAYMELSRLRSDDTAVYYCNEGTPTGPYYFDYWGQGTLVTVSS

SEQ ID NO: 134 - protein, 120 aa, Artificial Sequence
  MFE-H25
QVQLVQSGAEVKKPGASVKVSCKASGYTFKDSYMHWVRQAPGQGLEWMGWIDPENGDTEYAPKFQGRVTMTTDTSISTAYMELSRLRSDDTAVYYCNEGTPTGPYYFDYWGQGTLVTVSS

SEQ ID NO: 135 - protein, 120 aa, Artificial Sequence
  MFE-H26
QVQLVQSGAEVKKPGASVKVSCKASGFNIKDSYMHWVRQAPGQGLEWMGWIDPENGGTNYAQKFQGRVTMTTDTSISTAYMELSRLRSDDTAVYYCNEGTPTGPYYFDYWGQGTLVTVSS

SEQ ID NO: 136 - protein, 120 aa, Artificial Sequence
  MFE-H27
QVQLVQSGAEVKKPGASVKVSCKASGFNIKDSYMHWVRQAPGQGLEWMGWIDPENGDTEYAPKFQGRVTMTTDTSISTAYMELSRLRSDDTAVYYCARGTPTGPYYFDYWGQGTLVTVSS

SEQ ID NO: 137 - protein, 120 aa, Artificial Sequence
  MFE-H28
QVQLVQSGAEVKKPGASVKVSCKASGFNIKDSYMHWVRQAPGQGLEWMGWIDPENGDTEYAPKFQGRVTMTRDTSISTAYMELSRLRSDDTAVYYCNEGTPTGPYYFDYWGQGTLVTVSS

SEQ ID NO: 138 - protein, 120 aa, Artificial Sequence
  MFE-H29
QVQLVQSGAEVKKPGSSVKVSCKASGFNIKDSYMHWVRQAPGQGLEWMGWIDPENGDTEYAPKFQGRVTITTDESTSTAYMELSSLRSEDTAVYYCNEGTPTGPYYFDYWGQGTLVTVSS

SEQ ID NO: 139 - protein, 106 aa, Artificial Sequence
  MFE-L24
DIQMTQSPSSLSASVGDRVTITCRASSSVSYMHWYQQKPGKAPKLLIYSTSNLASGVPSRFSGSGSGTDFTLTISSLQPEDFATYYCQQRSSYPLTFGGGTKLEIK

SEQ ID NO: 140 - protein, 106 aa, Artificial Sequence
  MFE-L25
EIQMTQSPSSLSASVGDRVTITCRASSSVSYMHWYQQKPGKAPKLLIYSTSNLASGVPSRFSGSGSGTDFTLTISSLQPEDFATYYCQQRSSYPLTFGGGTKLEIK

SEQ ID NO: 141 - protein, 107 aa, Artificial Sequence
  MFE-L26
EIQMTQSPSSLSASVGDRVTITCRASQSISSYMHWYQQKPGKAPKLLIYSTSNLASGVPSRFSGSGSGTDFTLTISSLQPEDFATYYCQQRSSYPLTFGGGTKLEIK

SEQ ID NO: 142 - protein, 106 aa, Artificial Sequence
  MFE-L27
EIQMTQSPSSLSASVGDRVTITCRASSSVPYMHWYQQKPGKAPKLLIYSTSNLASGVPSRFSGSGSGTDFTLTISSVQPEDFATYYCQQRSSYPLTFGGGTKLEIK

SEQ ID NO: 143 - protein, 106 aa, Artificial Sequence
  MFE-L28
EIQMTQSPSSLSASVGDRVTITCRASSSVPYMHWLQQKPGKAPKLLIYSTSNLASGVPSRFSGSGSGTDFTLTISSVQPEDFATYYCQQRSSYPLTFGGGTKLEIK

SEQ ID NO: 144 - protein, 106 aa, Artificial Sequence
  MFE-L29
EIQMTQSPSSLSASVGDRVTITCRASSSVPYMHWLQQKPGKAPKLLIYSTSSLQSGVPSRFSGSGSGTDFTLTISSVQPEDFATYYCQQRSSYPLTFGGGTKLEIK

SEQ ID NO: 145 - protein, 10 aa, Artificial Sequence
  PD-L1 CDR-H1
GFTFSDSWIH

SEQ ID NO: 146 - protein, 18 aa, Artificial Sequence
  PD-L1 CDR-H2
AWISPYGGSTYYADSVKG

SEQ ID NO: 147 - protein, 9 aa, Artificial Sequence
  PD-L1 CDR-H3
RHWPGGFDY

SEQ ID NO: 148 - protein, 11 aa, Artificial Sequence
  PD-L1 CDR-L1
RASQDVSTAVA

SEQ ID NO: 149 - protein, 7 aa, Artificial Sequence
  PD-L1 CDR-L2
SASFLYS

SEQ ID NO: 150 - protein, 9 aa, Artificial Sequence
  PD-L1 CDR-L3
QQYLYHPAT

SEQ ID NO: 151 - protein, 118 aa, Artificial Sequence [sequence omitted]

SEQ ID NO: 152 - protein, 107 aa, Artificial Sequence [sequence omitted]

SEQ ID NO: 153 - protein, 98 aa, Homo sapiens
EVQLLESGGGLVQPGGSLRLSCAASGFTFSSYAMSWVRQAPGKGLEWVSAISGSGGSTYYGDSVKGRFTISRDNSKNTLYLQMNSLRAEDTAVYYCAK

SEQ ID NO: 154 - protein, 100 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTT

SEQ ID NO: 155 - protein, 121 aa, Artificial Sequence
  3-23A5-1 VH
EVQLLESGGGLVQPGGSLRLSCAASGFTFTDYYMNWVRQAPGKGLEWVGFIGNKANGYTTEYSASVKGRFTISRDNSKNTLYLQMNSLRAEDTAVYYCARDRGLRFYFDYWGQGTTVTVSS

SEQ ID NO: 156 - protein, 121 aa, Artificial Sequence
  3-23A5-2
EVQLLESGGGLVQPGGSLRLSCAASGFTFTDYYMNWVRQAPGKGLEWVGFIGNKANGYTTYYGDSVKGRFTISRDNSKNTLYLQMNSLRAEDTAVYYCARDRGLRFYFDYWGQGTTVTVSS

SEQ ID NO: 157 - protein, 119 aa, Artificial Sequence
  3-23A5-3 VH
EVQLLESGGGLVQPGGSLRLSCAASGFTFTDYYMNWVRQAPGKGLEWVGFIGNKGYTTEYSASVKGRFTISRDNSKNTLYLQMNSLRAEDTAVYYCARDRGLRFYFDYWGQGTTVTVSS

SEQ ID NO: 158 - protein, 121 aa, Artificial Sequence
  3-23A5-4 VH
EVQLLESGGGLVQPGGSLRLSCAASGFTFTDYYMSWVRQAPGKGLEWVGFIGNKANGYTTEYSASVKGRFTISRDNSKNTLYLQMNSLRAEDTAVYYCARDRGLRFYFDYWGQGTTVTVSS

SEQ ID NO: 159 - protein, 121 aa, Artificial Sequence
  3-23A5-1A (all_backmutations) VH
EVQLLESGGGLVQPGGSLRLSCAASGFTFTDYYMNWVRQAPGKGLEWLGFIGNKANGYTTEYSASVKGRFTISRDKSKNTLYLQMNSLRAEDTATYYCTRDRGLRFYFDYWGQGTTVTVSS

SEQ ID NO: 160 - protein, 121 aa, Artificial Sequence
  3-23A5-1C (A93T) VH
EVQLLESGGGLVQPGGSLRLSCAASGFTFTDYYMNWVRQAPGKGLEWVGFIGNKANGYTTEYSASVKGRFTISRDNSKNTLYLQMNSLRAEDTAVYYCTRDRGLRFYFDYWGQGTTVTVSS

SEQ ID NO: 161 - protein, 121 aa, Artificial Sequence
  3-23A5-1D (K73) VH
EVQLLESGGGLVQPGGSLRLSCAASGFTFTDYYMNWVRQAPGKGLEWVGFIGNKANGYTTEYSASVKGRFTISRDKSKNTLYLQMNSLRAEDTAVYYCARDRGLRFYFDYWGQGTTVTVSS

SEQ ID NO: 162 - protein, 121 aa, Artificial Sequence
  3-15A5-1 VH
EVQLVESGGGLVKPGGSLRLSCAASGFTFTDYYMNWVRQAPGKGLEWVGFIGNKANGYTTEYSASVKGRFTISRDDSKNTLYLQMNSLKTEDTAVYYCTRDRGLRFYFDYWGQGTTVTVSS

SEQ ID NO: 163 - protein, 121 aa, Artificial Sequence
  3-15A5-2 VH
EVQLVESGGGLVKPGGSLRLSCAASGFTFTDYYMNWVRQAPGKGLEWVGFIGNKANGYTTEYAAPVKGRFTISRDDSKNTLYLQMNSLKTEDTAVYYCTRDRGLRFYFDYWGQGTTVTVSS

SEQ ID NO: 164 - protein, 121 aa, Artificial Sequence
  3-15A5-3 VH
EVQLVESGGGLVKPGGSLRLSCAASGFTFTDYYMNWVRQAPGKGLEWVGFIGNKANGGTTDYAAPVKGRFTISRDDSKNTLYLQMNSLKTEDTAVYYCTRDRGLRFYFDYWGQGTTVTVSS

SEQ ID NO: 165 - protein, 95 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 166 - protein, 106 aa, Artificial Sequence
  A5-L1 VL
EIVLTQSPATLSLSPGERATLSCRASSSVTYIHWYQQKPGQAPRLLIYATSNLASGIPARFSGSGSGTDFTLTISSLEPEDFAVYYCQHWSSKPPTFGQGTKLEIK

SEQ ID NO: 167 - protein, 107 aa, Artificial Sequence
  A5-L2 VL
EIVLTQSPATLSLSPGERATLSCRASQSVSSYIHWYQQKPGQAPRLLIYATSNLASGIPARFSGSGSGTDFTLTISSLEPEDFAVYYCQHWSSKPPTFGQGTKLEIK

SEQ ID NO: 168 - protein, 106 aa, Artificial Sequence
  A5-L3 VL
EIVLTQSPATLSLSPGERATLSCRASSSVTYIHWYQQKPGQAPRLLIYDASNRATGIPARFSGSGSGTDFTLTISSLEPEDFAVYYCQHWSSKPPTFGQGTKLEIK

SEQ ID NO: 169 - protein, 106 aa, Artificial Sequence
  A5-L4 VL
EIVLTQSPATLSLSPGERATLSCRASSSVTYIHWYQQKPGQAPRLLIYATSNLASGIPARFSGSGSGTDFTLTISSLEPEDFAVYYCQQWSSKPPTFGQGTKLEIK

SEQ ID NO: 170 - protein, 106 aa, Artificial Sequence
  A5-L1A (all_backmutations) VL
QTVLTQSPATLSLSPGERATLSCRASSSVTYIHWYQQKPGSSPKSWIYATSNLASGIPARFSGSGSGTDYTLTISSLEPEDFAVYYCQHWSSKPPTFGQGTKLEIK

SEQ ID NO: 171 - protein, 106 aa, Artificial Sequence
  A5-L1B (Q1T2) VL
QTVLTQSPATLSLSPGERATLSCRASSSVTYIHWYQQKPGQAPRLLIYATSNLASGIPARFSGSGSGTDFTLTISSLEPEDFAVYYCQHWSSKPPTFGQGTKLEIK

SEQ ID NO: 172 - protein, 106 aa, Artificial Sequence
  A5-L1C (FR2) VL
EIVLTQSPATLSLSPGERATLSCRASSSVTYIHWYQQKPGSSPKSWIYATSNLASGIPARFSGSGSGTDFTLTISSLEPEDFAVYYCQHWSSKPPTFGQGTKLEIK

SEQ ID NO: 173 - protein, 671 aa, Artificial Sequence
  VLCH1 (CEA A5H1EL1D) VHCH1(EE) (20H4.9) -Heavy chain HC2 (Fc knob)
EIVLTQSPATLSLSPGERATLSCRASSSVTYIHWYQQKPGQAPRSWIYATSNLASGIPARFSGSGSGTDFTLTISSLEPEDFAVYYCQHWSSKPPTFGQGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 174 - protein, 228 aa, Artificial Sequence
  VHCL-Light chain (CEA A5H1EL1D)
EVQLLESGGGLVQPGGSLRLSCAASGFTFTDYYMNWVRQAPGKGLEWLGFIGNKANAYTTEYSASVKGRFTISRDKSKNTLYLQMNSLRAEDTATYYCTRDRGLRFYFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 96 aa, Homo sapiens
QLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 176 - protein, 98 aa, Homo Sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 177 - protein, 98 aa, Homo sapiens
QVQLVQSGAEVKKPGSSVKVSCKASGGTFSSYAISWVRQAPGQGLEWMGGIIPIFGTANYAQKFQGRVTITTDESTSTAYMELSSLRSEDTAVYYCAR

SEQ ID NO: 178 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 179 - protein, 671 aa, Artificial Sequence
  VLCH1 (CEA huMFE23-L28-H24) VHCH1(EE) (20H4.9) -Heavy chain HC2 (Fc knob)
EIQMTQSPSSLSASVGDRVTITCRASSSVPYMHWLQQKPGKAPKLLIYSTSNLASGVPSRFSGSGSGTDFTLTISSVQPEDFATYYCQQRSSYPLTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 180 - protein, 227 aa, Artificial Sequence
  VHCL-Light chain (CEA huMFE23-L28-H24)
QVQLVQSGAEVKKPGASVKVSCKASGFNIKDSYMHWVRQAPGQGLEWMGWIDPENGDTEYAPKFQGRVTMTTDTSISTAYMELSRLRSDDTAVYYCNEGTPTGPYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 671 aa, Artificial Sequence; identical to SEQ ID NO: 179
  VLCH1 (CEA huMFE23-L28-H28) VHCH1(EE) (20H4.9) -Heavy chain HC2 (Fc knob)

SEQ ID NO: 182 - protein, 227 aa, Artificial Sequence
  VHCL-Light chain (CEA huMFE23-L28-H28)
QVQLVQSGAEVKKPGASVKVSCKASGFNIKDSYMHWVRQAPGQGLEWMGWIDPENGDTEYAPKFQGRVTMTRDTSISTAYMELSRLRSDDTAVYYCNEGTPTGPYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - protein, 671 aa, Artificial Sequence; identical to SEQ ID NO: 179
  VLCH1 (CEA huMFE23-L28-H25) VHCH1(EE) (20H4.9) -Heavy chain HC2 (Fc knob)

SEQ ID NO: 184 - protein, 227 aa, Artificial Sequence
  VHCL-Light chain (CEA huMFE23-L28-H25)
QVQLVQSGAEVKKPGASVKVSCKASGYTFKDSYMHWVRQAPGQGLEWMGWIDPENGDTEYAPKFQGRVTMTTDTSISTAYMELSRLRSDDTAVYYCNEGTPTGPYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - protein, 671 aa, Artificial Sequence
  VLCH1 (CEA huMFE23- L27-H29) VHCH1(EE) (20H4.9) -Heavy chain HC2 (Fc knob)
EIQMTQSPSSLSASVGDRVTITCRASSSVPYMHWYQQKPGKAPKLLIYSTSNLASGVPSRFSGSGSGTDFTLTISSVQPEDFATYYCQQRSSYPLTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 186 - protein, 227 aa, Artificial Sequence
  VHCL-Light chain (CEA huMFE23- L27-H29)
QVQLVQSGAEVKKPGSSVKVSCKASGFNIKDSYMHWVRQAPGQGLEWMGWIDPENGDTEYAPKFQGRVTITTDESTSTAYMELSSLRSEDTAVYYCNEGTPTGPYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 187 - protein, 671 aa, Artificial Sequence; identical to SEQ ID NO: 185
  VLCH1 (CEA huMFE23-L27-H28) VHCH1(EE) (20H4.9) -Heavy chain HC2 (Fc knob)

SEQ ID NO: 188 - protein, 227 aa, Artificial Sequence; identical to SEQ ID NO: 182
  VHCL-Light chain (CEA huMFE23-L27-H28)

SEQ ID NO: 189 - protein, 671 aa, Artificial Sequence; identical to SEQ ID NO: 185
  VLCH1 (CEA huMFE23- L27-H26) VHCH1(EE) (20H4.9) -Heavy chain HC2 (Fc knob)

SEQ ID NO: 190 - protein, 227 aa, Artificial Sequence
  VHCL-Light chain (CEA huMFE23- L27-H26)
QVQLVQSGAEVKKPGASVKVSCKASGFNIKDSYMHWVRQAPGQGLEWMGWIDPENGGTNYAQKFQGRVTMTTDTSISTAYMELSRLRSDDTAVYYCNEGTPTGPYYFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - protein, 671 aa, Artificial Sequence; identical to SEQ ID NO: 185
  VLCH1 (CEA huMFE23-L27-H24) VHCH1(EE) (20H4.9) -Heavy chain HC2(Fc knob)

SEQ ID NO: 192 - protein, 227 aa, Artificial Sequence; identical to SEQ ID NO: 180
  VHCL-Light chain (CEA huMFE23-L27-H24)

SEQ ID NO: 193 - protein, 672 aa, Artificial Sequence
  VLCH1 (PD-L1) VHCH1(EE) (20H4.9) -Heavy chain HC2 (Fc knob)
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 194 - protein, 225 aa, Artificial Sequence
  VHCL-Light chain (PD-L1)
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 437 aa, Artificial Sequence
  VLCH1 (PD-L1) -Heavy chain HC2 (Fc knob)
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 196 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET